(19) United States
(12) Patent Application Publication
(10) Pub. No.: US 2026/0200897 A1
(43) Pub. Date: Jul. 16, 2026

(54) 4-((5-(3-(4-(PYRIDIN-2-YL)CYCLOPENTYL)-1H-PYRAZOL-3-YL)AMINO)-BENZENESULFONAMIDE DERIVATIVES AND SIMILAR COMPOUNDS AS CDK INHIBITORS FOR THE TREATMENT OF CANCER

(71) Applicant: BeOne Medicines I GmbH, Basel (CH)

(72) Inventors: Changxin Huo, Beijing (CN); Yu Zhao, Beijing (CN); Yucheng Wang, Beijing (CN); Huaqing Liu, Beijing (CN); Zhiwei Wang, Beijing (CN)

(73) Assignee: BeOne Medicines I GmbH, Basel (CH)

(21) Appl. No.: 19/566,011

(22) Filed: Mar. 13, 2026

Related U.S. Application Data
(63) Continuation of application No. PCT/IB2024/059102, filed on Sep. 19, 2024.

(30) Foreign Application Priority Data
Sep. 20, 2023 (WO) PCT/CN2023/120162

(51) Int. Cl. C07D 403/08; A61K 31/4155; A61K 31/4178; A61K 31/423; A61K 31/428; A61K 31/437; A61K 31/4439; A61K 31/501; A61K 31/5025; A61K 31/506; A61K 31/5415; A61K 31/635; C07D 401/08; C07D 409/14; C07D 413/08; C07D 413/14; C07D 417/12; C07D 417/14; C07D 471/04; C07D 498/04; C07D 513/04

(52) U.S. Cl. CPC C07D 403/08; A61K 31/4155; A61K 31/4178; A61K 31/423; A61K 31/428; A61K 31/437; A61K 31/4439; A61K 31/501; A61K 31/5025; A61K 31/506; A61K 31/5415; A61K 31/635; C07D 401/08; C07D 409/14; C07D 413/08; C07D 413/14; C07D 417/12; C07D 417/14; C07D 471/04; C07D 498/04; C07D 513/04

(57) ABSTRACT

Disclosed herein are compounds used as inhibitors of cyclin-dependent kinases. Disclosed herein is the use of these inhibitors for inhibiting cyclin-dependent kinases, and the use of such compounds for treating cancer.

### CROSS-REFERENCE TO RELATED APPLICATION

This application is a continuation of International Application No. PCT/IB2024/059102, filed on Sep. 19, 2024, which claims priority to International Application No. PCT/CN2023/120162, filed on Sep. 20, 2023, the disclosures of each of which are hereby incorporated by reference in their entireties.

### FIELD OF THE INVENTION

Disclosed herein are compounds used as inhibitors of cyclin-dependent kinases. Disclosed herein is the use of these inhibitors for inhibiting cyclin-dependent kinases, and the use of such compounds for treating cancer.

### BACKGROUND OF THE INVENTION

Cyclin-dependent kinases (CDKs), a family of Ser/Thr protein kinases, function as a driver of the cell cycle (Norbury, C. and Nurse, P., Annu Rev Biochem, 61, 441-470). During the cell cycle, CDK4 and CDK6 initiate the retinoblastoma (Rb) phosphorylation by binding to cyclinD and partially release the transcription factor E2F. Then the CDK2 forms a complex with cyclinE to fully phosphorylate Rb, entirely release E2F and initiate S-phase (Harbour, J. W. et al., Cell, 98(6), 859-869). Then in the S phase, CDK2 forms a complex with cyclinA (Echalier, A. et al., Biochem Biophys Acta, 1804(3), 511-519). Interestingly, in the normal cells, CDK2 is mostly not essential for the cell cycle, while in some cancer cells, CDK2 kinase activity plays a critical role in the abnormal growth process (Caruso, J. A. et al., Cancer Res, 78(19), 5481-5491).

CCNE gene amplification or cyclinE overexpressed forms of cancer cells over activates CDK2, dysregulating Rb phosphorylation and resulting in cancer cell proliferation (Wood, D. J. et al., Cell Chem Biol, 26(1), 121-130). Aberrant CCNE has been proved as a disease driver in multiple cancer types such as ovarian, esophageal, bladder, pancreatic and so on, which are associated with poor disease outcomes (Au-Yeung, G. et al., Clin Cancer Res, 23(7), 1862-1874; DeLair, D. F. et al., J Pathol, 243(2), 230-241; Fu, Y. P. et al., Cancer Res, 74(20), 5808-5818; Huber, A. R. et al., BMC Gastroenterol, 15, 80; Miller, C. T. et al., Clin Cancer Res, 9(13), 4819-4825). Besides, one of the mechanisms for the clinical drug resistance of CDK4/6i (such as Palbociclib, Ribociclib and Abemaciclib) in ER+ HER2− breast cancer patients is the overexpression of CCNE and the activation of CDK2 (Knudsen, E. S. et al., Cell Rep, 38(9), 110448). CDK2 siRNA knockdown in the Palbociclib resistant breast cancer cell lines and mouse model shows CDK2 inhibition can overcome the CDK4/6-inhibitor resistance (Pandey, K. et al., Cancers (Basel), 12 (12)). Another potential use of CDK2 inhibitors is based on the mechanism of trastuzumab resistance. There is around 35% trastuzumab resistance incidence of cyclinE amplification or overexpression in HER2+BC patients, which are associated with a worse clinical benefit while the trastuzumab-resistant cells are sensitive to CDK2 inhibition (Scaltriti, M. et al., Proc Natl Acad Sci USA, 108(9), 3761-3766). The CCNE1 amplification across broad cancer types predicts response to CDK2 inhibition, indicating CDK2 is a potentially impactful therapeutic target.

The low toxicity of targeting CDK2 has also been proved in the animal model. CDK2 knockout mice are viable and developed normally except for the abnormal germline cell (Berthet, C. et al., Curr Biol, 13(20), 1775-1785). While single knockout of CDK1 or double knockout of CDK4 and CDK6 mice are embryonic lethal (Satyanarayana, A. and Kaldis, P., Oncogene, 28(33), 2925-2939).

The crystal structure of CDK2 especially its kinase domain is highly similar to the CDK1 and the commercial CDK2 inhibitors are mostly with poor CDK1 selectivity (Wells, C. I. et al., Nat Commun, 11(1), 2743). It is desirable to achieve a cancerous specific drug so as to derive a high selective CDK2 inhibitor, sparing CDK family members to limit off-target CDK-driven toxicities, especially CDK1/CDK4/CDK6.

### SUMMARY OF THE INVENTION

In one embodiment, disclosed herein are compounds of Formula (I). The embodiment comprises the following aspects:

Aspect 1. A compound of Formula (I)

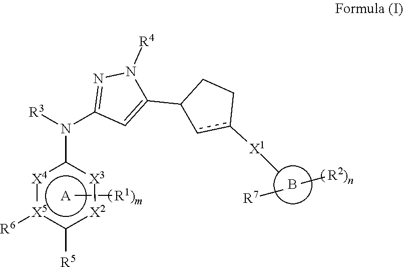

or a N-oxide thereof, or a pharmaceutically acceptable salt thereof, or a stereoisomer thereof, or a tautomer, or a deuterated analog thereof, or a prodrug thereof, wherein:
m is 0, 1, 2, 3, or 4, provided that the valency theory has been met;
n is 0, 1, 2, 3, 4, or 5, provided that the valency theory has been met;
[symbol] is independently a single bond or double bond;
ring A is selected from a heteroaryl ring and an aryl ring;
X¹ is a single bond or O;
X², X³, X⁴, and X⁵ are each independently selected from N and C;
R¹ is each independently selected from halogen, —C₁-C₈alkyl, —C₃-C₅cycloalkyl, 3- to 8-membered heterocyclyl, —CN, and —OH, wherein each of said —C₁-C₅alkyl, C₃-C₅cycloalkyl, or 3- to 8-membered heterocyclyl is optionally substituted with at least one substituent independently selected from halogen, —C₁-C₅alkyl, —C₂-C₅alkenyl, —C₂-C₅alkynyl, —C₃-C₅cycloalkyl, 3- to 8-membered heterocyclyl, —OH, —C₆-C₁₂aryl, 5- to 12-membered heteroaryl, and oxo (═O);
R⁵ is selected from —SO₂R^{5a} and —SO₂NR^{5a}R^{5b}, wherein R^{5a} and R^{5b} are each independently selected from hydrogen and —C₁-C₃alkyl;
R⁶ is selected from hydrogen, —C₁-C₃alkyl, and —C₃-C₅cycloalkyl; or
R⁵ and R⁶, together with the atoms to which they are attached, form a 5- to 12-membered ring, said ring comprising S(═O)₂ or S(═O), and additional 0-2 heteroatom(s) independently selected from nitrogen and oxygen as ring member(s); said ring is optionally substituted with at least one substituent R^{5c};
R^{5c} is each independently selected from halogen, —C₁-C₅alkyl, —C₃-C₅cycloalkyl, 3- to 8-membered heterocyclyl, —C₆-C₁₂aryl, 5- to 12-membered heteroaryl, and oxo (═O);
ring B is selected from 5- to 7-membered heterocyclyl, phenyl, and 5- to 7-membered heteroaryl;
R² is each independently selected from halogen, —C₁-C₅alkyl, —C₂-C₅alkenyl, —C₂-C₅alkynyl, —C₃-C₈cycloalkyl, 3- to 8-membered heterocyclyl, —C₆-C₁₂aryl, 5- to 12-membered heteroaryl, oxo (═O), —OR^{2a}, —CN, —C(O)R^{2a}, —CO₂R^{2a}, —NR^{2a}R^{2b}, —NR^{2a}COR^{2b}, and —NR^{2a}CO₂R^{2b}, wherein each of said —C₁-C₈alkyl, —C₂-C₅alkenyl, —C₂-C₅alkynyl, —C₃-C₅cycloalkyl, 3- to 8-membered heterocyclyl, —C₆-C₁₂aryl or 5- to 12-membered heteroaryl is optionally substituted with at least one substituent independently selected from halogen, —C₁-C₅alkyl, —C₂-C₅alkenyl, —C₂-C₅alkynyl, —C₃-C₅cycloalkyl, 3- to 8-membered heterocyclyl, C₆-C₁₂aryl, 5- to 12-membered heteroaryl, oxo (═O), —CN, —OR^{2c}, —C₁-C₅alkyl-OR^{2c}, —C(O)R^{2c}, —CO₂R^{2c}, —NR^{2c}R^{2d}, —NR^{2c}COR^{2d}, and —NR^{2c}CO₂R^{2d};
R^{2a}, R^{2b}, R^{2c} and R^{2d} are each independently selected from hydrogen, —C₁-C₅alkyl, —C₂-C₅alkenyl, —C₂-C₅alkynyl, C₃-C₅cycloalkyl, 3- to 8-membered heterocyclyl, C₆-C₁₂aryl, and 5- to 12-membered heteroaryl, wherein each of said —C₁-C₅alkyl, —C₂-C₅alkenyl, —C₂-C₅alkynyl, C₃-C₅cycloalkyl, 3- to 8-membered heterocyclyl, C₆-C₁₂aryl, or 5- to 12-membered heteroaryl is optionally substituted with at least one substituent selected from halogen, —C₁-C₅alkyl, —C₂-C₅alkenyl, —C₂-C₅alkynyl, C₃-C₅cycloalkyl, 3- to 8-membered heterocyclyl, C₆-C₁₂aryl, 5- to 12-membered heteroaryl, oxo (═O), —CN, —OH, and —C₁-C₈alkoxyl;
R⁷ is selected from hydrogen, —C₁-C₃alkyl, and —C₃-C₅cycloalkyl; or adjacent R² and R⁷, together with the atoms to which they are attached, form a 3- to 12-membered ring, said ring comprising 0-3 heteroatoms independently selected from nitrogen, oxygen and optionally oxidized sulfur as ring member(s); said ring is optionally substituted with at least one substituent R^{7a};
R^{7a} is each independently selected from halogen, —C₁-C₈alkyl, —C₃-C₅cycloalkyl, 3- to 8-membered heterocyclyl, —C₆-C₁₂aryl, 5- to 12-membered heteroaryl, and oxo (═O);
R³ and R⁴ are each independently selected from hydrogen, —C₁-C₈alkyl, C₃-C₅cycloalkyl, and —C(O)R^{3a}; wherein each of said —C₁-C₈alkyl and C₃-C₅cycloalkyl is optionally substituted with at least one substituent independently selected from halogen, oxo (═O), —CN, —OR^{3b} and —NR^{3b}R^{3c}; and
R^{3a}, R^{3b} and R^{3c} are each independently selected from hydrogen, —C₁-C₅alkyl, and C₃-C₅cycloalkyl; wherein each of said —C₁-C₅alkyl and C₃-C₅cycloalkyl is optionally substituted with at least one substituent independently selected from halogen, —C₁-C₈alkyl, C₃-C₅cycloalkyl, oxo (═O), —CN, —OH, and —C₁-C₅alkoxyl.

Aspect 2. The compound of Aspect 1, wherein the ring A is a 6-membered heteroaryl ring and three of X², X³, X⁴, and X⁵ are N; preferably, ring A is a 6-membered heteroaryl ring and two of X², X³, X⁴, and X⁵ are N; more preferably, ring A is a 6-membered heteroaryl ring and one of X², X³, X⁴, and X⁵ is N; even more preferably, ring A is a 6-membered heteroaryl ring and X² is N.

Aspect 3. The compound of Aspect 1, wherein the compound is selected from (IIa), (IIb), (IIc), and (IId):

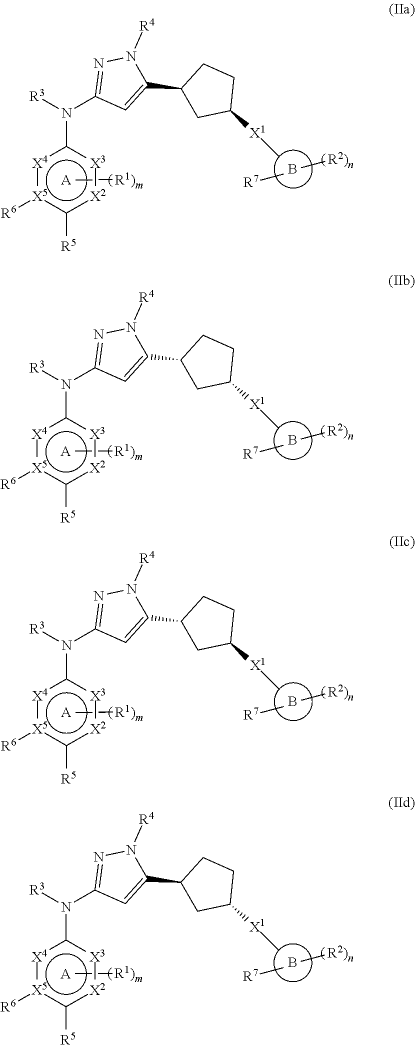

preferably, the compound is selected from (IIIa), (IIIb), (IIIc), and (IIId):

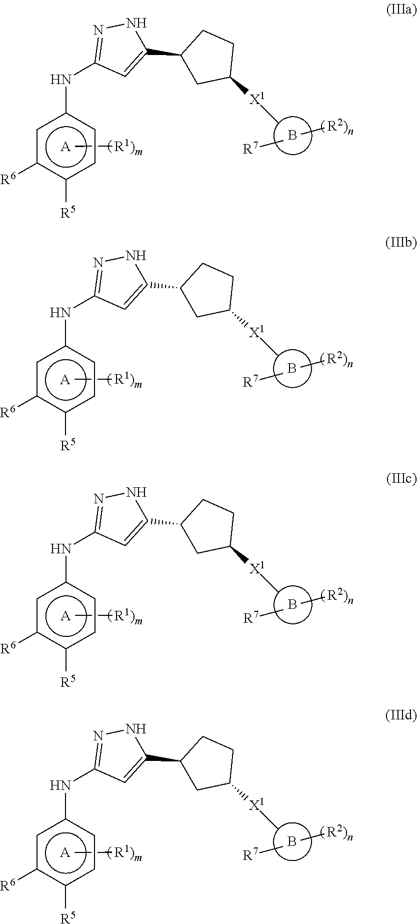

Aspect 4. The compound of any one of the preceding aspects, wherein
m is 0, 1, 2, 3, or 4, provided that the valency theory has been met;
R¹ is each independently selected from —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, —CN, and —OH, wherein each of said methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, or 3- to 8-membered heterocyclyl is optionally substituted with at least one substituent independently selected from —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, —C₂-C₅alkenyl, —C₂-C₈alkynyl, cyclopropyl, cyclobutyl, cyclopentyl, cyclohexyl, cycloheptyl, cyclooctyl, 3- to 8-membered heterocyclyl, —OH, —C₆-C₁₂aryl, 5- to 12-membered heteroaryl, and oxo (═O); preferably, m is 0, 1, 2, 3, or 4, provided that the valency theory has been met;
R¹ is each independently selected from —F, —Cl, methyl, ethyl, propyl (n-propyl or isopropyl), cyclopropyl, cyclobutyl, cyclopentyl, 3- to 8-membered heterocyclyl, —CN, and —OH, wherein each of said methyl, ethyl, propyl (n-propyl or isopropyl), cyclopropyl, cyclobutyl, cyclopentyl, or 3- to 8-membered heterocyclyl is optionally substituted with at least one substituent independently selected from —F, —Cl, —Br, —I, methyl, ethyl, propyl (n-propyl or isopropyl), —C₂-C₅alkenyl, —C₂-C₅alkynyl, cyclopropyl, cyclobutyl, 3- to 8-membered heterocyclyl, —OH, —C₆-C₁₂aryl, 5- to 12-membered heteroaryl, and oxo (═O);
more preferably, m is 0 or 1, provided that the valency theory has been met;
R¹ is each independently selected from —F, —Cl, methyl, ethyl, propyl (n-propyl or isopropyl), cyclopropyl, cyclobutyl, cyclopentyl, —CN, and —OH; wherein said methyl is substituted with —OH;
even more preferably, m is 0 or 1, provided that the valency theory has been met; R¹ is —F or CH₂OH.

Aspect 5. The compound of any one of the preceding aspects, wherein
R⁵ is selected from —SO₂R^{5a} and —SO₂NR^{5a}R^{5b}, wherein R^{5a} and R^{5b} are each independently selected from hydrogen, methyl, ethyl, and propyl (n-propyl or isopropyl);
preferably, R⁵ is selected from —SO₂R^{5a} and —SO₂NR^{5a}R^{5b}, wherein R^{5a} and R^{5b} are each independently selected from hydrogen and methyl;
more preferably, R⁵ is selected from —SO₂R^{5a} and —SO₂NR^{5a}R^{5b}, wherein R^{5a} and R^{5b} are each hydrogen;
even more preferably, R⁵ is —SO₂NH₂.

Aspect 6. The compound of any one of the preceding aspects, wherein
R⁶ is selected from hydrogen, methyl, ethyl, propyl (n-propyl or isopropyl), cyclopropyl, cyclobutyl, cyclopentyl, cyclohexyl, cycloheptyl, and cyclooctyl;
preferably, R⁶ is selected from hydrogen, methyl, ethyl, cyclopropyl, cyclobutyl, and cyclopentyl; more preferably, R⁶ is selected from hydrogen, methyl, ethyl, and cyclopropyl;
even more preferably, R⁶ is hydrogen.

Aspect 7. The compound of any one of aspects 1-4, wherein
adjacent R⁵ and R⁶, together with the atoms to which they are attached, form a 3- to 12-membered ring, said ring comprising S(═O)₂ or S(═O), and additional 0-2 heteroatom(s) independently selected from nitrogen and oxygen as ring member(s); said ring is optionally substituted with at least one substituent R^{5c};
R^{5c} is each independently selected from —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, —C₆-C₁₂aryl, 5- to 12-membered heteroaryl, and oxo (═O);
preferably, adjacent R⁵ and R⁶, together with the atoms to which they are attached, form a 4-, 5-, 6-, or 7-membered ring, said ring comprising S(═O)₂ or S(═O), and additional 0-2 heteroatom(s) independently selected from nitrogen and oxygen as ring member(s); said ring is optionally substituted with at least one substituent R^{5c};
R^{5c} is each independently selected from —F, —Cl, methyl, ethyl, propyl (n-propyl or isopropyl), butyl (n-butyl, sec-butyl, iso-butyl or tert-butyl), cyclopropyl, cyclobutyl, cyclopentyl, 3- to 8-membered heterocyclyl, —C₆-C₁₂aryl, 5- to 12-membered heteroaryl, and oxo (═O);
more preferably, adjacent R⁵ and R⁶, together with the atoms to which they are attached, form a 5- or 6-membered ring, said ring comprising S(═O)₂ or S(═O), and additional 0-1 nitrogen atom as ring member; said ring is optionally substituted with at least one substituent R^{5c};
R^{5c} is each independently selected from —F, methyl, ethyl, cyclopropyl, cyclobutyl, cyclopentyl, 3- to 8-membered heterocyclyl, 5- to 12-membered heteroaryl, and oxo (═O);
even more preferably, adjacent R⁵ and R⁶, together with the atoms to which they are attached, form a 5- or 6-membered ring, said ring comprising S(═O)₂ or S(═O), and additional 0-1 nitrogen atom as ring member; said ring is optionally substituted with methyl.

Aspect 8. The compound of any one of the preceding aspects, wherein
ring B is selected from 5- or 6-membered heterocyclyl, phenyl, and 5- or 6-membered heteroaryl;
preferably, ring B is selected from 5-membered heterocyclyl, 5-membered heteroaryl, 6-membered heteroaryl, and phenyl; wherein said 5-membered heterocyclyl, 5-membered heteroaryl, or 6-membered heteroaryl comprising 1-3 heteroatoms independently selected from nitrogen, oxygen, and optionally oxidized sulfur as ring member(s);
more preferably, ring B is selected from 5-membered heterocyclyl comprising 1-2 heteroatoms independently selected from oxygen and nitrogen as ring member(s), 5-membered heteroaryl comprising 1-3 heteroatoms independently selected from nitrogen, oxygen, and optionally oxidized sulfur as ring member(s), 6-membered heteroaryl comprising 1-2 nitrogen atoms as ring member(s), and phenyl;
even more preferably, ring B is selected from furanyl, pyridinyl, oxazolyl, pyrimidinyl, pyridazinyl, pyrazolyl, thiazolyl, oxadiazolyl, phenyl, tetrahydrofuranyl, imidazolyl, triazolyl, pyrrolyl, pyrrolidinyl, imidazolidinyl, isothiazolyl, and isoxazolyl.

Aspect 9. The compound of any one of the preceding aspects, wherein
n is 0, 1, 2, 3, 4, or 5, provided that the valency theory has been met;
R² is each independently selected from —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, —C₂-C₅alkenyl, —C₂-C₅alkynyl, cyclopropyl, cyclobutyl, cyclopentyl, cyclohexyl, cycloheptyl, cyclooctyl, 3- to 8-membered heterocyclyl, —C₆-C₁₂aryl, 5- to 12-membered heteroaryl, oxo (═O), —OR^{2a}, —CN, —C(O)R^{2a}, —CO₂R^{2a}, —NR^{2a}R^{2b}, —NR^{2a}COR^{2b}, and —NR^{2a}CO₂R^{2b}, wherein each of said methyl, ethyl, propyl (n-propyl or isopropyl), butyl (n-butyl, sec-butyl, iso-butyl or tert-butyl), pentyl, hexyl, heptyl, octyl, —C₂-C₅alkenyl, —C₂-C₅alkynyl, cyclopropyl, cyclobutyl, cyclopentyl, cyclohexyl, cycloheptyl, cyclooctyl, 3- to 8-membered heterocyclyl, —C₆-C₁₂aryl or 5- to 12-membered heteroaryl is optionally substituted with at least one substituent independently selected from —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, —C₂-C₅alkenyl, —C₂-C₅alkynyl, cyclopropyl, cyclobutyl, cyclopentyl, cyclohexyl, cycloheptyl, cyclooctyl, 3- to 8-membered heterocyclyl, C₆-C₁₂aryl, 5- to 12-membered heteroaryl, oxo (═O), —CN, —OR^{2c}, —C₁-C₈alkyl-OR^{2c}, —C(O)R^{2c}, —CO₂R^{2c}, —NR^{2c}R^{2d}, —NR^{2c}COR^{2d}, and —NR^{2c}CO₂R^{2d};
R^{2a}, R^{2b}, R^{2c} and R^{2d} are each independently selected from hydrogen, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₅alkynyl, cyclopropyl, cyclobutyl, cyclopentyl, cyclohexyl, cycloheptyl, cyclooctyl, 3- to 8-membered heterocyclyl, C₆-C₁₂aryl, and 5- to 12-membered heteroaryl, wherein each of said methyl, ethyl, propyl (n-propyl or isopropyl), butyl (n-butyl, sec-butyl, iso-butyl or tert-butyl), pentyl, hexyl, heptyl, octyl, —C₂-C₅alkenyl, —C₂-C₅alkynyl, cyclopropyl, cyclobutyl, cyclopentyl, cyclohexyl, cycloheptyl, cyclooctyl, 3- to 8-membered heterocyclyl, C₆-C₁₂aryl, or 5- to 12-membered heteroaryl is optionally substituted with at least one substituent independently selected from —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, —C₂-C₅alkenyl, —C₂-C₅alkynyl, cyclopropyl, cyclobutyl, cyclopentyl, cyclohexyl, cycloheptyl, cyclooctyl, 3- to 8-membered heterocyclyl, C₆-C₁₂aryl, 5- to 12-membered heteroaryl, oxo (═O), —CN, —OH, methoxy, ethoxy, propoxy, butoxy, pentoxy, hexoxy, hepthoxy, and octyloxy;
preferably, n is 0, 1, 2, 3, 4, or 5, provided that the valency theory has been met;
R² is each independently selected from —F, —Cl, methyl, ethyl, propyl (n-propyl or isopropyl), butyl (n-butyl, sec-butyl, iso-butyl or tert-butyl), —C₂-C₅alkenyl, cyclopropyl, cyclobutyl, cyclopentyl, cyclohexyl, 3- to 8-membered heterocyclyl, —C₆-C₁₂aryl, 5- to 12-membered heteroaryl, oxo (═O), —OR^{2a}, —CN, —C(O)R^{2a}, —CO₂R^{2a}, —NR^{2a}R^{2b}, —NR^{2a}COR^{2b}, and —NR^{2a}CO₂R^{2b}, wherein each of said methyl, ethyl, propyl (n-propyl or isopropyl), butyl (n-butyl, sec-butyl, iso-butyl or tert-butyl), —C₂-C₅alkenyl, cyclopropyl, cyclobutyl, cyclopentyl, cyclohexyl, 3- to 8-membered heterocyclyl, —C₆-C₁₂aryl or 5- to 12-membered heteroaryl is optionally substituted with at least one substituent independently selected from —F, —Cl, methyl, ethyl, propyl (n-propyl or isopropyl), butyl (n-butyl, sec-butyl, iso-butyl or tert-butyl), pentyl, hexyl, heptyl, octyl, —C₂-C₅alkenyl, —C₂-C₅alkynyl, cyclopropyl, cyclobutyl, 3- to 8-membered heterocyclyl, C₆-C₁₂aryl, 5- to 12-membered heteroaryl, oxo (═O), —CN, —OR^{2c}, —C₁-C₅alkyl-OR^{2c}, —C(O)R^{2c}, —CO₂R^{2c}, —NR^{2c}R^{2d}, —NR^{2c}COR^{2d}, and —NR^{2c}CO₂R^{2d};
R^{2a}, R^{2b}, R^{2c} and R^{2d} are each independently selected from hydrogen, methyl, ethyl, propyl (n-propyl or isopropyl), cyclopropyl, cyclobutyl, cyclopentyl, 3- to 8-membered heterocyclyl, C₆-C₁₂aryl, and 5- to 12-membered heteroaryl, wherein each of said methyl, ethyl, propyl (n-propyl or isopropyl), cyclopropyl, cyclobutyl, cyclopentyl, 3- to 8-membered heterocyclyl, C₆-C₁₂aryl, or 5- to 12-membered heteroaryl is optionally substituted with at least one substituent independently selected from —F, —Cl, methyl, ethyl, propyl (n-propyl or isopropyl), cyclopropyl, cyclobutyl, cyclopentyl, C₆-C₁₂aryl, oxo (═O), —CN, —OH, methoxy, and ethoxy;
more preferably, n is 0, 1, 2, 3, 4, or 5, provided that the valency theory has been met;
R² is each independently selected from —Cl, methyl, ethyl, isopropyl, tert-butyl, vinyl, cyclopropyl, cyclobutyl, phenyl, oxo (═O), —OR^{2a}, and —CO₂R^{2a}, wherein each of said methyl, ethyl, isopropyl, tert-butyl, vinyl, cyclopropyl, cyclobutyl, or phenyl is optionally substituted with at least one substituent independently selected from —F, methyl, —C₁-C₈alkyl-OR^{2c}, and —OR^{2c};
R^{2a} and R^{2c} are each independently selected from hydrogen, methyl, and ethyl;
even more preferably, n is 0, 1, 2, 3, 4, or 5, provided that the valency theory has been met;
R² is each independently selected from —Cl, methyl, ethyl, isopropyl, tert-butyl, vinyl, cyclopropyl, cyclobutyl, phenyl, oxo (═O), CF₃, methoxy, —OH, —CH₂OCH₃, CH₂OH, —CO₂CH₃, and —CO₂CH₂CH₃, wherein each of said ethyl, isopropyl, tert-butyl, vinyl, or cyclopropyl is optionally substituted with at least one substituent independently selected from methyl, —CH₂OH, and —OH.

Aspect 10. The compound of any one of the preceding aspects, wherein
R⁷ is selected from hydrogen, methyl, ethyl, propyl (n-propyl or isopropyl), cyclopropyl, cyclobutyl, cyclopentyl, cyclohexyl, cycloheptyl, and cyclooctyl;
preferably, R⁷ is selected from hydrogen, methyl, ethyl, cyclopropyl, cyclobutyl, and cyclopentyl;
more preferably, R⁷ is selected from hydrogen, methyl, ethyl, and cyclopropyl;
even more preferably, R⁷ is hydrogen.

Aspect 11. The compound of any one of aspects 1-9, wherein
n is 2, 3, 4, or 5, provided that the valency theory has been met; adjacent R² and R⁷, together with the atoms to which they are attached, form a 5-, 6-, 7-, 8-, or 9-membered ring, said ring comprising 0-3 heteroatoms independently selected from nitrogen, oxygen or optionally oxidized sulfur as ring member(s); said ring is optionally substituted with at least one substituent R^{7a};
R^{7a} is each independently selected from —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, —C₆-C₁₂aryl, 5- to 12-membered heteroaryl, and oxo (═O);
preferably, n is 2, 3, 4, or 5, provided that the valency theory has been met; adjacent R² and R⁷, together with the atoms to which they are attached, form a 5-, 6-, 7-, 8-, or 9-membered ring, said ring comprising 0-3 heteroatoms independently selected from nitrogen and oxygen as ring member(s); said ring is optionally substituted with at least one substituent R^{7a};
R^{7a} is each independently selected from —F, —Cl, methyl, ethyl, cyclopropyl, cyclobutyl, cyclopentyl, cyclohexyl, 3- to 8-membered heterocyclyl, —C₆-C₁₂aryl, and oxo (═O);
more preferably, n is 2, 3, or 4, provided that the valency theory has been met; adjacent R² and R⁷, together with the atoms to which they are attached, form a 5- or 6-membered ring, said ring comprising 0-1 heteroatom selected from nitrogen and oxygen atom as ring member; said ring is optionally substituted with at least one substituent R^{7a};
R^{7a} is each independently selected from —F, methyl, ethyl, cyclopropyl, cyclobutyl, —C₆-C₁₂aryl, and oxo (═O);
even more preferably, n is 2, provided that the valency theory has been met; adjacent R² and R⁷, together with the atoms to which they are attached, form cyclopentyl, cyclohexyl, oxanyl, tetrahydrofuranyl, phenyl or pyridinyl; said cyclopentyl, cyclohexyl, oxanyl, tetrahydrofuranyl is optionally substituted with two methyl substituents.

Aspect 12. The compound of any one of the preceding aspects, wherein
R³ and R⁴ are each independently selected from hydrogen, methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, or —C(O)R^{3a}; wherein each of said methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, or cyclooctyl is optionally substituted at least one substituent independently selected from with —F, —Cl, —Br, —I, —CN, —OR^{3b} and —NR^{3b}R^{3c};
R^{3a}, R^{3b} and R^{3c} are each independently selected from hydrogen, methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, or cyclooctyl; wherein each of said methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, or cyclooctyl is optionally substituted with at least one substituent independently selected from —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, oxo (═O), —CN, —OH, methoxy, ethoxy, propoxy, butoxy, pentoxy, hexoxy, hepthoxy and octyloxy;
preferably, R³ and R⁴ are each independently selected from hydrogen, methyl, ethyl, propyl (n-propyl or isopropyl), butyl (n-butyl, sec-butyl, iso-butyl or tert-butyl), cyclopropyl, cyclobutyl, cyclopentyl, and —C(O)R^{3a}; wherein each of said methyl, ethyl, propyl (n-propyl or isopropyl), butyl (n-butyl, sec-butyl, iso-butyl or tert-butyl), cyclopropyl, cyclobutyl, or cyclopentyl is optionally substituted with at least one substituent independently selected from —F and —NR^{3b}R^{3c};
R^{3a}, R^{3b} and R^{3c} are each independently selected from hydrogen, methyl, ethyl, propyl (n-propyl or isopropyl), butyl (n-butyl, sec-butyl, iso-butyl or tert-butyl), cyclopropyl, cyclobutyl, cyclopentyl and cyclohexyl;
more preferably, R³ and R⁴ are each independently selected from hydrogen, methyl, and —C(O)R^{3a}; R^{3a} is selected from hydrogen, methyl, and ethyl;
even more preferably, R³ and R⁴ are each independently hydrogen.

Aspect 13. The compound of any one of the preceding aspects, wherein the

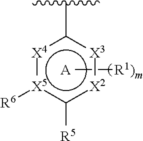

moiety is selected from

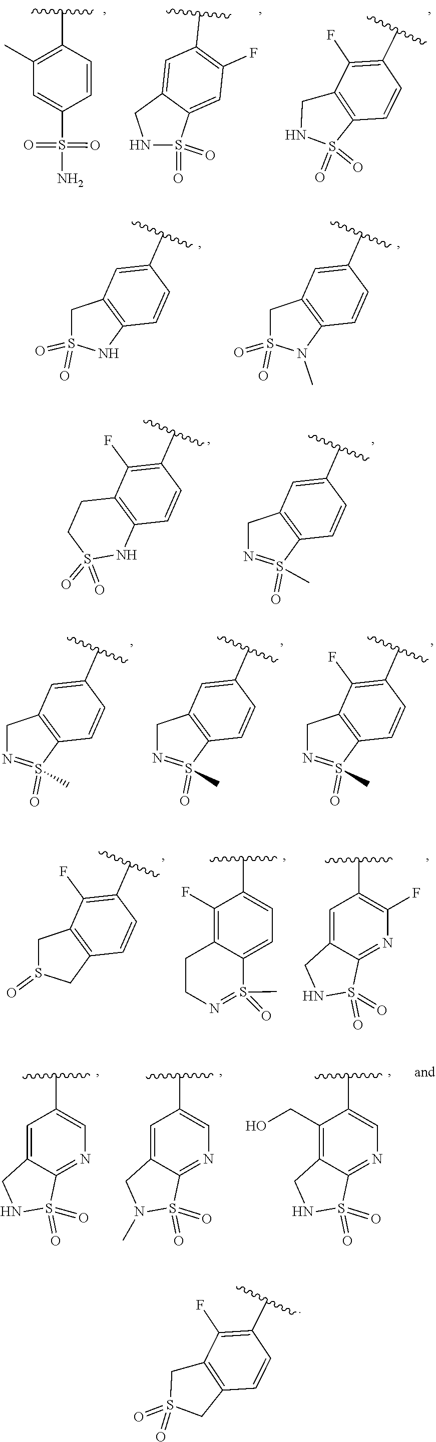

Aspect 14. The compound of any one of the preceding aspects, wherein the

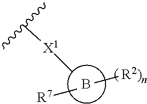

moiety is selected from

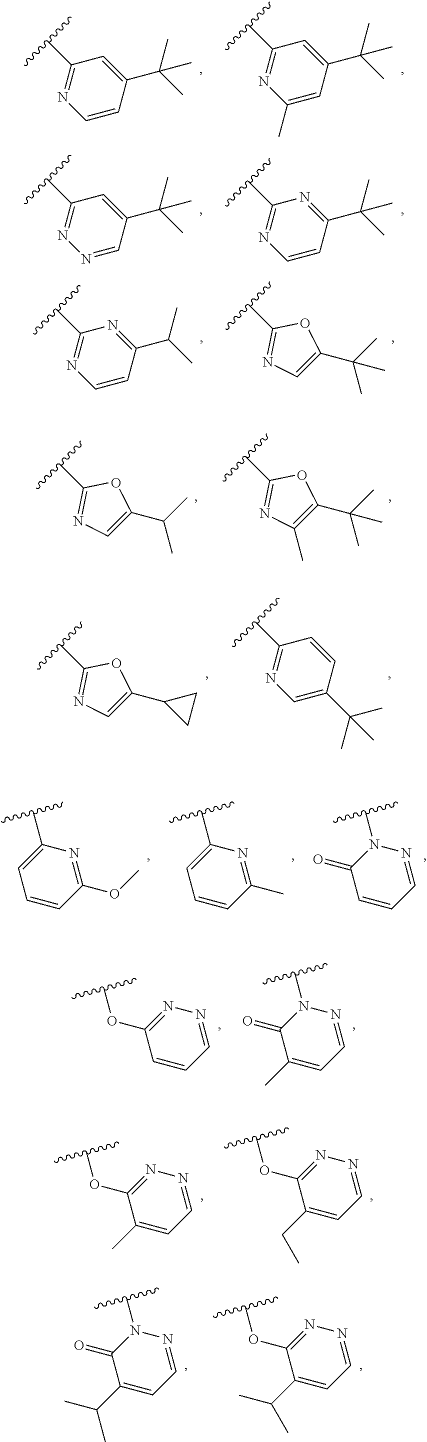

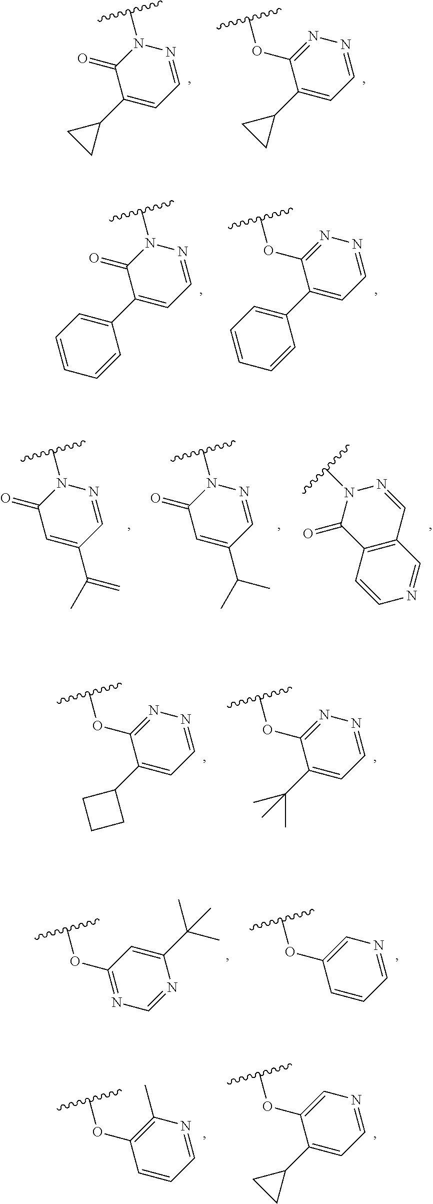

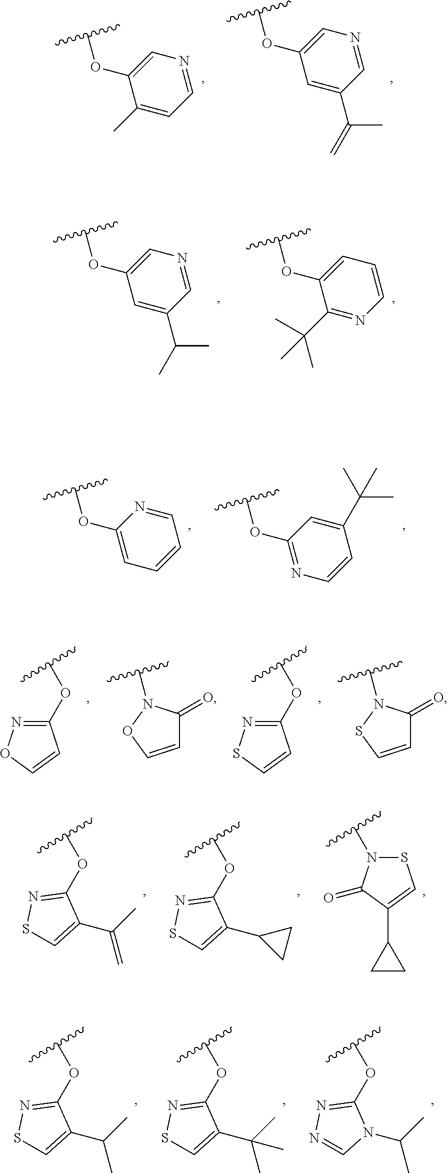

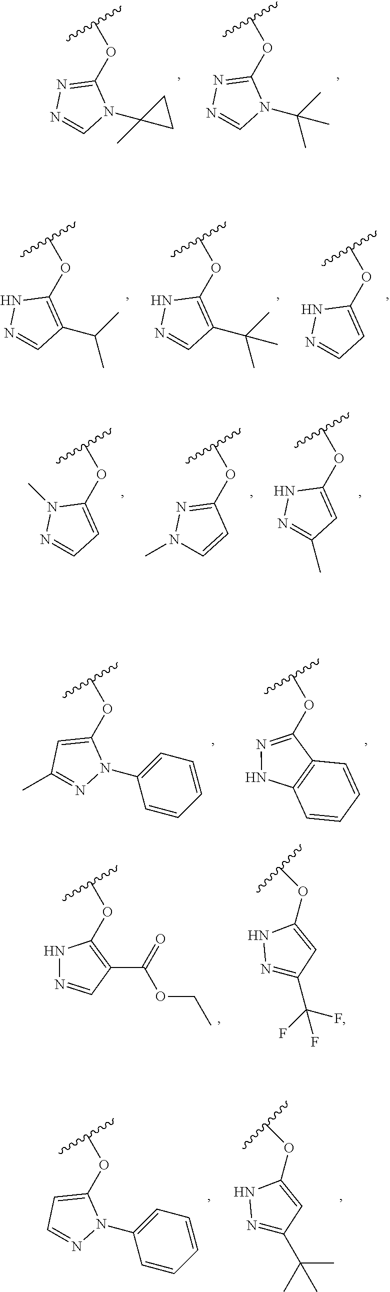

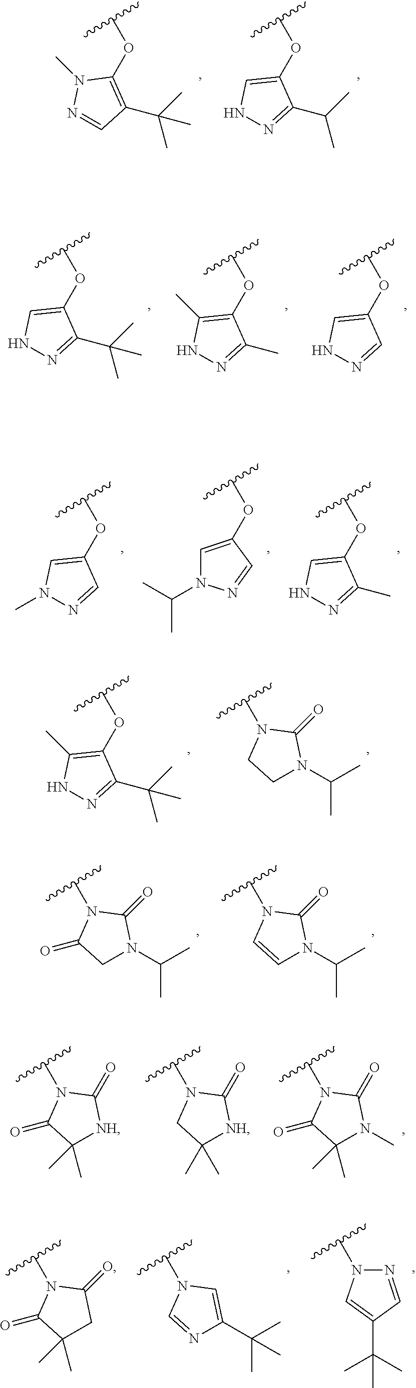

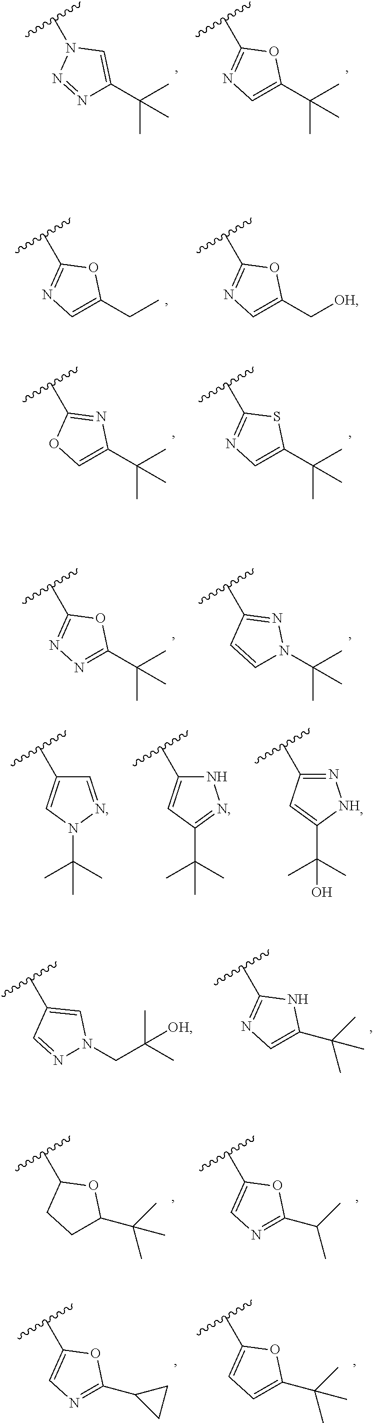

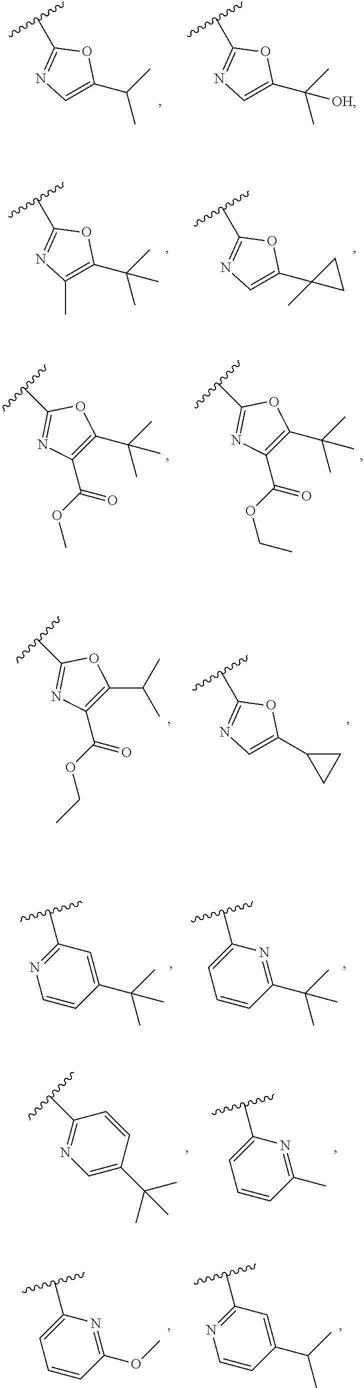

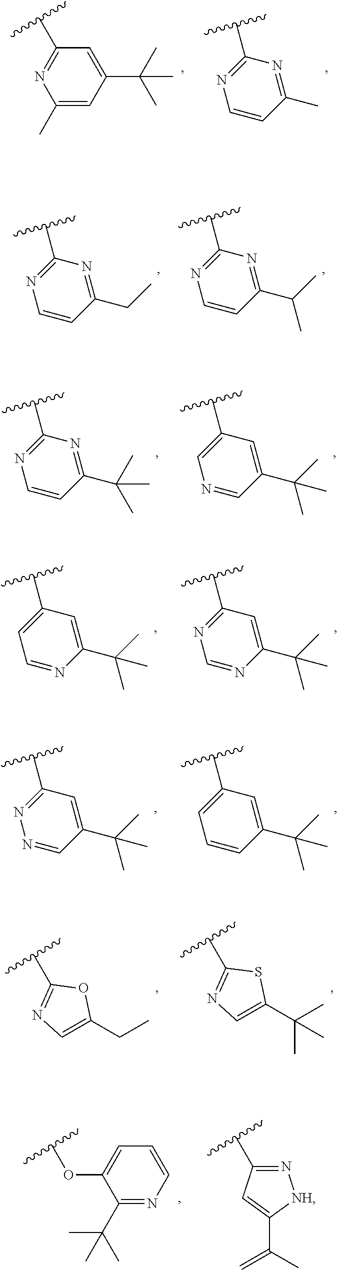

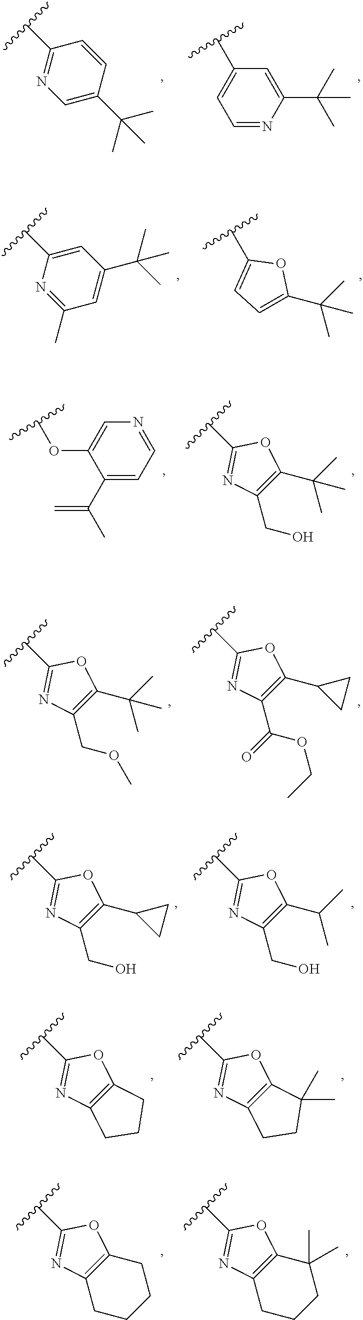

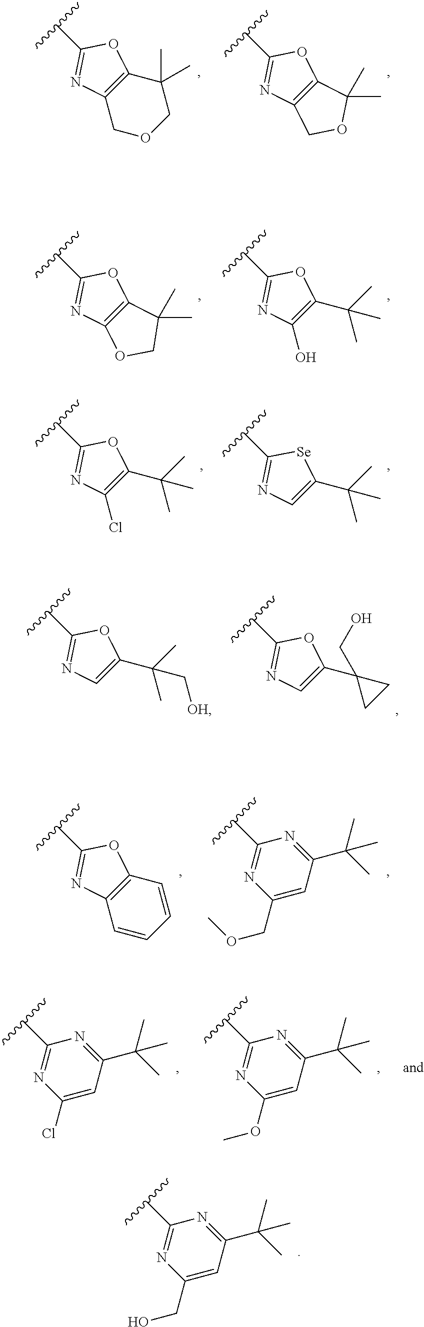

Aspect 15. The compound of any one of the preceding aspects, wherein the compound is selected from

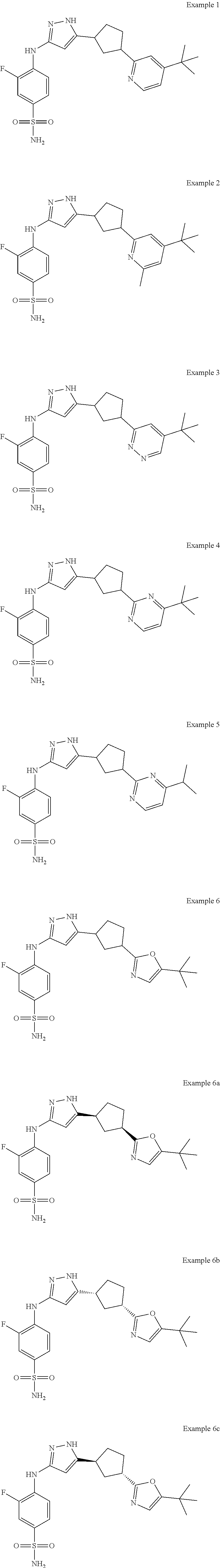

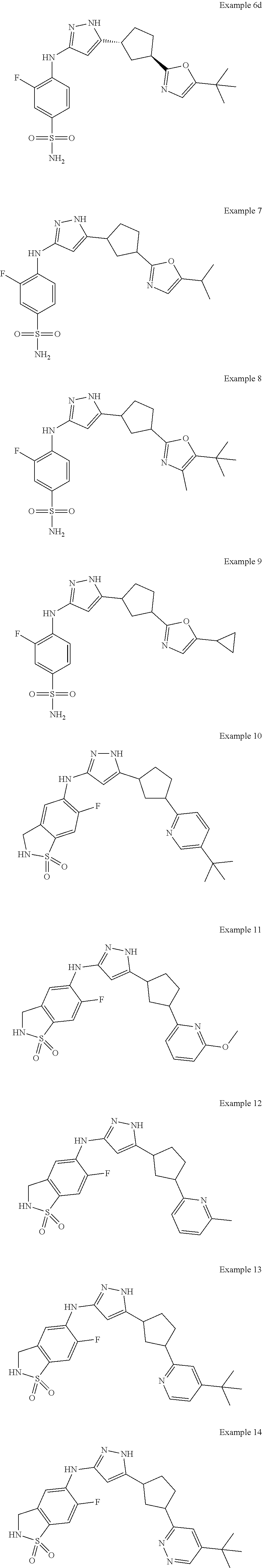

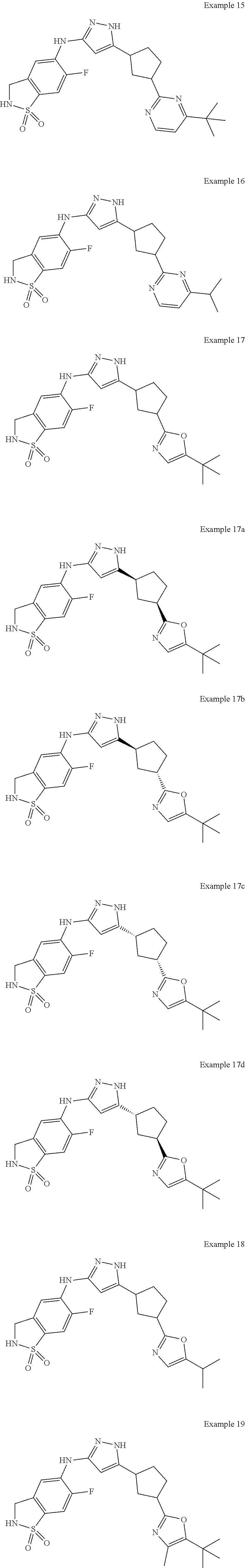

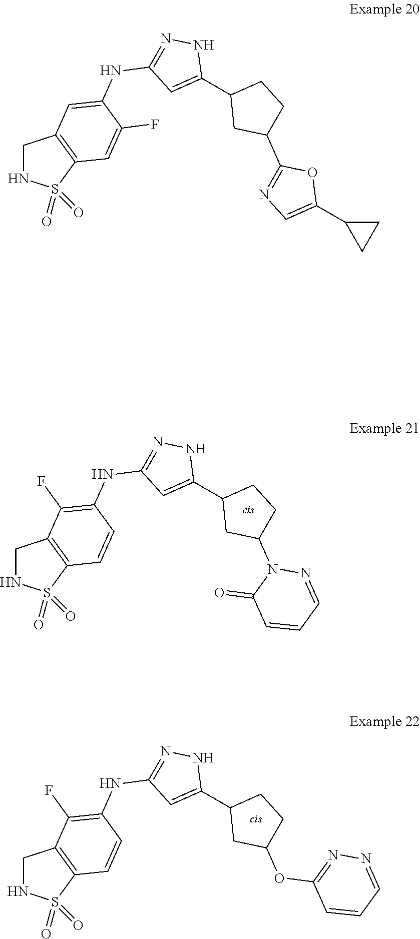 [SMILES: O=S1(=O)NCc2cc(Nc3cc(C4CCC(c5ncc(C6CC6)o5)C4)[nH]n3)c(F)cc21 ; O=c1cccnn1C1CCC(c2cc(Nc3ccc4c(c3F)CNS4(=O)=O)n[nH]2)C1 ; O=S1(=O)NCc2c1ccc(Nc1cc(C3CCC(Oc4cccnn4)C3)[nH]n1)c2F]

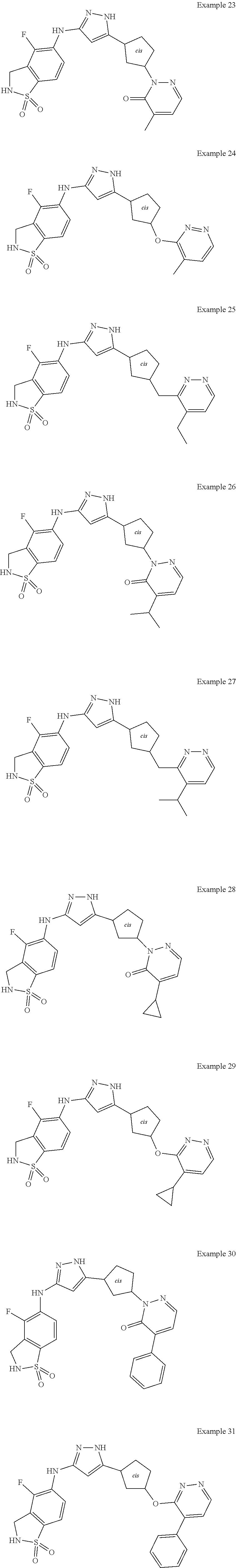

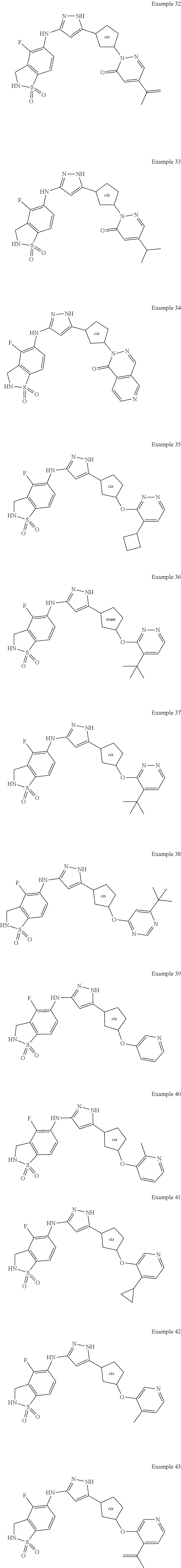

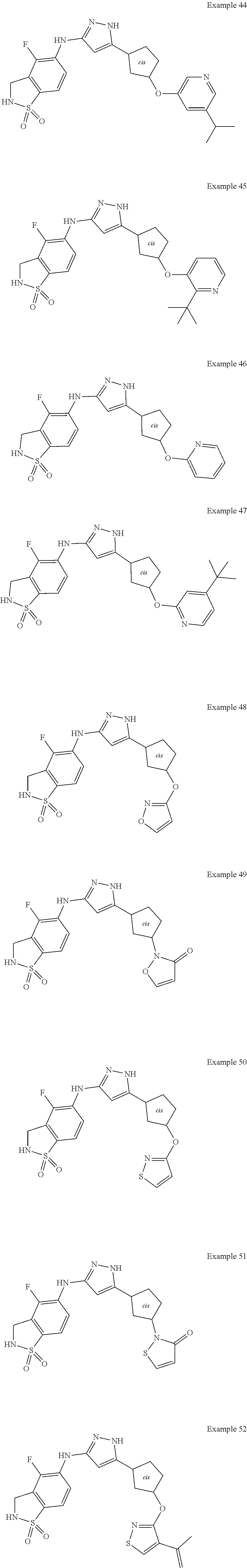

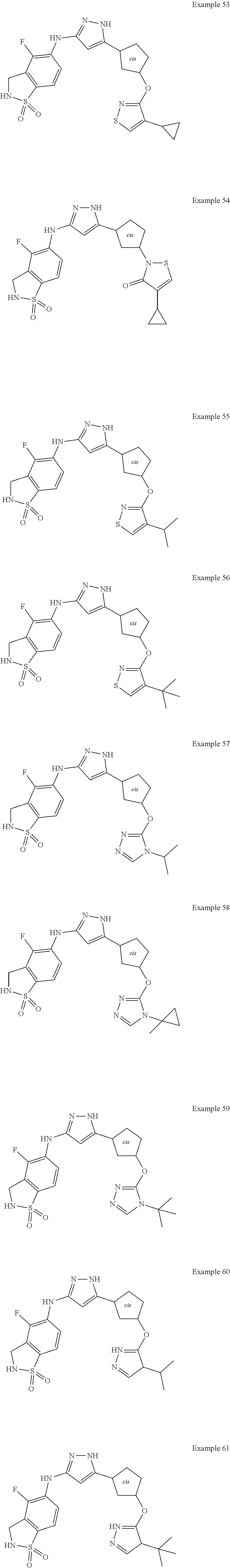

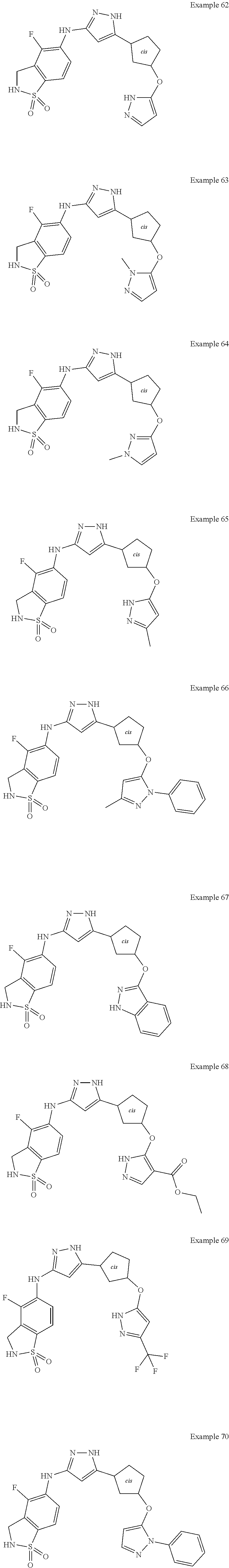

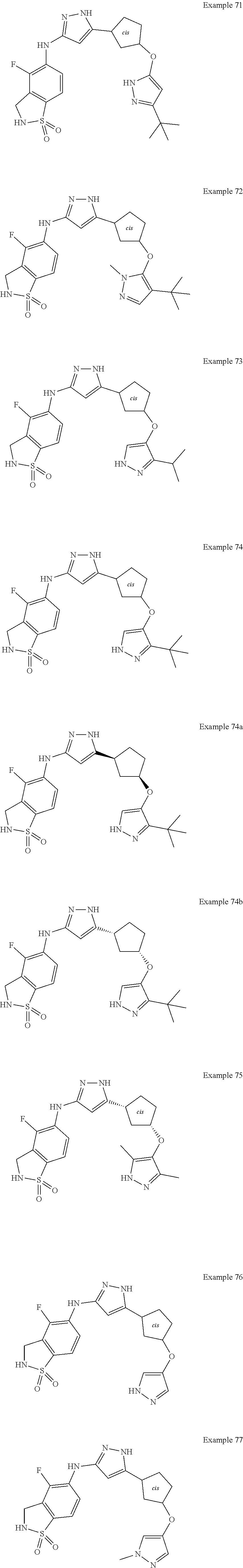

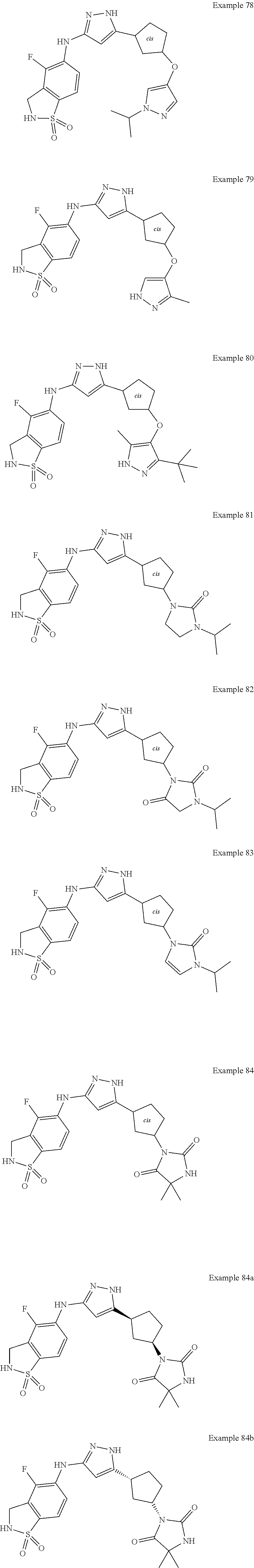

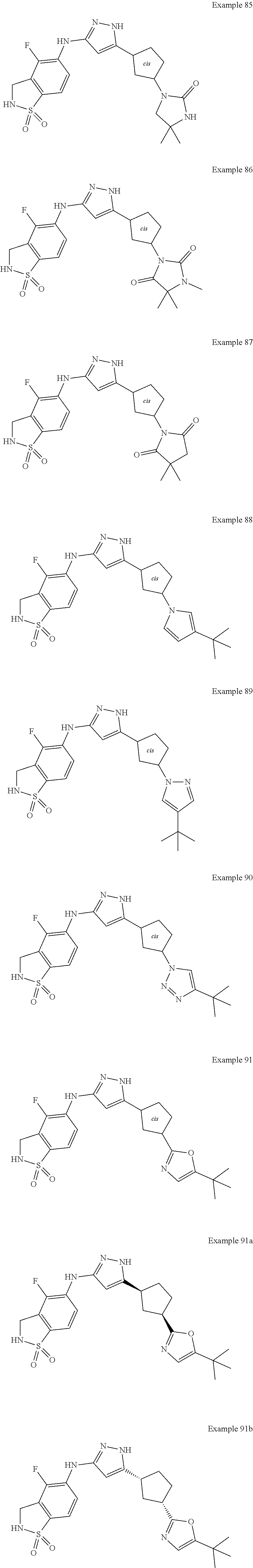

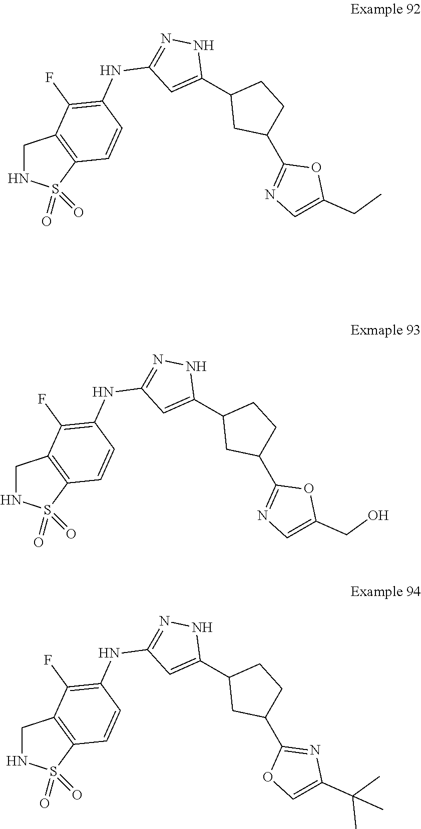 [SMILES: CCc1cnc(C2CCC(c3cc(Nc4ccc5c(c4F)CNS5(=O)=O)n[nH]3)C2)o1 ; O=S1(=O)NCc2c1ccc(Nc1cc(C3CCC(c4ncc(CO)o4)C3)[nH]n1)c2F ; CC(C)(C)c1coc(C2CCC(c3cc(Nc4ccc5c(c4F)CNS5(=O)=O)n[nH]3)C2)n1]

[chemical structure]

[chemical structure]

[chemical structure]

[chemical structure]

[chemical structure]

[chemical structure]

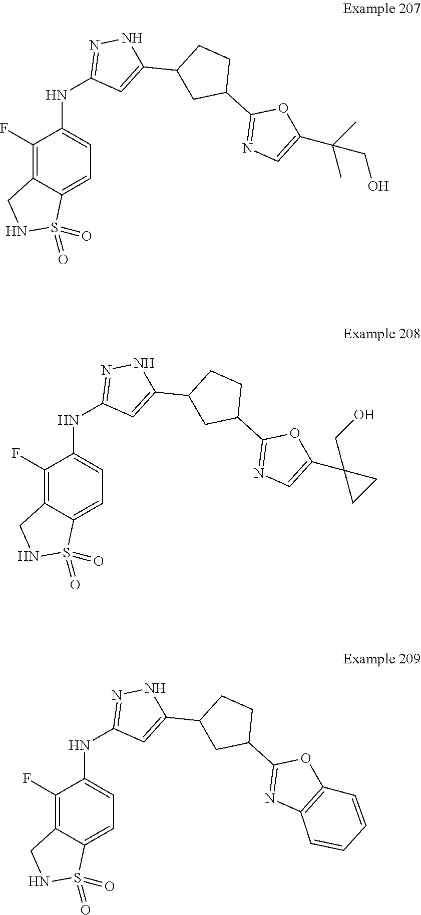 [SMILES: CC(C)(CO)c1cnc(C2CCC(c3cc(Nc4ccc5c(c4F)CNS5(=O)=O)n[nH]3)C2)o1 ; O=S1(=O)NCc2c1ccc(Nc1cc(C3CCC(c4ncc(C5(CO)CC5)o4)C3)[nH]n1)c2F ; O=S1(=O)NCc2c1ccc(Nc1cc(C3CCC(c4nc5ccccc5o4)C3)[nH]n1)c2F]

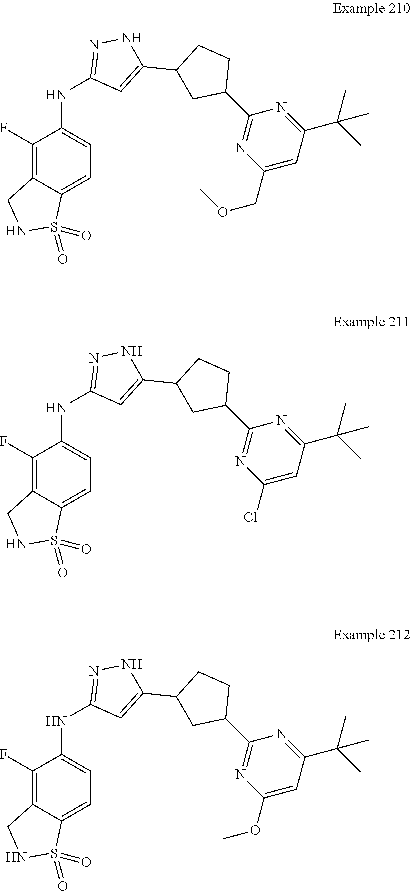 [SMILES: COCc1cc(C(C)(C)C)nc(C2CCC(c3cc(Nc4ccc5c(c4F)CNS5(=O)=O)n[nH]3)C2)n1 ; CC(C)(C)c1cc(Cl)nc(C2CCC(c3cc(Nc4ccc5c(c4F)CNS5(=O)=O)n[nH]3)C2)n1 ; COc1cc(C(C)(C)C)nc(C2CCC(c3cc(Nc4ccc5c(c4F)CNS5(=O)=O)n[nH]3)C2)n1]

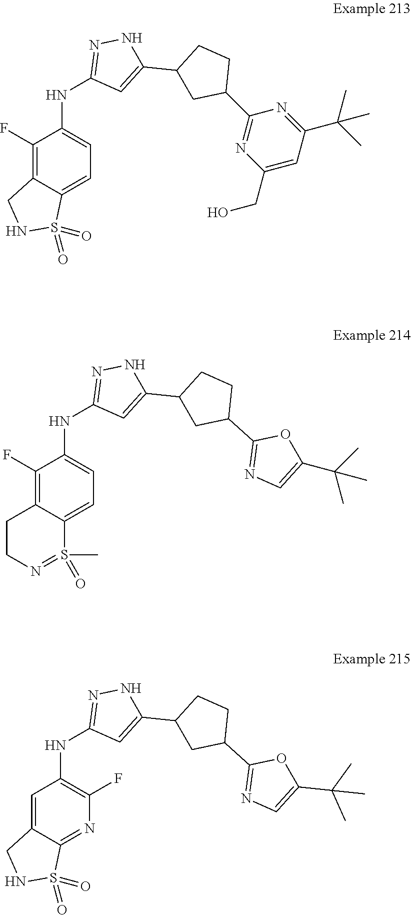 [SMILES: CC(C)(C)c1cc(CO)nc(C2CCC(c3cc(Nc4ccc5c(c4F)CNS5(=O)=O)n[nH]3)C2)n1 ; CC(C)(C)c1cnc(C2CCC(c3cc(Nc4ccc5c(c4F)CCN=S5(C)=O)n[nH]3)C2)o1 ; CC(C)(C)c1cnc(C2CCC(c3cc(Nc4cc5c(nc4F)S(=O)(=O)NC5)n[nH]3)C2)o1]

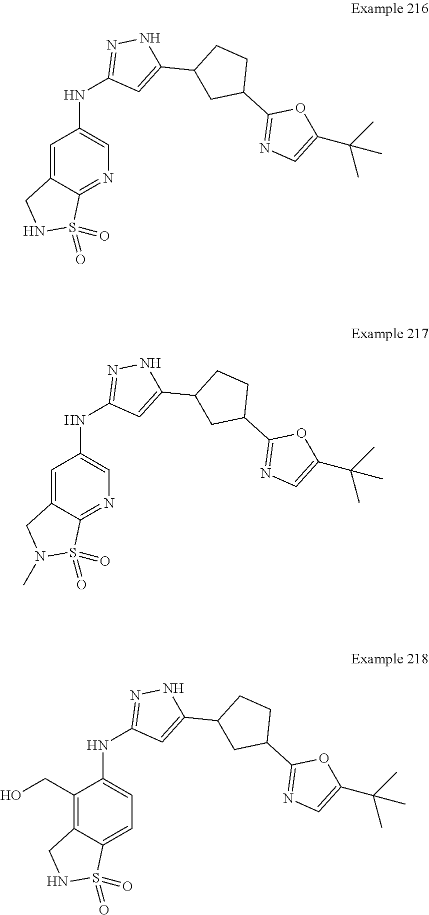

Aspect 16. A pharmaceutical composition comprising a compound of any one of aspects 1-15, or a pharmaceutically acceptable salt thereof, or a stereoisomer, a tautomer or a prodrug thereof, and at least one pharmaceutically acceptable carrier or excipient.

Aspect 17. A method of treating cancer, comprising administering to a subject in need thereof a compound of any one of aspects 1-15, or a pharmaceutically acceptable salt, or a stereoisomer, a tautomer or a prodrug thereof.

Aspect 18. A compound, a pharmaceutically acceptable salt, or a stereoisomer, a tautomer or a prodrug according to any one of aspects 1-15 for use in a method of treating cancer.

Aspect 19. The compound of aspect 18, wherein the cancer is breast cancer, ovarian cancer, bladder cancer, gastric cancer, uterine cancer, prostate cancer, lung cancer, esophageal cancer, head and neck cancer, colorectal cancer, kidney cancer, liver cancer, pancreatic cancer, and/or thyroid cancer.

### DETAILED DESCRIPTION OF THE INVENTION

The following terms have the indicated meanings throughout the specification:

As used herein, including the appended Aspects, the singular forms of words such as “a”, “an”, and “the”, include their corresponding plural references unless the context clearly dictates otherwise.

The term “or” is used to mean, and is used interchangeably with, the term “and/or” unless the context clearly dictates otherwise.

The term “alkyl” refers to a hydrocarbon group selected from linear and branched saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl ort-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “cycloalkyl” refers to a hydrocarbon group selected from saturated cyclic hydrocarbon groups, comprising monocyclic and polycyclic (e.g., bicyclic and tricyclic) groups including fused, bridged or spiro cycloalkyl.

The term “aryl” used alone or in combination with other terms refers to a group selected from:
5- and 6-membered carbocyclic aromatic rings, e.g., phenyl;
bicyclic ring systems such as 7- to 12-membered bicyclic ring systems, wherein at least one ring is carbocyclic and aromatic, e.g., naphthyl and indanyl; and,
tricyclic ring systems such as 10- to 15-membered tricyclic ring systems wherein at least one ring is carbocyclic and aromatic, e.g., fluorenyl.

The terms “aromatic hydrocarbon ring” and “aryl” are used interchangeable throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring include, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

The term “aryl-alkyl-” refers to an alkyl group as defined above which is further substituted by an aryl group. Examples of an aryl-alkyl group include aryl-C₁₋₈alkyl, such as phenylethyl, or phenylmethyl (benzyl).

The term “heteroaryl” refers to a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides. The term “C-linked heteroaryl” as used herein means that the heteroaryl group is connected to the core molecule by a bond from a C-atom of the heteroaryl ring

The terms “aromatic heterocyclic ring” and “heteroaryl” are used interchangeable throughout the disclosure herein. In some embodiments, a monocyclic or bicyclic aromatic heterocyclic ring has 5-, 6-, 7-, 8-, 9- or 10-ring forming members with 1, 2, 3, or 4 heteroatom ring members independently selected from nitrogen (N), sulfur (S) and oxygen (O) and the remaining ring members being carbon. In some embodiments, the monocyclic or bicyclic aromatic heterocyclic ring is a monocyclic or bicyclic ring comprising 1 or 2 heteroatom ring members independently selected from nitrogen (N), sulfur (S) and oxygen (O). In some embodiments, the monocyclic or bicyclic aromatic heterocyclic ring is a 5- to 6-membered heteroaryl ring, which is monocyclic and which has 1 or 2 heteroatom ring members independently selected from nitrogen (N), sulfur (S) and oxygen (O). In some embodiments, the monocyclic or bicyclic aromatic heterocyclic ring is an 8- to 10-membered heteroaryl ring, which is bicyclic and which has 1 or 2 heteroatom ring members independently selected from nitrogen, sulfur and oxygen.

“Heterocyclyl”, “heterocycle” or “heterocyclic” are interchangeable and refer to a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups. The term “optionally oxidized sulfur” used herein refers to S, SO or SO₂.

“Hydrogen” and “H” are interchangeable and refer to anyone of protium (¹H), deuterium (²H) or tritium (³H). “Deuterated analog” refers to one or more protiums (¹H) of the compound are substituted with equal numbers of deuteriums (²H).

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, the reference to one isomer applies to any of the possible isomers. Whenever the isomeric composition is unspecified, all possible isomers are included.

The term “substantially pure” as used herein means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

When compounds disclosed herein contain olefinic double bonds, unless specified otherwise, such double bonds are meant to include both E and Z geometric isomers.

When compounds disclosed herein contain a di-substituted cyclohexyl or cyclobutyl group, substituents found on cyclohexyl or cyclobutyl ring may adopt cis and trans formations. Cis formation means that both substituents are found on the upper side of the 2 substituent placements on the carbon, while trans would mean that they were on opposing sides.

It may be advantageous to separate reaction products from one another and/or from starting materials. The desired product of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

“Diastereomers” refers to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

Some of the compounds disclosed herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

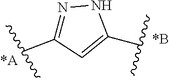 [SMILES: *c1cc(*)[nH]n1]

may undergo tautomerism to form

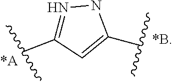 [SMILES: *c1cc(*)[nH]n1]

Wherein *A and *B refer to the position substituents connect to pyrazole.

“Pharmaceutically acceptable salts” refers to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and/or water and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” includes salts of at least one compound of Formula (I), and salts of the stereoisomers of the compound of Formula (I), such as salts of enantiomers, and/or salts of diastereomers.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as the contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as a compound that, when administered to a subject for treating a disease, or at least one of the clinical symptoms of a disease or disorder, is sufficient to affect such treatment for the disease, disorder, or symptom. The “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer thereof, and/or at least one pharmaceutically acceptable salt thereof disclosed herein effective to “treat” as defined above, a disease or disorder in a subject. In the case of combination therapy, the “therapeutically effective amount” refers to the total amount of the combination objects for the effective treatment of a disease, a disorder or a condition.

The pharmaceutical composition comprising the compound disclosed herein can be administrated via oral, inhalation, rectal, parenteral or topical administration to a subject in need thereof. For oral administration, the pharmaceutical composition may be a regular solid formulation such as tablets, powder, granule, capsules and the like, a liquid formulation such as water or oil suspension or other liquid formulation such as syrup, solution, suspension or the like; for parenteral administration, the pharmaceutical composition may be a solution, water solution, oil suspension concentrate, lyophilized powder or the like. Preferably, the formulation of the pharmaceutical composition is selected from a tablet, coated tablet, capsule, suppository, nasal spray or injection, more preferably tablet or capsule. The pharmaceutical composition can be a single unit administration with an accurate dosage. In addition, the pharmaceutical composition may further comprise additional active ingredients.

All formulations of the pharmaceutical composition disclosed herein can be produced by the conventional methods in the pharmaceutical field. For example, the active ingredient can be mixed with one or more excipients, then to make the desired formulation. The “pharmaceutically acceptable excipient” refers to conventional pharmaceutical carriers suitable for the desired pharmaceutical formulation, for example: a diluent, a vehicle such as water, various organic solvents, etc., a filler such as starch, sucrose, etc. a binder such as cellulose derivatives, alginates, gelatin and polyvinylpyrrolidone (PVP); a wetting agent such as glycerol; a disintegrating agent such as agar, calcium carbonate and sodium bicarbonate; an absorption enhancer such as quaternary ammonium compound; a surfactant such as hexadecanol; an absorption carrier such as Kaolin and soap clay; a lubricant such as talc, calcium stearate, magnesium stearate, polyethylene glycol, etc. In addition, the pharmaceutical composition further comprises other pharmaceutically acceptable excipients such as a decentralized agent, a stabilizer, a thickener, a complexing agent, a buffering agent, a permeation enhancer, a polymer, aromatics, a sweetener, and a dye.

The term “disease” refers to any disease, discomfort, illness, symptoms or indications, and can be interchangeable with the term “disorder” or “condition”.

Throughout this specification and the Aspects which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the Aspects which follow, the term “Cₙ₋ₘ” indicates a range which includes the endpoints, wherein n and m are integers and indicate the number of carbons. Examples include C₁₋₈, C₁₋₆, and the like.

Unless specifically defined elsewhere in this document, all other technical and scientific terms used herein have the meaning commonly understood by one of ordinary skill in the art to which this invention belongs.

ABBREVIATIONS

| | |
|---|---|
| AIBN | 2,2′-Azobis(2-methylpropionitrile) |
| ACN | Acetonitrile |
| ATP | Adenosine triphosphate |
| aq. | Aqueous |
| atm | standard atmosphere |
| Brettphos Pd | Methanesulfonato (2-dicyclohexylphosphino-3,6- |
| G3 | dimethoxy-2′,4′,6′-tri-i-propyl-1,1′-biphenyl)(2′-amino- |
| | 1,1′-biphenyl-2-yl)palladium(II) |
| BSA | Bis(trimethylsilyl)acetamide |
| B₂Pin₂ | Bis(pinacolato)diboron |
| BH₃—THF | Borane-tetrahydrofuran |
| CuBr | Copper(I) bromide |
| CO | Carbon monoxide |
| CMBP | (Tributylphosphoranylidene)acetonitrile |
| DTT | Dithiothreitol |
| DCM | dichloromethane |
| DIEA | diisopropylethylamine |
| DMAP | 4-dimethylaminopyridine |
| DMF | Dimethylformamide |
| DMEM | Dulbecco's Modified Eagle Medium |
| DMSO | Dimethyl sulfoxide |
| DIBAL—H | Diisobutylaluminium hydride |
| DIAD | Diisopropyl azodicarboxylate |
| DTT | Dithiothreitol |
| EA | Ethyl acetate |
| EA, EtOAc | Ethyl acetate |
| Eq. | equivalent |
| Et₃N, TEA | triethyl amine |
| EtOH | ethanol |
| FBS | Fetal bovine serum |
| FA | Formic acid |
| HCl | Hydrochloric Acid |
| Hex | Hexene |
| HEPES | 4-(2-hydroxyethyl)-1-piperazineethanesulfonic acid |
| IPA | Isopropyl alcohol |
| K₂CO₃ | Potassium carbonate |
| KOH | Potassium hydroxide |
| LiBHEt₃ | Lithium triethylborohydride |
| LC-MS (ESI) | Liquid Chromatography-Mass Spectrometry |
| | (Electrospray Ionization) |
| LDA | Lithium diisopropylamide |
| MtBE | Methyl tert-butyl ether |
| MeCN | acetonitrile |
| MeOH | Methanol |
| MgCl₂ | Magnesium dichloride |
| MHz | Megahertz |
| mg | Milligrams |
| mL | Milliliters |
| mmol | Millimole |
| m-CPBA | m-Chloroperbenzoic Acid |
| NaBH₄ | Sodium borohydride |
| NBS | N-Bromosuccinimide |
| NMR | Nuclear Magnetic Resonance |
| NaHCO₃ | Sodium bicarbonate |
| NH₄Cl | Ammonium chloride |
| NH₄HCO₃ | Ammonium bicarbonate |
| NIS | N-Iodosuccinimide |
| Na₂SO₄ | Sodium sulphate |
| N₂ | Nitrogen |
| NaOH | Sodium hydroxide |
| NH₃ | Ammonia |
| NaIO₄ | Sodium periodate |
| NaN₃ | Sodium azide |
| n-BuLi | n-Butyllithium |
| pH | Potential of Hydrogen |
| Pd(dppf)Cl₂ | [1,1′-Bis(diphenylphosphino)ferrocene] |
| | dichloropalladium(II) |
| PE | Petroleum ether |
| PCy₃ | Tricyclohexylphosphine |
| PS | Phosphatidylserine |
| PreP-HPLC | Preparative High-Performance Liquid Chromatography |
| PhCl | Chlorobenzene |
| Pd/C | Palladium on carbon |
| Pd₂dba₃ | Tris(dibenzylideneacetone)dipalladium |
| PTSA | 1,3,6,8-Pyrenetetrasulfonicacid, sodium salt |
| R_{f} | Retention Factor |
| RuCl₃ | Ruthenium(III) chloride |
| RT or rt | room temperature |
| SOCl₂ | Thionyl chloride |
| SEMCl | 2-(Trimethylsilyl)ethoxymethyl chloride |
| sat. | Saturated |
| TFA | trifluoroacetic acid |
| THF | tetrahydrofuran |
| TEA | Triethylamine |
| TFAA | Trifluoroacetic anhydride |
| TBSCI | tert-Butyldimethylsilyl chloride |
| t-BuOH | tert-butyl alcohol |
| t-BuONa | Sodium t-butoxide |

### EXAMPLES

The examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless indicated otherwise. Unless indicated otherwise, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

¹H NMR spectra were recorded on an Agilent instrument operating at 400 MHz. ¹HNMR spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05; (CD₃)₃CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

LCMS-1: LC-MS spectrometer (Agilent 1260 Infinity) Detector: MWD (190-400 nm), Mass detector: 6120 SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS-2: LC-MS spectrometer (Agilent 1290 Infinity II) Detector: MWD (190-400 nm), Mass detector: G6125C SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.2 mL/min Time (min) A (%) B (%)

| Time (min) | A(%) | B(%) |
|---|---|---|
| 0.00 | 90 | 10 |
| 1.5 | 5 | 95 |
| 2.0 | 5 | 95 |
| 2.1 | 90 | 10 |
| 3.0 | 90 | 10 |

LCMS-3: LC-MS spectrometer (Agilent 1260 Infinity II) Detector: MWD (190-400 nm), Mass detector: G6125C SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

UPLC-MS: UPLC: Waters Acquity UPLC H-Class, Detector: MWD (190-400 nm), Mass detector: Waters QDA, Mobile phase: A: water with 0.05% TFA, B: acetonitrile with 0.05% TFA, Column: Waters Acquity UPLC BEH C18, 2.1×50 mm, 1.7 pm, Gradient method: Flow: 0.8 mL/min Time (min) A (%) B (%)

| Time (min) | A (%) | B (%) |
|---|---|---|
| 0.00 | 95 | 5 |
| 4.00 | 5 | 95 |
| 4.50 | 5 | 95 |
| 4.51 | 95 | 5 |
| 5.50 | 95 | 5 |

Preparative HPLC was conducted on a column (150×21.2 mm ID, 5 pm, Gemini NXC 18) at a flow rate of 20 ml/min, injection volume 2 ml, at room temperature and UV Detection at 214 nm and 254 nm.

### Example 1: 4-((5-(3-(4-(tert-butyl)pyridin-2-yl)cyclopentyl)-1H-pyrazol-3-yl)amino)-3-fluorobenzenesulfonamide

### Step 1: methyl 1,4-dioxaspiro[4.4]nonane-7-carboxylate

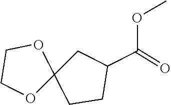

Two parallel reactions were performed. A solution of methyl 3-oxocyclopentanecarboxylate (50.0 g, 351.74 mmol) in toluene (500 mL) was treated with ethylene glycol (43.66 g, 703.47 mmol) and 4-toluenesulfonic acid (6.06 g, 35.17 mmol). The mixture was heated to reflux and stirred for 4 h under nitrogen atmosphere. Each batch was quenched with sat. aq NaHCO₃ separately, then the two bathes were combined and concentrated under reduced pressure. The residue was diluted with EA (2 L) and washed with aq NaHCO₃ (500 mL). The aqueous layer was further extracted with EA (500 mL×2). The combined organic layers were washed with brine (500 mL), dried over anhydrous Na₂SO₄, then filtered and concentrated under reduced pressure. The residue was purified by silica gel column chromatography, eluting with PE/EA (1:1; R_{f}=0.43) to afford the product (34 g, 26% yield). ¹H NMR (400 MHz, Chloroform-d) δ=4.00-3.84 (m, 4H), 3.68 (s, 3H), 2.98-2.85 (m, 1H), 2.09 (d, J=8.9 Hz, 2H), 2.06-1.88 (m, 3H), 1.85-1.78 (m, 1H).

### Step 2: 3-oxo-3-(1,4-dioxaspiro[4.4]nonan-7-yl)propanenitrile

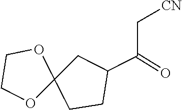 [SMILES: N#CCC(=O)C1CCC2(C1)OCCO2]

Two parallel reactions were performed. To a solution of methyl 1,4-dioxaspiro[4.4]nonane-7-carboxylate (16.0 g, 85.93 mmol) in THF (320 mL), acetonitrile (10.58 g, 257.78 mmol) and sodium hydride (10.31 g, 257.78 mmol, 60%) were added at 0° C. under nitrogen atmosphere. The resulting mixture was heated to reflux and stirred for 4 h. Two batches were combined and poured into sat. aq NH₄Cl (500 mL) at 0° C. and stirred for 30 mins. The layers separated, and the aqueous layer was extracted with EA (500 mL×3). The combined organic layer was washed with brine (200 mL), dried over anhydrous Na₂SO₄, filtered, and concentrated under vacuum. The residue was purified by silica gel chromatography, eluting with PE/EA (10:1; R_{f}=0.29) to afford the product (30.1 g, 90% yield). ¹H NMR (400 MHz, Chloroform-d) δ=3.96-3.86 (m, 4H), 3.54 (d, J=0.7 Hz, 2H), 3.25-3.10 (m, 1H), 2.11-2.00 (m, 3H), 1.98-1.78 (m, 3H).

### Step 3: 1-(tert-butyl)-3-(1,4-dioxaspiro[4.4]nonan-7-yl)-1H-pyrazol-5-amine

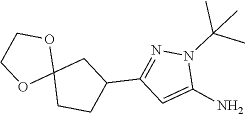 [SMILES: CC(C)(C)n1nc(C2CCC3(C2)OCCO3)cc1N]

To a solution of 3-oxo-3-(1,4-dioxaspiro[4.4]nonan-7-yl)propanenitrile (30.0 g, 153.68 mmol) and tert-butylhydrazine mono hydrochloride (57.45 g, 461.03 mmol) in ethyl alcohol (300 mL) was added triethylamine (46.65 g, 461.03 mmol) at 20° C. The resulting mixture was heated to reflux and stirred for 2 h under nitrogen atmosphere. The reaction mixture was concentrated under reduced pressure, then the residue was purified by silica gel chromatography, eluting with PE/EA (5:1; R_{f}=0.24) to afford the product (27 g, 64% yield). ¹H NMR (400 MHz, DMSO-d₆) δ=5.22 (s, 1H), 4.71 (s, 2H), 3.86-3.76 (m, 4H), 2.89 (tt, J=7.8, 9.9 Hz, 1H), 2.04 (dd, J=8.0, 13.4 Hz, 1H), 1.96-1.82 (m, 2H), 1.78-1.66 (m, 2H), 1.65-1.53 (m, 1H), 1.47 (s, 9H).

### Step 4: benzyl (1-(tert-butyl)-3-(1,4-dioxaspiro[4.4]nonan-7-yl)-1H-pyrazol-5-yl)carbamate

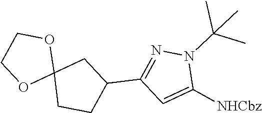 [SMILES: CC(C)(C)n1nc(C2CCC3(C2)OCCO3)cc1NC(=O)OCc1ccccc1]

To a solution of 1-(tert-butyl)-3-(1,4-dioxaspiro[4.4]nonan-7-yl)-1H-pyrazol-5-amine (18 g, 67.83 mmol) in acetone (1 L), benzyl carbonochloridate (23.14 g, 135.67 mmol) was added portion wise at 0° C. The mixture was stirred at room temperature for 2 h, then NaHCO₃ (18.24 g, 217.07 mmol) was added in portions. The mixture was further stirred at this temperature for 26 h. The reaction mixture was filtered and concentrated under reduced pressure to afford the crude product (28.5 g, >99% yield), which was used in the next step without further purification.

### Step 5: benzyl (1-(tert-butyl)-3-(3-oxocyclopentyl)-1H-pyrazol-5-yl)carbamate

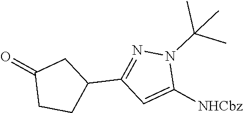 [SMILES: CC(C)(C)n1nc(C2CCC(=O)C2)cc1NC(=O)OCc1ccccc1]

A solution of benzyl (1-(tert-butyl)-3-(1,4-dioxaspiro[4.4]nonan-7-yl)-1H-pyrazol-5-yl)carbamate (27.5 g, 68.84 mmol) in acetone (3 L) and water (300 mL) was treated with 4-toluenesulfonic acid (1.54 g, 8.95 mmol). The mixture was stirred at 60° C. for 4 h under nitrogen atmosphere. The reaction mixture was concentrated under reduced pressure to remove the most of acetone. The aqueous residue was extracted with DCM (500 mL×3). The combined organic phase was washed with brine (500 mL), dried with anhydrous Na₂SO₄, then filtered and concentrated under vacuum. The residue was purified by silica gel chromatography, eluting with PE/EA (10:1) to afford the product (23 g, 87% yield). ¹H NMR (400 MHz, DMSO-d₆) δ=9.12 (br s, 1H), 7.44-7.28 (m, 5H), 6.03 (s, 1H), 5.12 (s, 2H), 3.35-3.32 (m, 1H), 2.48-2.41 (m, 1H), 2.33-2.18 (m, 4H), 1.97-1.87 (m, 1H), 1.48 (s, 9H).

### Step 6: 3-(5-(((benzyloxy)carbonyl)amino)-1-(tert-butyl)-1H-pyrazol-3-yl)cyclopent-1-en-1-yl trifluoromethanesulfonate

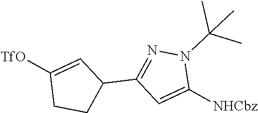 [SMILES: CC(C)(C)n1nc(C2C=C(OS(=O)(=O)C(F)(F)F)CC2)cc1NC(=O)OCc1ccccc1]

A solution of benzyl (1-(tert-butyl)-3-(3-oxocyclopentyl)-1H-pyrazol-5-yl)carbamate (10 g, 28.1 mmol) in THF (100 mL) was added Lithium bis(trimethylsilyl)amide (56 mmol, 1 M in THF) dropwise at −78° C. The solution was further stirred for 1 h at this temperature and then 1,1,1-trifluoro-N-phenyl-N-((trifluoromethyl)sulfonyl)methanesulfonamide (11 g, 31 mmol) in 50 ml THF was added through a syringe. The mixture was allowed to slowly recover to room temperature and stirred for overnight. The reaction was quenched by aq. NH₄Cl and extracted with EA (500 mL×3). The combined organic layer was washed with brine (200 mL), dried over anhydrous Na₂SO₄, filtered, and concentrated under vacuum. The residue was purified by silica gel chromatography, eluting with PE/EA (4:1) to afford the product (9.5 g, 69% yield). LC-MS (ESI): m/z [M+H]⁺=488.3.

### Step 7: benzyl (1-(tert-butyl)-3-(3-(4,4,5,5-tetramethyl-1,3,2-dioxaborolan-2-yl)cyclopent-2-en-1-yl)-1H-pyrazol-5-yl)carbamate

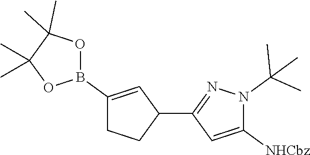 [SMILES: CC(C)(C)n1nc(C2C=C(B3OC(C)(C)C(C)(C)O3)CC2)cc1NC(=O)OCc1ccccc1]

A solution of 3-(5-(((benzyloxy)carbonyl)amino)-1-(tert-butyl)-1H-pyrazol-3-yl)cyclopent-1-en-1-yl trifluoromethanesulfonate (9.5 g, 19.5 mmol) in 100 mL dioxane was added Bis(pinacolato)diboron (10.1 g, 40 mmol), Pd(dppf)Cl₂ (1.46 mg, 2 mmol) and K₃PO₄ (12.4 g, 58.5 mmol). The mixture was heated to 100° C. and stirred for 4 h under nitrogen. The solution was removed under vacuum and the residue was purified by silica gel chromatography, eluting with PE/EA (4:1) to afford the product (5.1 g, 56% yield). LC-MS (ESI): m/z [M+H]⁺=466.3.

### Step 8: benzyl (1-(tert-butyl)-3-(3-(4-(tert-butyl)pyridin-2-yl)cyclopent-2-en-1-yl)-1H-pyrazol-5-yl)carbamate

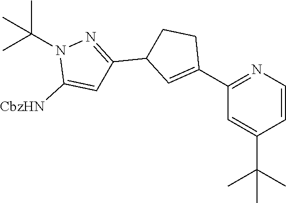 [SMILES: CC(C)(C)c1ccnc(C2=CC(c3cc(NC(=O)OCc4ccccc4)n(C(C)(C)C)n3)CC2)c1]

A solution of benzyl (1-(tert-butyl)-3-(3-(4,4,5,5-tetramethyl-1,3,2-dioxaborolan-2-yl)cyclopent-2-en-1-yl)-1H-pyrazol-5-yl)carbamate (930 mg, 2 mmol) in a mixture of dioxane and H₂O (10:1) was added 2-bromo-4-(tert-butyl)pyridine (639 mg, 3 mmol), Pd(dppf)Cl₂ (146 mg, 0.2 mmol) and K₂CO₃ (828 mg, 6 mmol). The mixture was heated to 100° C. and stirred for 3 h under nitrogen. The solution was removed under vacuum and the residue was purified by silica gel chromatography, eluting with PE/EA (1:1) to afford the product (566 mg, 60% yield). LC-MS (ESI): m/z [M+H]⁺=473.4.

### Step 9: 1-(tert-butyl)-3-(3-(4-(tert-butyl)pyridin-2-yl)cyclopentyl)-1H-pyrazol-5-amine

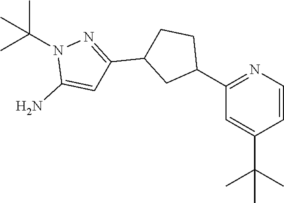 [SMILES: CC(C)(C)c1ccnc(C2CCC(c3cc(N)n(C(C)(C)C)n3)C2)c1]

To a solution of benzyl (1-(tert-butyl)-3-(3-(4-(tert-butyl)pyridin-2-yl)cyclopent-2-en-1-yl)-1H-pyrazol-5-yl)carbamate (566 mg, 1.2 mmol) in THF (20 mL) was added Pd/C (10%, wet, 200 mg). The suspension was degassed and purged with hydrogen 3 times. The resulting mixture was stirred at room temperature under a hydrogen balloon for 3 h. The mixture was filtered, and the filter cake was washed with EA (10 mL×3). The filtrate was concentrated under reduced pressure to afford the product (322 mg, 79% yield). LC-MS (ESI): m/z [M+H]⁺=341.4.

### Step 10: 4-((1-(tert-butyl)-3-(3-(4-(tert-butyl)pyridin-2-yl)cyclopentyl)-1H-pyrazol-5-yl)amino)-3-fluorobenzenesulfonamide

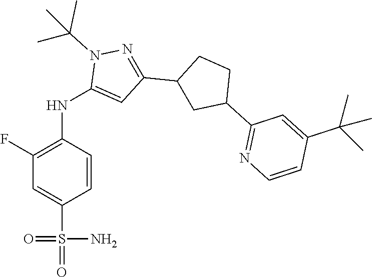 [SMILES: CC(C)(C)c1ccnc(C2CCC(c3cc(Nc4ccc(S(N)(=O)=O)cc4F)n(C(C)(C)C)n3)C2)c1]

To a mixture of 4-bromo-3-fluorobenzenesulfonamide (38 mg, 0.15 mmol) and 1-(tert-butyl)-3-(3-(4-(tert-butyl)pyridin-2-yl)cyclopentyl)-1H-pyrazol-5-amine (50 mg, 0.15 mmol) in andydrous t-BuOH (5 mL) was added BrettPhos Pd G3 (9 mg, 0.01 mmol) and K₂CO₃ (61 mg, 0.45 mmol). The reaction mixture was heated to 110° C. and stirred under nitrogen atmosphere for 16 hrs. The mixture was concentrated under reduced pressure. The residue was purified by silica gel column chromatography, eluting with PE/EA (0% to 70%) to afford the product (44 mg, 57%). LC-MS (ESI): m/z [M+H]⁺=514.4.

### Step 11: 4-((5-(3-(4-(tert-butyl)pyridin-2-yl)cyclopentyl)-1H-pyrazol-3-yl)amino)-3-fluorobenzenesulfonamide

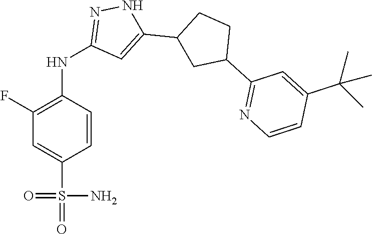 [SMILES: CC(C)(C)c1ccnc(C2CCC(c3cc(Nc4ccc(S(N)(=O)=O)cc4F)n[nH]3)C2)c1]

To a solution of 4-((1-(tert-butyl)-3-(3-(4-(tert-butyl)pyridin-2-yl)cyclopentyl)-1H-pyrazol-5-yl)amino)-3-fluorobenzenesulfonamide (44 mg, 0.09 mmol) in DCM (15 mL) was added TfOH (3 mL). The mixture was stirred at rt for 2 h. The mixture was quenched by aq NaHCO₃ (50 mL) at 0° C. and extracted with DCM (3×50 mL). The combined organic layers were combined, dried over Na₂SO₄, filtered and concentrated under vacuum. The residue was purified by silica gel chromatography, eluting with PE/EA (0% to 100%) to afford the product (7.8 mg, 19% yield). ¹H NMR (500 MHz, DMSO-d₆) δ 12.04 (s, 1H), 8.63 (d, J=2.2 Hz, 1H), 8.41 (d, J=5.2 Hz, 1H), 8.20 (s, 1H), 7.50 (d, J=9.7 Hz, 2H), 7.27 (t, J=2.9 Hz, 1H), 7.21 (dd, J=5.3, 1.8 Hz, 1H), 7.17 (s, 2H), 5.86 (s, 1H), 3.45-3.35 (m, 1H), 3.28-3.18 (m, 1H), 2.42-2.34 (m, 1H), 2.25-2.05 (m, 2H), 2.02-1.75 (m, 3H), 1.27 (s, 9H). LC-MS (ESI): m/z [M+H]⁺=458.36.

### Example 2: 4-((5-(3-(4-(tert-butyl)-6-methylpyridin-2-yl)cyclopentyl)-1H-pyrazol-3-yl)amino)-3-fluorobenzenesulfonamide

### Step 1: 4-tert-Butyl-2-methyl-pyridine 1-oxide

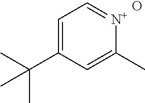

To a solution of 2-methyl-4-tert-butyl-pyridine (5.5 g, 37 mmol) in DCM (100 mL) was added methyltrioxorhenium (VII) (46 mg, 0.19 mmol) with vigorous stirring. 30% hydrogen peroxide (13 mL) was added and the mixture was stirred vigorously for 18 h. Quenched the reaction by adding drop wise aqueous manganese dioxide. The layers were separated and the aqueous was re-extracted with dichloromethane (3×30 mL). The combined organic layers were filtered, dried and concentrated to yield the product (5.5 g, 89%). LC-MS(ESI), m/z [M+H]⁺=166.

### Step 2: 2-chloro-6-methyl-4-tert-butyl-pyridine

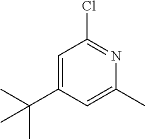

4-tert-butyl-2-methyl-pyridine 1-oxide (2.00 g, 12.10 mmol) was added to phosphorus oxychloride (15 mL) and stirred at 100° C. for 36 h. The mixture was concentrated under vacuum, treated with ice water, and basified with aqueous sodium bicarbonate. The mixture was extract with dichloromethane (3×75 mL). The combined organic layers were filtered, dried and concentrated. The residue was purified by silica gel column chromatography, eluting with EA/PE (0-10%) to give the product (812 mg, 36.6%). LC-MS (ESI): m/z [M+H]⁺=184.2

### Step 3: 4-((5-(3-(4-(tert-butyl)-6-methylpyridin-2-yl)cyclopentyl)-1H-pyrazol-3-yl)amino)-3-fluorobenzenesulfonamide

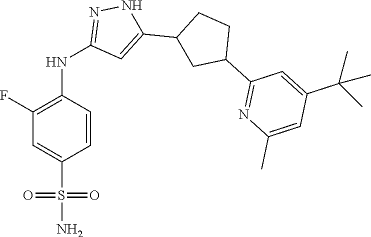 [SMILES: Cc1cc(C(C)(C)C)cc(C2CCC(c3cc(Nc4ccc(S(N)(=O)=O)cc4F)n[nH]3)C2)n1]

The titled compound was synthesized in the procedures similar to Example 1. ¹H NMR (500 MHz, DMSO-d₆) δ 12.15 (s, 1H), 8.64 (s, 1H), 8.18 (s, 1H), 7.53-7.50 (m, 1H), 7.49-7.48 (m, 1H), 7.26-7.05 (m, 4H), 5.88 (s, 1H), 3.29-3.20 (m, 2H), 2.51 (s, 3H), 2.44-2.34 (m, 1H), 2.25-2.07 (m, 2H), 2.01-1.79 (m, 3H), 1.27 (s, 9H). LC-MS (ESI): m/z [M+H]⁺=472.4.

### Example 3: 4-((5-(3-(5-(tert-butyl)pyridazin-3-yl)cyclopentyl)-1H-pyrazol-3-yl)amino)-3-fluorobenzenesulfonamide

### Step 1: benzyl (1-(tert-butyl)-3-(3-(5-(tert-butyl)-6-chloropyridazin-3-yl)cyclopent-2-en-1-yl)-1H-pyrazol-5-yl)carbamate

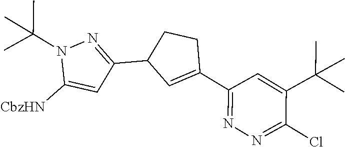 [SMILES: CC(C)(C)c1cc(C2=CC(c3cc(NC(=O)OCc4ccccc4)n(C(C)(C)C)n3)CC2)nnc1Cl]

A mixture of 4-(tert-butyl)-3,6-dichloropyridazine (510 mg, 2.5 mmol), benzyl (1-(tert-butyl)-3-(3-(4,4,5,5-tetramethyl-1,3,2-dioxaborolan-2-yl)cyclopent-2-en-1-yl)-1H-pyrazol-5-yl)carbamate (1.4 g, 3 mmol), Pd(dppf)Cl₂ (203 mg, 0.25 mmol) and Na₂CO₃ (795 mg, 7.5 mmol) in dioxane (20 mL) and H₂O (5 mL) was stirred at 80° C. for 16 h under a nitrogen atmosphere. The mixture was concentrated under vacuum. The residue was purified by silica gel column chromatography, eluting with EA/PE (0-45%) to afford the product (1.15 g, 90.6%). LC-MS (ESI): m/z [M+H]⁺=508.2.

### Step 2: 1-(tert-butyl)-3-(3-(5-(tert-butyl)pyridazin-3-yl)cyclopentyl)-1H-pyrazol-5-amine

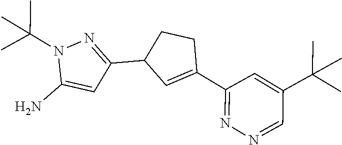 [SMILES: CC(C)(C)c1cnnc(C2=CC(c3cc(N)n(C(C)(C)C)n3)CC2)c1]

To a solution of benzyl (1-(tert-butyl)-3-(3-(5-(tert-butyl)-6-chloropyridazin-3-yl)cyclopent-2-en-1-yl)-1H-pyrazol-5-yl)carbamate (1.15 g, 2.3 mmol) in MeOH (20 mL) was added Pd/C (150 mg). The mixture was stirred at rt for 5 h under a hydrogen atmosphere. The mixture was filtered and the filtrate was concentrated under vacuum to afford the product (567 mg, crude). LC-MS (ESI): m/z [M+H]⁺=342.2.

### Step 3: 4-((1-(tert-butyl)-3-(3-(5-(tert-butyl)pyridazin-3-yl)cyclopentyl)-1H-pyrazol-5-yl)amino)-3-fluorobenzenesulfonamide

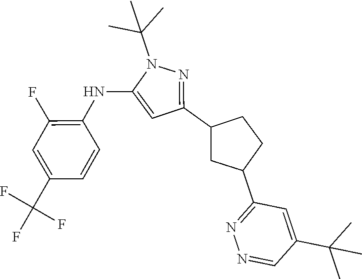 [SMILES: CC(C)(C)c1cnnc(C2CCC(c3cc(Nc4ccc(C(F)(F)F)cc4F)n(C(C)(C)C)n3)C2)c1]

A mixture of 4-bromo-3-fluorobenzenesulfonamide (48 mg, 0.19 mmol), 1-(tert-butyl)-3-(3-(5-(tert-butyl)pyridazin-3-yl)cyclopentyl)-1H-pyrazol-5-amine (45 mg, 0.13 mmol), Brettphos Pd G3 (17.2 mg, 0.02 mmol) and K₂CO₃ (79 mg, 0.57 mmol) in t-BuOH (5 mL) was stirred at 110° C. for 16 h under a nitrogen atmosphere. The mixture was concentrated under vacuum. The residue was purified by silica gel column chromatography, eluting with EA/PE (0-100%) to afford the product (58 mg, 59.3%). LC-MS (ESI): m/z [M+H]⁺=515.3.

### Step 4: 4-((5-(3-(5-(tert-butyl)pyridazin-3-yl)cyclopentyl)-1H-pyrazol-3-yl)amino)-3-fluorobenzenesulfonamide

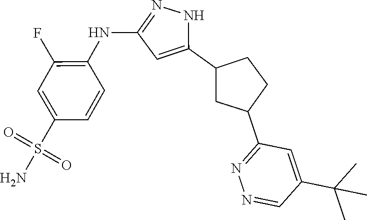 [SMILES: CC(C)(C)c1cnnc(C2CCC(c3cc(Nc4ccc(S(N)(=O)=O)cc4F)n[nH]3)C2)c1]

To a solution of 4-((1-(tert-butyl)-3-(3-(5-(tert-butyl)pyridazin-3-yl)cyclopentyl)-1H-pyrazol-5-yl)amino)-3-fluorobenzenesulfonamide (58 mg, 0.11 mmol) in DCM (5 mL) was added TfOH (1 mL). The mixture was stirred at rt for 2 h under a nitrogen atmosphere. The mixture was diluted with DCM (50 mL), washed with sat NaHCO₃ (3×30 mL). The combined organic layers were dried over Na₂SO₄, filtered and concentrated under vacuum. The residue was purified by Prep-HPLC to afford product (4.07 mg, 7.9%). ¹H NMR (500 MHz, DMSO-d₆) δ 12.06 (s, 1H), 9.21 (s, 1H), 8.65 (s, 1H), 8.24-8.15 (m, 1H), 7.56-7.47 (m, 3H), 7.17 (s, 2H), 5.88 (s, 1H), 3.63-3.45 (m, 1H), 3.31-3.22 (m, 1H), 2.48-2.44 (m, 1H), 2.31-2.12 (m, 2H), 2.07-1.94 (m, 2H), 1.93-1.84 (m, 1H), 1.31 (s, 9H). LC-MS (ESI): m/z [M+H]⁺=459.2.

### Example 4: 4-((5-(3-(4-(tert-butyl)pyrimidin-2-yl)cyclopentyl)-1H-pyrazol-3-yl)amino)-3-fluorobenzenesulfonamide

### Step 1: benzyl (1-(tert-butyl)-3-(3-(4-(tert-butyl)pyrimidin-2-yl)cyclopent-2-en-1-yl)-1H-pyrazol-5-vi)carbamate

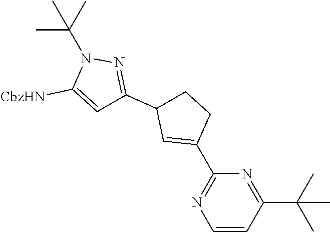 [SMILES: CC(C)(C)c1ccnc(C2=CC(c3cc(NC(=O)OCc4ccccc4)n(C(C)(C)C)n3)CC2)n1]

To a solution of benzyl (1-(tert-butyl)-3-(3-(4,4,5,5-tetramethyl-1,3,2-dioxaborolan-2-yl)cyclopent-2-en-1-yl)-1H-pyrazol-5-yl)carbamate (500 mg, 1.075 mmol) and 4-(tert-butyl)-2-chloropyrimidine (277.2 mg, 1.612 mmol) in dioxane/H₂O (10:2 mL) was added Pd(dppf)Cl₂ (99.6 mg, 0.107 mmol) and K₃PO₄ (683.7 mg, 3.225 mmol). The reaction mixture was stirred for 6 hours at 100° C. under nitrogen. The resulting mixture was cooled to room temperature, concentrated under reduce pressure, purified by silica gel column chromatography, eluting with PE/EA (1:1) to afford the product (400 mg, 78.5%). LC-MS (ESI): m/z [M+H]⁺=475.3.

### Step 2: 1-(tert-butyl)-3-(3-(4-(tert-butyl)pyrimidin-2-yl)cyclopentyl)-1H-pyrazol-5-amine

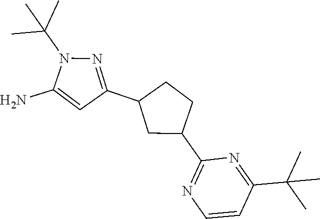 [SMILES: CC(C)(C)c1ccnc(C2CCC(c3cc(N)n(C(C)(C)C)n3)C2)n1]

To a solution of benzyl (1-(tert-butyl)-3-(3-(4-(tert-butyl)pyrimidin-2-yl)cyclopent-2-en-1-yl)-1H-pyrazol-5-yl)carbamate (400 mg, 0.84 mmol) in MeOH (10 mL) was added Pd/C (10% on carbon, 200 mg). The reaction mixture was degassed and purged with hydrogen 3 times and stirred for 2 hours at room temperature. The resulting mixture was filtered and the filtrated was concentrated in vacuum to afford the product (200 mg, 69%). LC-MS (ESI): m/z [M+H]⁺=343.3.

### Step 3: 4-((1-(tert-butyl)-3-(3-(4-(tert-butyl)pyrimidin-2-yl)cyclopentyl)-1H-pyrazol-5-yl)amino)-3-fluorobenzenesulfonamide

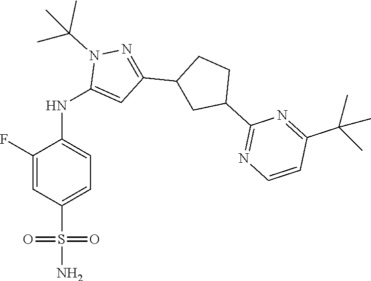 [SMILES: CC(C)(C)c1ccnc(C2CCC(c3cc(Nc4ccc(S(N)(=O)=O)cc4F)n(C(C)(C)C)n3)C2)n1]

To a solution of 1-(tert-butyl)-3-(3-(4-(tert-butyl)pyrimidin-2-yl)cyclopentyl)-1H-pyrazol-5-amine (50 mg, 0.146 mmol) and 4-bromo-3-fluorobenzenesulfonamide (36.9 mg, 0.146 mmol) in dioxane (5 mL) was added Pd₂dba₃ (13.5 mg, 0.015 mmol), Xantphos (17.28 mg, 0.030 mmol) and K₃PO₄ (92.86 mg, 0.438 mmol). The reaction mixture was stirred for 6 hours at 90° C. under nitrogen. The resulting mixture was cooled to room temperature, concentrated under reduce pressure, purified by silica gel column chromatography, eluting with EA to afford the product (300 mg, 44.1%). LC-MS (ESI): m/z [M+H]⁺=516.3.

### Step 4: 4-((5-(3-(4-(tert-butyl)pyrimidin-2-yl)cyclopentyl)-1H-pyrazol-3-yl)amino)-3-fluorobenzenesulfonamide

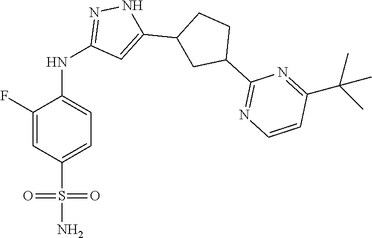 [SMILES: CC(C)(C)c1ccnc(C2CCC(c3cc(Nc4ccc(S(N)(=O)=O)cc4F)n[nH]3)C2)n1]

To a solution of 4-((1-(tert-butyl)-3-(3-(4-(tert-butyl)pyrimidin-2-yl)cyclopentyl)-1H-pyrazol-5-yl)amino)-3-fluorobenzenesulfonamide (90 mg, 0.138 mmol) in DCM (5 mL) was added TfOH (1 mL). The reaction mixture was stirred for 2 h at room temperature. The resulting mixture was neutralized with Na₂CO₃ aqueous at 0° C. and extracted with EA (3×10 mL). The combined organic layers were combined, washed with brine, dried over Na₂SO₄ and concentrated. The residue was purified by column chromatography on silica gel, eluting with 100% EA to afford the crude product (20 mg), which was further purified by prep HPLC (Waters XSelect C18: RD-CO-094 column, eluting with a gradient of acetonitrile/water containing 0.1% FA, 20%-40%) to afford the product (3.0 mg, 11.2%). ¹H NMR (500 MHz, DMSO-d₆) δ 12.04 (s, 1H), 8.64 (d, J=5.3 Hz, 2H), 8.17 (t, J=8.4 Hz, 1H), 7.50 (d, J=10.2 Hz, 2H), 7.34 (d, J=5.3 Hz, 1H), 7.17 (s, 2H), 5.84 (s, 1H), 3.50-3.40 (m, 1H), 3.27-3.13 (m, 1H), 2.48-2.43 (m, 2H), 2.20-1.95 (m, 4H), 1.86-1.79 (m, 1H), 1.29 (s, 9H). LC-MS (ESI): m/z [M+H]⁺=459.2.

### Example 5: 3-fluoro-4-((5-(3-(4-isopropylpyrimidin-2-yl)cyclopentyl)-1H-pyrazol-3-yl)amino)benzenesulfonamide

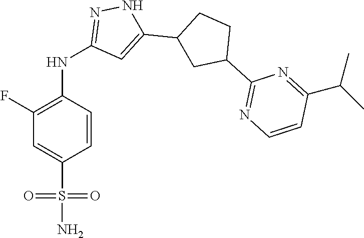 [SMILES: CC(C)c1ccnc(C2CCC(c3cc(Nc4ccc(S(N)(=O)=O)cc4F)n[nH]3)C2)n1]

The titled compound was synthesized in the procedures similar to Example 1. ¹H NMR (500 MHz, DMSO-d₆) δ 12.01 (s, 1H), 8.62 (d, J=5.1 Hz, 2H), 8.20 (t, J=8.6 Hz, 1H), 7.50 (d, J=9.9 Hz, 2H), 7.22 (d, J=5.2 Hz, 1H), 7.17 (s, 2H), 5.86 (d, J=10.8 Hz, 1H), 3.47-3.47 (m, 1H), 2.99-2.89 (m, 1H), 2.44 (s, 2H), 2.12 (dd, J=14.4, 7.2 Hz, 2H), 2.05 (d, J=8.5 Hz, 1H), 1.99 (dd, J=22.3, 10.9 Hz, 1H), 1.82 (s, 1H), 1.26-1.20 (m, 6H). LC-MS (ESI): m/z [M+H]⁺=445.2.

### Example 6: 4-((5-(3-(5-(tert-butyl)oxazol-2-yl)cyclopentyl)-1H-pyrazol-3-yl)amino)-3-fluorobenzenesulfonamide

### Step 1: cis-3-oxabicyclo[3.2.1]octane-2,4-dione

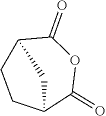

Into a 1 L round-bottom flask was added cis-cyclopentane-1,3-dicarboxylic acid (50.0 g, 316.45 mmol) and Ac₂O (100 mL) at room temperature. The resulting mixture was stirred overnight at 140° C. The resulting mixture was concentrated under reduced pressure, and the residue was purified by column chromatography on silica gel, eluting with with PE/EA (5:1) to afford the product (30.0 g, 67.71%).

### Step 2: cis-3-((3,3-dimethyl-2-oxobutyl)carbamoyl)cyclopentane-1-carboxylic acid

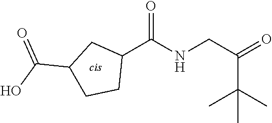 [SMILES: CC(C)(C)C(=O)CNC(=O)C1CCC(C(=O)O)C1]

To a solution of cis-3-oxabicyclo[3.2.1]octane-2,4-dione (30.0 g, 214.28 mmol) in THF (300 mL) was added 1-amino-3,3-dimethylbutan-2-one (49.28 g, 428.57 mmol) and TEA (43.29 g, 428.57 mmol) at room temperature. The resulting mixture was stirred 2 h at room temperature. The resulting mixture was concentrated under reduced pressure to afford the product (45.0 g, 82.35%). LC-MS (ESI): m/z [M−H]⁻=254.15.

### Step 3: cis-methyl 3-((3,3-dimethyl-2-oxobutyl)carbamoyl)cyclopentane-1-carboxylate

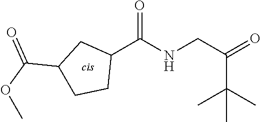 [SMILES: COC(=O)C1CCC(C(=O)NCC(=O)C(C)(C)C)C1]

To a solution of cis-3-((3,3-dimethyl-2-oxobutyl)carbamoyl)cyclopentane-1-carboxylic acid (45.0 g, 176.47 mmol) in MeOH (250 mL) was added H₂SO₄ (17.0 g, 176.47 mmol) at room temperature. The resulting mixture was stirred overnight at room temperature. The mixture was neutralized to PH=7 with sodium bicarbonate aqueous solution and extracted with 300 mL of DCM. The combined organic layers were washed with saturated aqueous sodium chloride solution and dried over anhydrous sodium sulfate. After filtration, the filtrate was concentrated under reduced pressure to afford the product (43.0 g, 90.58%). LC-MS (ESI): m/z [M+H]⁺=270.2.

### Step 4: cis-methyl 3-(5-(tert-butyl)oxazol-2-yl)cyclopentane-1-carboxylate

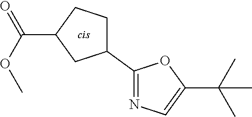 [SMILES: COC(=O)C1CCC(c2ncc(C(C)(C)C)o2)C1]

Into a 500-mL round-bottom flask were added cis-methyl 3-((3,3-dimethyl-2-oxobutyl)carbamoyl)cyclopentane-1-carboxylate (41.0 g, 152.41 mmol) and POCl₃ (50 mL) at room temperature. The resulting mixture was stirred 1 h at 60° C. The resulting mixture was concentrated under reduced pressure. The residue was purified by silica gel column chromatography, eluted with PE/EA (100:0~50:50) to afford the product (35.0 g, 91.49%). LC-MS (ESI): m/z [M+H]⁺=251.2.

### Step 5: 3-(3-(5-(tert-butyl)oxazol-2-yl)cyclopentyl)-3-oxopropanenitrile

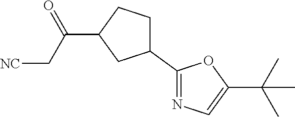 [SMILES: CC(C)(C)c1cnc(C2CCC(C(=O)CC#N)C2)o1]

Into a solution of CH₃CN (9.8 g, 239.02 mmol) in THF (100 mL) was added n-BuLi (2.5 M, 95.61 mL, 239.02 mmol) dropwised at −78° C. The resulting mixture was stirred for 30 min at −78° C. under nitrogen atmosphere. A solution of cis-methyl 3-(5-(tert-butyl)oxazol-2-yl)cyclopentane-1-carboxylate (30.0 g, 119.52 mmol) in THF (50 mL) was added dropwised at −78° C. and further stirred for 1 h at −78° C. The reaction was quenched with sat NH₄Cl (aq.) at 0° C. and extracted with ethyl acetate (3×400 mL). The combined organic layers were washed with brine, dried over anhydrous sodium sulfate, filtrated, and concentrated under reduced pressure. The residue was purified by column chromatography, eluting with petroleum ether:ethyl acetate (100:0~50:50) to give the product (25.0 g, 80.44% yield). LC-MS (ESI): m/z [M+H]⁺=260.2.

### Step 6: 5-(3-(5-(tert-butyl)oxazol-2-yl)cyclopentyl)-1H-pyrazol-3-amine

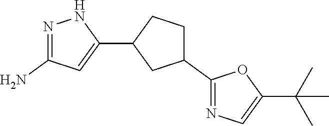 [SMILES: CC(C)(C)c1cnc(C2CCC(c3cc(N)n[nH]3)C2)o1]

To a solution of 3-(3-(5-(tert-butyl)oxazol-2-yl)cyclopentyl)-3-oxopropanenitrile (4.0 g, 15.38 mmol), NH₂NH₂·H₂O (0.85 g, 16.92 mmol), PTSA (160.0 mg) and EtOH (40 mL) at room temperature. The resulting mixture was stirred 1 h at 90° C. The resulting mixture was concentrated under reduced pressure, and the residue was purified by silica gel column chromatography, eluted with DCM/MeOH (100:0~95:5) to the product (1.9024 g, 45.13%). ¹H NMR (300 MHz, DMSO-d₆) δ 6.65 (d, J=1.2 Hz, 1H), 5.21 (d, J=8.3 Hz, 1H), 3.33 (s, 2H), 2.35 (dt, J=13.6 Hz, 1H), 2.01 (s, 5H), 2.08-1.73 (m, 1H), 1.66 (s, 1H), 1.23 (s, 9H). LC-MS (ESI): m/z [M+H]⁺=274.3.

### Step 7: 4-((5-(3-(5-(tert-butyl)oxazol-2-yl)cyclopentyl)-1H-pyrazol-3-yl)amino)-3-fluorobenzenesulfonamide

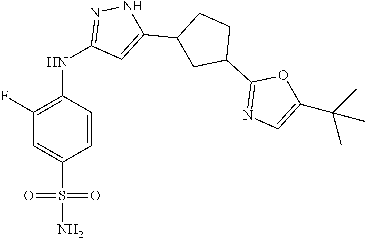 [SMILES: CC(C)(C)c1cnc(C2CCC(c3cc(Nc4ccc(S(N)(=O)=O)cc4F)n[nH]3)C2)o1]

The titled compound was synthesized in the procedures similar to Example 1, step 10 in a racemic form, which was further separated by Chiral Prep-HPLC to give:

Enantiomer 1 (Example 6a, 100% ee); Retention time: 4.965 min. ¹H NMR (500 MHz, DMSO-d₆) δ 12.01 (s, 1H), 8.63 (d, J=1.8, 1H), 8.19 (t, J=8.5, 1H), 7.51 (s, 1H), 7.49 (s, 1H), 7.17 (s, 2H), 6.66 (s, 1H), 5.83 (s, 1H), 3.40-3.35 (m, 1H), 3.23-3.13 (m, 1H), 2.48-2.40 (m, 1H), 2.15-2.06 (m, 2H), 1.97 (m, 1H), 1.89 (m, 1H), 1.78-1.68 (m, 1H), 1.23 (s, 9H). LC-MS (ESI): m/z [M+H]⁺=448.34.

Enantiomer 2 (Example 6b, 100% ee); Retention time: 4.485 min. ¹H NMR (500 MHz, DMSO-d₆) δ 12.01 (s, 1H), 8.63 (d, J=1.8, 1H), 8.19 (t, J=8.5, 1H), 7.51 (s, 1H), 7.49 (s, 1H), 7.17 (s, 2H), 6.66 (s, 1H), 5.83 (s, 1H), 3.40-3.35 (m, 1H), 3.23-3.13 (m, 1H), 2.48-2.40 (m, 1H), 2.15-2.06 (m, 2H), 1.97 (m, 1H), 1.89 (m, 1H), 1.78-1.68 (m, 1H), 1.23 (s, 9H). LC-MS (ESI): m/z [M+H]⁺=448.34;

Enantiomer 3 (Example 6c, 100% ee); Retention time: 4.980 min. ¹H NMR (500 MHz, DMSO-d₆) δ 12.00 (s, 1H), 8.64 (s, 1H), 8.19 (t, J=7.8, 1H), 7.51 (s, 1H), 7.49 (s, 1H), 7.18 (s, 2H), 6.67 (s, 1H), 5.86 (s, 1H), 3.44 (m, 1H), 3.27-3.22 (m, 1H), 2.28 (m, 1H), 2.22-2.11 (m, 2H), 2.08-1.99 (m, 1H), 1.98-1.88 (m, 1H), 1.77-1.66 (m, 1H), 1.24 (s, 9H). LC-MS (ESI): m/z [M+H]⁺=448.30.

Enantiomer 4 (Example 6d, 100% ee); Retention time: 4.860 min. ¹H NMR (500 MHz, DMSO-d₆) δ 12.00 (s, 1H), 8.64 (s, 1H), 8.19 (t, J=7.8, 1H), 7.51 (s, 1H), 7.49 (s, 1H), 7.18 (s, 2H), 6.67 (s, 1H), 5.86 (s, 1H), 3.44 (m, 1H), 3.27-3.22 (m, 1H), 2.28 (m, 1H), 2.22-2.11 (m, 2H), 2.08-1.99 (m, 1H), 1.98-1.88 (m, 1H), 1.77-1.66 (m, 1H), 1.24 (s, 9H). LC-MS (ESI): m/z [M+H]⁺=448.33;

Chiral analytical method: Column: CHIRALPAK IE 4.6*250 mm 5 μm; Mobile phase: A for MtBE (0.1% 2M NH₃ MeOH) and B for EtOH; Gradient: Mobile Phase A: Mobile Phase B=20:80 (v/v); HPLC Equipment: HPLC-Agilent; Column temperature: 35° C.

Chiral Prep-HPLC Condition: CHIRALPAK ID 20*250 mm 5 μm; Mobile phase: A for MtBE and B for MeOH:DCM=50:50; Gradient: Mobile Phase A: Mobile Phase B=60:40 (v/v); Flow Rate: 20 mL/min, Wavelength: UV 220 nm, Prep-HPLC Equipment: Prep-HPLC-Gilson; Column temperature: 25° C.

### Example 7: 3-fluoro-4-((5-(3-(5-isopropyloxazol-2-yl)cyclopentyl)-1H-pyrazol-3-yl)amino)benzenesulfonamide

### Step 1: cis-3-((3-methyl-2-oxobutyl)carbamoyl)cyclopentane-1-carboxylic acid

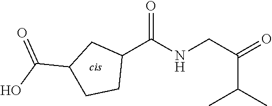 [SMILES: CC(C)C(=O)CNC(=O)C1CCC(C(=O)O)C1]

To a solution of cis-3-oxabicyclo[3.2.1]octane-2,4-dione (3.85 g, 27.5 mmol) in THF (100 mL) were added 1-amino-3-methylbutan-2-one hydrochloride (4.9 g, 35.7 mmol) and TEA (5.55 g, 55 mmol) at 0° C. The reaction was stirred at rt for 2 h. The reaction was concentrated under reduced pressure to give the crude product (6.6 g). LC-MS (ESI): m/z [M−H]—=242.

### Step 2: cis-methyl 3-((3-methyl-2-oxobutyl)carbamoyl)cyclopentane-1-carboxylate

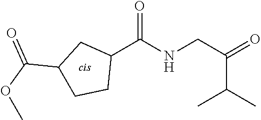 [SMILES: COC(=O)C1CCC(C(=O)NCC(=O)C(C)C)C1]

To a solution of cis-3-((3-methyl-2-oxobutyl)carbamoyl)cyclopentane-1-carboxylic acid (6.6 g, 27.38 mmol) in MeOH (100 mL) was added H₂SO₄ (1 mL), then stirred at 65° C. for 2 h. The mixture was concentrated under vacuum and the residue was purified by silica gel column chromatography, eluting with PE:EA (100:0 to 70:30) to give the product (7.2 g, 95% yield). LCMS (ESI) m/z [M+H]⁺=256.

### Step 3: cis-methyl 3-(5-isopropyloxazol-2-yl)cyclopentane-1-carboxylate

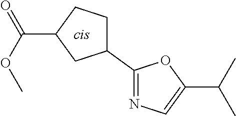 [SMILES: COC(=O)C1CCC(c2ncc(C(C)C)o2)C1]

cis-methyl 3-((3-methyl-2-oxobutyl)carbamoyl)cyclopentane-1-carboxylate (7.2 g, 28.2 mmol) was dissolved in POCl₃ (40 mL). The resulting solution was stirred for 1 h at 60° C. The resulting mixture was concentrated under vacuum and H₂O (100 mL) was added. The mixture was adjusted to Ph=8 with Na₂CO₃ solution (2M) and extracted with 2×80 mL of DCM. The resulting mixture was concentrated under vacuum and the residue was purified by silica gel column chromatography, eluted with PE:EA (100:0 to 85:15) to give the product (6.2 g, 92.6%). LC-MS (ESI): m/z [M+H]⁺=238.

### Step 4: 3-(3-(5-isopropyloxazol-2-yl)cyclopentyl)-3-oxopropanenitrile

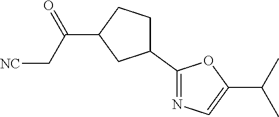 [SMILES: CC(C)c1cnc(C2CCC(C(=O)CC#N)C2)o1]

Into a solution of CH₃CN (1.38 g, 33.75 mmol) in THF (100 mL) was added n-BuLi (2.5 M, 95.61 mL, 239.02 mmol) dropwised at −78° C. The resulting mixture was stirred for 30 min at −78° C. under nitrogen atmosphere. A solution of cis-methyl 3-(5-isopropyloxazol-2-yl)cyclopentane-1-carboxylate (30.0 g, 119.52 mmol) in THF (50 mL) was then added dropwised and further stirred for 1 h at −78° C. The reaction was quenched with sat NH₄Cl (aq.) at 0° C. and extracted with ethyl acetate (3×400 mL). The combined organic layers were washed with brine, dried over anhydrous sodium sulfate, filtrated, and concentrated under reduced pressure. The residue was purified by column chromatography, eluting with petroleum ether:ethyl acetate (100:0~72:28) to give the product (3.9 g, 93.8% yield). LC-MS (ESI): m/z [M+H]⁺=247.2.

### Step 5: 5-(3-(5-isopropyloxazol-2-yl)cyclopentyl)-1H-pyrazol-3-amine

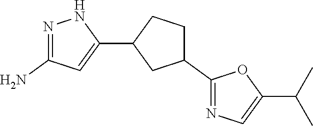 [SMILES: CC(C)c1cnc(C2CCC(c3cc(N)n[nH]3)C2)o1]

To a solution of 3-(3-(5-isopropyloxazol-2-yl)cyclopentyl)-3-oxopropanenitrile (3.9 g, 15.8 mmol) in EtOH (80 mL) were added hydrazine hydrate (868 mg, 17.3 mmol) and TsOH (50 mg). The resulting mixture was stirred at 90° C. for 1 h. The reaction was concentrated under vacuum and the residue was purified by silica gel column chromatography, eluting with PE:EA (100:0 to 70:30) to give the product (2.2 g, 53.4%). ¹H NMR (500 MHz, DMSO-d₆) δ 11.14 (s, 1H), 6.68 (s, 1H), 5.21 (s, 1H), 4.35 (s, 2H), 4.10 (q, J=5.2 Hz, 1H), 3.29 (q, J=8.5 Hz, 1H), 3.18 (d, J=5.5 Hz, 2H), 3.06-3.00 (m, 1H), 2.93 (p, J=6.5 Hz, 1H), 2.36 (d, J=13.0 Hz, 1H), 2.04 (tq, J=14.6, 5.9, 4.5 Hz, 2H), 2.00-1.89 (m, 1H), 1.82 (q, J=11.0 Hz, 1H), 1.68 (dd, J=11.3, 6.5 Hz, 1H), 1.19 (d, J=6.5 Hz, 6H). LCMS (ESI) m/z [M+H]⁺=261.

### Step 6: 3-fluoro-4-((5-(3-(5-isopropyloxazol-2-yl)cyclopentyl)-1H-pyrazol-3-yl)amino)benzenesulfonamide

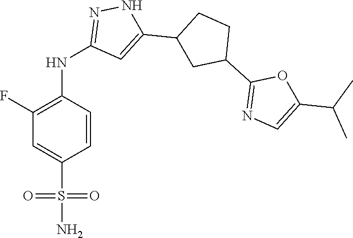 [SMILES: CC(C)c1cnc(C2CCC(c3cc(Nc4ccc(S(N)(=O)=O)cc4F)n[nH]3)C2)o1]

The titled compound was synthesized in the procedures similar to Example 1, step 10. ¹H NMR (500 MHz, DMSO-d₆) δ 8.64 (s, 1H), 8.16 (t, J=8.4 Hz, 1H), 7.50 (d, J=9.9 Hz, 2H), 7.18 (d, J=9.5 Hz, 2H), 6.69 (s, 1H), 5.83 (s, 1H), 3.36 (dt, J=16.7, 8.2 Hz, 2H), 3.18 (dt, J=9.9, 7.7 Hz, 1H), 2.93 (dq, J=13.8, 6.9 Hz, 1H), 2.48-2.41 (m, 1H), 2.18-2.06 (m, 2H), 2.02-1.94 (m, 1H), 1.89 (dd, J=22.5, 10.3 Hz, 1H), 1.78-1.66 (m, 1H), 1.19 (t, J=5.7 Hz, 6H). LC-MS (ESI): m/z [M+H]⁺=434.2.

### Example 8: 4-((5-(3-(5-(tert-butyl)-4-methyloxazol-2-yl)cyclopentyl)-1H-pyrazol-3-yl)amino)-3-fluorobenzenesulfonamide

### Step 1: tert-butyl (4,4-dimethyl-3-oxopentan-2-yl)carbamate

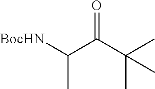 [SMILES: CC(NC(=O)OC(C)(C)C)C(=O)C(C)(C)C]

To a solution of tert-butyl (1-(methoxy(methyl)amino)-1-oxopropan-2-yl)carbamate (10.50 g, 45.26 mmol) in Et₂O (150 mL) was added n-BuLi (45.26 mL, 113.15 mmol) dropwised at −78° C. under nitrogen atmosphere. The resulting mixture was stirred for 2 h at −78° C. The reaction was quenched with sat NH₄Cl(aq.) at 0° C. and then extracted with EtOAc (3×300 mL). The combined organic layers were washed with brine, dried over anhydrous Na₂SO₄ and filtrated. The filtrate was concentrated under reduced pressure and the residue was purified by silica gel column chromatography, eluted with PE/EA (100:0~90:10) to afford the product (1.8 g, 17.37%). LC-MS (ESI): m/z [M+H]⁺=230.3.

### Step 2: 4-amino-2,2-dimethylpentan-3-one chloride

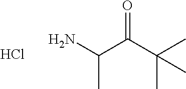

Into a 250-mL round-bottom flask were added tert-butyl (4,4-dimethyl-3-oxopentan-2-yl)carbamate (1.8 g, 13.95 mmol) and HCl-dioxane (4M, 20 mL) at room temperature. The resulting mixture was stirred for 2 h at room temperature. The resulting mixture was concentrated under reduced pressure to afford the product (1.0 g, 98.62% yield). LC-MS (ESI): m/z [M+H]⁺=130.3.

### Step 3: cis-3-((4,4-dimethyl-3-oxopentan-2-yl)carbamoyl)cyclopentane-1-carboxylic acid

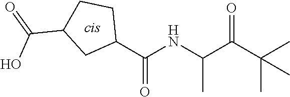 [SMILES: CC(NC(=O)C1CCC(C(=O)O)C1)C(=O)C(C)(C)C]

To a solution of cis-3-oxabicyclo[3.2.1]octane-2,4-dione (1.09 g, 7.75 mmol) in THF (30 mL) was added 4-amino-2,2-dimethylpentan-3-one (1.0 g, 7.75 mmol) and TEA (1.57 g, 15.5 mmol). The resulting mixture was stirred 2 h at room temperature. The solution was concentrated under reduced pressure to afford the product (2.0 g, 95.91%). LC-MS (ESI): m/z [M+H]⁺=270.3.

### Step 4: cis-methyl 3-((4,4-dimethyl-3-oxopentan-2-yl)carbamoyl)cyclopentane-1-carboxylate

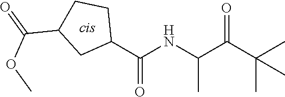 [SMILES: COC(=O)C1CCC(C(=O)NC(C)C(=O)C(C)(C)C)C1]

To a solution of cis-3-((4,4-dimethyl-3-oxopentan-2-yl)carbamoyl)cyclopentane-1-carboxylic acid (2.05 g, 7.62 mmol) in MeOH (50 mL) was added H₂SO₄ (1.49 g, 15.24 mmol). The resulting mixture was stirred for 3 h at room temperature. The mixture was neutralized to PH=7 with Na₂CO₃(aq.) aqueous solution and extracted with 150 mL of EtOAc. The combined organic layers were washed with brine, dried over anhydrous Na₂SO₄ and filtrated. The filtrate was concentrated under reduced pressure and the residue was purified by silica gel column chromatography, eluted with PE/EtOAc (100:0~70:30) to afford the product (1.90 g, 88% yield). LC-MS (ESI): m/z [M+H]⁺=284.3.

### Step 5: cis-methyl 3-(5-(tert-butyl)-4-methyloxazol-2-yl)cyclopentane-1-carboxylate

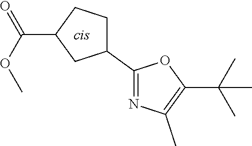 [SMILES: COC(=O)C1CCC(c2nc(C)c(C(C)(C)C)o2)C1]

cis-methyl 3-((4,4-dimethyl-3-oxopentan-2-yl)carbamoyl)cyclopentane-1-carboxylate (1.90 g, 6.71 mmol) was added to POCl₃ (40 mL). The resulting mixture was stirred for 5 h at 60° C. The resulting mixture was concentrated under reduced pressure. The residue was purified by silica gel column chromatography, eluted with PE/EtOAc (100:0-40:60) to afford the product (1.60 g, 89.93%). LC-MS (ESI): m/z [M+H]⁺=266.2.

### Step 6: 3-(3-(5-(tert-butyl)-4-methyloxazol-2-yl)cyclopentyl)-3-oxopropanenitrile

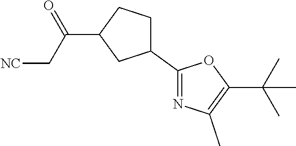 [SMILES: Cc1nc(C2CCC(C(=O)CC#N)C2)oc1C(C)(C)C]

Into a 100-mL 3-necked round-bottom flask purged with an inert atmosphere of nitrogen was placed CH₃CN (496.1 mg, 12.1 mmol) in THF 20 mL. Then n-BuLi (4.84 mL, 12.1 mmol) was added dropwise at −78° C. The resulting mixture was stirred for 30 min at −78° C., then added to a solution of cis-methyl 3-(5-(tert-butyl)-4-methyloxazol-2-yl)cyclopentane-1-carboxylate (1.60 g, 6.04 mmol) in THF (50 mL) dropwise at −78° C. The resulting mixture was stirred for 2 h at −78° C. The reaction was quenched with sat NH₄Cl(aq.) at 0° C. The resulting mixture was extracted with EtOAc (100 mL). The combined organic layers were washed with brine, dried over anhydrous Na₂SO₄ and filtrated. The filtrate was concentrated under reduced pressure and the residue was purified by silica gel column chromatography, eluted with PE/EA (100:0~60:40) to afford the product (0.92 g, 55.61% yield). LC-MS (ESI): m/z [M+H]⁺=275.3.

### Step 7: 5-(3-(5-(tert-butyl)-4-methyloxazol-2-yl)cyclopentyl)-1H-pyrazol-3-amine

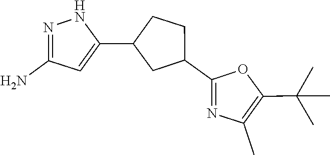 [SMILES: Cc1nc(C2CCC(c3cc(N)n[nH]3)C2)oc1C(C)(C)C]

To a solution of 3-(3-(5-(tert-butyl)-4-methyloxazol-2-yl)cyclopentyl)-3-oxopropanenitrile (900.0 mg, 3.28 mmol) in EtOH (20 mL) were added hydrazine hydrate (180.5 mg, 3.61 mmol)) and TsOH (50 mg). The resulting mixture was stirred at 90° C. for 1 h. The reaction was concentrated under vacuum and the residue was purified by silica gel column chromatography, eluting with PE:EA (100:0 to 80:20) to give the product (844.6 mg, 89.28% yield). ¹H NMR (300 MHz, DMSO-d₆) δ 5.19 (s, 1H), 3.21 (p, J=8.1 Hz, 1H), 3.10-2.94 (m, 1H), 2.39-2.24 (m, 1H), 2.10 (s, 4H), 2.08-1.85 (m, 3H), 1.88-1.59 (m, 1H), 1.27 (s, 9H). LC-MS (ESI): m/z [M+H]⁺=289.3.

### Step 8: 4-((5-(3-(5-(tert-butyl)-4-methyloxazol-2-yl)cyclopentyl)-1H-pyrazol-3-yl)amino)-3-fluorobenzenesulfonamide

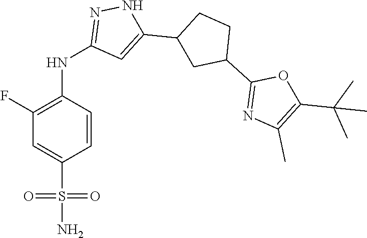 [SMILES: Cc1nc(C2CCC(c3cc(Nc4ccc(S(N)(=O)=O)cc4F)n[nH]3)C2)oc1C(C)(C)C]

The titled compound was synthesized in the procedures similar to Example 1, step 10. ¹H NMR (500 MHz, DMSO-d₆) δ 12.00 (s, 1H), 8.65-8.59 (m, 1H), 8.22-8.12 (m, 1H), 7.52-7.49 (m, 1H), 7.48 (s, 1H), 7.17 (s, 2H), 5.82 (s, 1H), 3.30-3.21 (m, 1H), 3.20-3.12 (m, 1H), 2.45-2.37 (m, 1H), 2.17-2.01 (m, 5H), 2.00-1.90 (m, 1H), 1.89-1.81 (m, 1H), 1.77-1.67 (m, 1H), 1.27 (s, 9H). LC-MS (ESI): m/z [M+H]⁺=462.3.

### Example 9: 4-((5-(3-(5-cyclopropyloxazol-2-yl)cyclopentyl)-1H-pyrazol-3-yl)amino)-3-fluorobenzenesulfonamide

### Step 1: cis-3-carbamoylcyclopentane-1-carboxylic acid

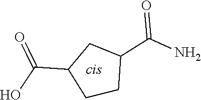 [SMILES: NC(=O)C1CCC(C(=O)O)C1]

To a solution of cis-3-oxa-bicyclo[3.2.1]octane-2,4-dione (14.50 g, 103.57 mmol) in THF (200 mL) was added NH₃-MeOH (59.18 mL, 414.29 mmol). The resulting mixture was stirred for 2 h at room temperature. The resulting mixture was concentrated under reduced pressure to afford the product (16.00 g, 98.40%). LC-MS (ESI): m/z [M+H]⁺=158.2.

### Step 2: cis-methyl 3-carbamoylcyclopentane-1-carboxylate

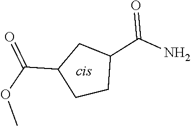

To a solution of cis-3-carbamoylcyclopentane-1-carboxylic acid (16.00 g, 101.91 mmol) in MeOH (100 mL) was added H₂SO₄ (39.95 g, 407.64 mmol). The resulting mixture was stirred overnight at room temperature. The mixture was neutralized to PH=7 with Na₂CO₃(aq.) aqueous solution and extracted with 300 mL of EtOAc twice. The combined organic layers were washed with brine, dried over anhydrous Na₂SO₄ and filtrated. The filtrate was concentrated under reduced pressure and the residue was purified by silica gel column chromatography, eluted with DCM/MeOH (100:0-90:10) to afford the product (6.40 g, 36.73% yield). LC-MS (ESI): m/z [M+H]⁺=172.2.

### Step 3: cis-methyl 3-cyanocyclopentane-1-carboxylate

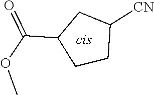

To a solution of cis-methyl 3-carbamoylcyclopentane-1-carboxylate (6.40 g, 37.43 mmol) in DCM (200 mL) was added TEA (11.34 g, 112.28 mmol) and TFAA (17.29 g, 82.35 mmol). The resulting mixture was stirred 2 h at room temperature. Water (200 mL) was added, and the resulting mixture was extracted with DCM (400 mL) twice. The combined organic layers were washed with brine, dried over anhydrous Na₂SO₄ and filtrated. The filtrate was concentrated under reduced pressure and the residue was purified by silica gel column chromatography, eluted with PE/EtOAc (100:0-80:20) to afford the product (5.14 g, 89.76%). ¹H NMR (300 MHz, Chloroform-d) δ 3.71 (s, 3H), 2.94-2.72 (m, 2H), 2.38 (dt, J=13.3 Hz, 1H), 2.27-1.90 (m, 5H). LC-MS (ESI): m/z [M+H]⁺=154.2.

### Step 4: methyl 3-(5-cyclopropyloxazol-2-yl)cyclopentane-1-carboxylate

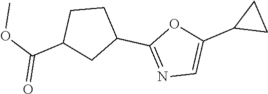 [SMILES: COC(=O)C1CCC(c2ncc(C3CC3)o2)C1]

To a solution of methyl 3-cyanocyclopentane-1-carboxylate (3 g, 19.59 mmol) in trimethyl((1-methylcyclopropyl)ethynyl)silane (5.4 g, 39.18 mmol) were added Ph₃PAuNTf₂ (724 mg, 0.979 mmol) and 8-methylquinoline 1-oxide (4.05 g, 25.46 mmol). The resulting mixture was stirred at 60° C. for 4 h under N₂ atmosphere. The resulting mixture was concentrated under vacuum and the residue was purified by silica gel column chromatography, eluted with PE/EtOAc (100:0 to 90:10) to give the product (1.5 g, 32.6%). LC-MS (ESI): m/z [M+H]⁺=236.

### Step 5: 3-(3-(5-cyclopropyloxazol-2-yl)cyclopentyl)-3-oxopropanenitrile

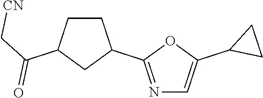 [SMILES: N#CCC(=O)C1CCC(c2ncc(C3CC3)o2)C1]

To a solution of CH₃CN (453 mg, 11.06 mmol) in THF (35 mL) was added n-BuLi (2.5 M) (4.4 mL, 11.06 mmol) at −78° C. and stirred for 1 h under N₂ atmosphere. The methyl 3-(5-cyclopropyloxazol-2-yl)cyclopentane-1-carboxylate (1.3 g, 5.53 mmol) was added in it at −78° C. and stirred for another 1 h. The reaction was quenched with NH₄Cl solution (50 mL) and extracted with EtOAc (3×35 mL). The combined organic layers were washed with brine, dried over Na₂SO₄ and concentrated. The residue was purified by silica gel column chromatography, eluted with PE/EtOAc (100:0 to 65:35) to give the product (550 mg, 40.7% yield). LC-MS (ESI): m/z [M+H]⁺=245.

### Step 6: 5-(3-(5-cyclopropyloxazol-2-yl)cyclopentyl)-1H-pyrazol-3-amine

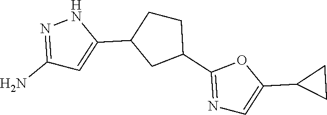 [SMILES: Nc1cc(C2CCC(c3ncc(C4CC4)o3)C2)[nH]n1]

To a solution of 3-(3-(5-cyclopropyloxazol-2-yl)cyclopentyl)-3-oxopropanenitrile (550 mg, 2.25 mmol) in EtOH (10 mL) were added hydrazine hydrate (124 mg, 2.48 mmol) and TsOH (15 mg). The resulting solution was stirred at 90° C. for 1 h. The reaction was concentrated under vacuum and the residue was purified by silica gel column chromatography, eluting with PE:EA (100:0 to 63:35) to give the product (296.8 mg, 51% yield). ¹H NMR (300 MHz, Chloroform-d) δ 6.62 (d, J=0.9 Hz, 1H), 5.49 (s, 1H), 4.78 (s, 2H), 3.39 (q, J=6.6 Hz, 1H), 3.25 (q, J=8.1 Hz, 1H), 2.48 (dt, J=13.5, 8.7 Hz, 1H), 2.30-2.16 (m, 1H), 2.15-1.97 (m, 3H), 1.94-1.73 (m, 2H), 1.00-0.87 (m, 2H), 0.82-0.71 (m, 2H). LC-MS (ESI): m/z [M+H]⁺=259.2.

### Step 7: 4-((5-(3-(5-cyclopropyloxazol-2-yl)cyclopentyl)-1H-pyrazol-3-yl)amino)-3-fluorobenzenesulfonamide

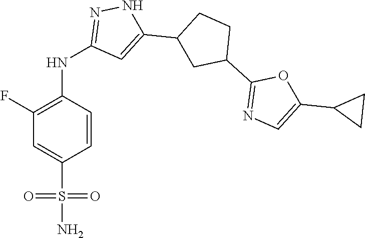 [SMILES: NS(=O)(=O)c1ccc(Nc2cc(C3CCC(c4ncc(C5CC5)o4)C3)[nH]n2)c(F)c1]

The titled compound was synthesized in the procedures similar to Example 1, step 10. ¹H NMR (500 MHz, DMSO-d₆) δ 12.00 (s, 1H), 8.63 (s, 1H), 8.19 (t, J=8.6 Hz, 1H), 7.50 (d, J=9.8 Hz, 2H), 7.17 (s, 2H), 6.70 (s, 1H), 5.82 (s, 1H), 3.39-3.34 (m, 1H), 3.21-3.13 (m, 1H), 2.45-2.39 (m, 1H), 2.14-2.04 (m, 2H), 1.99-1.83 (m, 3H), 1.77-1.68 (m, 1H), 0.94-0.86 (m, 2H), 0.71-0.66 (m, 2H). LC-MS (ESI): m/z [M+H]⁺=432.3.

### Example 10: 5-((3-(3-(5-(tert-butyl)pyridin-2-yl)cyclopentyl)-1H-pyrazol-5-yl)amino)-6-fluoro-2,3-dihydrobenzo[d]isothiazole 1,1-dioxide

### Step 1: methyl 5-bromo-2-(chlorosulfonyl)-4-fluorobenzoate

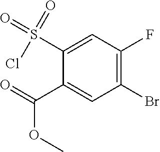 [SMILES: COC(=O)c1cc(Br)c(F)cc1S(=O)(=O)Cl]

To a suspension of methyl 2-amino-5-bromo-4-fluorobenzoate (3.0 g, 12.1 mmol) in acetic acid (20 mL) and hydrochloric acid (12 M, 54 mL), a solution of sodium nitrite (918.4 mg, 13.31 mmol) in 8 mL water was added dropwise at 0° C. The resulting mixture was kept below 5° C. and stirred for 30 mins after addition. To the reaction mixture CuCl₂ (813 mg, 6.05 mmol), sodium bisulfite (18.9 g, 181.5 mmol) and hydrochloric acid solution (6 M, 88 mL) were added sequentially. The reaction mixture was warmed to room temperature and stirred for 3 h. The resulting mixture was extracted with EA (50 mL×3). The combined organic layers were washed with brine, dried over Na₂SO₄, and filtered. The filtrated was concentrated and dried over in vacuum to afford the crude product (3.0 g), which was used in the next step without further purification.

### Step 2: 5-bromo-6-fluorobenzo[d]isothiazol-3 (2H)-one 1,1-dioxide

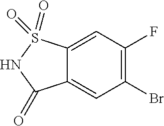 [SMILES: O=C1NS(=O)(=O)c2cc(F)c(Br)cc21]

To a mixture of methyl 5-bromo-2-(chlorosulfonyl)-4-fluorobenzoate (3 g crude) was dissolved in THF (10 mL) was added, aqueous ammonium (25%, 20 mL). The reaction mixture was stirred at room temperature for 16 h. The resulting mixture was concentrated under vacuum, and the residue was triturated with EA and filtered to afford the crude product (2.0 g). LC-MS (ESI): m/z [M−H]—=277.9.

### Step 3: 5-bromo-6-fluoro-2,3-dihydrobenzo[d]isothiazole 1,1-dioxide

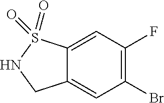 [SMILES: O=S1(=O)NCc2cc(Br)c(F)cc21]

To a stirring solution of 5-bromo-6-fluorobenzo[d]isothiazol-3 (2H)-one 1,1-dioxide (300 mg, 1.08 mmol) in THF (10 mL) was added BH₃-Me₂S (2 M, 2.7 mL, 5.4 mmol) dropwise at 0° C. under nitrogen atmosphere. The solution was heated to reflux and stirred for 16 h. The resulting mixture was cooled to 0° C., quenched with MeOH (10 mL), and concentrated under reduced pressure. The residue was purified by silica gel column chromatography, eluting with PE/EA (1:1) to afford the product (260 mg, 91%). LC-MS (ESI): m/z [M+H]⁺=266/268.

### Step 4: 5-((5-(3-(5-(tert-butyl)pyridin-2-yl)cyclopentyl)-1H-pyrazol-3-yl)amino)-6-fluoro-2,3-dihydrobenzo[d]isothiazole 1,1-dioxide

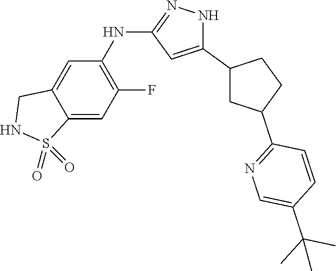 [SMILES: CC(C)(C)c1ccc(C2CCC(c3cc(Nc4cc5c(cc4F)S(=O)(=O)NC5)n[nH]3)C2)nc1]

The titled compound was synthesized in the procedures similar to Example 1. ¹H NMR (500 MHz, DMSO-d₆) δ 12.05 (s, 1H), 8.74 (d, J=10 Hz, 1H), 8.56 (d, J=10 Hz, 1H), 8.19-8.11 (m, 1H), 7.74-7.68 (m, 1H), 7.66-7.59 (m, 2H), 7.22 (d, J=10 Hz, 1H), 5.89 (s, 1H), 4.30 (d, J=5 Hz, 2H), 3.37-3.33 (m, 1H), 3.28-3.18 (m, 1H), 2.41-2.33 (m, 1H), 2.19-2.03 (m, 2H), 1.98-1.85 (m, 2H), 1.84-1.73 (m, 1H), 1.30 (s, 9H). LC-MS (ESI): m/z [M+H]⁺=470.2.

### Example 11: 6-fluoro-5-((5-(3-(6-methoxypyridin-2-yl)cyclopentyl)-1H-pyrazol-3-yl)amino)-2,3-dihydrobenzo[d]isothiazole 1,1-dioxide

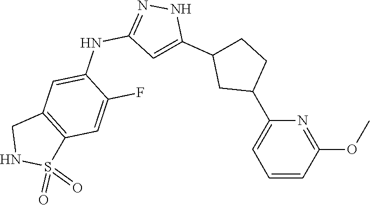 [SMILES: COc1cccc(C2CCC(c3cc(Nc4cc5c(cc4F)S(=O)(=O)NC5)n[nH]3)C2)n1]

The titled compound was synthesized in the procedures similar to Example 1. ¹H NMR (500 MHz, DMSO-d₆) δ 12.02 (s, 1H), 8.76-8.72 (m, 1H), 8.19-8.12 (m, 1H), 7.66-7.56 (m, 3H), 6.89-6.85 (m, 1H), 6.61 (d, J=10 Hz, 1H), 5.88 (s, 1H), 4.32-4.27 (m, 2H), 3.84 (s, 3H), 3.30-3.18 (m, 2H), 2.42-2.34 (m, 1H), 2.18-2.01 (m, 2H), 1.99-1.72 (m, 3H). LC-MS (ESI): m/z [M+H]⁺=444.1.

### Example 12: 6-fluoro-5-((5-(3-(6-methylpyridin-2-yl)cyclopentyl)-1H-pyrazol-3-yl)amino)-2,3-dihydrobenzo[d]isothiazole 1,1-dioxide

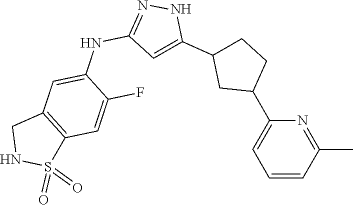 [SMILES: Cc1cccc(C2CCC(c3cc(Nc4cc5c(cc4F)S(=O)(=O)NC5)n[nH]3)C2)n1]

The titled compound was synthesized in the procedures similar to Example 1. ¹H NMR (500 MHz, DMSO-d₆) δ 12.12 (s, 1H), 8.77-8.72 (m, 1H), 8.19-8.10 (m, 1H), 7.68-7.56 (m, 3H), 7.13-7.03 (m, 2H), 5.89 (s, 1H), 4.32-4.28 (m, 2H), 3.31-3.28 (m, 1H), 3.27-3.19 (m, 1H), 2.45 (s, 3H), 2.41-2.34 (m, 1H), 2.24-2.04 (m, 2H), 1.98-1.76 (m, 3H). LC-MS (ESI): m/z [M+H]⁺=428.1.

### Example 13: 5-((5-(3-(4-(tert-butyl)pyridin-2-yl)cyclopentyl)-1H-pyrazol-3-yl)amino)-6-fluoro-2,3-dihydrobenzo[d]isothiazole 1,1-dioxide

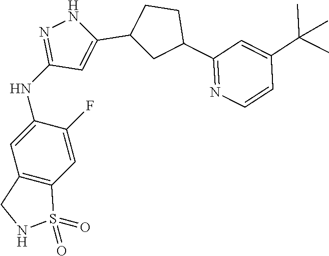 [SMILES: CC(C)(C)c1ccnc(C2CCC(c3cc(Nc4cc5c(cc4F)S(=O)(=O)NC5)n[nH]3)C2)c1]

The titled compound was synthesized in the procedures similar to Example 1. ¹H NMR (500 MHz, DMSO-d₆) δ 12.06 (s, 1H), 8.79 (d, J=2.5 Hz, 1H), 8.65 (d, J=5.9 Hz, 1H), 8.14 (d, J=7.3 Hz, 1H), 7.79 (d, J=11.5 Hz, 2H), 7.65 (d, J=10.0 Hz, 2H), 5.93 (s, 1H), 4.30 (s, 2H), 3.60-3.50 (m, 1H), 3.35-3.22 (m, 1H), 2.48-2.43 (m, 1H), 2.32-2.15 (m, 2H), 2.05-1.85 (m, 3H), 1.35 (d, J=5.0 Hz, 9H). LC-MS (ESI): m/z [M+H]⁺=470.34.

### Example 14: 5-((5-(3-(4-(tert-butyl)pyrimidin-2-yl)cyclopentyl)-1H-pyrazol-3-yl)amino)-6-fluoro-2,3-dihydrobenzo[d]isothiazole 1,1-dioxide

### Step 1: benzyl (1-(tert-butyl)-3-(3-(5-(tert-butyl)-6-chloropyridazin-3-yl)cyclopent-2-en-1-yl)-1H-pyrazol-5-yl)carbamate

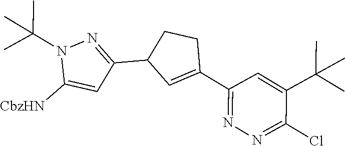 [SMILES: CC(C)(C)c1cc(C2=CC(c3cc(NC(=O)OCc4ccccc4)n(C(C)(C)C)n3)CC2)nnc1Cl]

A mixture of 4-(tert-butyl)-3,6-dichloropyridazine (510 mg, 2.5 mmol), benzyl (1-(tert-butyl)-3-(3-(4,4,5,5-tetramethyl-1,3,2-dioxaborolan-2-yl)cyclopent-2-en-1-yl)-1H-pyrazol-5-yl)carbamate (1.4 g, 3 mmol), Pd(dppf)Cl₂ (203 mg, 0.25 mmol) and Na₂CO₃ (795 mg, 7.5 mmol) in dioxane (20 mL) and H₂O (5 mL) was stirred at 80° C. for 16 h under a nitrogen atmosphere. The mixture was concentrated under vacuum. The residue was purified by silica gel column chromatography, eluting with EA/PE (0-45%) to afford the product (1.15 mg, 90.6%). LC-MS (ESI): m/z [M+H]⁺=508.2.

### Step 2: 1-(tert-butyl)-3-(3-(5-(tert-butyl)pyridazin-3-yl)cyclopentyl)-1H-pyrazol-5-amine

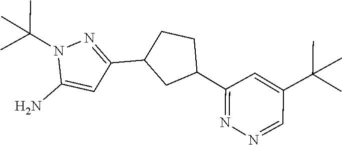 [SMILES: CC(C)(C)c1cnnc(C2CCC(c3cc(N)n(C(C)(C)C)n3)C2)c1]

To a solution of benzyl (1-(tert-butyl)-3-(3-(5-(tert-butyl)-6-chloropyridazin-3-yl)cyclopent-2-en-1-yl)-1H-pyrazol-5-yl)carbamate (1.15 g, 2.3 mmol) in MeOH (20 mL) was added Pd/C (150 mg). The mixture was stirred at rt for 5 h under a hydrogen atmosphere. The mixture was filtered and the filtrate was concentrated under vacuum to afford (567 mg, crude). The residue was used in the next step directly without further purification. LC-MS (ESI): m/z [M+H]⁺=342.2.

### Step 3: 5-((5-(3-(5-(tert-butyl)pyridazin-3-yl)cyclopentyl)-1H-pyrazol-3-yl)amino)-6-fluoro-2,3-dihydrobenzo[d]isothiazole 1,1-dioxide

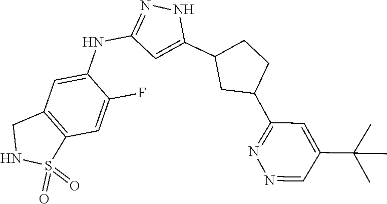 [SMILES: CC(C)(C)c1cnnc(C2CCC(c3cc(Nc4cc5c(cc4F)S(=O)(=O)NC5)n[nH]3)C2)c1]

The titled compound was synthesized in the procedures similar to Example 1. ¹H NMR (500 MHz, DMSO-d₆) δ 12.08 (s, 1H), 9.20 (d, J=5 Hz, 1H), 8.78-8.75 (m, 1H), 8.19-8.13 (m, 1H), 7.67-7.61 (m, 2H), 7.54-7.51 (m, 1H), 5.91 (s, 1H), 4.34-4.28 (m, 2H), 3.62-3.48 (m, 1H), 3.33-3.24 (m, 1H), 2.48-2.45 (m, 1H), 2.32-2.15 (m, 2H), 2.09-1.83 (m, 3H), 1.31 (s, 9H). LC-MS (ESI): m/z [M+H]⁺=471.2.

### Example 15: 4-((5-(3-(4-(tert-butyl)pyrimidin-2-yl)cyclopentyl)-1H-pyrazol-3-yl)amino)-3-fluorobenzenesulfonamide

### Step 1: benzyl (1-(tert-butyl)-3-(3-(4-(tert-butyl)pyrimidin-2-yl)cyclopent-2-en-1-yl)-1H-pyrazol-5-yl)carbamate

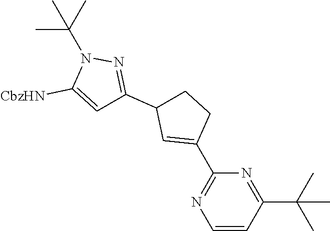 [SMILES: CC(C)(C)c1ccnc(C2=CC(c3cc(NC(=O)OCc4ccccc4)n(C(C)(C)C)n3)CC2)n1]

To a solution of benzyl (1-(tert-butyl)-3-(3-(4,4,5,5-tetramethyl-1,3,2-dioxaborolan-2-yl)cyclopent-2-en-1-yl)-1H-pyrazol-5-yl)carbamate (500 mg, 1.075 mmol) and 4-(tert-butyl)-2-chloropyrimidine (277.2 mg, 1.612 mmol) in dioxane/H₂O (10:2 mL) was added Pd(dppf)Cl₂ (99.6 mg, 0.107 mmol) and K₃PO₄ (683.7 mg, 3.225 mmol). The reaction mixture was stirred for 6 hours at 100° C. under nitrogen. The resulting mixture was cooled to room temperature, concentrated under reduce pressure, purified by silica gel column chromatography, eluting with PE/EA (1:1) to afford the product (400 mg, 78.5%). LC-MS (ESI): m/z [M+H]⁺=475.3.

### Step 2: 1-(tert-butyl)-3-(3-(4-(tert-butyl)pyrimidin-2-yl)cyclopentyl)-1H-pyrazol-5-amine

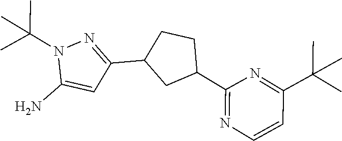 [SMILES: CC(C)(C)c1ccnc(C2CCC(c3cc(N)n(C(C)(C)C)n3)C2)n1]

benzyl (1-(tert-butyl)-3-(3-(4-(tert-butyl)pyrimidin-2-yl)cyclopent-2-en-1-yl)-1H-pyrazol-5-yl)carbamate (400 mg, 0.84 mmol) was dissolved in MeOH (10 mL), Pd/C (10% on carbon, 200 mg) was added. The reaction mixture was degassed and purged with hydrogen 3 times and stirred for 2 hours at room temperature. The resulting mixture was filtered and the filtrated was concentrated in vacuum to afford the product (200 mg, 69%). LC-MS (ESI): m/z [M+H]⁺=343.3.

### Step 3: 5-((5-(3-(4-(tert-butyl)pyrimidin-2-yl)cyclopentyl)-1H-pyrazol-3-yl)amino)-6-fluoro-2,3-dihydrobenzo[d]isothiazole 1,1-dioxide

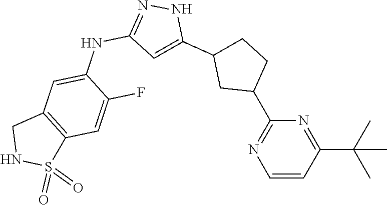 [SMILES: CC(C)(C)c1ccnc(C2CCC(c3cc(Nc4cc5c(cc4F)S(=O)(=O)NC5)n[nH]3)C2)n1]

The titled compound was synthesized in the procedures similar to Example 1. ¹H NMR (500 MHz, DMSO-d₆) δ=12.03 (s, 1H), 8.74 (d, J=2.6 Hz, 1H), 8.63 (d, J=5.3, 3.0 Hz, 1H), 8.16 (s, 1H), 7.81-7.55 (m, 2H), 7.33 (d, J=5.3 Hz, 1H), 5.87 (s, 1H), 4.30 (d, J=4.8 Hz, 2H), 3.52-3.44 (m, 3H), 3.26-3.20 (m, 1H), 2.48-2.42 (m, 1H), 2.25-1.96 (m, 4H), 1.89-1.76 (m, 1H), 1.30 (s, 9H). LC-MS (ESI): m/z [M+H]⁺=471.2.

### Example 16: 6-fluoro-5-((5-(3-(4-isopropylpyrimidin-2-yl)cyclopentyl)-1H-pyrazol-3-yl)amino)-2,3-dihydrobenzo[d]isothiazole 1,1-dioxide

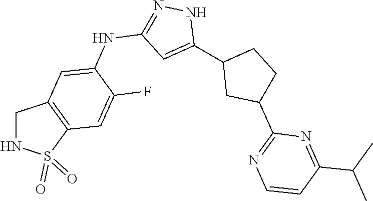 [SMILES: CC(C)c1ccnc(C2CCC(c3cc(Nc4cc5c(cc4F)S(=O)(=O)NC5)n[nH]3)C2)n1]

The titled compound was synthesized in the procedures similar to Example 1. ¹H NMR (500 MHz, DMSO-d₆) δ 12.04 (s, 1H), 8.75 (d, J=2.5 Hz, 1H), 8.67-8.56 (m, 1H), 8.16 (d, J=5.6 Hz, 1H), 7.63 (d, J=10.0 Hz, 2H), 7.22 (d, J=5.2 Hz, 1H), 5.88 (s, 1H), 4.23 (d, J=73.5 Hz, 2H), 3.46 (dt, J=15.9, 8.1 Hz, 1H), 3.28-3.19 (m, 1H), 3.01-2.90 (m, 1H), 2.44 (dd, J=12.9, 6.3 Hz, 1H), 2.24-2.09 (m, 2H), 2.09-2.03 (m, 1H), 1.99 (dd, J=22.7, 10.7 Hz, 1H), 1.88-1.72 (m, 1H), 1.23 (dd, J=6.9, 3.5 Hz, 6H). LC-MS (ESI): m/z [M+H]⁺=457.2.

### Example 17: 5-((5-(3-(5-(tert-butyl)oxazol-2-yl)cyclopentyl)-1H-pyrazol-3-yl)amino)-6-fluoro-2,3-dihydrobenzo[d]isothiazole 1,1-dioxide

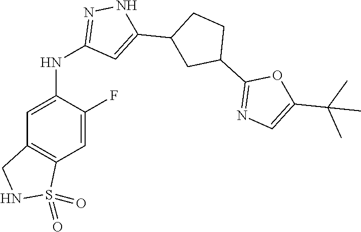 [SMILES: CC(C)(C)c1cnc(C2CCC(c3cc(Nc4cc5c(cc4F)S(=O)(=O)NC5)n[nH]3)C2)o1]

The titled compound was synthesized in the procedures similar to Example 6 in a racemic form, which was further separated by Chiral Prep-HPLC to give four isomers:

Enantiomer 1 (Example 17a, 100% ee); Retention time: 2.861 min. ¹H NMR (500 MHz, DMSO-d₆) δ 12.03 (s, 1H), 8.74 (s, 1H), 8.14 (d, J=5 Hz, 1H), 7.66-7.61 (m, 2H), 6.67 (s, 1H), 5.85 (s, 1H), 4.30 (d, J=5 Hz, 2H), 3.41-3.33 (m, 1H), 3.24-3.15 (m, 1H), 2.48-2.40 (m, 1H), 2.16-2.04 (m, 2H), 2.02-1.84 (m, 2H), 1.78-1.68 (m, 1H), 1.23 (s, 9H). LC-MS (ESI): m/z [M+H]⁺=460.2.

Enantiomer 2 (Example 17b, 99.1% ee); Retention time: 4.871 min. ¹H NMR (500 MHz, DMSO-d₆) δ 12.03 (s, 1H), 8.74 (s, 1H), 8.14 (d, J=5 Hz, 1H), 7.66-7.61 (m, 2H), 6.67 (s, 1H), 5.85 (s, 1H), 4.30 (d, J=5 Hz, 2H), 3.41-3.33 (m, 1H), 3.24-3.15 (m, 1H), 2.48-2.40 (m, 1H), 2.16-2.04 (m, 2H), 2.02-1.84 (m, 2H), 1.78-1.68 (m, 1H), 1.23 (s, 9H). LC-MS (ESI): m/z [M+H]⁺=460.2.

Enantiomer 3 (Example 17c, 98.3% ee); Retention time: 6.269 min. ¹H NMR (500 MHz, DMSO-d₆) δ 12.03 (s, 1H), 8.74 (s, 1H), 8.14 (d, J=5 Hz, 1H), 7.66-7.61 (m, 2H), 6.67 (s, 1H), 5.85 (s, 1H), 4.30 (d, J=5 Hz, 2H), 3.41-3.33 (m, 1H), 3.24-3.15 (m, 1H), 2.48-2.40 (m, 1H), 2.16-2.04 (m, 2H), 2.02-1.84 (m, 2H), 1.78-1.68 (m, 1H), 1.23 (s, 9H). LC-MS (ESI): m/z [M+H]⁺=460.2.

Enantiomer 4 (Example 17d, 99.7% ee); Retention time: 5.628 min. ¹H NMR (500 MHz, DMSO-d₆) δ 12.03 (s, 1H), 8.74 (s, 1H), 8.14 (d, J=5 Hz, 1H), 7.66-7.61 (m, 2H), 6.67 (s, 1H), 5.85 (s, 1H), 4.30 (d, J=5 Hz, 2H), 3.41-3.33 (m, 1H), 3.24-3.15 (m, 1H), 2.48-2.40 (m, 1H), 2.16-2.04 (m, 2H), 2.02-1.84 (m, 2H), 1.78-1.68 (m, 1H), 1.23 (s, 9H). LC-MS (ESI): m/z [M+H]⁺=460.2.

Chiral analytical method: Column: CHIRALPAK IA-3, 4.6*50 mm 3 m; Mobile phase: A for Hex (0.1% FA) and B for EtOH; Gradient: Mobile Phase A: Mobile Phase B=70:30 (v/v); Flow Rate: 20 mL/min, Wave Length: UV 254 nm and 220 nm, Prep-HPLC Equipment: Prep-HPLC-Gilson, Back pressure: 100 bar; Column temperature: 25° C.

Chiral Prep-HPLC Condition: CHIRALPAK IA, 2*250 mm 5 μm; Mobile phase: A for Hex (0.1% FA) and B for EtOH; Gradient: Mobile Phase A: Mobile Phase B=70:30 (v/v); Flow Rate: 20 mL/min, Wavelength: UV 254 nm and 220 nm, Prep-HPLC Equipment: Prep-HPLC-Gilson, Back pressure: 100 bar; Column temperature: 25° C.

### Example 18: 6-fluoro-5-((5-(3-(5-isopropyloxazol-2-yl)cyclopentyl)-1H-pyrazol-3-yl)amino)-2,3-dihydrobenzo[d]isothiazole 1,1-dioxide

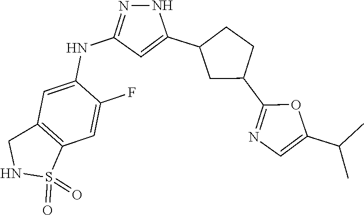 [SMILES: CC(C)c1cnc(C2CCC(c3cc(Nc4cc5c(cc4F)S(=O)(=O)NC5)n[nH]3)C2)o1]

The titled compound was synthesized in the procedures similar to Example 7. ¹H NMR (500 MHz, DMSO-d₆) δ 12.04 (s, 1H), 8.75 (d, J=2.3 Hz, 1H), 8.15 (d, J=6.0 Hz, 1H), 7.71-7.53 (m, 2H), 6.69 (d, J=1.0 Hz, 1H), 5.87 (d, J=11.7 Hz, 1H), 4.30 (d, J=4.5 Hz, 2H), 3.42 (dd, J=16.8, 10.9 Hz, 1H), 3.19 (dt, J=17.5, 8.8 Hz, 1H), 2.93 (dq, J=13.9, 7.0 Hz, 1H), 2.48-2.41 (m, 1H), 2.20-2.05 (m, 2H), 1.98 (dt, J=12.2, 7.9 Hz, 1H), 1.89 (dd, J=22.5, 10.3 Hz, 1H), 1.77-1.66 (m, 1H), 1.19 (dd, J=6.8, 4.5 Hz, 6H). LC-MS (ESI): m/z [M+H]⁺=446.2.

### Example 19: 5-((5-(3-(5-(tert-butyl)-4-methyloxazol-2-yl)cyclopentyl)-1H-pyrazol-3-yl)amino)-6-fluoro-2,3-dihydrobenzo[d]isothiazole 1,1-dioxide

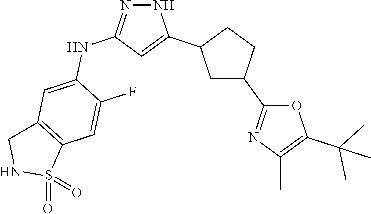 [SMILES: Cc1nc(C2CCC(c3cc(Nc4cc5c(cc4F)S(=O)(=O)NC5)n[nH]3)C2)oc1C(C)(C)C]

The titled compound was synthesized in the procedures similar to Example 8. ¹H NMR (500 MHz, DMSO-d₆) δ 12.03 (s, 1H), 8.77-8.71 (m, 1H), 8.20-8.09 (m, 1H), 7.68-7.59 (m, 2H), 5.85 (s, 1H), 4.30 (d, J=5 Hz, 2H), 3.30-3.22 (m, 1H), 3.21-3.12 (m, 1H), 2.45-2.38 (m, 1H), 2.18-2.02 (m, 5H), 1.99-1.90 (m, 1H), 1.89-1.81 (m, 1H), 1.78-1.67 (m, 1H), 1.27 (s, 9H). LC-MS (ESI): m/z [M+H]⁺=474.2.

### Example 20: 5-((5-(3-(5-cyclopropyloxazol-2-yl)cyclopentyl)-1H-pyrazol-3-yl)amino)-6-fluoro-2,3-dihydrobenzo[d]isothiazole 1,1-dioxide

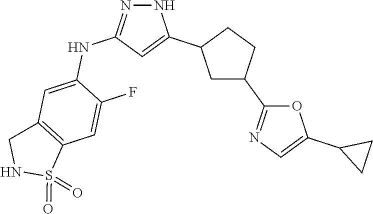 [SMILES: O=S1(=O)NCc2cc(Nc3cc(C4CCC(c5ncc(C6CC6)o5)C4)[nH]n3)c(F)cc21]

The titled compound was synthesized in the procedures similar to Example 9. ¹H NMR (500 MHz, DMSO-d₆) δ 12.03 (s, 1H), 8.75 (d, J=2.4 Hz, 1H), 8.14 (s, 1H), 7.67-7.62 (m, 2H), 6.70 (s, 1H), 5.85 (s, 1H), 4.30 (d, J=5.0 Hz, 2H), 3.36-3.32 (m, 1H), 3.23-3.15 (m, 1H), 2.47-2.39 (m, 1H), 2.13-2.04 (m, 2H), 2.00-1.83 (m, 3H), 1.76-1.69 (m, 1H), 0.92-0.86 (m, 2H), 0.71-0.65 (m, 2H). LC-MS (ESI): m/z [M+H]⁺=444.3

### Example 21: cis-2-(3-(3-((4-fluoro-1,1-dioxido-2,3-dihydrobenzo[d]isothiazol-5-yl)amino)-1H-pyrazol-5-yl)cyclopentyl)pyridazin-3 (2H)-one

### Step 1: methyl 6-amino-3-bromo-2-fluorobenzoate

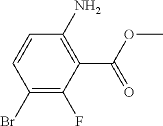

To a solution of 6-amino-3-bromo-2-fluorobenzoic acid (1.0 g, 4.27 mmol) in methanol (20 mL) was added SOCl₂ (2.5 g, 21.3 mmol) dropwise. The reaction mixture was stirred under refluxed for 16 hours. The resulting mixture was cooled to room temperature, concentrated, neutralized with sat. aq NaHCO₃ and extracted with EA. The combined organic layers were washed with brine, dried over Na₂SO₄, and concentrated under vacuum. The residue was purified by silica gel column chromatography, eluting with PE/EA (5:1) to afford the product (346 mg, 33%). LC-MS (ESI): m/z [M+H]⁺=248.0.

### Step 2: methyl 3-bromo-6-(chlorosulfonyl)-2-fluorobenzoate

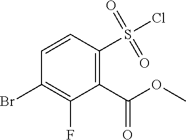 [SMILES: COC(=O)c1c(S(=O)(=O)Cl)ccc(Br)c1F]

To a suspension of methyl 6-amino-3-bromo-2-fluorobenzoate (3.0 g, 12.1 mmol) in hydrochloric acid (12 M, 60 mL), a solution of sodium nitrite (835 mg, 12.1 mmol) in 5 mL water was added dropwise at 0° C. The resulting mixture was kept below 5° C. and stirred for 30 mins after addition. To the reaction mixture CuCl₂ (813 mg, 6.05 mmol), sodium bisulfite (18.9 g, 181.5 mmol) and hydrochloric acid solution (5.5 M, 20 mL) were added sequentially. The reaction mixture was warmed to room temperature and stirred for 3 h. The resulting mixture was extracted with EA (50 mL×3). The combined organic layers were washed with brine, dried over Na₂SO₄, and filtered. The filtrated was concentrated and dried over in vacuum to afford the crude product (3.2 g), which was used in the next step without further purification.

### Step 3: 5-bromo-4-fluorobenzo[d]isothiazol-3 (2H)-one 1,1-dioxide

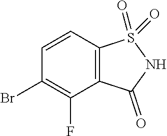 [SMILES: O=C1NS(=O)(=O)c2ccc(Br)c(F)c21]

To a solution of methyl 3-bromo-6-(chlorosulfonyl)-2-fluorobenzoate (3.2 g crude) in THF (50 mL) was added aqueous ammonia (25%, 10 mL) dropwise. The reaction mixture was stirred at room temperature for 16 h. The resulting mixture was concentrated under vacuum, and the residue was triturated with EA and filtered to afford the crude product (3.0 g, LC-MS (ESI): m/z [M+H]⁺=279.9), which was used in the next step without further purification.

### Step 4: 5-bromo-4-fluoro-2,3-dihydrobenzo[d]isothiazole 1,1-dioxide

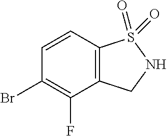 [SMILES: O=S1(=O)NCc2c1ccc(Br)c2F]

To a stirring solution of 5-bromo-4-fluorobenzo[d]isothiazol-3 (2H)-one 1,1-dioxide (210 mg, 0.75 mmol) in THF (5 mL) was added BH₃-Me₂S (2 M, 1.5 mL, 3.0 mmol) dropwise at 0° C. under nitrogen atmosphere. The solution was heated to reflux and stirred for 3 h. The resulting mixture was cooled to 0° C., quenched with MeOH (1.0 mL), and concentrated under reduced pressure. The residue was purified by silica gel column chromatography, eluting with DCM/MeOH (5:1) to afford the product (184 mg, 93%). LC-MS (ESI): m/z [M+H]⁺=265.8

### Step 5: 5-bromo-4-fluoro-2-(4-methoxybenzyl)-2,3-dihydrobenzo[d]isothiazole 1,1-dioxide

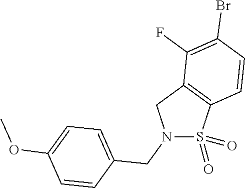 [SMILES: COc1ccc(CN2Cc3c(ccc(Br)c3F)S2(=O)=O)cc1]

To a solution of 5-bromo-4-fluoro-2,3-dihydrobenzo[d]isothiazole 1,1-dioxide (13 g, 50 mmol) in 100 mL DMF was added K₂CO₃ (13.8 g, 100 mL) and 1-(chloromethyl)-4-methoxybenzene (8.6 g, 55 mmol). The resulting mixture was stirred at room temperature for overnight. The mixture was poured into water and filtrated. The filtrate was washed by water and dried to obtain the product (15.3 g, 85% yield). LC-MS (ESI): m/z [M+H]⁺=385.9.

### Step 6: cis-benzyl (1-(tert-butyl)-3-(3-hydroxycyclopentyl)-1H-pyrazol-5-yl)carbamate

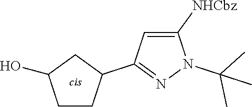 [SMILES: CC(C)(C)n1nc(C2CCC(O)C2)cc1NC(=O)OCc1ccccc1]

A solution of benzyl (1-(tert-butyl)-3-(3-oxocyclopentyl)-1H-pyrazol-5-yl)carbamate (19 g, 53.46 mmol) in THF (200 mL) was cooled to −65° C. A solution of LiBHEt₃ (1 M, 106.9 mL) was added dropwise and the resulting mixture was stirred at −65° C. for 1.5 h under nitrogen atmosphere. The reaction mixture was quenched with sat. aq. NaHCO₃ (50 mL). Water (200 mL) was added, and the mixture was extracted with EA (100 mL×3). The combined organic phase was washed with brine (200 mL), dried over anhydrous Na₂SO₄, then filtered and concentrated under vacuum. The residue was purified by prep-HPLC (column; mobile phase: [water (NH₄HCO₃)-acetone]; B %: 40%-65%, 20 min). The product (11 g, 57% yield) was obtained. ¹H NMR (400 MHz, DMSO-d₆) δ=9.06 (br s, 1H), 7.57-7.12 (m, 5H), 5.92 (s, 1H), 5.75 (s, 1H), 5.12 (s, 2H), 4.57 (d, J=4.4 Hz, 1H), 4.22-4.06 (m, 1H), 2.89 (q, J=8.6 Hz, 1H), 2.25-2.13 (m, 1H), 1.89-1.80 (m, 1H), 1.76-1.66 (m, 2H), 1.60-1.55 (m, 1H), 1.54-1.41 (m, 9H).

### Step 7: trans-3-(5-(((benzyloxy)carbonyl)amino)-1-(tert-butyl)-1H-pyrazol-3-yl)cyclopentyl 4-nitrobenzoate

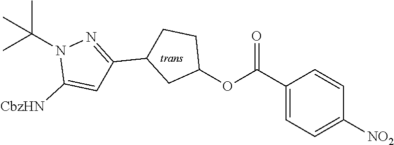 [SMILES: CC(C)(C)n1nc(C2CCC(OC(=O)c3ccc([N+](=O)[O-])cc3)C2)cc1NC(=O)OCc1ccccc1]

To a solution of cis-benzyl (1-(tert-butyl)-3-(3-hydroxycyclopentyl)-1H-pyrazol-5-yl)carbamate (2.14 g, 6 mmol) in THF (100 mL) was added PPh₃ (3.1 g, 12 mmol). Then the solution of DIAD (2.38 g, 12 mmol) in THF (20 mL) was added to the reaction through a syringe at 0° C. The resulting solution was stirred for 1 h at rt under nitrogen atmosphere. Water (100 mL) was added, and the resulting mixture was extracted with EtOAc (3×100 mL). The resulting mixture was concentrated under vacuum. The residue was purified by silica gel column chromatography, eluting with EtOAc/PE=25% to give the product (2.27 g, 75%). LCMS (ESI) m/z [M+H]⁺=507.3.

### Step 8: trans-benzyl (1-(tert-butyl)-3-(3-hydroxycyclopentyl)-1H-pyrazol-5-yl)carbamate

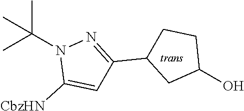 [SMILES: CC(C)(C)n1nc(C2CCC(O)C2)cc1NC(=O)OCc1ccccc1]

To a solution of trans-3-(5-(((benzyloxy)carbonyl)amino)-1-(tert-butyl)-1H-pyrazol-3-yl)cyclopentyl 4-nitrobenzoate (2.27 g, 4.5 mmol) in MeOH (100 mL) was added K₂CO₃ (621 mg, 4.5 mmol). The resulting solution was stirred for 3 h at rt at room temperature. The resulting mixture was filtrated, and the filtrate was concentrated under vacuum. The residue was purified by silica gel column chromatography, eluting with EtOAc/PE=45% to afford the product (1.17 g, 82%). LCMS (ESI) m/z [M+H]⁺=358.3.

### Step 9: trans-3-(5-amino-1-(tert-butyl)-1H-pyrazol-3-yl)cyclopentan-1-ol

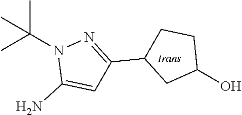 [SMILES: CC(C)(C)n1nc(C2CCC(O)C2)cc1N]

To a solution of trans-3-(5-(((benzyloxy)carbonyl)amino)-1-(tert-butyl)-1H-pyrazol-3-yl)cyclopentyl 4-nitrobenzoate (1.17 g, 3.3 mmol) in THF (100 mL) was added Pd/C (10%, wet, 1 g). The suspension was degassed and purged with hydrogen 3 times. The resulting mixture was stirred at room temperature under a hydrogen balloon for 3 h. The mixture was filtered, and the filter cake was washed with MeOH (50 mL×3). The filtrate was concentrated under reduced pressure to afford the product (677 mg, 92% yield). LC-MS (ESI): m/z [M+H]⁺=224.3.

### Step 10: trans-5-((1-(tert-butyl)-3-(3-hydroxycyclopentyl)-1H-pyrazol-5-yl)amino)-4-fluoro-2-(4-methoxybenzyl)-2,3-dihydrobenzo[d]isothiazole 1,1-dioxide

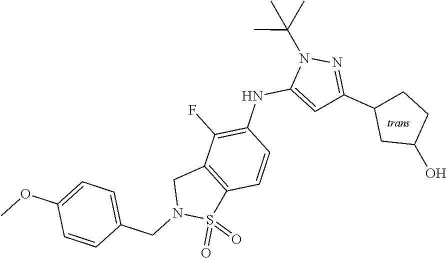 [SMILES: COc1ccc(CN2Cc3c(ccc(Nc4cc(C5CCC(O)C5)nn4C(C)(C)C)c3F)S2(=O)=O)cc1]

A mixture of 5-bromo-4-fluoro-2-(4-methoxybenzyl)-2,3-dihydrobenzo[d]isothiazole 1,1-dioxide (1.16 g, 3.0 mmol), trans-3-(5-amino-1-(tert-butyl)-1H-pyrazol-3-yl)cyclopentan-1-ol (677 mg, 3.0 mmol), Brettphos Pd G3 (272 mg, 0.3 mmol), K₂CO₃ (1.2 g, 9 mmol) in t-BuOH (100 mL) was stirred at 110° C. for 16 h under N₂ atmosphere. The mixture was concentrated under reduced pressure, and the residue was purified by silica gel column chromatography, eluting with EA/PE (4:1) to obtain the product (618 mg, 39% yield). LC-MS (ESI): m/z [M+H]⁺=529.3.

### Step 11: cis-2-(3-(1-(tert-butyl)-5-((4-fluoro-2-(4-methoxybenzyl)-1,1-dioxido-2,3-dihydrobenzo[d]isothiazol-5-yl)amino)-1H-pyrazol-3-yl)cyclopentyl)pyridazin-3 (2H)-one

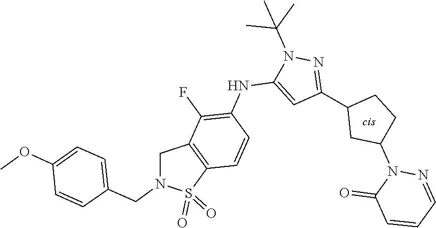 [SMILES: COc1ccc(CN2Cc3c(ccc(Nc4cc(C5CCC(n6ncccc6=O)C5)nn4C(C)(C)C)c3F)S2(=O)=O)cc1]

A mixture of trans-5-((1-(tert-butyl)-3-(3-hydroxycyclopentyl)-1H-pyrazol-5-yl)amino)-4-fluoro-2-(4-methoxybenzyl)-2,3-dihydrobenzo[d]isothiazole 1,1-dioxide (0.1 g, 0.189 mmol), pyridazin-3 (2H)-one (0.055 g, 0.573 mmol) and Cyanomethylenetributylphosphorane (0.137 g, 0.568 mmol) in toluene (5 mL) was heated at 95° C. under N₂ for 15 hrs. After cooled to 25° C., the mixture was concentrated under reduced pressure. The residue was purified by silica gel column chromatography, eluting with PE/EtOAc (1:1) to afford the product (0.077 g, 66.95%). LC-MS (ESI): m/z [M+H]⁺=607.3

### Step 12: cis-2-(3-(3-((4-fluoro-1,1-dioxido-2,3-dihydrobenzo[d]isothiazol-5-yl)amino)-1H-pyrazol-5-yl)cyclopentyl)pyridazin-3 (2H)-one

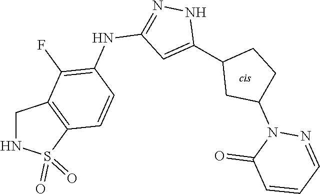 [SMILES: O=c1cccnn1C1CCC(c2cc(Nc3ccc4c(c3F)CNS4(=O)=O)n[nH]2)C1]

To a stirred solution of cis-2-(3-(1-(tert-butyl)-5-((4-fluoro-2-(4-methoxybenzyl)-1,1-dioxido-2,3-dihydrobenzo[d]isothiazol-5-yl)amino)-1H-pyrazol-3-yl)cyclopentyl)pyridazin-3 (2H)-one (77 mg, 0.127 mmol) in DCM (5 mL) was added triflic acid (2 mL) dropwise. The solution was stirred at room temperature for 2 h. The solution was diluted with DCM (20 mL) and basified to pH=8 with sat. aq NaHCO₃ at 0° C. The layers separated and the aqueous layer was extracted with DCM (30 mL×3). The combined organic layers were dried over anhydrous Na₂SO₄, filtered, and concentrated under reduced pressure. The residue was purified by prep HPLC (Waters SunFire C18: RD-CO-058 column, eluting with 10%-49% of water (containing 0.1% FA) in acetonitrile to afford the product (12 mg, 21.94%). ¹H NMR (500 MHz, DMSO-d₆) δ 12.06 (s, 1H), 8.73 (d, J=2.0 Hz, 1H), 8.27 (s, 1H), 7.99 (dd, J=3.8, 1.6 Hz, 1H), 7.70 (t, J=5.1 Hz, 1H), 7.48 (d, J=8.6 Hz, 1H), 7.40 (dd, J=9.4, 3.8 Hz, 1H), 6.92 (dd, J=9.4, 1.7 Hz, 1H), 5.88 (s, 1H), 5.49-5.42 (m, 1H), 4.41 (s, 2H), 3.25-3.18 (m, 1H), 2.48-2.41 (m, 1H), 2.16-2.07 (m, 2H), 2.00-1.84 (m, 3H). LC-MS (ESI): m/z [M+H]⁺=431.3

### Example 22: cis-4-fluoro-5-((5-(3-(pyridazin-3-yloxy)cyclopentyl)-1H-pyrazol-3-yl)amino)-2,3-dihydrobenzo[d]isothiazole 1,1-dioxide

### Step 1: cis-5-((1-(tert-butyl)-3-(3-(pyridazin-3-yloxy)cyclopentyl)-1H-pyrazol-5-yl)amino)-4-fluoro-2-(4-methoxybenzyl)-2,3-dihydrobenzo[d]isothiazole 1,1-dioxide

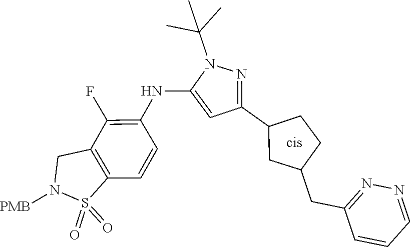

A mixture of trans-5-((1-(tert-butyl)-3-(3-hydroxycyclopentyl)-1H-pyrazol-5-yl)amino)-4-fluoro-2-(4-methoxybenzyl)-2,3-dihydrobenzo[d]isothiazole 1,1-dioxide (0.1 g, 0.189 mmol), pyridazin-3 (2H)-one (0.055 g, 0.573 mmol) and Cyanomethylenetributylphosphorane (0.137 g, 0.568 mmol) in toluene (5 mL) was heated at 95° C. under N₂ for 15 hrs. After cooled to 25° C., the mixture was concentrated under reduced pressure. The residue was purified by silica gel column chromatography, eluting with PE/EtOAc (1:1) to afford the product (22.1 mg, 19.4%). LC-MS (ESI): m/z [M+H]⁺=607.3

### Step 2: cis-4-fluoro-5-((5-(3-(pyridazin-3-yloxy)cyclopentyl)-1H-pyrazol-3-yl)amino)-2,3-dihydrobenzo[d]isothiazole 1,1-dioxide

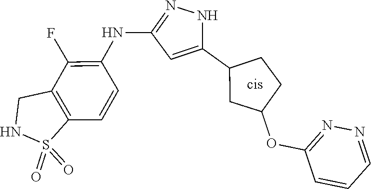 [SMILES: O=S1(=O)NCc2c1ccc(Nc1cc(C3CCC(Oc4cccnn4)C3)[nH]n1)c2F]

To a stirred solution of cis-5-((1-(tert-butyl)-3-(3-(pyridazin-3-yloxy)cyclopentyl)-1H-pyrazol-5-yl)amino)-4-fluoro-2-(4-methoxybenzyl)-2,3-dihydrobenzo[d]isothiazole 1,1-dioxide (22.1 mg, 0.037 mmol) in DCM (5 mL) was added triflic acid (2 mL) dropwise. The solution was stirred at room temperature for 2 h. The solution was diluted with DCM (20 mL) and basified to pH=8 with sat. aq NaHCO₃ at 0° C. The layers separated and the aqueous layer was extracted with DCM (30 mL×3). The combined organic layers were dried over anhydrous Na₂SO₄, filtered, and concentrated under reduced pressure. The residue was purified by prep HPLC (Waters SunFire C18: RD-CO-058 column, eluting with 10%-49% of water (containing 0.1% FA) in acetonitrile to afford the product (12 mg, 21.94%). ¹H NMR (500 MHz, DMSO-d₆) δ 12.03 (s, 1H), 8.87 (d, J=3.3 Hz, 1H), 8.72 (s, 1H), 8.27 (s, 1H), 7.69 (s, 1H), 7.61 (dd, J=8.9, 4.4 Hz, 1H), 7.47 (d, J=8.6 Hz, 1H), 7.17 (d, J=8.9 Hz, 1H), 5.85 (s, 1H), 5.60 (s, 1H), 4.40 (d, J=5.1 Hz, 2H), 3.24-3.13 (m, 1H), 2.74-2.66 (m, 1H), 2.17-2.08 (m, 2H), 1.96-1.78 (m, 3H). LC-MS (ESI): m/z [M+H]⁺=431.3.

### Example 23: cis-2-(3-(3-((4-fluoro-1,1-dioxido-2,3-dihydrobenzo[d]isothiazol-5-yl)amino)-1H-pyrazol-5-yl)cyclopentyl)-4-methylpyridazin-3 (2H)-one

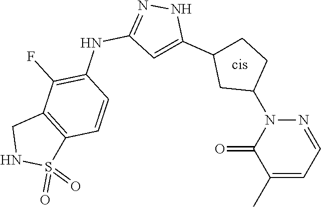 [SMILES: Cc1ccnn(C2CCC(c3cc(Nc4ccc5c(c4F)CNS5(=O)=O)n[nH]3)C2)c1=O]

### Step 1: cis-2-(3-(1-(tert-butyl)-5-((4-fluoro-2-(4-methoxybenzyl)-1,1-dioxido-2,3-dihydrobenzo[d]isothiazol-5-yl)amino)-1H-pyrazol-3-yl)cyclopentyl)-4-methylpyridazin-3 (2H)-one

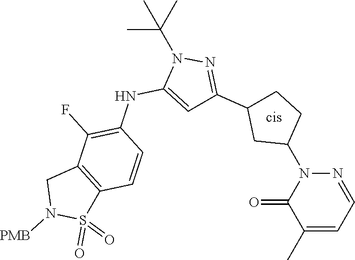

To a solution of trans-5-((1-(tert-butyl)-3-(3-hydroxycyclopentyl)-1H-pyrazol-5-yl)amino)-4-fluoro-2-(4-methoxybenzyl)-2,3-dihydrobenzo[d]isothiazole 1,1-dioxide (100 mg, 0.19 mmol) and 4-methylpyridazin-3-ol (21 mg, 0.19 mmol) in toluene (3 mL) was added CMBP (137 mg, 0.57 mmol). The reaction mixture was stirred for 16 h at 100° C. under nitrogen. The resulting mixture was cooled to room temperature, concentrated, purified by silica gel column chromatography, eluting with PE/EA (1:1) to afford the product (110 mg, 93%). LC-MS (ESI): m/z [M+H]⁺=621.4.

### Step 2: cis-2-(3-(3-((4-fluoro-1,1-dioxido-2,3-dihydrobenzo[d]isothiazol-5-yl)amino)-1H-pyrazol-5-yl)cyclopentyl)-4-methylpyridazin-3 (2H)-one

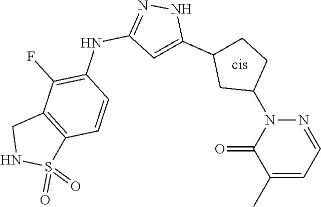 [SMILES: Cc1ccnn(C2CCC(c3cc(Nc4ccc5c(c4F)CNS5(=O)=O)n[nH]3)C2)c1=O]

The titled compound was synthesized in the procedures similar to Example 21, step 12. ¹H NMR (500 MHz, DMSO-d₆) δ=12.06 (s, 1H), 8.73 (s, 1H), 8.26 (s, 1H), 7.88 (d, J=3.9, 1H), 7.69 (s, 1H), 7.48 (d, J=8.6, 1H), 7.28 (dd, J=3.9, 1.1, 1H), 5.87 (s, 1H), 5.53-5.41 (m, 1H), 4.41 (s, 2H), 3.26-3.17 (m, 1H), 2.41 (m, 1H), 2.16-2.05 (m, 5H), 1.96 (m, 2H), 1.87 (m, 1H). LC-MS (ESI): m/z [M+H]+=445.1.

### Example 24: cis-4-fluoro-5-((5-(3-((4-methylpyridazin-3-yl)oxy)cyclopentyl)-1H-pyrazol-3-yl)amino)-2,3-dihydrobenzo[d]isothiazole 1,1-dioxide

### Step 1: cis-3-(5-amino-1-(tert-butyl)-1H-pyrazol-3-yl)cyclopentan-1-ol

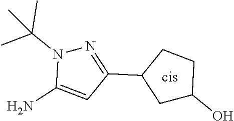 [SMILES: CC(C)(C)n1nc(C2CCC(O)C2)cc1N]

To a solution of cis-benzyl (1-(tert-butyl)-3-(3-hydroxycyclopentyl)-1H-pyrazol-5-yl)carbamate (3.6 g, 10 mmol) in THF (100 mL) was added Pd/C (10%, wet, 2 g). The suspension was degassed and purged with hydrogen 3 times. The resulting mixture was stirred at room temperature under a hydrogen balloon for 3 h. The mixture was filtered, and the filter cake was washed with EA (50 mL×3). The filtrate was concentrated under reduced pressure to afford the product (2.1 g, 91% yield). LC-MS (ESI): m/z [M+H]⁺=224.3.

### Step 2: cis-5-((1-(tert-butyl)-3-(3-hydroxycyclopentyl)-1H-pyrazol-5-yl)amino)-4-fluoro-2-(4-methoxybenzyl)-2,3-dihydrobenzo[d]isothiazole 1,1-dioxide

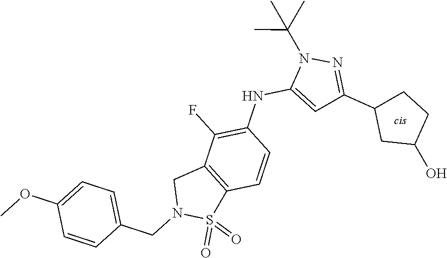 [SMILES: COc1ccc(CN2Cc3c(ccc(Nc4cc(C5CCC(O)C5)nn4C(C)(C)C)c3F)S2(=O)=O)cc1]

A mixture of 5-bromo-4-fluoro-2-(4-methoxybenzyl)-2,3-dihydrobenzo[d]isothiazole 1,1-dioxide (1.16 g, 3.0 mmol), cis-3-(5-amino-1-(tert-butyl)-1H-pyrazol-3-yl)cyclopentan-1-ol (677 mg, 3.0 mmol), Brettphos Pd G3 (272 mg, 0.3 mmol), K₂CO₃ (1.2 g, 9 mmol) in t-BuOH (100 mL) was stirred at 110° C. for 16 h under N₂ atmosphere. The mixture was concentrated under reduced pressure, and the residue was purified by silica gel column chromatography, eluting with EA/PE (4:1) to obtain the product (618 mg, 39% yield). LC-MS (ESI): m/z [M+H]⁺=529.3.

### Step 3: cis-5-((1-(tert-butyl)-3-(3-((4-methylpyridazin-3-yl)oxy)cyclopentyl)-1H-pyrazol-5-yl)amino)-4-fluoro-2-(4-methoxybenzyl)-2,3-dihydrobenzo[d]isothiazole 1,1-dioxide

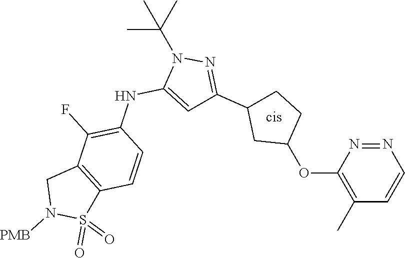

To a solution of cis-5-((1-(tert-butyl)-3-(3-hydroxycyclopentyl)-1H-pyrazol-5-yl)amino)-4-fluoro-2-(4-methoxybenzyl)-2,3-dihydrobenzo[d]isothiazole 1,1-dioxide (150 mg, 0.284 mmol) and 3-chloro-4-methylpyridazine (37 mg, 0.284 mmol) in toluene (5 mL) was added Pd₂dba₃ (26 mg, 0.028 mmol), Binap (35 mg, 0.057 mmol) and t-BuONa (82 mg, 0.852 mmol). The reaction mixture was stirred for 4 hours at 95° C. under nitrogen. The resulting mixture was cooled to room temperature, concentrated under reduce pressure, purified by silica gel column chromatography, eluting with PE/EA (1:1) to afford the product (60 mg, 34%). LC-MS (ESI): m/z [M+H]⁺=621.3.

### Step 4: cis-4-fluoro-5-((5-(3-((4-methylpyridazin-3-yl)oxy)cyclopentyl)-1H-pyrazol-3-yl)amino)-2,3-dihydrobenzo[d]isothiazole 1,1-dioxide

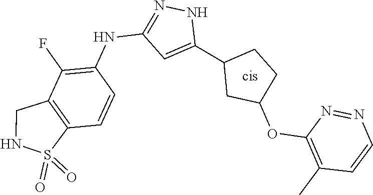 [SMILES: Cc1ccnnc1OC1CCC(c2cc(Nc3ccc4c(c3F)CNS4(=O)=O)n[nH]2)C1]

The titled compound was synthesized in the procedures similar to Example 21, step 12. ¹H NMR (500 MHz, DMSO-d₆) δ=12.03 (s, 1H), 8.72 (s, 1H), 8.69 (d, J=4.6, 1H), 8.27 (d, J=9.0, 1H), 7.69 (s, 1H), 7.47 (d, J=8.6, 1H), 7.44 (d, J=4.5, 1H), 5.84 (s, 1H), 5.63 (s, 1H), 4.40 (d, J=4.5, 2H), 3.25-3.16 (m, 1H), 2.71-2.61 (m, 1H), 2.13 (s, 3H), 2.09 (m, 2H), 1.95 (m, 1H), 1.91-1.79 (m, 2H). LC-MS (ESI): m/z [M+H]⁺=445.1.

### Example 25: cis-5-((5-(3-((4-ethylpyridazin-3-yl)oxy)cyclopentyl)-1H-pyrazol-3-yl)amino)-4-fluoro-2,3-dihydrobenzo[d]isothiazole 1,1-dioxide

### Step 1: cis-5-((1-(tert-butyl)-3-(3-((4-ethylpyridazin-3-yl)oxy)cyclopentyl)-1H-pyrazol-5-yl)amino)-4-fluoro-2-(4-methoxybenzyl)-2,3-dihydrobenzo[d]isothiazole 1,1-dioxide

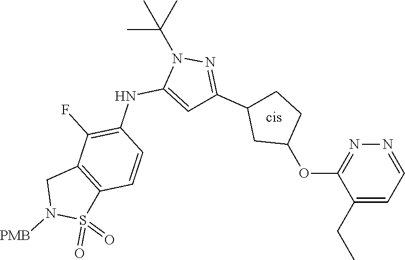

To a solution of cis-5-((1-(tert-butyl)-3-(3-hydroxycyclopentyl)-1H-pyrazol-5-yl)amino)-4-fluoro-2-(4-methoxybenzyl)-2,3-dihydrobenzo[d]isothiazole 1,1-dioxide (150 mg, 0.284 mmol) and 3-chloro-4-ethylpyridazine (61 mg, 0.284 mmol) in toluene (15 mL) was added Pd₂dba₃ (26 mg, 0.028 mmol), Binap (35 mg, 0.059 mmol) and t-BuONa (82 mg, 0.852 mmol), the reaction mixture was stirred for 16 hours at 100° C. under nitrogen. The resulting mixture was cooled to room temperature, concentrated, purified by silica gel column chromatography, eluting with PE/EA (1:1) to afford the product (90 mg, 48%). LC-MS (ESI): m/z [M+H]⁺=636.3.

### Step 2: cis-5-((5-(3-((4-ethylpyridazin-3-yl)oxy)cyclopentyl)-1H-pyrazol-3-yl)amino)-4-fluoro-2,3-dihydrobenzo[d]isothiazole 1,1-dioxide

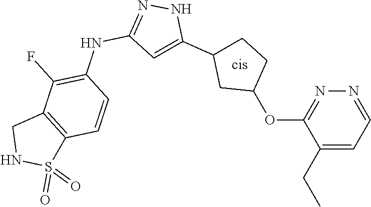 [SMILES: CCc1ccnnc1OC1CCC(c2cc(Nc3ccc4c(c3F)CNS4(=O)=O)n[nH]2)C1]

To a stirring solution of cis-5-((1-(tert-butyl)-3-(3-((4-ethylpyridazin-3-yl)oxy)cyclopentyl)-1H-pyrazol-5-yl)amino)-4-fluoro-2-(4-methoxybenzyl)-2,3-dihydrobenzo[d]isothiazole 1,1-dioxide (50 mg, 0.086 mmol) was added FA (10 mL), The solution was stirred for 12 h at 75° C. The mixture was concentrated under reduced pressure. The residue was purified by silica gel column chromatography, eluting with 100% EA to afford the crude product, which was further purified by prep HPLC (Waters XSelect C18: RD-CO-094 column, eluting with a gradient of acetonitrile/water containing 0.1% FA, 20%-40%) to afford the product (5 mg, 17.8%). ¹H NMR (500 MHz, DMSO-d₆) δ 12.03 (s, 1H), 8.76-8.65 (m, 1H), 8.26 (s, OH), 7.69 (t, J=4.9 Hz, 1H), 7.47 (d, J=8.5 Hz, OH), 7.41 (d, J=4.5 Hz, 1H), 5.83 (s, 1H), 5.64 (s, 1H), 4.40 (d, J=5.0 Hz, 2H), 3.25-3.13 (m, 1H), 2.74-2.63 (m, 1H), 2.16-2.06 (m, 1H), 2.00-1.80 (m, 2H), 1.12 (t, J=7.5 Hz, 2H). LC-MS (ESI): m/z [M+H]⁺=459.2.

### Example 26: cis-2-(3-(3-((4-fluoro-1,1-dioxido-2,3-dihydrobenzo[d]isothiazol-5-yl)amino)-1H-pyrazol-5-yl)cyclopentyl)-4-isopropylpyridazin-3 (2H)-one

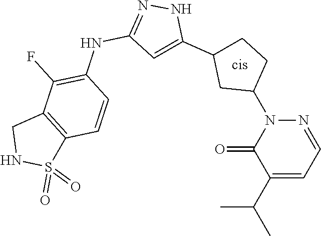 [SMILES: CC(C)c1ccnn(C2CCC(c3cc(Nc4ccc5c(c4F)CNS5(=O)=O)n[nH]3)C2)c1=O]

The titled compound was synthesized in the procedures similar to Example 21. ¹H NMR (500 MHz, DMSO-d₆) δ 12.05 (s, 1H), 8.73 (s, 1H), 8.27 (s, 1H), 7.94 (d, J=4.1 Hz, 1H), 7.69 (t, J=5.0 Hz, 1H), 7.48 (d, J=8.5 Hz, 1H), 7.20 (d, J=4.1 Hz, 1H), 5.88 (s, 1H), 5.50 (dd, J=15.4, 7.7 Hz, 1H), 4.41 (d, J=5.3 Hz, 2H), 3.26-3.18 (m, 1H), 3.11-3.03 (m, 1H), 2.48-2.41 (m, 1H), 2.16-2.05 (m, 2H), 2.00-1.85 (m, 3H), 1.14 (d, J=6.9 Hz, 6H). LC-MS (ESI): m/z [M+H]⁺=473.3

### Example 27: cis-4-fluoro-5-((5-(3-((4-isopropylpyridazin-3-yl)oxy)cyclopentyl)-1H-pyrazol-3-yl)amino)-2,3-dihydrobenzo[d]isothiazole 1,1-dioxide

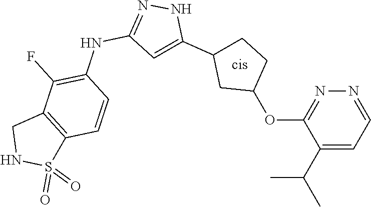 [SMILES: CC(C)c1ccnnc1OC1CCC(c2cc(Nc3ccc4c(c3F)CNS4(=O)=O)n[nH]2)C1]

The titled compound was synthesized in the procedures similar to Example 22. ¹H NMR (500 MHz, DMSO-d₆) δ 12.03 (s, 1H), 8.76 (d, J=4.7 Hz, 1H), 8.72 (s, 1H), 8.26 (s, 1H), 7.69 (t, J=4.8 Hz, 1H), 7.47 (d, J=8.6 Hz, 1H), 7.42 (d, J=4.7 Hz, 1H), 5.83 (s, 1H), 5.66 (s, 1H), 4.40 (d, J=5.2 Hz, 2H), 3.24-3.18 (m, 1H), 3.03-2.97 (m, 1H), 2.72-2.65 (m, 1H), 2.17-2.08 (m, 2H), 2.01-1.82 (m, 3H), 1.14 (dd, J=6.7, 4.4 Hz, 6H). LC-MS (ESI): m/z [M+H]⁺=473.3.

### Example 28: cis-4-cyclopropyl-2-(3-(3-((4-fluoro-1,1-dioxido-2,3-dihydrobenzo[d]isothiazol-5-yl)amino)-1H-pyrazol-5-yl)cyclopentyl)pyridazin-3 (2H)-one

### Step 1: cis-2-(3-(1-(tert-butyl)-5-((4-fluoro-2-(4-methoxybenzyl)-1,1-dioxido-2,3-dihydrobenzo[d]isothiazol-5-yl)amino)-1H-pyrazol-3-yl)cyclopentyl)-4-chloropyridazin-3 (2H)-one

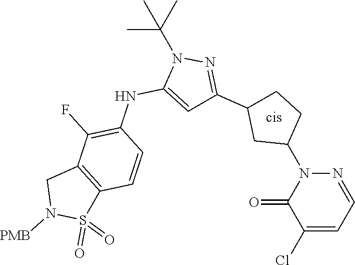

To a mixture of trans-5-((1-(tert-butyl)-3-(3-hydroxycyclopentyl)-1H-pyrazol-5-yl)amino)-4-fluoro-2-(4-methoxybenzyl)-2,3-dihydrobenzo[d]isothiazole 1,1-dioxide (200 mg, 0.38 mmol) and 4-chloropyridazin-3-ol (103 mg, 0.76 mmol) in toluene (10 mL) was added CMBP (274 mg, 1.14 mmol). The mixture was stirred at 95° C. for 16 h under a nitrogen atmosphere. The mixture was concentrated under vacuum. The residue was purified by silica gel column chromatography, eluting with EA/PE (0-65%) to afford the product (153 mg, 62.9%). LC-MS (ESI): m/z [M+H]⁺=641.

### Step 2: cis-2-(3-(1-(tert-butyl)-5-((4-fluoro-2-(4-methoxybenzyl)-1,1-dioxido-2,3-dihydrobenzo[d]isothiazol-5-yl)amino)-1H-pyrazol-3-yl)cyclopentyl)-4-cyclopropylpyridazin-3 (2H)-one

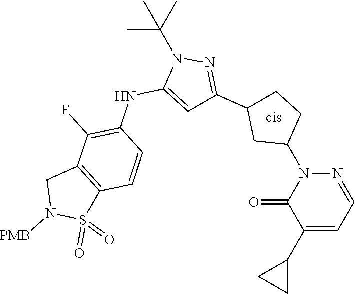

To a solution of cis-2-(3-(1-(tert-butyl)-5-((4-fluoro-2-(4-methoxybenzyl)-1,1-dioxido-2,3-dihydrobenzo[d]isothiazol-5-yl)amino)-1H-pyrazol-3-yl)cyclopentyl)-4-chloropyridazin-3 (2H)-one (153 mg, 0.24 mmol) in dioxane (10 mL) and H₂O (1 mL) were added cyclopropylboronic acid (31 mg, 0.36 mmol), Pd (OAc)₂ (2.8 mg, 0.012 mmol) and PCy₃ (8 mg, 0.03 mmol). The mixture was stirred at 110° C. for 16 h under a nitrogen atmosphere. The mixture was concentrated under vacuum. The residue was purified by silica gel column chromatography, eluting with EA/PE (0-80%) to afford the product (135 mg, 87%). LC-MS (ESI): m/z [M+H]⁺=647.

### Step 3: cis-4-cyclopropyl-2-(3-(3-((4-fluoro-1,1-dioxido-2,3-dihydrobenzo[d]isothiazol-5-yl)amino)-1H-pyrazol-5-yl)cyclopentyl)pyridazin-3 (2H)-one

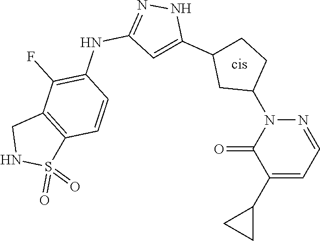 [SMILES: O=c1c(C2CC2)ccnn1C1CCC(c2cc(Nc3ccc4c(c3F)CNS4(=O)=O)n[nH]2)C1]

The titled compound was synthesized in the procedures similar to Example 21. ¹H NMR (500 MHz, DMSO-d₆) δ 12.05 (s, 1H), 8.72 (d, J=5 Hz, 1H), 8.29-8.20 (m, 1H), 7.84 (d, J=5 Hz, 1H), 7.71-7.65 (m, 1H), 7.48 (d, J=5 Hz, 1H), 6.87 (d, J=5 Hz, 1H), 5.87 (s, 1H), 5.52-5.43 (m, 1H), 4.40 (s, 2H), 3.25-3.18 (m, 1H), 2.46-2.30 (m, 1H), 2.20-2.04 (m, 3H), 2.01-1.82 (m, 2H), 1.15-0.97 (m, 2H), 0.84-0.78 (m, 2H). LC-MS (ESI): m/z [M+H]⁺=471.2.

### Example 29: cis-5-((5-(3-((4-cyclopropylpyridazin-3-yl)oxy)cyclopentyl)-1H-pyrazol-3-yl)amino)-4-fluoro-2,3-dihydrobenzo[d]isothiazole 1,1-dioxide

### Step 1: cis-5-((1-(tert-butyl)-3-(3-((4-chloropyridazin-3-yl)oxy)cyclopentyl)-1H-pyrazol-5-yl)amino)-4-fluoro-2-(4-methoxybenzyl)-2,3-dihydrobenzo[d]isothiazole 1,1-dioxide

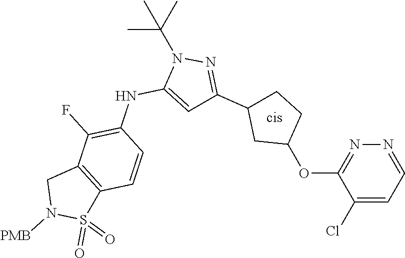

To a mixture of trans-5-((1-(tert-butyl)-3-(3-hydroxycyclopentyl)-1H-pyrazol-5-yl)amino)-4-fluoro-2-(4-methoxybenzyl)-2,3-dihydrobenzo[d]isothiazole 1,1-dioxide (200 mg, 0.38 mmol) and 4-chloropyridazin-3-ol (103 mg, 0.76 mmol) in toluene (10 mL) was added CMBP (274 mg, 1.14 mmol). The mixture was stirred at 95° C. for 16 h under a nitrogen atmosphere. The mixture was concentrated under vacuum. The residue was purified by silica gel column chromatography, eluting with EA/PE (0-65%) to afford the product (50 mg, 20.4%). LC-MS (ESI): m/z [M+H]⁺=641.

### Step 2: cis-5-((1-(tert-butyl)-3-(3-((4-cyclopropylpyridazin-3-yl)oxy)cyclopentyl)-1H-pyrazol-5-yl)amino)-4-fluoro-2-(4-methoxybenzyl)-2,3-dihydrobenzo[d]isothiazole 1,1-dioxide

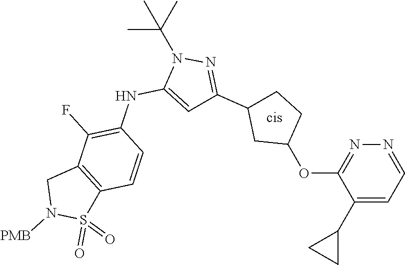

To a solution of cis-5-((1-(tert-butyl)-3-(3-((4-chloropyridazin-3-yl)oxy)cyclopentyl)-1H-pyrazol-5-yl)amino)-4-fluoro-2-(4-methoxybenzyl)-2,3-dihydrobenzo[d]isothiazole 1,1-dioxide (50 mg, 0.078 mmol) in dioxane (2.5 mL) and H₂O (0.5 mL) were added cyclopropylboronic acid (33.5 mg, 0.39 mmol), Pd (OAc)₂ (1.3 mg, 0.007 mmol), PCy3 (4.4 mg, 0.016 mmol). The mixture was stirred at 110° C. for 16 h under a nitrogen atmosphere. The mixture was concentrated under vacuum. The residue was purified by silica gel column chromatography, eluting with EA/PE (0-80%) to afford the product (46 mg, 91.1%). LC-MS (ESI): m/z [M+H]⁺=647.

### Step 3: cis-5-((5-(3-((4-cyclopropylpyridazin-3-yl)oxy)cyclopentyl)-1H-pyrazol-3-yl)amino)-4-fluoro-2,3-dihydrobenzo[d]isothiazole 1,1-dioxide

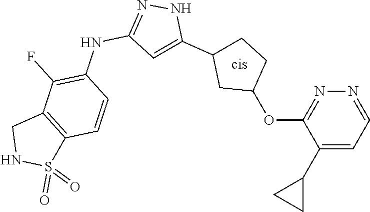 [SMILES: O=S1(=O)NCc2c1ccc(Nc1cc(C3CCC(Oc4nnccc4C4CC4)C3)[nH]n1)c2F]

To a solution of cis-5-((1-(tert-butyl)-3-(3-((4-cyclopropylpyridazin-3-yl)oxy)cyclopentyl)-1H-pyrazol-5-yl)amino)-4-fluoro-2-(4-methoxybenzyl)-2,3-dihydrobenzo[d]isothiazole 1,1-dioxide (46 mg, 0.07 mmol) in DCM (10 mL) was added TfOH (1 mL). The mixture was stirred at rt for 2 h. The mixture was diluted with DCM (50 mL), washed with sat NaHCO₃ (3×30 mL). The combined organic layers were dried over Na₂SO₄, filtered and concentrated under vacuum. The residue was purified by Prep-HPLC to afford product (2.5 mg, 7.5%). ¹H NMR (500 MHz, DMSO-d₆) δ 12.05 (s, 1H), 8.74 (d, J=5 Hz, 1H), 8.71-8.68 (m, 1H), 8.28-8.17 (m, 1H), 7.75-7.62 (m, 1H), 7.46 (d, J=10 Hz, 1H), 7.19 (d, J=5 Hz, 1H), 5.85 (s, 1H), 5.66-5.58 (m, 1H), 4.40 (s, 2H), 3.27-3.14 (m, 1H), 2.75-2.63 (m, 1H), 2.18-2.08 (m, 2H), 2.05-1.94 (m, 2H), 1.93-1.83 (m, 2H), 1.10-1.00 (m, 2H), 0.90-0.83 (m, 2H). LC-MS (ESI): m/z [M+H]+=471.2.

### Example 30: cis-2-(3-(3-((4-fluoro-1,1-dioxido-2,3-dihydrobenzo[d]isothiazol-5-yl)amino)-1H-pyrazol-5-yl)cyclopentyl)-4-phenylpyridazin-3 (2H)-one

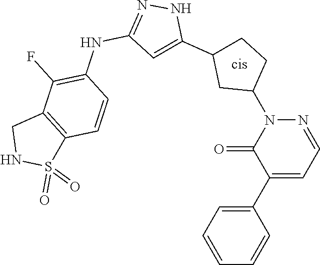 [SMILES: O=c1c(-c2ccccc2)ccnn1C1CCC(c2cc(Nc3ccc4c(c3F)CNS4(=O)=O)n[nH]2)C1]

The titled compound was synthesized in the procedures similar to Example 21. ¹H NMR (500 MHz, DMSO-d₆) δ 12.04 (s, 1H), 8.93 (d, J=5 Hz, 1H), 8.71-8.65 (m, 1H), 8.29-8.16 (m, 1H), 7.72-7.61 (m, 4H), 7.50-7.38 (m, 4H), 5.79 (s, 1H), 5.73-5.66 (m, 1H), 4.40 (d, J=5 Hz, 2H), 3.24-3.15 (m, 1H), 2.75-2.66 (m, 1H), 2.21-2.06 (m, 2H), 2.00-1.90 (m, 1H), 1.90-1.77 (m, 2H). LC-MS (ESI): m/z [M+H]+=507.2.

### Example 31: cis-4-fluoro-5-((5-(3-((4-phenylpyridazin-3-yl)oxy)cyclopentyl)-1H-pyrazol-3-yl)amino)-2,3-dihydrobenzo[d]isothiazole 1,1-dioxide

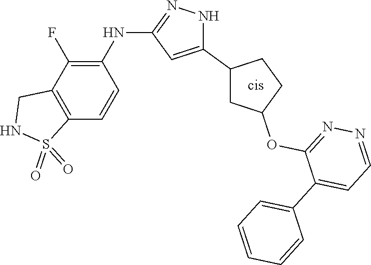 [SMILES: O=S1(=O)NCc2c1ccc(Nc1cc(C3CCC(Oc4nnccc4-c4ccccc4)C3)[nH]n1)c2F]

The titled compound was synthesized in the procedures similar to Example 22. ¹H NMR (500 MHz, DMSO-d₆) δ 12.05 (s, 1H), 8.70 (s, 1H), 8.30-8.20 (m, 1H), 8.09-8.04 (m, 1H), 7.85-7.78 (m, 2H), 7.69-7.62 (m, 1H), 7.58-7.53 (m, 1H), 7.50-7.40 (m, 4H), 5.90 (s, 1H), 5.60-5.50 (m, 1H), 4.40 (s, 2H), 3.30-3.19 (m, 1H), 3.47-3.42 (m, 1H), 2.20-2.10 (m, 2H), 2.08-1.97 (m, 2H), 1.96-1.85 (m, 1H). LC-MS (ESI): m/z [M+H]⁺=507.2.

### Example 32: cis-2-(3-(3-((4-fluoro-1,1-dioxido-2,3-dihydrobenzo[d]isothiazol-5-yl)amino)-1H-pyrazol-5-yl)cyclopentyl)-5-(prop-1-en-2-yl)pyridazin-3 (2H)-one

### Step 1: cis-2-(3-(1-(tert-butyl)-5-((4-fluoro-2-(4-methoxybenzyl)-1,1-dioxido-2,3-dihydrobenzo[d]isothiazol-5-yl)amino)-1H-pyrazol-3-yl)cyclopentyl)-5-chloropyridazin-3 (2H)-one

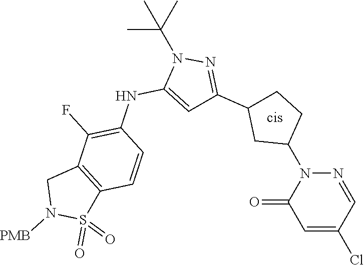

To a mixture of trans-5-((1-(tert-butyl)-3-(3-hydroxycyclopentyl)-1H-pyrazol-5-yl)amino)-4-fluoro-2-(4-methoxybenzyl)-2,3-dihydrobenzo[d]isothiazole 1,1-dioxide (200 mg, 0.38 mmol) and 4-chloropyridazin-3-ol (103 mg, 0.76 mmol) in toluene (10 mL) was added CMBP (274 mg, 1.14 mmol). The mixture was stirred at 95° C. for 16 h under a nitrogen atmosphere. The mixture was concentrated under vacuum. The residue was purified by silica gel column chromatography, eluting with EA/PE (0-65%) to afford the product (50 mg, 20.4%). LC-MS (ESI): m/z [M+H]⁺=641.

### Step 2: cis-2-(3-(1-(tert-butyl)-5-((4-fluoro-1,1-dioxido-2,3-dihydrobenzo[d]isothiazol-5-yl)amino)-1H-pyrazol-3-yl)cyclopentyl)-5-(prop-1-en-2-yl)pyridazin-3 (2H)-one

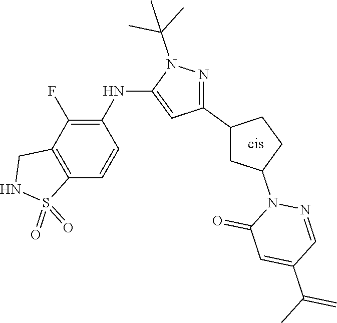 [SMILES: C=C(C)c1cnn(C2CCC(c3cc(Nc4ccc5c(c4F)CNS5(=O)=O)n(C(C)(C)C)n3)C2)c(=O)c1]

To a solution of cis-2-(3-(1-(tert-butyl)-5-((4-fluoro-2-(4-methoxybenzyl)-1,1-dioxido-2,3-dihydrobenzo[d]isothiazol-5-yl)amino)-1H-pyrazol-3-yl)cyclopentyl)-5-chloropyridazin-3 (2H)-one (50 mg, 0.08 mmol) in dioxane/H₂O (5/1 mL) was added potassium isopropenyltrifluoroborate (15 mg, 0.1 mmol), Pd(dppf)Cl₂ (7 mg, 0.01 mmol) and K₂CO₃ (33 mg, 0.24 mmol). The mixture was stirred at 100° C. for 4 h under nitrogen. The mixture was concentrated under vacuum and the residue was purified by silica gel column chromatography, eluting with EA/PE (0-50%) to afford the product (25.6 mg, 61%). LC-MS (ESI): m/z [M+H]⁺=527.4.

### Step 3: cis-2-(3-(3-((4-fluoro-1,1-dioxido-2,3-dihydrobenzo[d]isothiazol-5-yl)amino)-1H-pyrazol-5-yl)cyclopentyl)-5-(prop-1-en-2-yl)pyridazin-3 (2H)-one

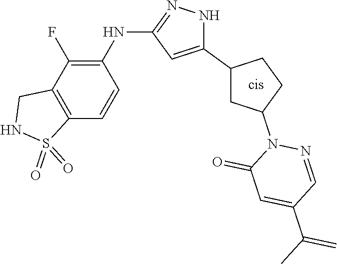 [SMILES: C=C(C)c1cnn(C2CCC(c3cc(Nc4ccc5c(c4F)CNS5(=O)=O)n[nH]3)C2)c(=O)c1]

The titled compound was synthesized in the procedures similar to Example 1, step 11. H NMR (500 MHz, DMSO-d₆) δ 12.06 (s, 1H), 8.73 (s, 1H), 8.31 (d, J=5 Hz, 1H), 8.30-8.23 (m, 1H), 7.72-7.66 (m, 1H), 7.48 (d, J=10 Hz, 1H), 6.81 (d, J=5 Hz, 1H), 5.86 (d, J=5 Hz, 2H), 5.47 (s, 1H), 5.45-5.38 (m, 1H), 4.44-4.38 (m, 2H), 3.28-3.16 (m, 1H), 2.47-2.39 (m, 1H), 2.18-2.07 (m, 2H), 2.04 (s, 3H), 2.00-1.83 (m, 3H). LC-MS (ESI): m/z [M+H]⁺=471.2.

### Example 33: cis-2-(3-(3-((4-fluoro-1,1-dioxido-2,3-dihydrobenzo[d]isothiazol-5-yl)amino)-1H-pyrazol-5-yl)cyclopentyl)-5-isopropylpyridazin-3 (2H)-one

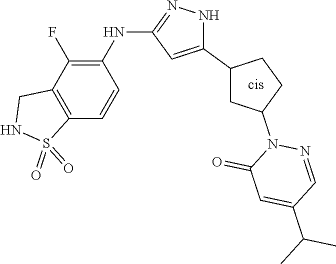 [SMILES: CC(C)c1cnn(C2CCC(c3cc(Nc4ccc5c(c4F)CNS5(=O)=O)n[nH]3)C2)c(=O)c1]

cis-2-(3-(3-((4-fluoro-1,1-dioxido-2,3-dihydrobenzo[d]isothiazol-5-yl)amino)-1H-pyrazol-5-yl)cyclopentyl)-5-(prop-1-en-2-yl)pyridazin-3 (2H)-one (20 mg, 0.038 mmol) was dissolved in THF (20 mL), Pd/C (10% on carbon, 10 mg) was added, the reaction mixture was stirred for 1 hour at room temperature under 1 atm H₂. The resulting mixture was filtered, the filtrated was concentrated and purified by silica gel column chromatography, eluting with EA/PE (0-100%) to afford the product (14 mg, 78%). H NMR (500 MHz, DMSO-d₆) δ 12.06 (s, 1H), 8.73 (s, 1H), 8.33-8.23 (m, 1H), 8.02-7.98 (m, 1H), 7.69 (t, J=5 Hz, 1H), 7.48 (d, J=10 Hz, 1H), 6.68-6.66 (m, 1H), 5.87 (s, 1H), 5.46-5.37 (m, 1H), 4.41 (d, J=5 Hz, 2H), 3.28-3.16 (m, 1H), 2.84-2.75 (m, 1H), 2.45-2.35 (m, 1H), 2.17-2.03 (m, 2H), 2.00-1.81 (m, 3H), 1.17 (d, J=5 Hz, 6H). LC-MS (ESI): m/z [M+H]⁺=473.2.

### Example 34: cis-2-(3-(3-((4-fluoro-1,1-dioxido-2,3-dihydrobenzo[d]isothiazol-5-yl)amino)-1H-pyrazol-5-yl)cyclopentyl)pyrido[3,4-d]pyridazin-1 (2H)-one

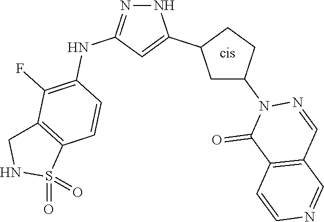 [SMILES: O=c1c2ccncc2cnn1C1CCC(c2cc(Nc3ccc4c(c3F)CNS4(=O)=O)n[nH]2)C1]

The titled compound was synthesized in the procedures similar to Example 21. ¹H NMR (500 MHz, DMSO-d₆) δ 12.07 (s, 1H), 9.34 (s, 1H), 8.99 (d, J=5.3 Hz, 1H), 8.74 (s, 1H), 8.68 (s, 1H), 8.29 (t, J=7.9 Hz, 1H), 8.11 (d, J=5.3 Hz, 1H), 7.69 (s, 1H), 7.48 (d, J=8.6 Hz, 1H), 5.90 (s, 1H), 5.62-5.53 (m, 1H), 4.41 (d, J=3.3 Hz, 2H), 3.30-3.21 (m, 1H), 2.47-2.43 (m, 1H), 2.20-2.11 (m, 2H), 2.09-1.99 (m, 2H), 1.97-1.89 (m, 1H). LC-MS (ESI): m/z [M+H]⁺=482.3

### Example 35: cis-5-((5-(3-((4-cyclobutylpyridazin-3-yl)oxy)cyclopentyl)-1H-pyrazol-3-yl)amino)-4-fluoro-2,3-dihydrobenzo[d]isothiazole 1,1-dioxide

### Step 1: 3-chloro-4-cyclobutylpyridazine

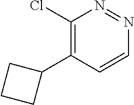 [SMILES: Clc1nnccc1C1CCC1]

To a solution of 3-chloropyridazine hydrochloride (15 g, 99.3 mmol) in MeCN (708 mL) and H₂O (708 mL) was added cyclobutanecarboxylic acid (9.9 g, 99.3 mmol), (NH₄)₂S₂O₈ (113.2 g, 496.5 mmol) and AgNO₃ (67.5 g, 397.2 mmol). The resulting solution was stirred for 2 h at 55° C. under nitrogen atmosphere. The solids were filtered out and the filtrate was extracted with EtOAc (3×150 mL). The combined organic layers were concentrated under vacuum. The residue was purified by silica gel column chromatography (EtOAc/PE=22%) to give the product (1.3 g, 7.8%). ¹H NMR (300 MHz, DMSO-d₆) δ 9.18 (d, J=5.0 Hz, 1H), 7.76 (m, J=5.1, 1.1 Hz, 1H), 3.77-3.62 (m, 1H), 2.45-2.32 (m, 2H), 2.22-2.03 (m, 3H), 1.92-1.75 (m, 1H).

### Step 2: cis-5-((5-(3-((4-cyclobutylpyridazin-3-yl)oxy)cyclopentyl)-1H-pyrazol-3-yl)amino)-4-fluoro-2,3-dihydrobenzo[d]isothiazole 1,1-dioxide

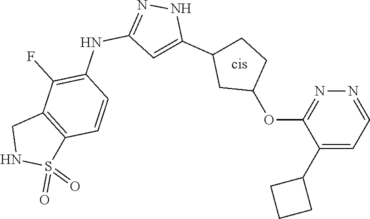 [SMILES: O=S1(=O)NCc2c1ccc(Nc1cc(C3CCC(Oc4nnccc4C4CCC4)C3)[nH]n1)c2F]

The titled compound was synthesized in the procedures similar to Example 29. ¹H NMR (500 MHz, DMSO-d₆) δ=12.03 (s, 1H), 8.84-8.70 (m, 2H), 8.27 (s, 1H), 7.69 (t, J=5.0 Hz, 1H), 7.47 (d, J=8.6 Hz, 1H), 7.40 (d, J=4.6 Hz, 1H), 5.83 (s, 1H), 5.62 (s, 1H), 4.40 (d, J=5.1 Hz, 2H), 3.52-3.44 (m, 1H), 3.24-3.14 (m, 1H), 2.71-2.62 (m, 1H), 2.28-2.17 (m, 2H), 2.13-1.99 (m, 4H), 1.98-1.90 (m, 2H), 1.90-1.79 (m, 2H), 1.79-1.69 (m, 1H). LC-MS (ESI): m/z [M+H]⁺=485.2.

### Example 36: trans-5-((5-(3-((4-(tert-butyl)pyridazin-3-yl)oxy)cyclopentyl)-1H-pyrazol-3-yl)amino)-4-fluoro-2,3-dihydrobenzo[d]isothiazole 1,1-dioxide

### Step 1: 4-(tert-butyl)-3-chloropyridazine

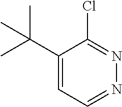

To a solution of 3-chloropyridazine hydrochloride (10 g, 66.2 mmol) in MeCN (475 mL) and H₂O (475 mL) were added pivalic acid (6.1 g, 59.6 mmol), (NH₄)₂S₂O₈ (75.5 g, 331 mmol), AgNO₃ (45 g, 264.8 mmol). The resulting solution was stirred for 2 h at 55° C. under nitrogen atmosphere. The solids were filtered out. The resulting mixture was concentrated under vacuum. Then the resulting solution was extracted with 3×100 mL of EtOAc. The organic layers were concentrated under vacuum. The residue was purified by silica gel column chromatography (EtOAc/PE=16%) to give the product (2 g, 17.7%). ¹H NMR (300 MHz, DMSO-d₆) δ 9.15 (d, J=5.2 Hz, 1H), 7.76 (d, J=5.2 Hz, 1H), 1.44 (s, 9H).

### Step 2: trans-5-((5-(3-((4-(tert-butyl)pyridazin-3-yl)oxy)cyclopentyl)-1H-pyrazol-3-yl)amino)-4-fluoro-2,3-dihydrobenzo[d]isothiazole 1,1-dioxide

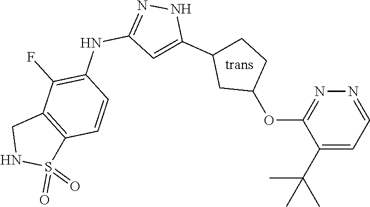 [SMILES: CC(C)(C)c1ccnnc1OC1CCC(c2cc(Nc3ccc4c(c3F)CNS4(=O)=O)n[nH]2)C1]

The titled compound was synthesized in the procedures similar to Example 24. ¹H NMR (500 MHz, DMSO-d₆) δ=12.07 (s, 1H), 8.76 (d, J=4.9 Hz, 2H), 8.27 (t, J=7.9 Hz, 1H), 7.69 (s, 1H), 7.48 (d, J=8.6 Hz, 1H), 7.42 (d, J=4.9 Hz, 1H), 5.88 (s, 1H), 5.79 (s, 1H), 4.41 (d, J=4.4 Hz, 2H), 3.41-3.35 (m, 1H), 2.40-2.30 (m, 6.4 Hz, 2H), 2.28-2.22 (m, 1H), 2.12-2.04 (m, 1H), 1.94-1.83 (m, 1H), 1.79-1.68 (m, 1H), 1.36 (s, 9H). LC-MS (ESI): m/z [M+H]⁺=487.2.

### Example 37: cis-5-((5-(3-((4-(tert-butyl)pyridazin-3-yl)oxy)cyclopentyl)-1H-pyrazol-3-yl)amino)-4-fluoro-2,3-dihydrobenzo[d]isothiazole 1,1-dioxide

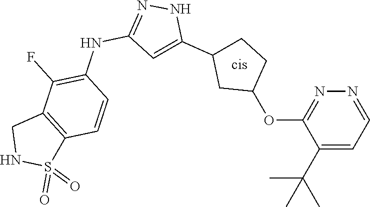 [SMILES: CC(C)(C)c1ccnnc1OC1CCC(c2cc(Nc3ccc4c(c3F)CNS4(=O)=O)n[nH]2)C1]

The titled compound was synthesized in the procedures similar to Example 24. 1H NMR (500 MHz, DMSO-d₆) δ=12.06 (s, 1H), 8.82-8.58 (m, 2H), 8.25 (s, 1H), 7.69 (s, 1H), 7.47 (d, J=8.6 Hz, 1H), 7.40 (d, J=4.9 Hz, 1H), 5.82 (s, 1H), 5.76-5.57 (m, 1H), 4.40 (d, J=4.3 Hz, 2H), 3.23-3.15 (m, 1H), 2.79-2.71 (m, 1H), 2.20-2.06 (m, 2H), 2.04-1.96 (m, 1H), 1.92-1.79 (m, 2H), 1.31 (s, 9H). LC-MS (ESI): m/z [M+H]⁺=487.2.

### Example 38: cis-5-((5-(3-((6-(tert-butyl)pyrimidin-4-yl)oxy)cyclopentyl)-1H-pyrazol-3-yl)amino)-4-fluoro-2,3-dihydrobenzo[d]isothiazole 1,1-dioxide

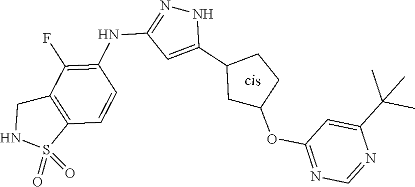 [SMILES: CC(C)(C)c1cc(OC2CCC(c3cc(Nc4ccc5c(c4F)CNS5(=O)=O)n[nH]3)C2)ncn1]

The titled compound was synthesized in the procedures similar to Example 24. ¹H NMR (500 MHz, DMSO-d₆) δ=12.03 (s, 1H), 8.72 (d, J=2.2, 1H), 8.71 (d, J=1.0, 1H), 8.26 (s, 1H), 7.69 (t, J=5.2, 1H), 7.47 (d, J=8.6, 1H), 6.79 (d, J=1.0, 1H), 5.85 (s, 1H), 5.49 (s, 1H), 4.40 (d, J=5.1, 2H), 3.15 (m, 1H), 2.70-2.60 (m, 1H), 2.08 (m, 2H), 1.90 (m, 1H), 1.86-1.68 (m, 2H), 1.26 (s, 9H). LC-MS (ESI): m/z [M+H]⁺=487.3.

### Example 39: cis-4-fluoro-5-((5-(3-(pyridin-3-yloxy)cyclopentyl)-1H-pyrazol-3-yl)amino)-2,3-dihydrobenzo[d]isothiazole 1,1-dioxide

### Step 1: cis-5-((1-(tert-butyl)-3-(3-(pyridin-3-yloxy)cyclopentyl)-1H-pyrazol-5-yl)amino)-4-fluoro-2-(4-methoxybenzyl)-2,3-dihydrobenzo[d]isothiazole 1,1-dioxide

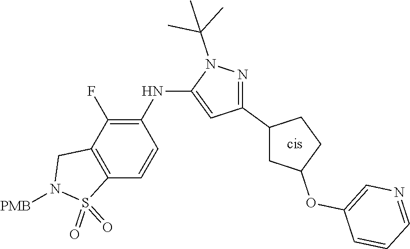

To a solution of trans-5-((1-(tert-butyl)-3-(3-hydroxycyclopentyl)-1H-pyrazol-5-yl)amino)-4-fluoro-2-(4-methoxybenzyl)-2,3-dihydrobenzo[d]isothiazole 1,1-dioxide (100 mg, 0.19 mmol) and pyridin-3-ol-1-methane (1/1) (21 mg, 0.19 mmol) in toluene (3 mL) was added CMBP (137 mg, 0.57 mmol). The reaction mixture was stirred for 16 hour at 100° C. under nitrogen. The resulting mixture was cooled to room temperature, concentrated, purified by silica gel column chromatography, eluting with PE/EA (1:1) to afford the product (110 mg, 93%). LC-MS (ESI): m/z [M+H]⁺=607.4

### Step 2: cis-4-fluoro-5-((5-(3-(pyridin-3-yloxy)cyclopentyl)-1H-pyrazol-3-yl)amino)-2,3-dihydrobenzo[d]isothiazole 1,1-dioxide

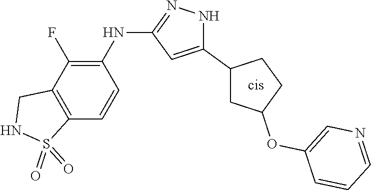 [SMILES: O=S1(=O)NCc2c1ccc(Nc1cc(C3CCC(Oc4cccnc4)C3)[nH]n1)c2F]

To a stirring solution of cis-5-((1-(tert-butyl)-3-(3-(pyridin-3-yloxy)cyclopentyl)-1H-pyrazol-5-yl)amino)-4-fluoro-2-(4-methoxybenzyl)-2,3-dihydrobenzo[d]isothiazole 1,1-dioxide (50 mg, 0.082 mmol) was added FA (10 mL), The solution was stirred for 12 h at 75° C. The mixture was concentrated under reduced pressure. The residue was purified by silica gel column chromatography, eluting with EA to afford the product 40 mg. The residue 40 mg was purified by prep HPLC (Waters XSelect C18: RD-CO-094 column, eluting with a gradient of acetonitrile/water containing 0.1% FA, 20%-40%) to afford the product (5 mg, 17.8%). 1H NMR (500 MHz, DMSO-d₆) δ=12.03 (s, 1H), 8.20 (d, J=2.4 Hz, 1H), 8.10 (d, J=4.3 Hz, 1H), 7.86 (s, 1H), 7.44-7.32 (m, 4H), 5.92 (s, 1H), 5.06-4.96 (m, 1H), 4.44 (s, 2H), 3.80 (s, 1H), 3.29-3.20 (m, 2H), 2.74-2.60 (m, 1H), 2.24-2.16 (m, 1H), 2.12-1.90 (m, 5H). LC-MS (ESI): m/z [M+H]⁺=430.2.

### Example 40: cis-4-fluoro-5-((5-(3-((2-methylpyridin-3-yl)oxy)cyclopentyl)-1H-pyrazol-3-yl)amino)-2,3-dihydrobenzo[d]isothiazole 1,1-dioxide

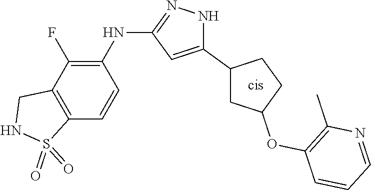 [SMILES: Cc1ncccc1OC1CCC(c2cc(Nc3ccc4c(c3F)CNS4(=O)=O)n[nH]2)C1]

The titled compound was synthesized in the procedures similar to Example 22. ¹H NMR (500 MHz, DMSO-d₆) δ=12.04 (s, 1H), 8.72 (s, 1H), 8.25 (s, 1H), 8.11 (d, J=4.6, 1H), 7.69 (t, J=5.0, 1H), 7.59 (s, 1H), 7.47 (d, J=8.6, 1H), 7.40 (s, 1H), 5.84 (s, 1H), 5.02 (s, 1H), 4.40 (d, J=4.8, 2H), 3.21-3.17 (m, 1H), 2.63 (m, 1H), 2.38 (s, 3H), 2.14-1.98 (m, 2H), 1.96-1.76 (m, 3H). LC-MS (ESI): m/z [M+H]⁺=444.2.

### Example 41: cis-5-((5-(3-((4-cyclopropylpyridin-3-yl)oxy)cyclopentyl)-1H-pyrazol-3-yl)amino)-4-fluoro-2,3-dihydrobenzo[d]isothiazole 1,1-dioxide

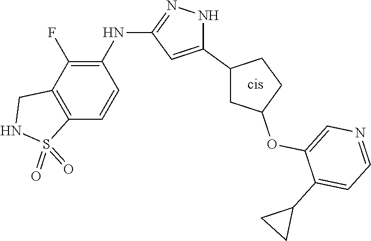 [SMILES: O=S1(=O)NCc2c1ccc(Nc1cc(C3CCC(Oc4cnccc4C4CC4)C3)[nH]n1)c2F]

The titled compound was synthesized in the procedures similar to Example 22. ¹H NMR (500 MHz, DMSO-d₆) δ 12.04 (s, 1H), 8.71 (s, 1H), 8.29-8.20 (m, 2H), 8.02 (d, J=5 Hz, 1H), 7.69 (t, J=10 Hz, 1H), 7.47 (d, J=5 Hz, 1H), 6.77 (d, J=5 Hz, 1H), 5.85 (s, 1H), 5.08-5.00 (m, 1H), 4.40 (d, J=5 Hz, 2H), 3.22-3.12 (m, 1H), 2.69-2.58 (m, 1H), 2.15-2.00 (m, 3H), 2.00-1.79 (m, 3H), 1.02-0.89 (m, 2H), 0.76-0.66 (m, 2H). LC-MS (ESI): m/z [M+H]⁺=470.2.

### Example 42: cis-4-fluoro-5-((5-(3-((4-methylpyridin-3-yl)oxy)cyclopentyl)-1H-pyrazol-3-yl)amino)-2,3-dihydrobenzo[d]isothiazole 1,1-dioxide

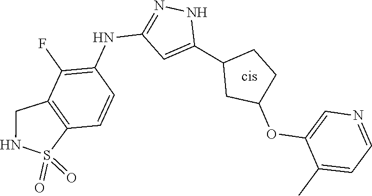 [SMILES: Cc1ccncc1OC1CCC(c2cc(Nc3ccc4c(c3F)CNS4(=O)=O)n[nH]2)C1]

The titled compound was synthesized in the procedures similar to Example 22. ¹H NMR (500 MHz, DMSO-d₆) δ=12.02 (s, 1H), 8.72 (s, 1H), 8.48 (s, 1H), 8.38 (d, J=5.1, 1H), 8.22 (s, 1H), 7.74 (s, 1H), 7.70 (s, 1H), 7.47 (d, J=8.6, 1H), 5.85 (s, 1H), 5.14 (s, 1H), 4.40 (s, 2H), 3.27-3.15 (m, 1H), 2.70-2.61 (m, 1H), 2.30 (s, 3H), 2.17-2.04 (m, 2H), 1.96 (m, 1H), 1.93-1.77 (m, 2H). LC-MS (ESI): m/z [M+H]⁺=444.34.

### Example 43: cis-4-fluoro-5-((5-(3-((4-(prop-1-en-2-yl)pyridin-3-yl)oxy)cyclopentyl)-1H-pyrazol-3-yl)amino)-2,3-dihydrobenzo[d]isothiazole 1,1-dioxide

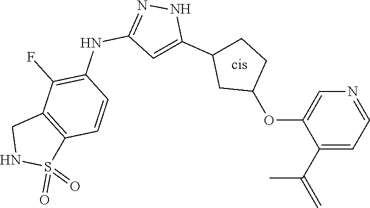 [SMILES: C=C(C)c1ccncc1OC1CCC(c2cc(Nc3ccc4c(c3F)CNS4(=O)=O)n[nH]2)C1]

### Step 1: cis-5-((3-(3-((4-bromopyridin-3-yl)oxy)cyclopentyl)-1-(tert-butyl)-1H-pyrazol-5-yl)amino)-4-fluoro-2-(4-methoxybenzyl)-2,3-dihydrobenzo[d]isothiazole 1,1-dioxide

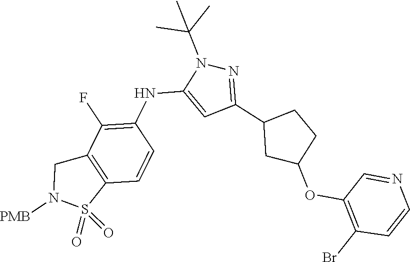

To a solution of trans-5-((1-(tert-butyl)-3-(3-hydroxycyclopentyl)-1H-pyrazol-5-yl)amino)-4-fluoro-2-(4-methoxybenzyl)-2,3-dihydrobenzo[d]isothiazole 1,1-dioxide (100 mg, 0.19 mmol) and 4-bromopyridin-3-ol (33 mg, 0.19 mmol) in toluene (3 mL) was added CMBP (137 mg, 0.57 mmol). The reaction mixture was stirred for 16 hour at 100° C. under nitrogen. The resulting mixture was cooled to room temperature, concentrated, purified by silica gel column chromatography, eluting with PE/EA (1:1) to afford the product (117 mg, 90%). LC-MS (ESI): m/z [M+H]⁺=684.7

### Step 2: cis-5-((1-(tert-butyl)-3-(3-((4-(prop-1-en-2-yl)pyridin-3-yl)oxy)cyclopentyl)-1H-pyrazol-5-yl)amino)-4-fluoro-2-(4-methoxybenzyl)-2,3-dihydrobenzo[d]isothiazole 1,1-dioxide

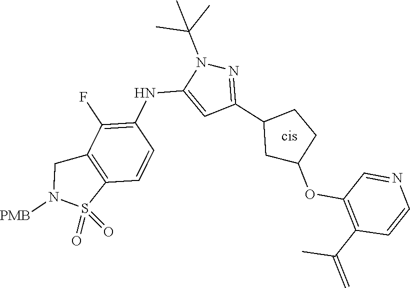

To a solution of cis-5-((3-(3-((4-bromopyridin-3-yl)oxy)cyclopentyl)-1-(tert-butyl)-1H-pyrazol-5-yl)amino)-4-fluoro-2-(4-methoxybenzyl)-2,3-dihydrobenzo[d]isothiazole 1,1-dioxide (117 mg, 0.171 mmol) and 4,4,5,5-tetramethyl-2-(prop-1-en-2-yl)-1,3,2-dioxaborolane (43 mg, 0.257 mmol) in dioxane (10 mL) was added Pd(dppf)Cl₂ (13 mg, 0.017 mmol) and K₂CO₃ (71 mg, 0.513 mmol) in H2O (1 mL). The reaction mixture was stirred for 16 hours at 100° C. under nitrogen. The resulting mixture was cooled to room temperature, concentrated, purified by silica gel column chromatography, eluting with PE/EA (1:1) to afford the product (73 mg, 66%). LC-MS (ESI): m/z [M+H]⁺=646.4.

### Step 3: cis-4-fluoro-5-((5-(3-((4-(prop-1-en-2-yl)pyridin-3-yl)oxy)cyclopentyl)-1H-pyrazol-3-yl)amino)-2,3-dihydrobenzo[d]isothiazole 1,1-dioxide

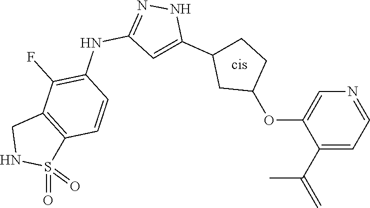 [SMILES: C=C(C)c1ccncc1OC1CCC(c2cc(Nc3ccc4c(c3F)CNS4(=O)=O)n[nH]2)C1]

The titled compound was synthesized in the procedures similar to Example 1, step 11. ¹H NMR (500 MHz, DMSO-d₆) δ=12.03 (s, 1H), 8.71 (s, 1H), 8.32 (s, 1H), 8.24 (s, 1H), 8.15 (d, J=4.7, 1H), 7.69 (t, J=5.1, 1H), 7.47 (d, J=8.6, 1H), 7.18 (d, J=4.7, 1H), 5.82 (s, 1H), 5.24 (s, 2H), 5.06 (d, J=3.7, 1H), 4.40 (d, J=5.1, 2H), 3.20-3.09 (m, 1H), 2.74-2.60 (m, 1H), 2.14-1.99 (m, 5H), 1.92 (m, 1H), 1.87-1.73 (m, 2H). LC-MS (ESI): m/z [M+H]⁺=470.3.

### Example 44: cis-4-fluoro-5-((5-(3-((5-isopropylpyridin-3-yl)oxy)cyclopentyl)-1H-pyrazol-3-yl)amino)-2,3-dihydrobenzo[d]isothiazole 1,1-dioxide

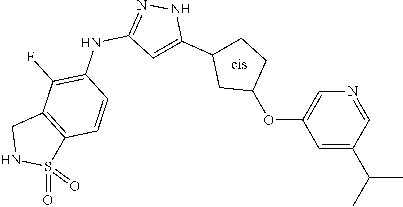 [SMILES: CC(C)c1cncc(OC2CCC(c3cc(Nc4ccc5c(c4F)CNS5(=O)=O)n[nH]3)C2)c1]

### Step 1: cis-5-((1-(tert-butyl)-3-(3-((5-isopropylpyridin-3-yl)oxy)cyclopentyl)-1H-pyrazol-5-yl)amino)-4-fluoro-2-(4-methoxybenzyl)-2,3-dihydrobenzo[d]isothiazole 1,1-dioxide

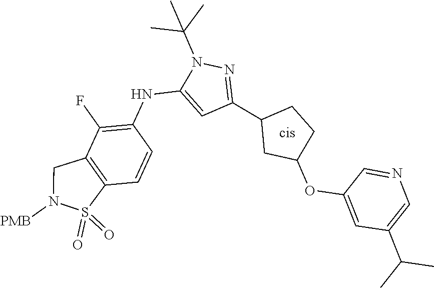

cis-5-((1-(tert-butyl)-3-(3-((5-(prop-1-en-2-yl)pyridin-3-yl)oxy)cyclopentyl)-1H-pyrazol-5-yl)amino)-4-fluoro-2-(4-methoxybenzyl)-2,3-dihydrobenzo[d]isothiazole 1,1-dioxide (120 mg, 0.185 mmol) was dissolved in THF (20 mL), Pd/C (10% on carbon, 10 mg) was added, the reaction mixture was stirred for 1 hour at room temperature under 1 atm H₂. The resulting mixture was filtered, the filtrated was concentrated under reduce pressure to afford the product (120 mg, 99%). LC-MS (ESI): m/z [M+H]⁺=648.5.

### Step 2: cis-4-fluoro-5-((5-(3-((5-isopropylpyridin-3-yl)oxy)cyclopentyl)-1H-pyrazol-3-yl)amino)-2,3-dihydrobenzo[d]isothiazole 1,1-dioxide

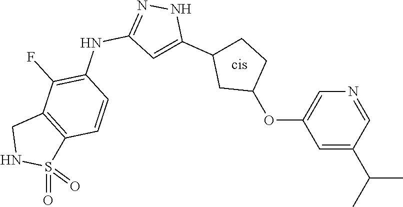 [SMILES: CC(C)c1cncc(OC2CCC(c3cc(Nc4ccc5c(c4F)CNS5(=O)=O)n[nH]3)C2)c1]

The titled compound was synthesized in the procedures similar to Example 1, step 11. ¹H NMR (500 MHz, DMSO-d₆) δ=12.02 (s, 1H), 8.73 (s, 1H), 8.27 (s, 1H), 8.10 (d, J=2.7, 1H), 8.07 (s, 1H), 7.69 (t, J=5.2, 1H), 7.47 (d, J=8.6, 1H), 7.20 (s, 1H), 5.86 (s, 1H), 5.00 (s, 1H), 4.40 (d, J=5.2, 2H), 3.21-3.10 (m, 1H), 2.92 (m, 1H), 2.67-2.59 (m, 1H), 2.13-2.01 (m, 2H), 1.88 (m, 1H), 1.82 (m, 1H), 1.75 (m, 1H), 1.22 (d, J=6.9, 6H). LC-MS (ESI): m/z [M+H]⁺=472.36.

### Example 45: cis-5-((5-(3-((2-(tert-butyl)pyridin-3-yl)oxy)cyclopentyl)-1H-pyrazol-3-yl)amino)-4-fluoro-2,3-dihydrobenzo[d]isothiazole 1,1-dioxide

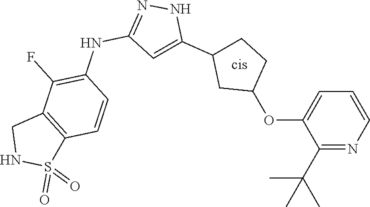 [SMILES: CC(C)(C)c1ncccc1OC1CCC(c2cc(Nc3ccc4c(c3F)CNS4(=O)=O)n[nH]2)C1]

The titled compound was synthesized in the procedures similar to Example 22. ¹H NMR (500 MHz, DMSO-d₆) δ=12.05 (s, 1H), 8.72 (d, J=1.9, 1H), 8.27 (s, 1H), 8.01 (m, 1H), 7.69 (t, J=5.1, 1H), 7.47 (d, J=8.6, 1H), 7.31 (d, J=8.3, 1H), 7.18 (m, 1H), 5.80 (s, 1H), 5.05-4.91 (m, 1H), 4.40 (d, J=5.1, 2H), 3.21-3.07 (m, 1H), 2.77-2.67 (m, 1H), 2.16-2.10 (m, 1H), 2.10-2.02 (m, 1H), 2.00-1.90 (m, 1H), 1.83 (m, 1H), 1.75 (m, 1H), 1.34 (s, 9H). LC-MS (ESI): m/z [M+H]⁺=486.33.

### Example 46: cis-4-fluoro-5-((5-(3-(pyridin-2-yloxy)cyclopentyl)-1H-pyrazol-3-yl)amino)-2,3-dihydrobenzo[d]isothiazole 1,1-dioxide

### Step 1: cis-5-((1-(tert-butyl)-3-(3-(pyridin-2-yloxy)cyclopentyl)-1H-pyrazol-5-yl)amino)-4-fluoro-2-(4-methoxybenzyl)-2,3-dihydrobenzo[d]isothiazole 1,1-dioxide

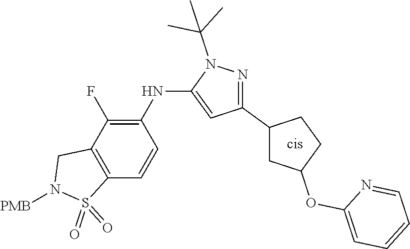

To a solution of cis-5-((1-(tert-butyl)-3-(3-hydroxycyclopentyl)-1H-pyrazol-5-yl)amino)-4-fluoro-2-(4-methoxybenzyl)-2,3-dihydrobenzo[d]isothiazole 1,1-dioxide (170 mg, 0.322 mmol) in DMF (8 mL) was added NaH (38 mg, 0.966 mmol), stirred for 1 hour at room temperature, 2-fluoropyridine (62 mg, 0.644 mmol) in DMF (2 mL) was added, the reaction mixture was stirred for 16 hours at 65° C. The resulting mixture was cooled to room temperature, diluted with water, extracted with EA, the combined organic phase was washed with water, then brine, dried over Na₂SO₄, purified by column chromatography on silica gel (petroleum ether:ethyl acetate=1:1) to afford the product (80 mg, 41%). LC-MS (ESI): m/z [M+H]⁺=606.2.

### Step 2: cis-4-fluoro-5-((5-(3-(pyridin-2-yloxy)cyclopentyl)-1H-pyrazol-3-yl)amino)-2,3-dihydrobenzo[d]isothiazole 1,1-dioxide

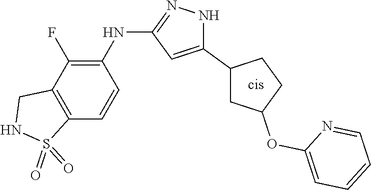 [SMILES: O=S1(=O)NCc2c1ccc(Nc1cc(C3CCC(Oc4ccccn4)C3)[nH]n1)c2F]

The titled compound was synthesized in the procedures similar to Example 1, step 11. ¹H NMR (500 MHz, DMSO-d₆) δ=12.02 (s, 1H), 8.72 (d, J=2.0, 1H), 8.26 (s, 1H), 8.16 (dd, J=5.0, 1.8, 1H), 7.68 (m, 2H), 7.47 (d, J=8.6, 1H), 6.95 (dd, J=6.9, 5.1, 1H), 6.78 (d, J=8.3, 1H), 5.85 (s, 1H), 5.46-5.38 (m, 1H), 4.40 (d, J=5.0, 2H), 3.19-3.10 (m, 1H), 2.65-2.57 (m, 1H), 2.12-2.00 (m, 2H), 1.91-1.78 (m, 2H), 1.78-1.71 (m, 1H). LC-MS (ESI): m/z [M+H]⁺=430.31.

### Example 47: cis-5-((5-(3-((4-(tert-butyl)pyridin-2-yl)oxy)cyclopentyl)-1H-pyrazol-3-yl)amino)-4-fluoro-2,3-dihydrobenzo[d]isothiazole 1,1-dioxide

### Step 1: cis-5-((1-(tert-butyl)-3-(3-(pyridin-2-yloxy)cyclopentyl)-1H-pyrazol-5-yl)amino)-4-fluoro-2-(4-methoxybenzyl)-2,3-dihydrobenzo[d]isothiazole 1,1-dioxide

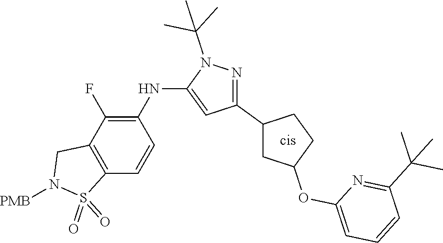

To a solution of cis-5-((1-(tert-butyl)-3-(3-hydroxycyclopentyl)-1H-pyrazol-5-yl)amino)-4-fluoro-2-(4-methoxybenzyl)-2,3-dihydrobenzo[d]isothiazole 1,1-dioxide (150 mg, 0.284 mmol) and 2-bromo-4-(tert-butyl)pyridine (61 mg, 0.284 mmol) in toluene (15 mL) was added Pd₂dba₃ (26 mg, 0.028 mmol), Binap (35 mg, 0.059 mmol) and t-BuONa (82 mg, 0.852 mmol), the reaction mixture was stirred for 16 hours at 100° C. under nitrogen. The resulting mixture was cooled to room temperature, concentrated, purified by silica gel column chromatography, eluting with PE/EA (1:1) to afford the product (90 mg, 48%). LC-MS (ESI): m/z [M+H]⁺=662.5.

### Step 2: cis-5-((5-(3-((4-(tert-butyl)pyridin-2-yl)oxy)cyclopentyl)-1H-pyrazol-3-yl)amino)-4-fluoro-2,3-dihydrobenzo[d]isothiazole 1,1-dioxide

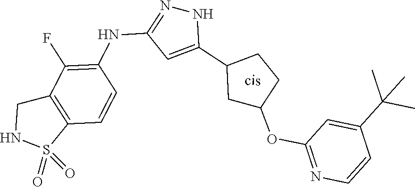 [SMILES: CC(C)(C)c1ccnc(OC2CCC(c3cc(Nc4ccc5c(c4F)CNS5(=O)=O)n[nH]3)C2)c1]

The titled compound was synthesized in the procedures similar to Example 1, step 11. ¹H NMR (500 MHz, DMSO-d₆) δ=12.02 (s, 1H), 8.72 (d, J=2.0, 1H), 8.25 (t, J=7.4, 1H), 8.05 (d, J=5.5, 1H), 7.69 (t, J=5.0, 1H), 7.47 (d, J=8.6, 1H), 6.98 (dd, J=5.5, 1.4, 1H), 6.68 (d, J=1.3, 1H), 5.86 (s, 1H), 5.47-5.32 (m, 1H), 4.40 (d, J=4.8, 2H), 3.16-3.11 (m, 1H), 2.66-2.57 (m, 1H), 2.14-1.99 (m, 2H), 1.91-1.77 (m, 2H), 1.77-1.69 (m, 1H), 1.24 (s, 9H). LC-MS (ESI): m/z [M+H]⁺=486.33.

### Example 48: cis-4-fluoro-5-((5-(3-(isoxazol-3-yloxy)cyclopentyl)-1H-pyrazol-3-yl)amino)-2,3-dihydrobenzo[d]isothiazole 1,1-dioxide

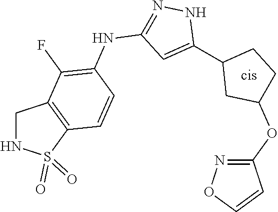 [SMILES: O=S1(=O)NCc2c1ccc(Nc1cc(C3CCC(Oc4ccon4)C3)[nH]n1)c2F]

The titled compound was synthesized in the procedures similar to Example 22. ¹H NMR (500 MHz, DMSO-d₆) δ 11.96 (s, 1H), 8.66 (s, 1H), 8.59 (d, J=1.8 Hz, 1H), 8.18 (s, 1H), 7.62 (t, J=5.1 Hz, 1H), 7.41 (d, J=8.6 Hz, 1H), 6.23 (d, J=1.8 Hz, 1H), 5.77 (s, 1H), 5.01 (dd, J=8.8, 4.6 Hz, 1H), 4.34 (d, J=5.4 Hz, 2H), 3.11-3.03 (m, 1H), 2.61-2.52 (m, 1H), 2.05-1.85 (m, 3H), 1.77-1.69 (m, 2H). LC-MS (ESI): m/z [M+H]⁺=420.3

### Example 49: cis-2-(3-(3-((4-fluoro-1,1-dioxido-2,3-dihydrobenzo[d]isothiazol-5-yl)amino)-1H-pyrazol-5-yl)cyclopentyl)isoxazol-3 (2H)-one

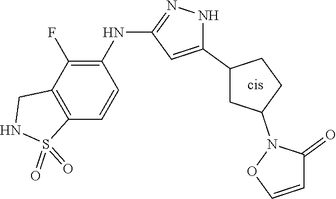 [SMILES: O=c1ccon1C1CCC(c2cc(Nc3ccc4c(c3F)CNS4(=O)=O)n[nH]2)C1]

The titled compound was synthesized in the procedures similar to Example 21. ¹H NMR (500 MHz, DMSO-d₆) δ 12.07 (s, 1H), 8.74 (s, 1H), 8.53 (dd, J=2.6, 1.0 Hz, 1H), 8.25 (s, 1H), 7.70 (s, 1H), 7.48 (d, J=8.6 Hz, 1H), 5.94 (d, J=2.6 Hz, 1H), 5.87 (s, 1H), 4.89-4.81 (m, 1H), 4.41 (d, J=5.4 Hz, 2H), 3.18-3.02 (m, 1H), 2.44-2.38 (m, 1H), 2.12-2.01 (m, 2H), 1.86-1.74 (m, 3H). LC-MS (ESI): m/z [M+H]⁺=420.3

### Example 50: cis-4-fluoro-5-((5-(3-(isothiazol-3-yloxy)cyclopentyl)-1H-pyrazol-3-yl)amino)-2,3-dihydrobenzo[d]isothiazole 1,1-dioxide

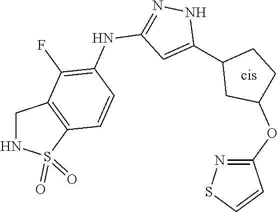 [SMILES: O=S1(=O)NCc2c1ccc(Nc1cc(C3CCC(Oc4ccsn4)C3)[nH]n1)c2F]

The titled compound was synthesized in the procedures similar to Example 22. ¹H NMR (500 MHz, DMSO-d₆) δ 11.95 (s, 1H), 8.81 (d, J=4.7 Hz, 1H), 8.65 (s, 1H), 8.19 (s, 1H), 7.62 (t, J=5.1 Hz, 1H), 7.41 (d, J=8.6 Hz, 1H), 6.68 (d, J=4.7 Hz, 1H), 5.77 (s, 1H), 5.24 (dd, J=9.5, 5.0 Hz, 1H), 4.34 (d, J=5.3 Hz, 2H), 3.12-3.04 (m, 1H), 2.62-2.53 (m, 1H), 2.05-1.96 (m, 2H), 1.90-1.81 (m, 1H), 1.76-1.67 (m, 2H). LC-MS (ESI): m/z [M+H]⁺=436.3

### Example 51: cis-2-(3-(3-((4-fluoro-1,1-dioxido-2,3-dihydrobenzo[d]isothiazol-5-yl)amino)-1H-pyrazol-5-yl)cyclopentyl)isothiazol-3 (2H)-one

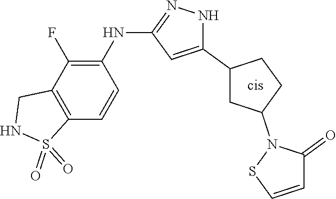 [SMILES: O=c1ccsn1C1CCC(c2cc(Nc3ccc4c(c3F)CNS4(=O)=O)n[nH]2)C1]

The titled compound was synthesized in the procedures similar to Example 21. ¹H NMR (500 MHz, DMSO-d₆) δ 12.10 (s, 1H), 8.75 (s, 1H), 8.49 (d, J=6.2 Hz, 1H), 8.26 (s, 1H), 7.70 (s, 1H), 7.49 (d, J=8.6 Hz, 1H), 6.19 (d, J=6.2 Hz, 1H), 5.89 (s, 1H), 4.83 (d, J=8.0 Hz, 1H), 4.41 (d, J=5.2 Hz, 2H), 3.24-3.18 (m, 1H), 2.20-2.10 (m, 2H), 1.90-1.74 (m, 3H), 1.52-1.38 (m, 1H). LC-MS (ESI): m/z [M+H]⁺=436.3

### Example 52: cis-4-fluoro-5-((5-(3-((4-(prop-1-en-2-yl)isothiazol-3-yl)oxy)cyclopentyl)-1H-pyrazol-3-yl)amino)-2,3-dihydrobenzo[d]isothiazole 1,1-dioxide

### Step 1: 3-(benzyloxy)isothiazole

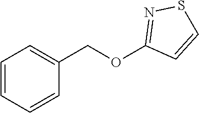 [SMILES: c1ccc(COc2ccsn2)cc1]

To a solution of isothiazol-3-ol (1.5 g, 14.8 mmol) and (bromomethyl)benzene (2.54 g, 14.8 mmol) in DMF (50 mL) was added K₂CO₃ (4.1 g, 29.7 mmol), the reaction mixture was stirred for 16 hours at room temperature. The resulting mixture was diluted with water, extracted with EA, the combined organic phase was washed with water, then brine, dried over Na₂SO₄, purified by column chromatography on silica gel (petroleum ether:ethyl acetate=20:1) to afford the product (1.89 g, 67%). LC-MS (ESI): m/z [M+H]⁺=192.1.

### Step 2: 3-(benzyloxy)-4-bromoisothiazole

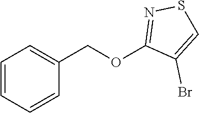 [SMILES: Brc1csnc1OCc1ccccc1]

To a solution of 3-(benzyloxy)isothiazole (1.0 g, 5.24 mmol) in DMF (10 mL) was added NBS (932 mg, 5.24 mmol), the reaction mixture was stirred for 16 hours at room temperature. The resulting mixture was diluted with water, extracted with EA, the combined organic phase was washed with water, then brine, dried over Na₂SO₄, filtered, the filtrated was concentrated under reduce pressure, dried over in vacuum, to afford the crude product (1.25 g crude). LC-MS (ESI): m/z [M+H]⁺=270.0.

### Step 3: 3-(benzyloxy)-4-cyclopropylisothiazole

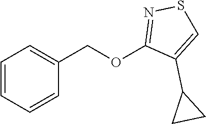 [SMILES: c1ccc(COc2nscc2C2CC2)cc1]

To a solution of 3-(benzyloxy)-4-bromoisothiazole (500 mg, 1.86 mmol) and cyclopropylboronic acid (1.6 g, 18.6 mmol) in dioxane (20 mL) and H₂O (2 mL) was added Pd(dppf)Cl₂ (136 mg, 0.186 mmol) and Cs₂CO₃ (1.8 g, 5.58 mmol), the reaction mixture was stirred for 16 hours at 100° C. under nitrogen. The resulting mixture was cooled to room temperature, concentrated, purified by silica gel column chromatography, eluting with PE/EA (5:1) to afford the product (222 mg, 52%). LC-MS (ESI): m/z [M+H]⁺=232.0.

### Step 4: 4-cyclopropylisothiazol-3-ol

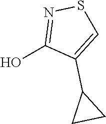

3-(benzyloxy)-4-cyclopropylisothiazole (222 mg, 0.96 mmol) was dissolved in conc. HCl aqueous (5 mL), the reaction mixture was stirred for 2 hours at 50° C. The resulting mixture was concentrated under reduce pressure, dried over in vacuum, to afford the product (126 mg, 93%), LC-MS (ESI): m/z [M+H]⁺=141.7.

### Step 5: cis-5-((1-(tert-butyl)-3-(3-((4-cyclopropylisothiazol-3-yl)oxy)cyclopentyl)-1H-pyrazol-5-yl)amino)-4-fluoro-2-(4-methoxybenzyl)-2,3-dihydrobenzo[d]isothiazole 1,1-dioxide

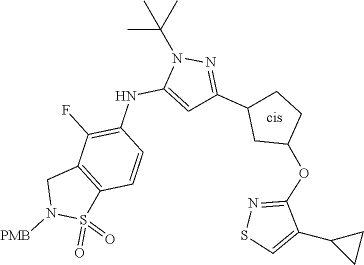

To a solution of trans-5-((1-(tert-butyl)-3-(3-hydroxycyclopentyl)-1H-pyrazol-5-yl)amino)-4-fluoro-2-(4-methoxybenzyl)-2,3-dihydrobenzo[d]isothiazole 1,1-dioxide (100 mg, 0.19 mmol) and 4-cyclopropylisothiazol-3-ol (27 mg, 0.19 mmol) in toluene (3 mL) was added CMBP (137 mg, 0.57 mmol). The reaction mixture was stirred for 16 h at 95° C. under nitrogen. The resulting mixture was cooled to room temperature, concentrated, purified by silica gel column chromatography, eluting with PE/EA (1:1) to afford the product (90 mg, 73%). LC-MS (ESI): m/z [M+H]⁺=652.4

### Step 6: cis-4-fluoro-5-((5-(3-((4-(prop-1-en-2-yl)isothiazol-3-yl)oxy)cyclopentyl)-1H-pyrazol-3-yl)amino)-2,3-dihydrobenzo[d]isothiazole 1,1-dioxide

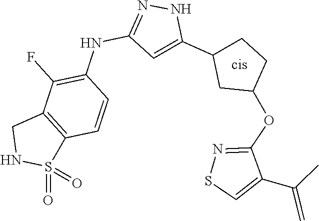 [SMILES: C=C(C)c1csnc1OC1CCC(c2cc(Nc3ccc4c(c3F)CNS4(=O)=O)n[nH]2)C1]

To a solution of cis-5-((1-(tert-butyl)-3-(3-((4-cyclopropylisothiazol-3-yl)oxy)cyclopentyl)-1H-pyrazol-5-yl)amino)-4-fluoro-2-(4-methoxybenzyl)-2,3-dihydrobenzo[d]isothiazole 1,1-dioxide (90 mg, 0.138 mmol) was dissolved in DCM (1 mL), TfOH (1 mL) was added, the reaction mixture was stirred for 30 min at room temperature. The resulting mixture was neutralized with Na₂CO₃ aqueous, extracted with EA, the combined organic phase was washed with brine, dried over Na₂SO₄, purified by column chromatography on silica gel (dichloromethane:methanol=10:1) to give the product (6 mg, 9%). ¹H NMR (500 MHz, DMSO-d₆) δ=12.03 (s, 1H), 8.72 (s, 1H), 8.71 (s, 1H), 8.24 (s, 1H), 7.69 (s, 1H), 7.47 (d, J=8.6, 1H), 6.26 (M, 2H), 5.84 (s, 1H), 5.36 (m, 1H), 4.40 (s, 2H), 3.16 (m, 1H), 2.66-2.59 (m, 1H), 2.13-2.02 (m, 2H), 1.93 (m, 1H), 1.88-1.80 (m, 2H), 1.76 (d, J=6.0, 3H). LC-MS (ESI): m/z [M+H]⁺=476.2.

### Example 53: cis-5-((5-(3-((4-cyclopropylisothiazol-3-yl)oxy)cyclopentyl)-1H-pyrazol-3-yl)amino)-4-fluoro-2,3-dihydrobenzo[d]isothiazole 1,1-dioxide

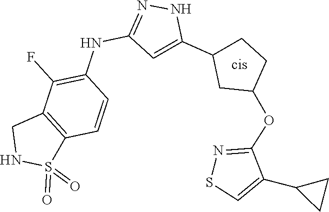 [SMILES: O=S1(=O)NCc2c1ccc(Nc1cc(C3CCC(Oc4nscc4C4CC4)C3)[nH]n1)c2F]

Cis-5-((1-(tert-butyl)-3-(3-((4-cyclopropylisothiazol-3-yl)oxy)cyclopentyl)-1H-pyrazol-5-yl)amino)-4-fluoro-2-(4-methoxybenzyl)-2,3-dihydrobenzo[d]isothiazole 1,1-dioxide (70 mg, 0.11 mmol) was dissolved in formic acid (3 mL), the reaction mixture was stirred for 16 hours at 85° C. The resulting mixture was cooled to room temperature, the residue was redissolved in methanol (3 mL), K₂CO₃ (80 mg, 0.55 mmol) was added, stirred for another 30 min. The resulting mixture was filtered, the filtrated was concentrated under reduce pressure, purified by column chromatography on silica gel (dichloromethane:methanol=10:1) to afford the product (9 mg, 17%). ¹H NMR (500 MHz, DMSO-d₆) δ=12.03 (s, 1H), 8.71 (s, 1H), 8.27 (s, 1H), 8.24 (s, 1H), 7.69 (s, 1H), 7.47 (d, J=8.6, 1H), 5.85 (s, 1H), 5.33 (s, 1H), 4.40 (d, J=3.0, 2H), 3.21-3.10 (m, 1H), 2.69-2.59 (m, 1H), 2.06 (m, 2H), 1.95 (m, 1H), 1.89-1.78 (m, 2H), 1.77-1.62 (m, 1H), 0.83 (m, 2H), 0.64 (m, 2H). LC-MS (ESI): m/z [M+H]⁺=476.4.

### Example 54: cis-4-cyclopropyl-2-(3-(3-((4-fluoro-1,1-dioxido-2,3-dihydrobenzo[d]isothiazol-5-yl)amino)-1H-pyrazol-5-yl)cyclopentyl)isothiazol-3 (2H)-one

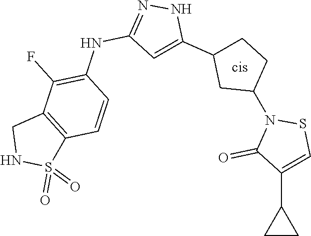 [SMILES: O=c1c(C2CC2)csn1C1CCC(c2cc(Nc3ccc4c(c3F)CNS4(=O)=O)n[nH]2)C1]

The titled compound was synthesized in the procedures similar to Example 21. LC-MS (ESI): m/z [M+H]⁺=476.3.

### Example 55: cis-4-fluoro-5-((5-(3-((4-isopropylisothiazol-3-yl)oxy)cyclopentyl)-1H-pyrazol-3-yl)amino)-2,3-dihydrobenzo[d]isothiazole 1,1-dioxide

### Step 1: 3-(benzyloxy)-4-cyclopropylisothiazole

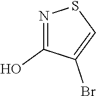

3-(benzyloxy)-4-bromoisothiazole (1.34 g, 5 mmol) was dissolved in conc. HCl aqueous (15 mL), the reaction mixture was stirred for 2 hours at 50° C. The resulting mixture was concentrated under reduced pressure, dried over in vacuum, to afford the product (850 mg, 95% yield). LC-MS (ESI): m/z [M+H]⁺=180.1.

### Step 2: cis-5-((3-(3-((4-bromoisothiazol-3-yl)oxy)cyclopentyl)-1-(tert-butyl)-1H-pyrazol-5-yl)amino)-4-fluoro-2-(4-methoxybenzyl)-2,3-dihydrobenzo[d]isothiazole 1,1-dioxide

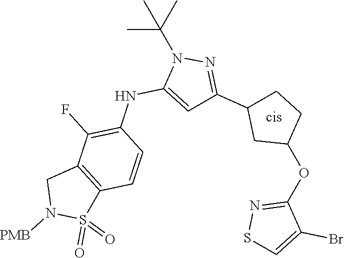

To a solution of trans-5-((1-(tert-butyl)-3-(3-hydroxycyclopentyl)-1H-pyrazol-5-yl)amino)-4-fluoro-2-(4-methoxybenzyl)-2,3-dihydrobenzo[d]isothiazole 1,1-dioxide (1 g, 1.9 mmol) and 3-(benzyloxy)-4-cyclopropylisothiazole (340 mg, 1.9 mmol) in toluene (15 mL) was added CMBP (1.37 g, 5.7 mmol). The reaction mixture was stirred for 16 h at 95° C. under nitrogen. The resulting mixture was cooled to room temperature, concentrated, purified by silica gel column chromatography, eluting with PE/EA (1:1) to afford the product (916 mg, 70%). LC-MS (ESI): m/z [M+H]⁺=690.2

### Step 3: cis-5-((1-(tert-butyl)-3-(3-((4-(prop-1-en-2-yl)isothiazol-3-yl)oxy)cyclopentyl)-1H-pyrazol-5-yl)amino)-4-fluoro-2-(4-methoxybenzyl)-2,3-dihydrobenzo[d]isothiazole 1,1-dioxide

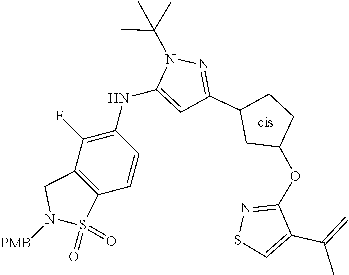

A mixture of cis-5-((3-(3-((4-bromoisothiazol-3-yl)oxy)cyclopentyl)-1-(tert-butyl)-1H-pyrazol-5-yl)amino)-4-fluoro-2-(4-methoxybenzyl)-2,3-dihydrobenzo[d]isothiazole 1,1-dioxide (270 mg, 0.392 mmol), potassium trifluoro(prop-1-en-2-yl)borate (116 mg, 0.784 mmol), XPhos Pd G2 (31 mg, 0.039 mmol) and Cs₂CO₃ (383 mg, 1.176 mmol) in 1,4-dioxane (10 mL) and H₂O (1 mL) was stirred for 3 h at 100° C. in the sealed tube under N₂ atmosphere. The solution was concentrated under reduced pressure. The residue was purified by silica gel column chromatography, eluting with PE/EtOAc (1:1) to afford the product (240 mg, 94%). LC-MS (ESI): m/z [M+H]⁺=652.3.

### Step 4: cis-5-((1-(tert-butyl)-3-(3-((4-isopropylisothiazol-3-yl)oxy)cyclopentyl)-1H-pyrazol-5-yl)amino)-4-fluoro-2-(4-methoxybenzyl)-2,3-dihydrobenzo[d]isothiazole 1,1-dioxide

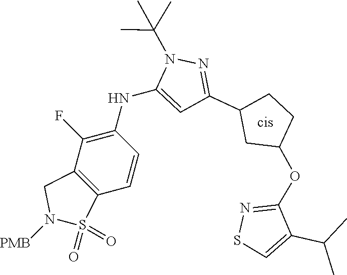

A mixture of cis-5-((1-(tert-butyl)-3-(3-((4-(prop-1-en-2-yl)isothiazol-3-yl)oxy)cyclopentyl)-1H-pyrazol-5-yl)amino)-4-fluoro-2-(4-methoxybenzyl)-2,3-dihydrobenzo[d]isothiazole 1,1-dioxide (60 mg, 0.092 mmol) and Pd/C (10 mg) in THF (10 mL) was stirred for 2 h at 20° C. under H₂ atmosphere. The mixture was filtered to afford the product (50 mg, 83%). LC-MS (ESI): m/z [M+H]⁺=654.4.

### Step 5: cis-4-fluoro-5-((5-(3-((4-isopropylisothiazol-3-yl)oxy)cyclopentyl)-1H-pyrazol-3-yl)amino)-2,3-dihydrobenzo[d]isothiazole 1,1-dioxide

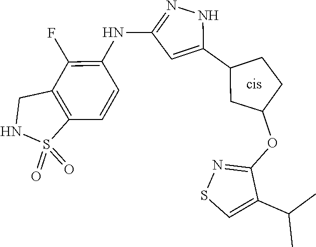 [SMILES: CC(C)c1csnc1OC1CCC(c2cc(Nc3ccc4c(c3F)CNS4(=O)=O)n[nH]2)C1]

The titled compound was synthesized in the procedures similar to Example 1, step 11. ¹H NMR (500 MHz, DMSO-d₆) δ 12.03 (s, 1H), 8.72 (s, 1H), 8.45 (d, J=0.6 Hz, 1H), 8.25 (s, 1H), 7.69 (t, J=4.8 Hz, 1H), 7.47 (d, J=8.5 Hz, 1H), 5.83 (s, 1H), 5.40-5.30 (m, 1H), 4.40 (d, J=4.6 Hz, 2H), 3.21-3.10 (m, 1H), 2.85-2.73 (m, 1H), 2.68-2.59 (m, 1H), 2.13-1.99 (m, 2H), 1.97-1.89 (m 1H), 1.86-1.75 (m, 2H), 1.14 (dd, J=6.7, 3.8 Hz, 6H). LC-MS (ESI): m/z [M+H]⁺=478.23.

### Example 56: cis-5-((5-(3-((4-(tert-butyl)isothiazol-3-yl)oxy)cyclopentyl)-1H-pyrazol-3-yl)amino)-4-fluoro-2,3-dihydrobenzo[d]isothiazole 1,1-dioxide

### Step 1: 3-(benzyloxy)-4-(tert-butyl)isothiazole

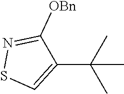 [SMILES: CC(C)(C)c1csnc1OCc1ccccc1]

To a solution of 3-(benzyloxy)-4-iodoisothiazole (5 g, 15.7 mmol) in THF (75 mL) were added CuI (150 mg, 0.785 mmol) and t-BuMgBr (30 ml, 15.7 mmol) at 0° C. The mixture was stirred for 2 h at 50° C. The reaction was quenched with NH₄Cl solution (80 mL). The resulting solution was extracted with 3×50 mL of EtOAc, dried over Na₂SO₄ and concentrated under vacuum. The residue was purified by silica gel column chromatography, eluting with PE:EA (100:0 to 60:40). The residue was product (400 mg, 12%). LCMS (ESI) m/z [M+H]⁺=248

### Step 2: 4-(tert-butyl)isothiazol-3-ol

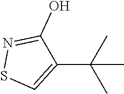

To a solution of 3-(benzyloxy)-4-(tert-butyl)isothiazole (400 mg, 1.6 mmol) in DCM (10 mL) was added BBr₃ (8 mL, 8 mmol) at 0° C. for 1 h. The mixture was stirred for 12 h at rt. The resulting mixture was concentrated under vacuum. The residue was purified by silica gel column chromatography, eluting with PE:EA (100:0 to 60:40) to give the product (136 mg, 53%). H NMR (300 MHz, DMSO-d₆) δ 11.46 (s, 1H), 8.20 (d, J=37.8 Hz, 1H), 1.27 (s, 9H). LCMS (ESI) m/z [M+H]⁺=158.

### Step 3: cis-5-((5-(3-((4-(tert-butyl)isothiazol-3-yl)oxy)cyclopentyl)-1H-pyrazol-3-yl)amino)-4-fluoro-2,3-dihydrobenzo[d]isothiazole 1,1-dioxide

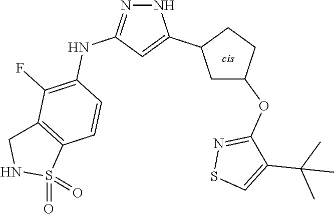 [SMILES: CC(C)(C)c1csnc1OC1CCC(c2cc(Nc3ccc4c(c3F)CNS4(=O)=O)n[nH]2)C1]

The titled compound was synthesized in the procedures similar to Example 21. ¹H NMR (500 MHz, DMSO-d₆) δ=12.04 (s, 1H), 8.72 (d, J=1.6 Hz, 1H), 8.40 (s, 1H), 8.26 (s, 1H), 7.69 (t, J=5.0 Hz, 1H), 7.47 (d, J=8.6 Hz, 1H), 5.81 (s, 1H), 5.42-5.23 (m, 1H), 4.40 (d, J=4.9 Hz, 2H), 3.19-3.10 (m, 1H), 2.72-2.61 (m, 1H), 2.15-1.99 (m, 2H), 1.98-1.90 (m, 1H), 1.86-1.76 (m, 2H), 1.24 (s, 9H). LC-MS (ESI): m/z [M+H]⁺=492.2.

### Example 57: cis-4-fluoro-5-((5-(3-((4-isopropyl-4H-1,2,4-triazol-3-yl)oxy)cyclopentyl)-1H-pyrazol-3-yl)amino)-2,3-dihydrobenzo[d]isothiazole 1,1-dioxide

### Step 1: phenyl isopropylcarbamate

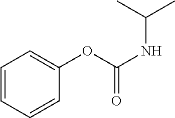

To a stirred solution propan-2-amine (2 g) in DCM (100 mL) was added phenyl carbonochloridate (2 g, 12.7 mmol) dropwise at 0° C. The above mixture was stirred for 2 hours at 0° C. Desired product could be detected by LCMS. The precipitated solids were collected by filtration and washed with water (2×50 mL). The resulting solid was dried under nitrogen atmosphere to afford phenyl isopropylcarbamate (2 g, 87.7%). The crude product was used in the next step directly without further purification.

### Step 2: N-isopropylhydrazinecarboxamide

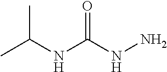

A solution of phenyl isopropylcarbamate (2 g, 17.1 mmol) and NH₂NH₂—H₂O (1.71 g, 34.2 mmol) in EtOH (20 mL) was stirred for 3 h at 80° C. Desired product could be detected by LCMS. The resulting mixture was concentrated under reduced pressure to afford N-isopropylhydrazinecarboxamide (1 g, 76.9%). The crude product was used in the next step directly without further purification.

### Step 3: 4-isopropyl-4H-1,2,4-triazol-3-ol

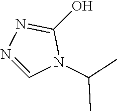

A solution of 1-amino-3-methylurea (2.5 g, 28.059 mmol, 1 equiv.) in HCOOH (20 mL) was stirred for 36 h at 105° C. Desired product could be detected by LCMS. The resulting solution was concentrated under reduced pressure. The residue was purified by reverse phase flash chromatography and concentrated under reduced pressure to afford 4-isopropyl-4H-1,2,4-triazol-3-ol (180 mg, 16.7%).

### Step 4: cis-4-fluoro-5-((5-(3-((4-isopropyl-4H-1,2,4-triazol-3-yl)oxy)cyclopentyl)-1H-pyrazol-3-yl)amino)-2,3-dihydrobenzo[d]isothiazole 1,1-dioxide

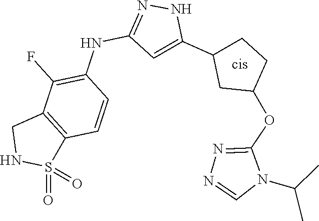 [SMILES: CC(C)n1cnnc1OC1CCC(c2cc(Nc3ccc4c(c3F)CNS4(=O)=O)n[nH]2)C1]

The titled compound was synthesized in the procedures similar to Example 22. ¹H NMR (500 MHz, DMSO-d₆) δ 10.26 (s, 1H), 7.99 (s, 1H), 7.93 (d, J=64.6 Hz, 2H), 7.86 (s, 1H), 6.70 (s, 1H), 6.60 (d, J=8.6 Hz, 1H), 5.71 (d, J=12.4 Hz, 1H), 4.60 (s, 2H), 3.24-3.11 (m, 2H), 2.66-2.59 (m, 3H), 2.46-2.38 (m, 1H), 2.20-2.11 (m, 1H), 2.08 (dd, J=14.3, 7.0 Hz, 1H), 2.02-1.93 (m, 1H), 1.88 (dd, J=22.7, 10.3 Hz, 1H), 1.79-1.66 (m, 1H), 1.16 (td, J=7.5, 3.9 Hz, 3H). LC-MS (ESI): m/z [M+H]⁺=462.2.

### Example 58: cis-4-fluoro-5-((5-(3-((4-(1-methylcyclopropyl)-4H-1,2,4-triazol-3-yl)oxy)cyclopentyl)-1H-pyrazol-3-yl)amino)-2,3-dihydrobenzo[d]isothiazole 1,1-dioxide

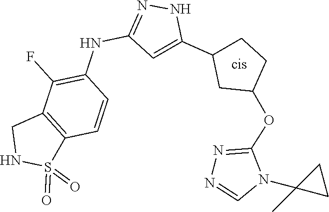 [SMILES: CC1(n2cnnc2OC2CCC(c3cc(Nc4ccc5c(c4F)CNS5(=O)=O)n[nH]3)C2)CC1]

The titled compound was synthesized in the procedures similar to Example 57. ¹H NMR (500 MHz, DMSO-d₆) δ 11.97 (s, 1H), 8.67 (s, 1H), 8.20 (s, 1H), 7.90 (s, 1H), 7.62 (s, 1H), 7.41 (d, J=8.6 Hz, 1H), 5.79 (s, 1H), 4.60-4.49 (m, 1H), 4.34 (s, 2H), 3.13-3.02 (m, 1H), 2.28 (dt, J=14.4, 7.4 Hz, 1H), 2.06-1.97 (m, 1H), 1.96-1.89 (m, 1H), 1.82 (dt, J=23.9, 11.9 Hz, 2H), 1.73 (dd, J=18.3, 9.5 Hz, 1H), 1.31 (s, 3H), 0.91 (t, J=5.8 Hz, 2H), 0.73 (t, J=6.0 Hz, 2H). LC-MS (ESI): m/z [M+H]⁺=474.2.

### Example 59: (cis)-5-((5-(3-((4-(tert-butyl)-4H-1,2,4-triazol-3-yl)oxy)cyclopentyl)-1H-pyrazol-3-yl)amino)-4-fluoro-2,3-dihydrobenzo[d]isothiazole 1,1-dioxide

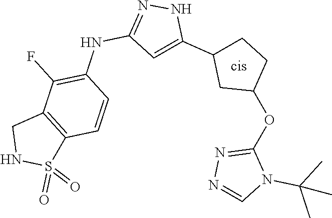 [SMILES: CC(C)(C)n1cnnc1OC1CCC(c2cc(Nc3ccc4c(c3F)CNS4(=O)=O)n[nH]2)C1]

The titled compound was synthesized in the procedures similar to Example 57. ¹H NMR (500 MHz, DMSO-d₆) δ 12.02 (s, 1H), 8.74 (s, 1H), 8.27 (t, J=7.5 Hz, 1H), 8.20 (s, 1H), 7.69 (s, 1H), 7.47 (d, J=8.6 Hz, 1H), 5.85 (s, 1H), 5.10 (s, 1H), 4.40 (d, J=4.8 Hz, 2H), 3.18-3.04 (m, 1H), 2.69-2.56 (m, 1H), 2.10-1.95 (m, 2H), 1.89 (d, J=13.4 Hz, 1H), 1.84-1.68 (m, 2H), 1.48 (s, 9H). LC-MS (ESI): m/z [M+H]⁺=476.2.

### Example 60: cis-4-fluoro-5-((5-(3-((4-isopropyl-1H-pyrazol-5-yl)oxy)cyclopentyl)-1H-pyrazol-3-yl)amino)-2,3-dihydrobenzo[d]isothiazole 1,1-dioxide

### Step 1: 4-isopropyl-1H-pyrazol-3-ol

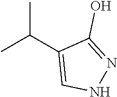

To an round bottom flask with stir bar, under nitrogen atmosphere, was added ethyl 2-cyano-3-methylbut-2-enoate (1.53 g, 10 mmol), anhydrous THF (30 mL) and triethylamine (204 mg, 2 mmol). The mixture was cooled in an ice/water bath. DIBAL-H (30 mL, 30 mmol, 1.0M in THF) was then added dropwise over 10-15 min. Upon completion of addition, the cooling bath was removed, and the reaction was stirred for an additional 1 h before recooling in an ice/water bath. The reaction was then quenched by aq. NaOH and water. The suspension was filtered through celite, washed with EA and then filtrate concentrated in vacuo. The residue was dissolved in 2-propanol (15 mL) and hydrazine (2-4 equiv.) was added. The mixture was heated and stirred at 70° C. The reaction was concentrated under vacuum and the residue was purified by silica gel chromatography, eluting with DCM/MeOH (20:1 to 5:1) to give the product (806 mg, 64% yield). LC-MS (ESI): m/z [M+H]⁺=127.3.

### Step 2: tert-butyl 3-hydroxy-4-isopropyl-1H-pyrazole-1-carboxylate

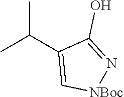 [SMILES: CC(C)c1cn(C(=O)OC(C)(C)C)nc1O]

A solution of 4-isopropyl-1H-pyrazol-3-ol (806 mg, 6.4 mmol), TEA (1.96 g, 19.2 mmol) and di-tert-butyl dicarbonate (1.4 g, 6.4 mmol) in EtOH (20 mL) was stirred for 3 h at 80° C. Desired product could be detected by LCMS. The resulting mixture was concentrated under reduced pressure to afford crude Product, the crude was washed with EA and PE to give the title compound (1.05 g, 73%). LC-MS (ESI): m/z [M+H]⁺=227.4.

### Step 3: cis-tert-butyl 3-((3-(1-(tert-butyl)-5-((4-fluoro-2-(4-methoxybenzyl)-1,1-dioxido-2,3-dihydrobenzo[d]isothiazol-5-yl)amino)-1H-pyrazol-3-yl)cyclopentyl)oxy)-4-isopropyl-1H-pyrazole-1-carboxylate

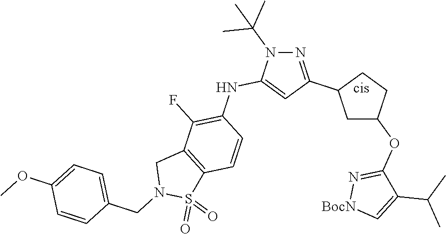 [SMILES: COc1ccc(CN2Cc3c(ccc(Nc4cc(C5CCC(Oc6nn(C(=O)OC(C)(C)C)cc6C(C)C)C5)nn4C(C)(C)C)c3F)S2(=O)=O)cc1]

A mixture of trans-5-((1-(tert-butyl)-3-(3-hydroxycyclopentyl)-1H-pyrazol-5-yl)amino)-4-fluoro-2-(4-methoxybenzyl)-2,3-dihydrobenzo[d]isothiazole 1,1-dioxide (100 mg, 0.189 mmol), tert-butyl 3-hydroxy-4-isopropyl-1H-pyrazole-1-carboxylate (136 mg, 0.6 mmol) and Cyanomethylenetributylphosphorane (0.137 g, 0.568 mmol) in toluene (5 mL) was heated at 95° C. under N₂ for 15 hrs. After cooled to 25° C., the mixture was concentrated under reduced pressure. The residue was purified by silica gel column chromatography, eluting with PE/EtOAc (1:1) to afford the product (24 mg, 17.2%). LC-MS (ESI): m/z [M+H]⁺=737.4.

### Step 4: cis-4-fluoro-5-((5-(3-((4-isopropyl-1H-pyrazol-5-yl)oxy)cyclopentyl)-1H-pyrazol-3-yl)amino)-2,3-dihydrobenzo[d]isothiazole 1,1-dioxide

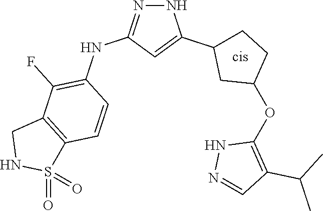 [SMILES: CC(C)c1cn[nH]c1OC1CCC(c2cc(Nc3ccc4c(c3F)CNS4(=O)=O)n[nH]2)C1]

The titled compound was synthesized in the procedures similar to Example 1, step 11. ¹H NMR (500 MHz, MeOD-d₄) δ 7.82 (s, 1H), 7.37 (d, J=8.6 Hz, 1H), 7.21 (d, J=9.3 Hz, 1H), 5.92 (s, 1H), 4.44 (s, 2H), 2.71 (dt, J=13.5, 6.7 Hz, 1H), 2.64-2.54 (m, 1H), 2.25 (dt, J=14.8, 7.2 Hz, 1H), 2.19-2.13 (m, 1H), 2.03 (d, J=9.6 Hz, 2H), 1.98-1.93 (m, 2H), 1.58 (d, J=8.2 Hz, 1H), 1.29 (m, 6H). LC-MS (ESI): m/z [M+H]⁺=462.2.

### Example 61: cis-5-((5-(3-((4-(tert-butyl)-1H-pyrazol-5-yl)oxy)cyclopentyl)-1H-pyrazol-3-yl)amino)-4-fluoro-2,3-dihydrobenzo[d]isothiazole 1,1-dioxide

### Step 1: ethyl 2-formyl-3,3-dimethylbutanoate

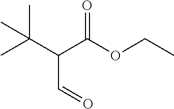

To a solution of ethyl 3,3-dimethylbutanoate (1.44 g, 10 mmol) in dry THF (20 ml) was added LDA (11 mL, 11 mmol) at −70° C. The mixture solution was stirred at −70° C. for 30 min. A solution of compound ethyl formate (740 mg, 11 mmol) in dry THF (3 mL) was added slowly. The mixture solution was stirred at −70° C. for 2 h and raise to room temperature overnight. The mixture was poured into NH₄Cl(sat.) and extracted with DCM (20 mL×3). The combined organic Layer was washed with brine, dried over Na₂SO₄, concentrated in vacuo. The crude product was used to next step without purification.

### Step 2: 4-(tert-butyl)-1H-pyrazol-3-ol

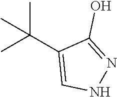

A solution of ethyl 2-formyl-3,3-dimethylbutanoate (2 g, 11.6 mmol) and NH₂NH₂—H₂O (2 g, 40 mmol) in EtOH (20 mL) was stirred for 3 h at 80° C. Desired product could be detected by LCMS. The resulting mixture was concentrated under reduced pressure to afford 4-(tert-butyl)-1H-pyrazol-3-ol (0.8 g, 49.1%).

### Step 3: tert-butyl 4-(tert-butyl)-3-hydroxy-1H-pyrazole-1-carboxylate

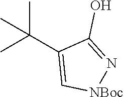 [SMILES: CC(C)(C)OC(=O)n1cc(C(C)(C)C)c(O)n1]

A solution of 4-(tert-butyl)-1H-pyrazol-3-ol (0.8 g, 5.7 mmol), TEA (1.73 g, 17.1 mmol) and Di-tert-butyl dicarbonate (1.3 g, 5.7 mmol) in EtOH (20 mL) was stirred for 3 h at 80° C. Desired product could be detected by LCMS. The resulting mixture was concentrated under reduced pressure to afford crude Product, the crude was washed with EA and PE to give the title compound (lg, 76.9% yield).

### Step 4: cis-5-((5-(3-((4-(tert-butyl)-1H-pyrazol-5-yl)oxy)cyclopentyl)-1H-pyrazol-3-yl)amino)-4-fluoro-2,3-dihydrobenzo[d]isothiazole 1,1-dioxide

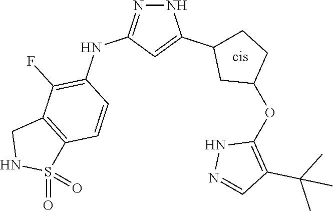 [SMILES: CC(C)(C)c1cn[nH]c1OC1CCC(c2cc(Nc3ccc4c(c3F)CNS4(=O)=O)n[nH]2)C1]

The titled compound was synthesized in the procedures similar to Example 60. ¹H NMR (500 MHz, DMSO-d₆) δ 12.01 (s, 1H), 11.43 (s, 1H), 8.71 (d, J=2.0 Hz, 1H), 8.23 (s, 1H), 7.69 (s, 1H), 7.47 (d, J=8.6 Hz, 1H), 7.19 (s, 1H), 5.81 (s, 1H), 5.16-4.98 (m, 1H), 4.40 (d, J=4.4 Hz, 2H), 3.16-3.05 (m, 1H), 2.59-2.55 (m, 1H), 2.09-2.02 (m, 1H), 1.99-1.88 (m, 2H), 1.77 (ddd, J=14.7, 13.8, 7.6 Hz, 2H), 1.17 (s, 9H). LC-MS (ESI): m/z [M+H]⁺=475.2.

### Example 62: cis-5-((5-(3-((1H-pyrazol-5-yl)oxy)cyclopentyl)-1H-pyrazol-3-yl)amino)-4-fluoro-2,3-dihydrobenzo[d]isothiazole 1,1-dioxide

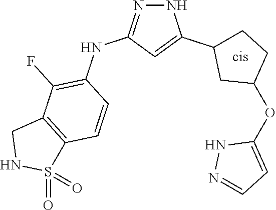 [SMILES: O=S1(=O)NCc2c1ccc(Nc1cc(C3CCC(Oc4ccn[nH]4)C3)[nH]n1)c2F]

The titled compound was synthesized in the procedures similar to Example 24. ¹H NMR (500 MHz, DMSO-d₆) δ 11.92 (d, J=85.1 Hz, 2H), 8.73 (s, 1H), 8.24 (d, J=16.2 Hz, 1H), 7.69 (t, J=5.1 Hz, 1H), 7.54-7.46 (m, 2H), 5.84 (s, 1H), 5.65 (d, J=2.2 Hz, 1H), 4.94 (s, 1H), 4.40 (d, J=4.7 Hz, 2H), 3.15-3.05 (m, 1H), 2.59-2.52 (m, 1H), 2.07-1.85 (m, 3H), 1.82-1.71 (m, 2H). LC-MS (ESI): m/z [M+H]⁺=419.3.

### Example 63: cis-4-fluoro-5-((5-(3-((1-methyl-1H-pyrazol-5-yl)oxy)cyclopentyl)-1H-pyrazol-3-yl)amino)-2,3-dihydrobenzo[d]isothiazole 1,1-dioxide

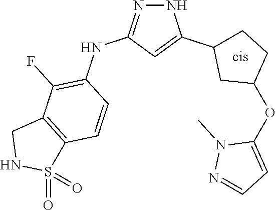 [SMILES: Cn1nccc1OC1CCC(c2cc(Nc3ccc4c(c3F)CNS4(=O)=O)n[nH]2)C1]

The titled compound was synthesized in the procedures similar to Example 24. ¹H NMR (500 MHz, DMSO-d₆) δ 12.04 (s, 1H), 8.74 (s, 1H), 8.27 (t, J=8.0 Hz, 1H), 7.69 (s, 1H), 7.48 (d, J=8.6 Hz, 1H), 7.20 (d, J=1.9 Hz, 1H), 5.84 (s, 1H), 5.62 (d, J=1.9 Hz, 1H), 4.79 (d, J=2.6 Hz, 1H), 4.40 (d, J=4.4 Hz, 2H), 3.49 (s, 3H), 3.20-3.12 (m, 1H), 2.62-2.57 (m, 1H), 2.12-2.04 (m, 1H), 2.02-1.93 (m, 2H), 1.87-1.80 (m, 2H). LC-MS (ESI): m/z [M+H]⁺=433.3.

### Example 64: cis-4-fluoro-5-((5-(3-((1-methyl-1H-pyrazol-3-yl)oxy)cyclopentyl)-1H-pyrazol-3-yl)amino)-2,3-dihydrobenzo[d]isothiazole 1,1-dioxide

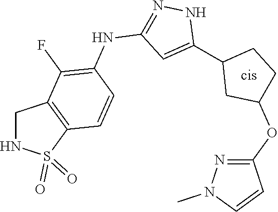 [SMILES: Cn1ccc(OC2CCC(c3cc(Nc4ccc5c(c4F)CNS5(=O)=O)n[nH]3)C2)n1]

The titled compound was synthesized in the procedures similar to Example 24. 1H NMR (500 MHz, DMSO-d₆) δ=11.99 (s, 1H), 8.78 (s, 1H), 8.29 (s, 1H), 7.54 (d, J=8.6 Hz, 1H), 6.98 (d, J=7.3 Hz, 1H), 5.84 (s, 1H), 5.00 (s, 1H), 4.43 (s, 2H), 4.29 (s, 1H), 3.67 (s, 1H), 3.12-3.02 (m, 1H), 2.78 (s, 3H), 2.50-2.40 (m, 1H), 2.10-2.00 (m, 1H), 1.97-1.84 (m, 1H), 1.78-1.66 (m, 2H), 1.64-1.52 (m, 5H), 1.48-1.38 (m, 4H). LC-MS (ESI): m/z [M+H]⁺=433.2.

### Example 65: cis-4-fluoro-5-((5-(3-((3-methyl-1H-pyrazol-5-yl)oxy)cyclopentyl)-1H-pyrazol-3-yl)amino)-2,3-dihydrobenzo[d]isothiazole 1,1-dioxide

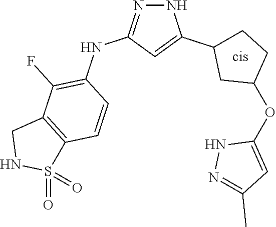 [SMILES: Cc1cc(OC2CCC(c3cc(Nc4ccc5c(c4F)CNS5(=O)=O)n[nH]3)C2)[nH]n1]

The titled compound was synthesized in the procedures similar to Example 24. ¹H NMR (500 MHz, DMSO-d₆) δ=11.99 (s, 1H), 11.50 (s, 1H), 8.72 (s, 1H), 8.27 (s, 1H), 7.69 (t, J=5.1, 1H), 7.47 (d, J=8.6, 1H), 5.83 (s, 1H), 5.41 (s, 1H), 4.89 (s, 1H), 4.40 (d, J=5.1, 2H), 3.08 (m, 1H), 2.64 (m, 1H), 2.13 (s, 3H), 2.02 (m, 1H), 1.94 (m, 1H), 1.90-1.83 (m, 1H), 1.81-1.67 (m, 2H). LC-MS (ESI): m/z [M+H]⁺=433.31.

### Example 66: cis-4-fluoro-5-((5-(3-((3-methyl-1-phenyl-1H-pyrazol-5-yl)oxy)cyclopentyl)-1H-pyrazol-3-yl)amino)-2,3-dihydrobenzo[d]isothiazole 1,1-dioxide

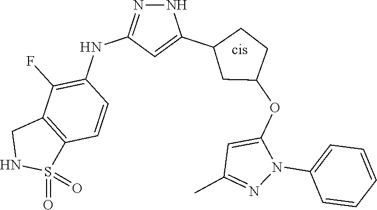 [SMILES: Cc1cc(OC2CCC(c3cc(Nc4ccc5c(c4F)CNS5(=O)=O)n[nH]3)C2)n(-c2ccccc2)n1]

The titled compound was synthesized in the procedures similar to Example 24. 1H NMR (500 MHz, DMSO-d₆) δ=12.06 (s, 1H), 8.71 (d, J=2.2 Hz, 1H), 8.23 (s, 1H), 7.69 (t, J=5.1 Hz, 1H), 7.61-7.55 (m, 2H), 7.46 (d, J=8.6 Hz, 1H), 7.40-7.34 (m, 2H), 7.22-7.15 (m, 1H), 5.82 (s, 1H), 5.70 (s, 1H), 4.88 (dd, J=7.5, 4.4 Hz, 1H), 4.40 (d, J=5.0 Hz, 2H), 3.23-3.14 (m, 1H), 2.68-2.57 (m, 1H), 2.16 (s, 3H), 2.13-2.08 (m, 1H), 2.06-1.98 (m, 2H), 1.95-1.88 (m, 1H), 1.88-1.78 (m, 1H). LC-MS (ESI): m/z [M+H]⁺=509.2.

### Example 67: cis-5-((5-(3-((1H-indazol-3-yl)oxy)cyclopentyl)-1H-pyrazol-3-yl)amino)-4-fluoro-2,3-dihydrobenzo[d]isothiazole 1,1-dioxide

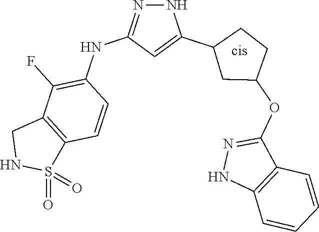 [SMILES: O=S1(=O)NCc2c1ccc(Nc1cc(C3CCC(Oc4n[nH]c5ccccc45)C3)[nH]n1)c2F]

The titled compound was synthesized in the procedures similar to Example 24. ¹H NMR (500 MHz, DMSO-d₆) δ 11.99 (brs, 1H), 8.75 (s, 1H), 8.34-8.17 (m, 1H), 7.70 (s, 1H), 7.61 (d, J=8.0 Hz, 1H), 7.56 (d, J=8.1 Hz, 1H), 7.53-7.45 (m, 2H), 7.37-7.28 (m, 2H), 6.99 (t, J=7.4 Hz, 1H), 5.89 (s, 1H), 5.35-5.25 (m, 1H), 5.17-5.03 (m, 1H), 4.41 (d, J=2.8 Hz, 2H), 3.28-3.10 (m, 1H), 2.72-2.62 (m, 1H), 2.20-1.98 (m, 4H), 1.98-1.82 (m, 2H). LC-MS (ESI): m/z [M+H]⁺=469.30.

### Example 68: cis-ethyl 5-((3-(3-((4-fluoro-1,1-dioxido-2,3-dihydrobenzo[d]isothiazol-5-yl)amino)-1H-pyrazol-5-yl)cyclopentyl)oxy)-1H-pyrazole-4-carboxylate

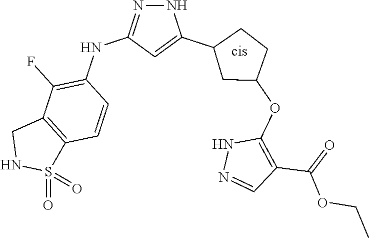 [SMILES: CCOC(=O)c1cn[nH]c1OC1CCC(c2cc(Nc3ccc4c(c3F)CNS4(=O)=O)n[nH]2)C1]

The titled compound was synthesized in the procedures similar to example 24. ¹H NMR (500 MHz, DMSO-d₆) δ 12.54 (s, 1H), 11.96 (s, 1H), 8.71 (d, J=1.9 Hz, 1H), 8.22 (t, J=8.0 Hz, 1H), 8.08 (s, 1H), 7.68 (s, 1H), 7.46 (d, J=8.6 Hz, 1H), 5.87 (d, J=2.1 Hz, 1H), 5.18-5.06 (m, 1H), 4.40 (d, J=4.3 Hz, 2H), 4.13 (q, J=7.1 Hz, 2H), 3.20-3.08 (m, 1H), 2.60-2.57 (m, 1H), 2.13-2.04 (m, 1H), 2.03-1.89 (m, 2H), 1.86-1.75 (m, 2H), 1.21 (t, J=7.1 Hz, 3H). LC-MS (ESI): m/z [M+H]⁺=491.27.

### Example 69: cis-4-fluoro-5-((5-(3-((3-(trifluoromethyl)-1H-pyrazol-5-yl)oxy)cyclopentyl)-1H-pyrazol-3-yl)amino)-2,3-dihydrobenzo[d]isothiazole 1,1-dioxide

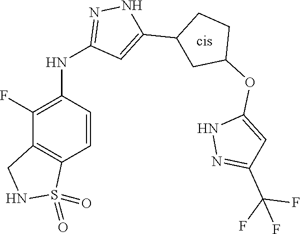 [SMILES: O=S1(=O)NCc2c1ccc(Nc1cc(C3CCC(Oc4cc(C(F)(F)F)n[nH]4)C3)[nH]n1)c2F]

The titled compound was synthesized in the procedures similar to Example 24. ¹H NMR (500 MHz, DMSO-d₆) δ=13.24 (s, 1H), 12.03 (s, 1H), 8.72 (d, J=2.0, 1H), 8.25 (s, 1H), 7.69 (t, J=5.0, 1H), 7.48 (d, J=8.6, 1H), 6.14 (s, 1H), 5.84 (s, 1H), 4.90 (s, 1H), 4.40 (d, J=4.9, 2H), 3.21-3.09 (m, 1H), 2.63 (m, 1H), 2.13-2.06 (m, 1H), 2.02 (m, 1H), 1.93 (m, 1H), 1.85-1.61 (m, 2H). LC-MS (ESI): m/z [M+H]⁺=487.21.

### Example 70: (cis)-4-fluoro-5-((5-(3-((1-phenyl-1H-pyrazol-5-yl)oxy)cyclopentyl)-1H-pyrazol-3-yl)amino)-2,3-dihydrobenzo[d]isothiazole 1,1-dioxide

### Step 1:1-phenyl-1H-pyrazol-5-ol

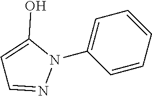 [SMILES: Oc1ccnn1-c1ccccc1]

To a solution of 3-methocyacriylic acid methyl ester (3 g, 23.1 mmol) was added phenylhydrazine (2.49 g, 23.1 mmol). The reaction was stirred at 110° C. for 12 hours then cooled to room temperature and concentrated to give the crude product, The residue was purified by silica gel column chromatography, eluted with PE/EtOAc (10:1 to 1:1) to afford 1-phenyl-1H-pyrazol-5-ol (400 mg, 10.8%). LC-MS (ESI): m/z [M+H]⁺=161.2.

### Step 2: cis-4-fluoro-5-((5-(3-((1-phenyl-1H-pyrazol-5-yl)oxy)cyclopentyl)-1H-pyrazol-3-yl)amino)-2,3-dihydrobenzo[d]isothiazole 1,1-dioxide

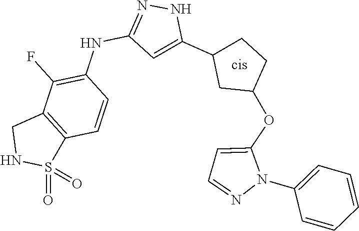 [SMILES: O=S1(=O)NCc2c1ccc(Nc1cc(C3CCC(Oc4ccnn4-c4ccccc4)C3)[nH]n1)c2F]

The titled compound was synthesized in the procedures similar to Example 22. ¹H NMR (500 MHz, DMSO-d₆) δ 12.04 (s, 1H), 8.73 (s, 1H), 8.33 (d, J=2.6 Hz, 1H), 8.27 (t, J=8.1 Hz, 1H), 7.73 (d, J=7.8 Hz, 2H), 7.69 (s, 1H), 7.46 (dd, J=16.9, 9.0 Hz, 3H), 7.22 (t, J=7.4 Hz, 1H), 6.03 (d, J=2.6 Hz, 1H), 5.87 (s, 1H), 5.10 (s, 1H), 4.40 (d, J=2.9 Hz, 2H), 3.19-3.09 (m, 1H), 2.74-2.61 (m, 1H), 2.15-2.03 (m, 2H), 1.95 (d, J=12.9 Hz, 1H), 1.88-1.69 (m, 2H). LC-MS (ESI): m/z [M+H]⁺=495.2.

### Example 71: cis-5-((5-(3-((3-(tert-butyl)-1H-pyrazol-5-yl)oxy)cyclopentyl)-1H-pyrazol-3-yl)amino)-4-fluoro-2,3-dihydrobenzo[d]isothiazole 1,1-dioxide

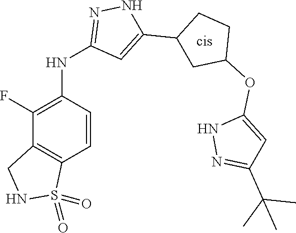 [SMILES: CC(C)(C)c1cc(OC2CCC(c3cc(Nc4ccc5c(c4F)CNS5(=O)=O)n[nH]3)C2)[nH]n1]

The titled compound was synthesized in the procedures similar to Example 24. 1H NMR (500 MHz, DMSO-d₆) δ=12.00 (s, 1H), 11.53 (s, 1H), 8.73 (s, 1H), 8.26 (s, 1H), 7.69 (t, J=5.0 Hz, 1H), 7.47 (d, J=8.6 Hz, 1H), 5.84 (s, 1H), 5.42 (s, 1H), 4.90 (s, 1H), 4.40 (d, J=5.0 Hz, 2H), 3.10 (dd, J=16.6, 8.9 Hz, 1H), 2.58-2.54 (m, 1H), 2.06-2.00 (m, 1H), 1.98-1.92 (m, 1H), 1.90-1.84 (m, 1H), 1.80-1.68 (m, 2H), 1.21 (s, 9H). LC-MS (ESI): m/z [M+H]⁺=475.2.

### Example 72: cis-5-((5-(3-((4-(tert-butyl)-1-methyl-1H-pyrazol-5-yl)oxy)cyclopentyl)-1H-pyrazol-3-yl)amino)-4-fluoro-2,3-dihydrobenzo[d]isothiazole 1,1-dioxide

### Step 1: cis-tert-butyl 4-(tert-butyl)-3-((3-(1-(tert-butyl)-5-((4-fluoro-2-(4-methoxybenzyl)-1,1-dioxido-2,3-dihydrobenzo[d]isothiazol-5-yl)amino)-1H-pyrazol-3-yl)cyclopentyl)oxy)-1H-pyrazole-1-carboxylate

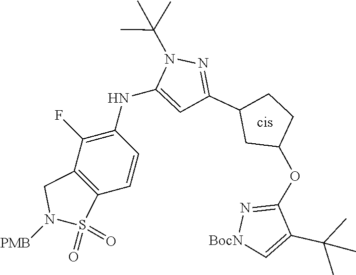

To a solution of trans-5-((1-(tert-butyl)-3-(3-hydroxycyclopentyl)-1H-pyrazol-5-yl)amino)-4-fluoro-2-(4-methoxybenzyl)-2,3-dihydrobenzo[d]isothiazole 1,1-dioxide (264 mg, 0.5 mmol) in toluene (10 mL) were added tert-butyl 4-(tert-butyl)-3-hydroxy-1H-pyrazole-1-carboxylate (140 mg, 1 mmol) and CMBP (361 mg, 1.5 mmol). The mixture was stirred at 95° C. for 16 h under a nitrogen atmosphere. The mixture was concentrated under vacuum. The residue was purified by silica gel column chromatography, eluting with EA/PE (0-55%) to afford the product (190 mg, 50.5%). LC-MS (ESI): m/z [M+H]⁺=751.

### Step 2: cis-5-((1-(tert-butyl)-3-(3-((4-(tert-butyl)-1H-pyrazol-5-yl)oxy)cyclopentyl)-1H-pyrazol-5-yl)amino)-4-fluoro-2-(4-methoxybenzyl)-2,3-dihydrobenzo[d]isothiazole 1,1-dioxide

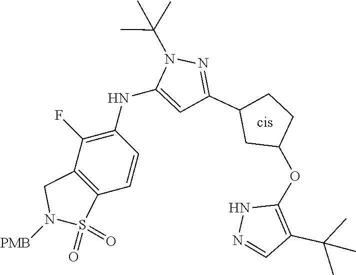

A mixture of cis-tert-butyl 4-(tert-butyl)-3-((3-(1-(tert-butyl)-5-((4-fluoro-2-(4-methoxybenzyl)-1,1-dioxido-2,3-dihydrobenzo[d]isothiazol-5-yl)amino)-1H-pyrazol-3-yl)cyclopentyl)oxy)-1H-pyrazole-1-carboxylate (190 mg, 0.25 mmol) in 4 N HCl in dioxane (10 mL) was stirred at rt for 3 h. The mixture was concentrated under vacuum to afford product (200 mg, crude), which was used in the next step directly without further purification. LC-MS (ESI): m/z [M+H]⁺=651.

### Step 3: cis-5-((1-(tert-butyl)-3-(3-((4-(tert-butyl)-1-methyl-1H-pyrazol-5-yl)oxy)cyclopentyl)-1H-pyrazol-5-yl)amino)-4-fluoro-2-(4-methoxybenzyl)-2,3-dihydrobenzo[d]isothiazole 1,1-dioxide

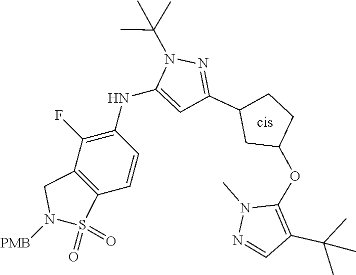

To a solution of cis-5-((1-(tert-butyl)-3-(3-((4-(tert-butyl)-1H-pyrazol-5-yl)oxy)cyclopentyl)-1H-pyrazol-5-yl)amino)-4-fluoro-2-(4-methoxybenzyl)-2,3-dihydrobenzo[d]isothiazole 1,1-dioxide (150 mg, 0.23 mmol) in DMF (5 mL) were added Cs₂CO₃ (150 mg, 0.46 mmol) and CH₃I (49 mg, 0.28 mmol). The mixture was stirred at rt for 16 h. The mixture was diluted with H₂O (50 mL), extracted with EA (3×30 mL). The combined organic layers were dried over Na₂SO₄, filtered and concentrated under vacuum. The residue was purified by silica gel column chromatography, eluting with EA/PE (0-65%) to afford the product (65 mg, 39.2%). LC-MS (ESI): m/z [M+H]⁺=665.3.

### Step 4: cis-5-((5-(3-((4-(tert-butyl)-1-methyl-1H-pyrazol-5-yl)oxy)cyclopentyl)-1H-pyrazol-3-yl)amino)-4-fluoro-2,3-dihydrobenzo[d]isothiazole 1,1-dioxide

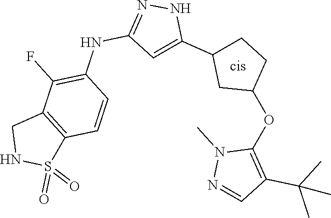 [SMILES: Cn1ncc(C(C)(C)C)c1OC1CCC(c2cc(Nc3ccc4c(c3F)CNS4(=O)=O)n[nH]2)C1]

A mixture of cis-5-((1-(tert-butyl)-3-(3-((4-(tert-butyl)-1-methyl-1H-pyrazol-5-yl)oxy)cyclopentyl)-1H-pyrazol-5-yl)amino)-4-fluoro-2-(4-methoxybenzyl)-2,3-dihydrobenzo[d]isothiazole 1,1-dioxide in FA (10 mL) was stirred at 75° C. for 16 h. The mixture was concentrated under vacuum. The residue was dissolved in MeOH (10 mL) and K₂CO₃ (58 mg) was added. The mixture was stirred at rt for 30 min. The mixture was concentrated under vacuum. The residue was purified by Prep-HPLC to afford product (10.55 mg, 22.1%). ¹H NMR (500 MHz, DMSO-d₆) δ 11.42 (s, 1H), 7.96-7.83 (m, 1H), 7.59-7.53 (m, 1H), 7.44 (t, J=5 Hz, 1H), 7.17 (s, 1H), 5.62 (s, 1H), 5.07-5.01 (m, 1H), 4.38 (s, 2H), 3.27 (s, 3H), 3.13-3.03 (m, 1H), 2.57-2.55 (m, 2H), 2.08-1.99 (m, 1H), 1.96-1.84 (m, 2H), 1.82-1.71 (m, 2H), 1.12 (s, 9H). LC-MS (ESI): m/z [M+H]⁺=489.2.

### Example 73: cis-4-fluoro-5-((5-(3-((3-isopropyl-1H-pyrazol-4-yl)oxy)cyclopentyl)-1H-pyrazol-3-yl)amino)-2,3-dihydrobenzo[d]isothiazole 1,1-dioxide

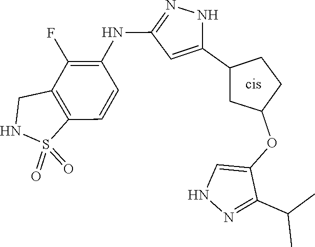 [SMILES: CC(C)c1n[nH]cc1OC1CCC(c2cc(Nc3ccc4c(c3F)CNS4(=O)=O)n[nH]2)C1]

The titled compound was synthesized in the procedures similar to Example 24. ¹H NMR (500 MHz, DMSO-d₆) δ 12.00 (s, 1H), 8.72 (s, 1H), 8.31-8.20 (m, 1H), 7.69 (t, J=5 Hz, 1H), 7.47 (t, J=10 Hz, 1H), 7.26 (s, 1H), 5.83 (s, 1H), 4.54-4.47 (m, 1H), 4.40 (d, J=5 Hz, 2H), 3.15-3.04 (m, 1H), 2.93-2.81 (m, 1H), 2.49-2.46 (m, 2H), 2.09-1.99 (m, 1H), 1.94-1.83 (m, 2H), 1.82-1.70 (m, 2H), 1.20-1.12 (m, 6H). LC-MS (ESI): m/z [M+H]⁺=461.2.

### Example 74: cis-5-((5-(3-((3-(tert-butyl)-1H-pyrazol-4-yl)oxy)cyclopentyl)-1H-pyrazol-3-yl)amino)-4-fluoro-2,3-dihydrobenzo[d]isothiazole 1,1-dioxide

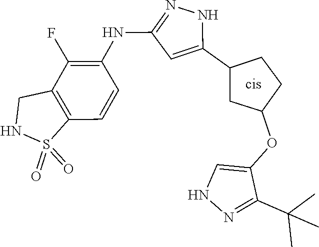 [SMILES: CC(C)(C)c1n[nH]cc1OC1CCC(c2cc(Nc3ccc4c(c3F)CNS4(=O)=O)n[nH]2)C1]

The titled compound was synthesized in the procedures similar to Example 24 in a racemic form, which was further separated by Chiral Prep-HPLC to give:

Enantiomer 1 (Example 74a, 97.2% ee); Retention time: 11.153 min. ¹H NMR (500 MHz, DMSO-d₆) 12.00 (s, 1H), 11.90-11.6 (m, 1H), 8.72 (s, 1H), 8.26 (t, J=10 Hz, 1H), 7.68 (t, J=5 Hz, 1H), 7.46 (d, J=5 Hz, 1H), 7.28 (s, 1H), 5.81 (s, 1H), 4.58-4.50 (m, 1H), 4.40 (d, J=10 Hz, 2H), 3.13-3.04 (m, 1H), 2.61-2.55 (m, 1H), 2.10-2.00 (m, 1H), 1.95-1.84 (m, 2H), 1.82-1.69 (m, 2H), 1.23 (m, 9H). LC-MS (ESI): m/z [M+H]⁺=475.2.

Enantiomer 2 (Example 74b, 100% ee); Retention time: 13.671 min. ¹H NMR (500 MHz, DMSO-d₆) 12.00 (s, 1H), 11.77 (s, 1H), 8.72 (s, 1H), 8.26 (t, J=5 Hz, 1H), 7.68 (t, J=5 Hz, 1H), 7.46 (d, J=5 Hz, 1H), 7.35-7.18 (m, 1H), 5.81 (s, 1H), 4.62-4.45 (m, 1H), 4.40 (d, J=10 Hz, 2H), 3.14-3.02 (m, 1H), 2.61-2.56 (m, 1H), 2.11-1.99 (m, 1H), 1.97-1.84 (m, 2H), 1.82-1.67 (m, 2H), 1.23 (m, 9H). LC-MS (ESI): m/z [M+H]⁺=475.2.

Chiral analytical method: Column: YMC Cellulose-C, 4.6*250 mm 5 μm; Mobile phase: A for Hex and B for EtOH (0.1% 2M NH₃ MeOH); Gradient: Mobile Phase A: Mobile Phase B=80:20 (v/v); HPLC Equipment: HPLC-Agilent, Back pressure: 100 bar; Column temperature: 35° C.

Chiral Prep-HPLC Condition: YMC Cellulose-C, 4.6*250 mm 5 μm; Mobile phase: A for Hex and B for EtOH (0.2% 2M NH₃ MeOH); Gradient: Mobile Phase A: Mobile Phase B=80:20 (v/v); Flow Rate: 18 mL/min, Wave Length: UV 254 nm and 280 nm, Prep-HPLC Equipment: Prep-HPLC-Gilson, Back pressure: 100 bar; Column temperature: 25° C.

### Example 75: cis-5-((5-(3-((3,5-dimethyl-1H-pyrazol-4-yl)oxy)cyclopentyl)-1H-pyrazol-3-yl)amino)-4-fluoro-2,3-dihydrobenzo[d]isothiazole 1,1-dioxide

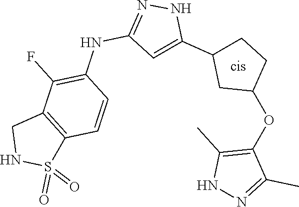 [SMILES: Cc1n[nH]c(C)c1OC1CCC(c2cc(Nc3ccc4c(c3F)CNS4(=O)=O)n[nH]2)C1]

The titled compound was synthesized in the procedures similar to Example 24. 1H NMR (500 MHz, DMSO-d₆) δ=11.99 (s, 2H), 8.74 (s, 1H), 8.27 (s, 1H), 7.69 (t, J=5.1 Hz, 1H), 7.48 (d, J=8.6 Hz, 1H), 5.87 (s, 1H), 4.41 (d, J=5.1 Hz, 3H), 3.12-3.02 (m, 1H), 2.42-2.35 (m, 2H), 2.06 (s, 6H), 2.02-1.99 (m, 1H), 1.92-1.71 (m, 5H). LC-MS (ESI): m/z [M+H]⁺=447.2.

### Example 76: cis-5-((5-(3-((1H-pyrazol-4-yl)oxy)cyclopentyl)-1H-pyrazol-3-yl)amino)-4-fluoro-2,3-dihydrobenzo[d]isothiazole 1,1-dioxide

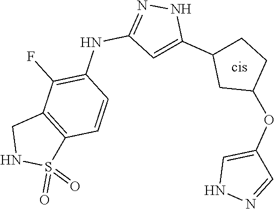 [SMILES: O=S1(=O)NCc2c1ccc(Nc1cc(C3CCC(Oc4cn[nH]c4)C3)[nH]n1)c2F]

The titled compound was synthesized in the procedures similar to Example 24. ¹H NMR (500 MHz, DMSO-d₆) δ=12.34 (s, 1H), 12.00 (s, 1H), 8.73 (s, 1H), 8.27 (t, J=7.2, 1H), 7.69 (t, J=4.5, 1H), 7.47 (d, J=8.6, 1H), 7.37 (s, 1H), 7.25 (s, 1H), 5.84 (s, 1H), 4.59-4.52 (m, 1H), 4.40 (d, J=4.3, 2H), 3.14-3.02 (m, 1H), 2.54 (m, 1H), 2.04 (m, 1H), 1.92 (m, 2H), 1.82-1.67 (m, 2H). C-MS (ESI): m/z [M+H]⁺=419.3.

### Example 77: cis-4-fluoro-5-((5-(3-((1-methyl-1H-pyrazol-4-yl)oxy)cyclopentyl)-1H-pyrazol-3-yl)amino)-2,3-dihydrobenzo[d]isothiazole 1,1-dioxide

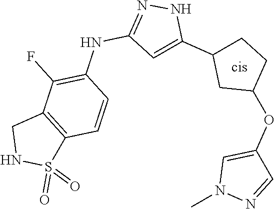 [SMILES: Cn1cc(OC2CCC(c3cc(Nc4ccc5c(c4F)CNS5(=O)=O)n[nH]3)C2)cn1]

The titled compound was synthesized in the procedures similar to Example 21. ¹H NMR (500 MHz, DMSO-d₆) δ=12.00 (s, 1H), 8.72 (s, 1H), 8.27 (t, J=7.7, 1H), 7.69 (t, J=5.1, 1H), 7.47 (d, J=8.6, 1H), 7.40 (s, 1H), 7.14 (s, 1H), 5.83 (s, 1H), 4.57-4.48 (m, 1H), 4.40 (d, J=5.1, 2H), 3.72 (s, 3H), 3.14-3.03 (m, 1H), 2.54 (m, 1H), 2.03 (m, 1H), 1.90 (m, 2H), 1.80-1.67 (m, 2H). LC-MS (ESI): m/z [M+H]⁺=433.28.

### Example 78: cis-4-fluoro-5-((5-(3-((1-isopropyl-1H-pyrazol-4-yl)oxy)cyclopentyl)-1H-pyrazol-3-yl)amino)-2,3-dihydrobenzo[d]isothiazole 1,1-dioxide

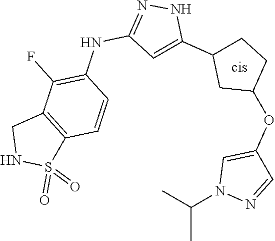 [SMILES: CC(C)n1cc(OC2CCC(c3cc(Nc4ccc5c(c4F)CNS5(=O)=O)n[nH]3)C2)cn1]

The titled compound was synthesized in the procedures similar to Example 21. 1H NMR (500 MHz, DMSO-d₆) δ=12.02 (s, 1H), 8.75 (d, J=1.9 Hz, 1H), 8.27 (s, 1H), 7.71 (s, 1H), 7.48 (d, J=10.7 Hz, 2H), 7.15 (s, 1H), 5.84 (s, 1H), 4.59-4.50 (m, 1H), 4.41 (d, J=3.5 Hz, 2H), 4.37-4.30 (m, 1H), 3.15-3.02 (m, 1H), 2.57-2.52 (m, 1H), 2.12-2.00 (m, 1H), 1.98-1.86 (m, 2H), 1.82-1.66 (m, 2H), 1.35 (d, J=6.7 Hz, 6H). LC-MS (ESI): m/z [M+H]⁺=461.2.

### Example 79: cis-4-fluoro-5-((5-(3-((3-methyl-1H-pyrazol-4-yl)oxy)cyclopentyl)-1H-pyrazol-3-yl)amino)-2,3-dihydrobenzo[d]isothiazole 1,1-dioxide

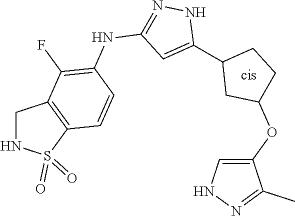 [SMILES: Cc1n[nH]cc1OC1CCC(c2cc(Nc3ccc4c(c3F)CNS4(=O)=O)n[nH]2)C1]

The titled compound was synthesized in the procedures similar to Example 24. ¹H NMR (500 MHz, DMSO-d₆) δ 11.93 (s, 2H), 8.66 (d, J=2.0 Hz, 1H), 8.18 (d, J=11.6 Hz, 1H), 7.62 (s, 1H), 7.40 (d, J=8.6 Hz, 1H), 7.21 (s, 1H), 5.78 (s, 1H), 4.46-4.40 (m, 1H), 4.34 (d, J=4.3 Hz, 2H), 3.02 (dt, J=17.7, 9.0 Hz, 1H), 2.42-2.37 (m, 1H), 2.01-1.93 (m, 4H), 1.85-1.77 (m, 2H), 1.76-1.64 (m, 2H). LC-MS (ESI): m/z [M+H]⁺=433.2

### Example 80: cis-5-((5-(3-((3-(tert-butyl)-5-methyl-1H-pyrazol-4-yl)oxy)cyclopentyl)-1H-pyrazol-3-yl)amino)-4-fluoro-2,3-dihydrobenzo[d]isothiazole 1,1-dioxide

### Step 1:1-(benzyloxy)-3,3-dimethylbutan-2-one

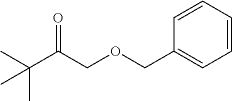

To a solution of phenylmethanol (4.5 g, 41.67 mmol) in THF (80 mL) was added NaH (60% dispersion in mineral oil, 2.0 g, 50.27 mmol) at 0° C., stirred for 30 minutes, 1-bromo-3,3-dimethylbutan-2-one (5.0 g, 27.93 mmol) in THF (20 mL) was added at 0° C., the reaction mixture was stirred for 2 hours at 0° C. The resulting mixture was quenched with saturated NH₄Cl aqueous, extracted with EA, dried over Na₂SO₄, purified by column chromatography on silica gel (petroleum ether:ethyl acetated=10:1) to give product (3.38 g, 59%). LC-MS (ESI): m/z [M+Na]⁺=229.1.

### Step 2: 2-(benzyloxy)-1-(dimethylamino)-4,4-dimethylpent-1-en-3-one

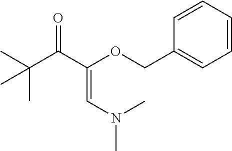 [SMILES: CN(C)/C=C(\OCc1ccccc1)C(=O)C(C)(C)C]

1-(benzyloxy)-3,3-dimethylbutan-2-one (3.38 g, 16.4 mmol) was dissolved in 1,1-dimethoxy-N,N-dimethylmethanamine (50 mL), stirred for 16 hours at 100° C. The resulting mixture was cooled to room temperature, concentrated under reduce pressure, dried over in vacuum, to give crude product (4.2 g crude). LC-MS (ESI): m/z [M+H]⁺=262.1.

### Step 3: 4-(benzyloxy)-3-(tert-butyl)-1H-pyrazole

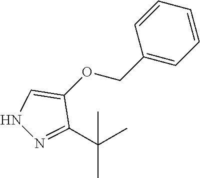 [SMILES: CC(C)(C)c1n[nH]cc1OCc1ccccc1]

2-(benzyloxy)-1-(dimethylamino)-4,4-dimethylpent-1-en-3-one (4.2 g crude) and hydrazine hydrate (5 mL) were dissolved in ethanol (50 mL), the reaction mixture was stirred for 3 hours at 55° C. The resulting mixture was cooled to room temperature, concentrated under reduce pressure, purified by column chromatography on silica gel (petroleum ether:ethyl acetated=1:1) to give product (550 mg, 15% for 2 steps). LC-MS (ESI): m/z [M+H]⁺=231.1.

### Step 4: 4-(benzyloxy)-5-bromo-3-(tert-butyl)-1H-pyrazole

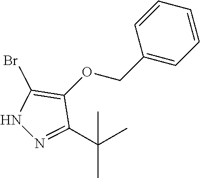 [SMILES: CC(C)(C)c1n[nH]c(Br)c1OCc1ccccc1]

To a solution of 4-(benzyloxy)-3-(tert-butyl)-1H-pyrazole (50 mg, 0.217 mmol) was dissolved in DMF (2 mL), NBS (41 mg, 0.228 mmol) was added, the reaction mixture was stirred for 2 hours at room temperature. The resulting mixture was diluted with water, extracted with EA, the combined organic phase was washed with water, then brine, dried over Na₂SO₄, purified by column chromatography on silica gel (petroleum ether:ethyl acetate=3:1) to afford the product (27 mg, 40%). LC-MS (ESI): m/z [M+H]⁺=309.2.

### Step 5: 4-(benzyloxy)-5-bromo-3-(tert-butyl)-1-((2-(trimethylsilyl)ethoxy)methyl)-1H-pyrazole and 4-(benzyloxy)-3-bromo-5-(tert-butyl)-1-((2-(trimethylsilyl)ethoxy)methyl)-1H-pyrazole

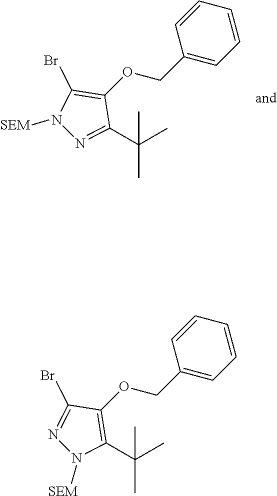 [SMILES: CC(C)(C)c1nn(COCC[Si](C)(C)C)c(Br)c1OCc1ccccc1 ; CC(C)(C)c1c(OCc2ccccc2)c(Br)nn1COCC[Si](C)(C)C]

To a solution of 4-(benzyloxy)-5-bromo-3-(tert-butyl)-1H-pyrazole (250 mg, 0.81 mmol) in DMF (5 mL) was added NaH (60% dispersion in mineral oil, 40 mg, 0.97 mmol) at 0° C., stirred for 30 minutes, SEMCl (150 mg, 0.89 mmol) was added, the reaction mixture was stirred for 30 min at room temperature. The resulting mixture was diluted with water, extracted with EA, the combined organic phase was washed with water, then brine, dried over Na₂SO₄, purified by column chromatography on silica gel (petroleum ether:ethyl acetate=4:1) to afford the product (300 mg, 85%). LC-MS (ESI): m/z [M+H]⁺=439.1.

### Step 6: 4-(benzyloxy)-3-(tert-butyl)-5-methyl-1-((2-(trimethylsilyl)ethoxy)methyl)-1H-pyrazole and 4-(benzyloxy)-5-(tert-butyl)-3-methyl-1-((2-(trimethylsilyl)ethoxy)methyl)-1H-pyrazole

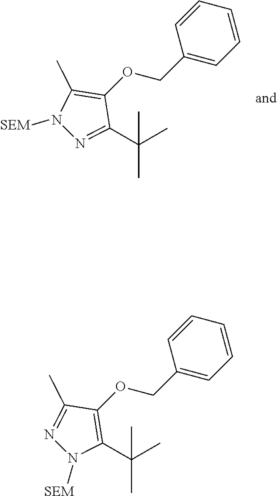 [SMILES: Cc1c(OCc2ccccc2)c(C(C)(C)C)nn1COCC[Si](C)(C)C ; Cc1nn(COCC[Si](C)(C)C)c(C(C)(C)C)c1OCc1ccccc1]

To a solution of 4-(benzyloxy)-5-bromo-3-(tert-butyl)-1-((2-(trimethylsilyl)ethoxy)methyl)-1H-pyrazole and 4-(benzyloxy)-3-bromo-5-(tert-butyl)-1-((2-(trimethylsilyl)ethoxy)methyl)-1H-pyrazole (300 mg, 0.68 mmol) and 2,4,6-trimethyl-1,3,5,2,4,6-trioxatriborinane (3.5 M in THF, 0.4 mL, 1.37 mmol) in THF (10 mL) was added Pd(PPh₃)₄ (80 mg, 0.068 mmol) and KOH (76 mg, 1.36 mmol) in H₂O (1 mL), the reaction mixture was stirred for 1 hour at 100° C. under nitrogen. The resulting mixture was cooled to room temperature, diluted with water, extracted with EA, the combined organic phase was washed with water, then brine, dried over Na₂SO₄, purified by column chromatography on silica gel (petroleum ether:ethyl acetate=4:1) to afford the product (93 mg, 37%). LC-MS (ESI): m/z [M+H]⁺=375.3.

### Step 7: 3-(tert-butyl)-5-methyl-1-((2-(trimethylsilyl)ethoxy)methyl)-1H-pyrazol-4-ol and 5-(tert-butyl)-3-methyl-1-((2-(trimethylsilyl)ethoxy)methyl)-1H-pyrazol-4-ol

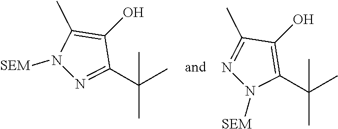 [SMILES: Cc1c(O)c(C(C)(C)C)nn1COCC[Si](C)(C)C ; Cc1nn(COCC[Si](C)(C)C)c(C(C)(C)C)c1O]

4-(benzyloxy)-3-(tert-butyl)-5-methyl-1-((2-(trimethylsilyl)ethoxy) methyl)-1H-pyrazole and 4-(benzyloxy)-5-(tert-butyl)-3-methyl-1-((2-(trimethylsilyl)ethoxy) methyl)-1H-pyrazole (93 mg, 0.25 mmol) was dissolved in THF (10 mL), Pd/C (10% on carbon, 30 mg) was added, the reaction mixture was stirred for 2 hours at room temperature under 1 atm H₂. The resulting mixture was filtered, the filtrated was concentrated, dried over in vacuum, to afford the product (61 mg, 86%). LC-MS (ESI): m/z [M+H]⁺=285.3.

### Step 8: cis-5-((5-(3-((3-(tert-butyl)-5-methyl-1H-pyrazol-4-yl)oxy)cyclopentyl)-1H-pyrazol-3-yl)amino)-4-fluoro-2,3-dihydrobenzo[d]isothiazole 1,1-dioxide

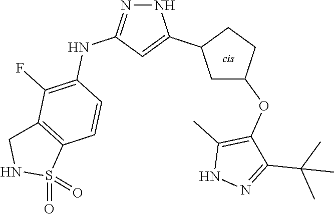 [SMILES: Cc1[nH]nc(C(C)(C)C)c1OC1CCC(c2cc(Nc3ccc4c(c3F)CNS4(=O)=O)n[nH]2)C1]

The titled compound was synthesized in the procedures similar to Example 60. ¹H NMR (500 MHz, DMSO-d₆) δ=12.02 (s, 1H), 11.70 (s, 1H), 8.74 (s, 1H), 8.28 (s, 1H), 7.69 (s, 1H), 7.48 (d, J=8.6, 1H), 5.85 (s, 1H), 4.56-4.49 (m, 1H), 4.41 (s, 2H), 3.09-3.01 (m, 1H), 2.47-2.43 (m, 1H), 2.13 (s, 3H), 2.06-1.99 (m, 1H), 1.95-1.90 (m, 1H), 1.90-1.85 (m, 1H), 1.84-1.76 (m, 2H), 1.26 (s, 9H). LC-MS (ESI): m/z [M+H]⁺=489.3.

### Example 81: cis-1-(3-(3-((4-fluoro-1,1-dioxido-2,3-dihydrobenzo[d]isothiazol-5-yl)amino)-1H-pyrazol-5-yl)cyclopentyl)-3,3-dimethylpyrrolidine-2,5-dione

### Step 1:1-isopropylimidazolidine-2,4-dione

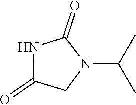

To a solution of N-carbamoyl-2-chloroacetamide (4.1 g, 30 mmol) in DMF (100 mL) was added isopropylamine (3.54 g, 60 mmol) at rt. The resulting mixture was stirred at 135° C. for 6 h under nitrogen. DMF was removed under vacuum and the residue was purified by column chromatography, eluting with DCM/EA=2:1 to give the product (1.07 g, 25% yield). LC-MS (ESI): m/z [M+H]⁺=143.3.

### Step 2: cis-benzyl (1-(tert-butyl)-3-(3-(3-isopropyl-2,5-dioxoimidazolidin-1-yl)cyclopentyl)-1H-pyrazol-5-yl)carbamate

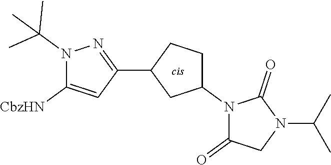 [SMILES: CC(C)N1CC(=O)N(C2CCC(c3cc(NC(=O)OCc4ccccc4)n(C(C)(C)C)n3)C2)C1=O]

To a solution of 1-isopropylimidazolidine-2,4-dione (596 mg, 4.2 mmol), trans-3-(5-(((benzyloxy)carbonyl)amino)-1-(tert-butyl)-1H-pyrazol-3-yl)cyclopentyl 4-nitrobenzoate (lg, 2.8 mmol) and triphenylphosphine (1.1 g, 4.2 mmol) in THF (50 mmol) was added DIAD (731 mg, 4.2 mmol) dropwise at 0° C. The mixture was stirred at rt for overnight. The solvent was removed under vacuum and the residue was purified by column chromatography, eluting with PE/EA=1:1 to give the product (1.09 g, 81% yield). LC-MS (ESI): m/z [M+H]⁺=482.4.

### Step 3: cis-benzyl (1-(tert-butyl)-3-(3-(3-isopropyl-2-oxo-2,3-dihydro-1H-imidazol-1-yl)cyclopentyl)-1H-pyrazol-5-yl)carbamate

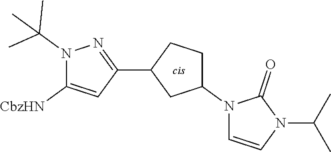 [SMILES: CC(C)n1ccn(C2CCC(c3cc(NC(=O)OCc4ccccc4)n(C(C)(C)C)n3)C2)c1=O]

To a solution of cis-benzyl (1-(tert-butyl)-3-(3-(3-isopropyl-2,5-dioxoimidazolidin-1-yl)cyclopentyl)-1H-pyrazol-5-yl)carbamate (100 mg, 0.2 mmol) in toluene (3 mL) was added sodium bis(2-methoxyethoxy)aluminiumhydride (0.6 mL, 2 mmol, 3.6 M in toluene) at 0° C. The resulting solution was stirred at rt for 1 h. The reaction was quenched with aq. NH₄Cl and extracted with EA (3×10 mL). The combined organic layers were dried over Na₂SO₄ and concentrated under vacuum. The residue was purified by column chromatography, eluting with PE/EA=2:3 to give the product (53 mg, 57% yield). LC-MS (ESI): m/z [M+H]⁺=466.4.

### Step 4: cis-1-(3-(5-amino-1-(tert-butyl)-1H-pyrazol-3-yl)cyclopentyl)-3-isopropylimidazolidin-2-one

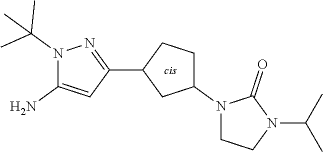 [SMILES: CC(C)N1CCN(C2CCC(c3cc(N)n(C(C)(C)C)n3)C2)C1=O]

cis-benzyl (1-(tert-butyl)-3-(3-(3-isopropyl-2-oxo-2,3-dihydro-1H-imidazol-1-yl)cyclopentyl)-1H-pyrazol-5-yl)carbamate (150 mg, 0.32 mmol) was dissolved in MeOH (10 mL), Pd/C (10% on carbon, 200 mg) was added, the reaction mixture was stirred for 2 hours at room temperature under 1 atm H₂. After filtration, the filtrated was concentrated to afford the product (103 mg, 91%). LC-MS (ESI): m/z [M+H]⁺=334.5.

### Step 5: cis-1-(3-(1-(tert-butyl)-5-((4-fluoro-1,1-dioxido-2,3-dihydrobenzo[d]isothiazol-5-yl)amino)-1H-pyrazol-3-yl)cyclopentyl)-3-isopropylimidazolidin-2-one

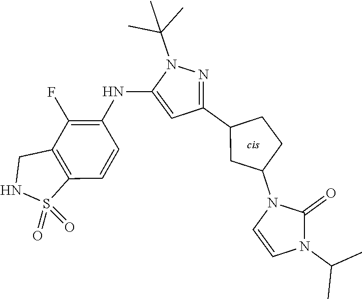 [SMILES: CC(C)n1ccn(C2CCC(c3cc(Nc4ccc5c(c4F)CNS5(=O)=O)n(C(C)(C)C)n3)C2)c1=O]

To a solution of cis-1-(3-(5-amino-1-(tert-butyl)-1H-pyrazol-3-yl)cyclopentyl)-3-isopropylimidazolidin-2-one (174 mg, 0.5 mmol) and 4-bromo-3-fluorobenzenesulfonamide (200 mg, 0.75 mmol) in dioxane (5 mL) was added Pd₂dba₃ (46 mg, 0.05 mmol), Xantphos (30 mg, 0.05 mmol) and K₃PO₄ (320 mg, 1.5 mmol). The reaction mixture was stirred for 6 hours at 90° C. under nitrogen. The resulting mixture was cooled to room temperature, concentrated under reduce pressure, purified by silica gel column chromatography, eluting with 100% EA to afford the product (96 mg, 37%). LC-MS (ESI): m/z [M+H]⁺=519.3.

### Step 6: cis-1-(3-(3-((4-fluoro-1,1-dioxido-2,3-dihydrobenzo[d]isothiazol-5-yl)amino)-1H-pyrazol-5-yl)cyclopentyl)-3-isopropylimidazolidin-2-one

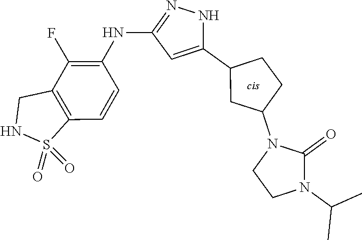 [SMILES: CC(C)N1CCN(C2CCC(c3cc(Nc4ccc5c(c4F)CNS5(=O)=O)n[nH]3)C2)C1=O]

To a solution of cis-1-(3-(1-(tert-butyl)-5-((4-fluoro-1,1-dioxido-2,3-dihydrobenzo[d]isothiazol-5-yl)amino)-1H-pyrazol-3-yl)cyclopentyl)-3-isopropylimidazolidin-2-one (96 mg, 0.185 mmol) in DCM (15 mL) was added TfOH (3 mL). The mixture was stirred at rt for 2 h. The mixture was quenched by aq NaHCO₃ (50 mL) at 0° C. and extracted with DCM (3×50 mL). The combined organic layers were combined, dried over Na₂SO₄, filtered and concentrated under vacuum. The residue was purified by silica gel chromatography, eluting with PE/EA (0% to 100%) to afford the product (9.4 mg, 11% yield). ¹H NMR (500 MHz, DMSO-d₆) δ 11.95 (s, 1H), 8.65 (d, J=2.6 Hz, 1H), 8.17 (s, 1H), 7.62 (t, J=5.2 Hz, 1H), 7.41 (d, J=8.6 Hz, 1H), 5.78 (s, 1H), 4.34 (d, J=5.4 Hz, 2H), 3.84 (p, J=6.7 Hz, 1H), 3.21-3.09 (m, 4H), 3.00 (p, J=8.0 Hz, 1H), 2.02 (dt, J=13.0, 6.9 Hz, 1H), 1.95 (d, J=7.7 Hz, 1H), 1.72 (dt, J=13.1, 8.0 Hz, 1H), 1.67-1.51 (m, 1H), 0.96 (dd, J=6.7, 2.0 Hz, 6H). LC-MS (ESI): m/z [M+H]⁺=463.4.

### Example 82: cis-3-(3-(3-((4-fluoro-1,1-dioxido-2,3-dihydrobenzo[d]isothiazol-5-yl)amino)-1H-pyrazol-5-yl)cyclopentyl)-1-isopropylimidazolidine-2,4-dione

### Step 1: cis-3-(3-(5-amino-1-(tert-butyl)-1H-pyrazol-3-yl)cyclopentyl)-1-isopropylimidazolidine-2,4-dione

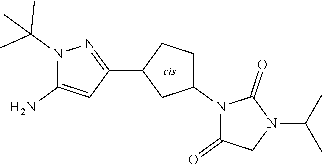 [SMILES: CC(C)N1CC(=O)N(C2CCC(c3cc(N)n(C(C)(C)C)n3)C2)C1=O]

cis-benzyl (1-(tert-butyl)-3-(3-(3-isopropyl-2,5-dioxoimidazolidin-1-yl)cyclopentyl)-1H-pyrazol-5-yl)carbamate (150 mg, 0.31 mmol) was dissolved in MeOH (10 mL), Pd/C (10 wt %, 100 mg) was added, the reaction mixture was stirred for 2 hours at room temperature under 1 atm H₂. After filtration, the filtrated was concentrated to afford the product (99 mg, 92%). LC-MS (ESI): m/z [M+H]⁺=348.5.

### Step 2: cis-3-(3-(3-((4-fluoro-1,1-dioxido-2,3-dihydrobenzo[d]isothiazol-5-yl)amino)-1H-pyrazol-5-yl)cyclopentyl)-1-isopropylimidazolidine-2,4-dione

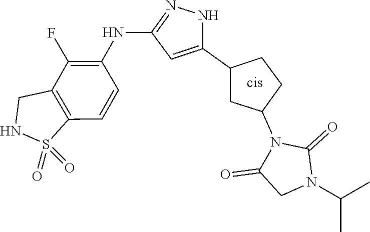 [SMILES: CC(C)N1CC(=O)N(C2CCC(c3cc(Nc4ccc5c(c4F)CNS5(=O)=O)n[nH]3)C2)C1=O]

The titled compound was synthesized in the procedures similar to Example 81. ¹H NMR (500 MHz, DMSO-d₆) δ 12.01 (s, 1H), 8.74 (d, J=2.5 Hz, 1H), 8.28 (s, 1H), 7.69 (t, J=5.2 Hz, 1H), 7.48 (d, J=8.6 Hz, 1H), 5.86 (s, 1H), 4.52-4.43 (m, 1H), 4.41 (d, J=5.1 Hz, 2H), 4.13 (p, J=6.7 Hz, 1H), 3.90 (s, 2H), 3.07 (q, J=8.8 Hz, 1H), 2.16 (t, J=9.1 Hz, 2H), 2.11-2.01 (m, 2H), 1.98-1.84 (m, 2H), 1.12 (d, J=6.7 Hz, 6H). LC-MS (ESI): m/z [M+H]⁺=477.3.

### Example 83: cis-1-(3-(3-((4-fluoro-1,1-dioxido-2,3-dihydrobenzo[d]isothiazol-5-yl)amino)-1H-pyrazol-5-yl)cyclopentyl)-3-isopropyl-1,3-dihydro-2H-imidazol-2-one

### Step 1: cis-1-(3-(5-amino-1-(tert-butyl)-1H-pyrazol-3-yl)cyclopentyl)-3-isopropyl-1,3-dihydro-2H-imidazol-2-one

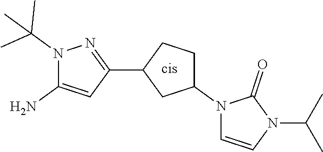 [SMILES: CC(C)n1ccn(C2CCC(c3cc(N)n(C(C)(C)C)n3)C2)c1=O]

cis-benzyl (1-(tert-butyl)-3-(3-(3-isopropyl-2-oxo-2,3-dihydro-1H-imidazol-1-yl)cyclopentyl)-1H-pyrazol-5-yl)carbamate (150 mg, 0.32 mmol) was dissolved in EA (10 mL), Pd/C (10% on carbon, 200 mg) was added, the reaction mixture was stirred for 1 hours at room temperature under 1 atm H₂. After filtration, the filtrated was concentrated to afford the product (88 mg, 83%). LC-MS (ESI): m/z [M+H]⁺=332.5.

### Step 2: 1-(3-(3-((4-fluoro-1,1-dioxido-2,3-dihydrobenzo[d]isothiazol-5-yl)amino)-1H-pyrazol-5-yl)cyclopentyl)-3-isopropyl-1,3-dihydro-2H-imidazol-2-one

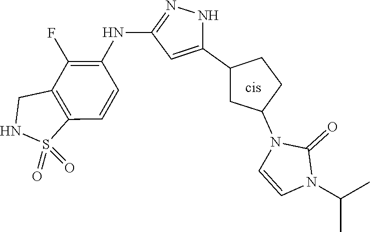 [SMILES: CC(C)n1ccn(C2CCC(c3cc(Nc4ccc5c(c4F)CNS5(=O)=O)n[nH]3)C2)c1=O]

The titled compound was synthesized in the procedures similar to Example 81. ¹H NMR (500 MHz, DMSO-d₆) δ 12.07 (s, 1H), 8.74 (d, J=2.6 Hz, 1H), 8.24 (s, 1H), 7.69 (t, J=5.2 Hz, 1H), 7.48 (d, J=8.6 Hz, 1H), 6.60 (s, 2H), 5.88 (s, 1H), 4.49 (p, J=8.0 Hz, 1H), 4.41 (d, J=5.1 Hz, 2H), 4.19 (h, J=6.8 Hz, 1H), 3.20-3.13 (m, 1H), 2.33 (dt, J=13.2, 7.2 Hz, 1H), 2.12-2.05 (m, 2H), 1.89-1.72 (m, 3H), 1.21 (d, J=6.8 Hz, 6H). LC-MS (ESI): m/z [M+H]⁺=461.3.

### Example 84: cis-3-(3-(3-((4-fluoro-1,1-dioxido-2,3-dihydrobenzo[d]isothiazol-5-yl)amino)-1H-pyrazol-5-yl)cyclopentyl)-5,5-dimethylimidazolidine-2,4-dione

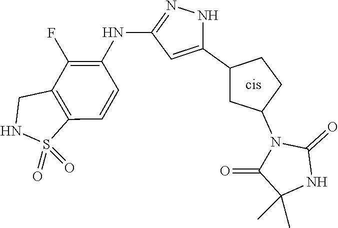 [SMILES: CC1(C)NC(=O)N(C2CCC(c3cc(Nc4ccc5c(c4F)CNS5(=O)=O)n[nH]3)C2)C1=O]

The titled compound was synthesized in the procedures similar to Example 81 in a racemic form, which was separated by chiral HPLC to give:

Enantiomer 1 (Example 84a, 100% ee); Retention time: 4.694 min. H NMR (500 MHz, DMSO-d₆) δ 12.00 (s, 1H), 8.73 (s, 1H), 8.28 (s, 2H), 7.68 (t, J=5.3 Hz, 1H), 7.47 (d, J=8.5 Hz, 1H), 5.87 (s, 1H), 4.45 (d, J=8.0 Hz, 1H), 4.40 (d, J=5.2 Hz, 2H), 3.09 (s, 1H), 2.20-2.10 (m, 2H), 2.09-2.02 (m, 2H), 1.96-1.89 (m, 2H), 1.26 (s, 6H). LC-MS (ESI): m/z [M+H]⁺=463.3.

Enantiomer 2 (Example 84b, 100% ee); Retention time: 11.253 min. ¹H NMR (500 MHz, DMSO-d₆) δ 12.00 (s, 1H), 8.73 (s, 1H), 8.28 (s, 2H), 7.68 (t, J=5.3 Hz, 1H), 7.47 (d, J=8.5 Hz, 1H), 5.87 (s, 1H), 4.45 (d, J=8.0 Hz, 1H), 4.40 (d, J=5.2 Hz, 2H), 3.09 (s, 1H), 2.20-2.10 (m, 2H), 2.09-2.02 (m, 2H), 1.96-1.89 (m, 2H), 1.26 (s, 6H). LC-MS (ESI): m/z [M+H]⁺=463.3.

Chiral analytical method: Column: CHIRALPAK IF-3 4.6*250 mm 5 μm; Mobile phase: A for MtBE (0.1% DEA) and B for MeOH:DCM=1:1; Gradient: Mobile Phase A: Mobile Phase B=50:50 (v/v); HPLC Equipment: HPLC-Agilent; Column temperature: 35° C.

Chiral Prep-HPLC Condition: CHIRALPAK IF-3 20*250 mm 5 μm; Mobile phase: A for MtBE (0.1% DEA) and B for MeOH:DCM=1:1; Gradient: Mobile Phase A: Mobile Phase B=50:50 (v/v); Flow Rate: 20 mL/min, Wavelength: UV 220 nm, Prep-HPLC Equipment: Prep-HPLC-Gilson; Column temperature: 25° C.

### Example 85: cis-1-(3-(3-((4-fluoro-1,1-dioxido-2,3-dihydrobenzo[d]isothiazol-5-yl)amino)-1H-pyrazol-5-yl)cyclopentyl)-4,4-dimethylimidazolidin-2-one

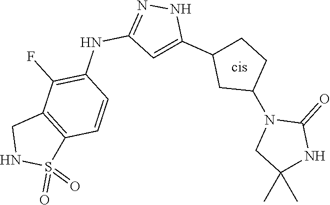 [SMILES: CC1(C)CN(C2CCC(c3cc(Nc4ccc5c(c4F)CNS5(=O)=O)n[nH]3)C2)C(=O)N1]

The titled compound was synthesized in the procedures similar to Example 81. ¹H NMR (500 MHz, DMSO-d₆) δ 12.01 (s, 1H), 8.71 (s, 1H), 8.25 (s, 1H), 7.68 (d, J=4.6 Hz, 1H), 7.47 (d, J=8.5 Hz, 1H), 6.39 (s, 1H), 5.85 (s, 1H), 4.40 (d, J=5.1 Hz, 2H), 4.23 (d, J=9.5 Hz, 1H), 3.11-3.02 (m, 2H), 2.48-2.41 (m, 1H), 2.11-2.00 (m, 2H), 1.82-1.74 (m, 1H), 1.69-1.59 (m, 3H), 1.17 (d, J=2.6 Hz, 6H). LC-MS (ESI): m/z [M+H]⁺=449.3

### Example 86: cis-3-(3-(3-((4-fluoro-1,1-dioxido-2,3-dihydrobenzo[d]isothiazol-5-yl)amino)-1H-pyrazol-5-yl)cyclopentyl)-1,5,5-trimethylimidazolidine-2,4-dione

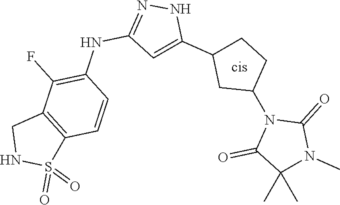 [SMILES: CN1C(=O)N(C2CCC(c3cc(Nc4ccc5c(c4F)CNS5(=O)=O)n[nH]3)C2)C(=O)C1(C)C]

The titled compound was synthesized in the procedures similar to Example 81. ¹H NMR (500 MHz, DMSO-d₆) δ 12.01 (s, 1H), 8.74 (d, J=2.6 Hz, 1H), 8.28 (s, 1H), 7.69 (t, J=5.2 Hz, 1H), 7.48 (d, J=8.6 Hz, 1H), 5.87 (s, 1H), 4.48 (ddd, J=14.5, 9.5, 7.0 Hz, 1H), 4.41 (d, J=5.1 Hz, 2H), 3.14-3.04 (m, 1H), 2.79 (s, 3H), 2.22-2.10 (m, 2H), 2.10-1.99 (m, 2H), 1.99-1.88 (m, 2H), 1.28 (d, J=2.4 Hz, 6H). LC-MS (ESI): m/z [M+H]⁺=477.3

### Example 87: cis-1-(3-(3-((4-fluoro-1,1-dioxido-2,3-dihydrobenzo[d]isothiazol-5-yl)amino)-1H-pyrazol-5-yl)cyclopentyl)-3,3-dimethylpyrrolidine-2,5-dione

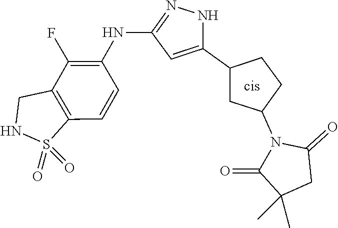 [SMILES: CC1(C)CC(=O)N(C2CCC(c3cc(Nc4ccc5c(c4F)CNS5(=O)=O)n[nH]3)C2)C1=O]

The titled compound was synthesized in the procedures similar to Example 21. ¹H NMR (500 MHz, DMSO-d₆) δ 12.02 (s, 1H), 8.74 (s, 1H), 8.29 (t, J=5 Hz, 1H), 7.69 (s, 1H), 7.48 (d, J=10 Hz, 1H), 5.86 (s, 1H), 4.58-4.49 (m, 1H), 4.45-4.37 (m, 2H), 3.13-3.04 (m, 1H), 2.55 (s, 2H), 2.21-1.87 (m, 6H), 1.23-1.17 (m, 6H). LC-MS (ESI): m/z [M+H]⁺=462.1.

### Example 88: cis-5-((5-(3-(4-(tert-butyl)-1H-imidazol-1-yl)cyclopentyl)-1H-pyrazol-3-yl)amino)-4-fluoro-2,3-dihydrobenzo[d]isothiazole 1,1-dioxide

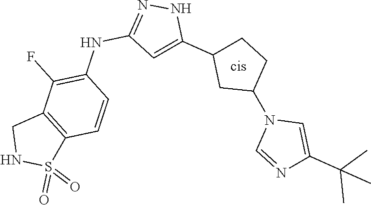 [SMILES: CC(C)(C)c1cn(C2CCC(c3cc(Nc4ccc5c(c4F)CNS5(=O)=O)n[nH]3)C2)cn1]

The titled compound was synthesized in the procedures similar to Example 21. ¹H NMR (500 MHz, DMSO-d₆) δ 12.08 (s, 1H), 8.75 (s, 1H), 8.24 (s, 1H), 7.69 (d, J=5.1 Hz, 1H), 7.48 (d, J=8.6 Hz, 1H), 7.27-7.02 (m, 1H), 5.89 (d, J=14.8 Hz, 1H), 4.68 (s, 1H), 4.41 (d, J=5.0 Hz, 2H), 3.28-3.19 (m, 2H), 2.61-2.53 (m, 1H), 2.31-2.12 (m, 2H), 2.00-1.87 (m, 3H), 1.22 (s, 9H). LC-MS (ESI): m/z [M+H]⁺=459.3.

### Example 89: cis-5-((5-(3-(4-(tert-butyl)-1H-pyrazol-1-yl)cyclopentyl)-1H-pyrazol-3-yl)amino)-4-fluoro-2,3-dihydrobenzo[d]isothiazole 1,1-dioxide

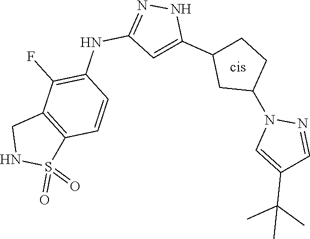 [SMILES: CC(C)(C)c1cnn(C2CCC(c3cc(Nc4ccc5c(c4F)CNS5(=O)=O)n[nH]3)C2)c1]

The titled compound was synthesized in the procedures similar to Example 21. ¹H NMR (500 MHz, DMSO-d₆) δ 12.07 (s, 1H), 8.74 (s, 1H), 8.27 (s, 1H), 7.69 (t, J=5.1 Hz, 1H), 7.57 (d, J=0.7 Hz, 1H), 7.48 (d, J=8.6 Hz, 1H), 7.34 (s, 1H), 5.88 (s, 1H), 4.74 (dd, J=15.3, 7.8 Hz, 1H), 4.41 (d, J=5.2 Hz, 2H), 3.23-3.15 (m, 1H), 2.46-2.43 (m, 1H), 2.19-1.98 (m, 4H), 1.93-1.82 (m, 1H), 1.20 (s, 9H). LC-MS (ESI): m/z [M+H]⁺=459.3.

### Example 90: cis-5-((5-(3-(4-(tert-butyl)-1H-1,2,3-triazol-1-yl)cyclopentyl)-1H-pyrazol-3-yl)amino)-4-fluoro-2,3-dihydrobenzo[d]isothiazole 1,1-dioxide

### Step 1: trans-3-(5-(((benzyloxy)carbonyl)amino)-1-(tert-butyl)-1H-pyrazol-3-yl)cyclopentyl methanesulfonate

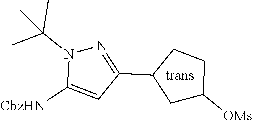 [SMILES: CC(C)(C)n1nc(C2CCC(OS(C)(=O)=O)C2)cc1NC(=O)OCc1ccccc1]

To a solution of trans-3-(5-(((benzyloxy)carbonyl)amino)-1-(tert-butyl)-1H-pyrazol-3-yl)cyclopentyl 4-nitrobenzoate (506 mg, 1 mmol) in DCM (20 mL) was added pyridine (160 mg, 2 mmol) and methane sufonyl chloride (115 mg, 1 mmol) at 0° C. The mixture was stirred at rt for 2 h. Water (20 mL) was added and the mixture was extracted by DCM (2×10 mL). The combined organic layers were washed with brine, dried over Na₂SO₄ and concentrated under vacuum. The residue was purified by column chromatography on silica gel, eluting with PE:EA (100:0 to 2:1) to give the product (360 mg, 83%). LC-MS (ESI): m/z [M+H]⁺=436.4.

### Step 2: cis-benzyl (3-(3-azidocyclopentyl)-1-(tert-butyl)-1H-pyrazol-5-yl)carbamate

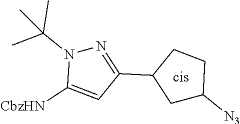 [SMILES: CC(C)(C)n1nc(C2CCC(N=[N+]=[N-])C2)cc1NC(=O)OCc1ccccc1]

To a solution of trans-3-(5-(((benzyloxy)carbonyl)amino)-1-(tert-butyl)-1H-pyrazol-3-yl)cyclopentyl methanesulfonate (360 mg, 0.83 mmol) in DMF (10 mL) was added sodium azide (267 mg, 4.1 mmol). The resulting mixture was stirred at rt for 3 h. Water (30 mL) was added and the mixture was extracted by DCM (2×20 mL). The combined organic layers were washed with brine, dried over Na₂SO₄ and concentrated under vacuum. The residue was purified by column chromatography on silica gel, eluting with PE:EA (100:0 to 2:1) to give the product (289 mg, 91%). LC-MS (ESI): m/z [M+H]⁺=383.4.

### Step 3: cis-benzyl (1-(tert-butyl)-3-(3-(4-(tert-butyl)-1H-1,2,3-triazol-1-yl)cyclopentyl)-1H-pyrazol-5-yl)carbamate

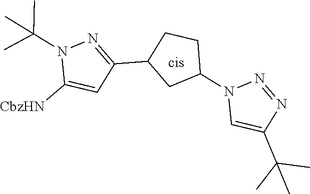 [SMILES: CC(C)(C)c1cn(C2CCC(c3cc(NC(=O)OCc4ccccc4)n(C(C)(C)C)n3)C2)nn1]

To a solution of cis-benzyl (3-(3-azidocyclopentyl)-1-(tert-butyl)-1H-pyrazol-5-yl)carbamate (402 mg, 1.1 mmol) in t-BuOH/H₂O (10/10 mL) was added 3,3-Dimethyl-1-butyne (130 mg, 1.5 mmol), sodium ascorbate (80 mg, 0.4 mmol) and Copper(II) sulfate pentahydrate (30 mg, 0.1 mmol). The resulting mixture was stirred at rt for 1 h. Water (10 mL) was added and the mixture was extracted by EA (2×20 mL). The combined organic layers were washed with brine, dried over Na₂SO₄ and concentrated under vacuum. The residue was purified by column chromatography on silica gel, eluting with PE:EA (100:0 to 1:1) to give the product (403 mg, 79%). LC-MS (ESI): m/z [M+H]⁺=465.4.

### Step 4: cis-1-(tert-butyl)-3-(3-(4-(tert-butyl)-1H-1,2,3-triazol-1-yl)cyclopentyl)-1H-pyrazol-5-amine

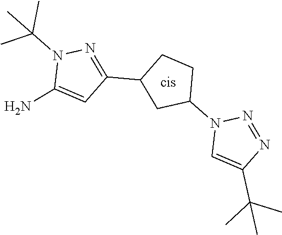 [SMILES: CC(C)(C)c1cn(C2CCC(c3cc(N)n(C(C)(C)C)n3)C2)nn1]

cis-benzyl (1-(tert-butyl)-3-(3-(4-(tert-butyl)-1H-1,2,3-triazol-1-yl)cyclopentyl)-1H-pyrazol-5-yl)carbamate (403 mg, 0.87 mmol) was dissolved in THF/H₂O (10/1 mL), Pd/C (10% on carbon, 200 mg) was added, the reaction mixture was stirred for 1 hours at room temperature under 1 atm H₂. After filtration, the filtrated was concentrated to afford the product (273 mg, 95%). LC-MS (ESI): m/z [M+H]⁺=331.4.

### Step 5: cis-5-((1-(tert-butyl)-3-(3-(4-(tert-butyl)-1H-1,2,3-triazol-1-yl)cyclopentyl)-1H-pyrazol-5-yl)amino)-4-fluoro-2,3-dihydrobenzo[d]isothiazole 1,1-dioxide

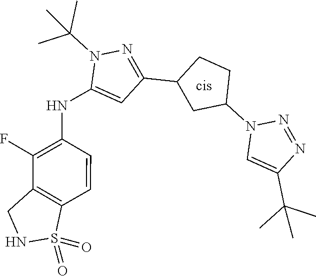 [SMILES: CC(C)(C)c1cn(C2CCC(c3cc(Nc4ccc5c(c4F)CNS5(=O)=O)n(C(C)(C)C)n3)C2)nn1]

A mixture of cis-1-(tert-butyl)-3-(3-(4-(tert-butyl)-1H-1,2,3-triazol-1-yl)cyclopentyl)-1H-pyrazol-5-amine (160 mg, 0.5 mmol), 5-bromo-4-fluoro-2,3-dihydrobenzo[d]isothiazole 1,1-dioxide (130 mg, 0.5 mmol), Pd₂dba₃ (48 mg, 0.05 mmol), Xantphos (58 mg, 0.1 mmol) and K₃PO₃ (320 mg, 2.5 mmol) in dioxane (10 mL) was stirred for 6 h at 100° C. in the sealed tube under N₂ atmosphere. The solution was concentrated under reduced pressure. The residue was purified by silica gel column chromatography, eluting with 100% EtOAc to afford the product (144 mg, 56%). LC-MS (ESI): m/z [M+H]⁺=516.3.

### Step 6: cis-5-((5-(3-(4-(tert-butyl)-1H-1,2,3-triazol-1-yl)cyclopentyl)-1H-pyrazol-3-yl)amino)-4-fluoro-2,3-dihydrobenzo[d]isothiazole 1,1-dioxide

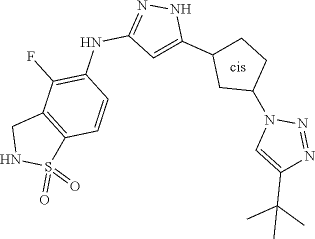 [SMILES: CC(C)(C)c1cn(C2CCC(c3cc(Nc4ccc5c(c4F)CNS5(=O)=O)n[nH]3)C2)nn1]

cis-5-((1-(tert-butyl)-3-(3-(4-(tert-butyl)-1H-1,2,3-triazol-1-yl)cyclopentyl)-1H-pyrazol-5-yl)amino)-4-fluoro-2,3-dihydrobenzo[d]isothiazole 1,1-dioxide (144 mg, 0.28 mmol) was dissolved in formic acid (10 mL) and stirred at 75° C. for 6 h. The mixture was concentrated under vacuum and the residue was purified by prep HPLC (Waters XSelect C18: RD-CO-094 column, eluting with a gradient of acetonitrile/water containing 0.1% FA, 20%-40%) to give the product (47.5 mg, 37%). ¹H NMR (500 MHz, DMSO-d₆) δ 12.1 (brs, 1H), 8.76 (d, J=2.6 Hz, 1H), 8.26 (s, 1H), 7.93 (s, 1H), 7.70 (s, 1H), 7.48 (d, J=8.6 Hz, 1H), 5.91 (s, 1H), 5.05 (tt, J=8.9, 7.0 Hz, 1H), 4.41 (s, 2H), 3.26 (ddd, J=17.8, 10.2, 7.7 Hz, 1H), 2.66 (dt, J=13.6, 7.2 Hz, 1H), 2.22-2.02 (m, 3H), 2.01-1.88 (m, 1H), 1.27 (s, 9H). LC-MS (ESI): m/z [M+H]⁺=460.2.

### Example 91: cis-5-((5-(3-(5-(tert-butyl)oxazol-2-yl)cyclopentyl)-1H-pyrazol-3-yl)amino)-4-fluoro-2,3-dihydrobenzo[d]isothiazole 1,1-dioxide

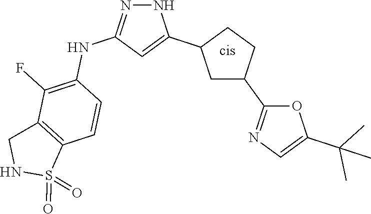 [SMILES: CC(C)(C)c1cnc(C2CCC(c3cc(Nc4ccc5c(c4F)CNS5(=O)=O)n[nH]3)C2)o1]

The titled compound was synthesized in the procedures similar to Example 6 in a racemic form, which was further separated by Chiral Prep-HPLC to give:

Enantiomer 1 (Example 91a, 100% ee); Retention time: 1.742 min. ¹H NMR (500 MHz, DMSO-d₆) δ=12.04 (s, 1H), 8.72 (s, 1H), 8.26 (t, J=7.5 Hz, 1H), 7.69 (t, J=5.1 Hz, 1H), 7.47 (d, J=8.6 Hz, 1H), 6.67 (s, 1H), 5.84 (s, 1H), 4.40 (d, J=5.2 Hz, 2H), 3.40-3.33 (m, 1H), 3.25-3.13 (m, 1H), 2.48-2.42 (m, 1H), 2.16-2.05 (m, 2H), 2.03-1.85 (m, 2H), 1.79-1.67 (m, 1H), 1.23 (s, 9H). LC-MS (ESI): m/z [M+H]⁺=460.2.

Enantiomer 2 (Example 91b, 99.35% ee); Retention time: 2.238 min. ¹H NMR (500 MHz, DMSO-d₆) δ=12.04 (s, 1H), 8.72 (s, 1H), 8.26 (t, J=7.5 Hz, 1H), 7.69 (t, J=5.1 Hz, 1H), 7.47 (d, J=8.6 Hz, 1H), 6.67 (s, 1H), 5.84 (s, 1H), 4.40 (d, J=5.2 Hz, 2H), 3.40-3.33 (m, 1H), 3.25-3.13 (m, 1H), 2.48-2.42 (m, 1H), 2.16-2.05 (m, 2H), 2.03-1.85 (m, 2H), 1.79-1.67 (m, 1H), 1.23 (s, 9H). LC-MS (ESI): m/z [M+H]⁺=460.2.

Chiral analytical method: Column: CHIRALPAK IF-3 4.6*250 mm 5 μm; Mobile phase: A for MtBE (0.1% DEA) and B for MeOH:DCM=1:1; Gradient: Mobile Phase A: Mobile Phase B=50:50 (v/v); HPLC Equipment: HPLC-Agilent; Column temperature: 35° C.

Chiral Prep-HPLC Condition: CHIRALPAK IF-3 20*250 mm 5 μm; Mobile phase: A for MtBE (0.1% DEA) and B for MeOH:DCM=1:1; Gradient: Mobile Phase A: Mobile Phase B=50:50 (v/v); Flow Rate: 20 mL/min, Wavelength: UV 220 nm, Prep-HPLC Equipment: Prep-HPLC-Gilson; Column temperature: 25° C.

### Example 92: 5-((5-(3-(5-ethyloxazol-2-yl)cyclopentyl)-1H-pyrazol-3-yl)amino)-4-fluoro-2,3-dihydrobenzo[d]isothiazole 1,1-dioxide

### Step 1: tert-butyl (2-oxobutyl)carbamate

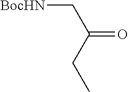

To a solution of tert-butyl (2-(methoxy(methyl)amino)-2-oxoethyl)carbamate (10 g, 45.8 mmol) in THF (100 mL) was added ethylmagnesium bromide (46 mL, 137.6 mmol) at 0° C. in 15 min. The reaction was stirred at rt for 45 min. The reaction was quenched with NH₄Cl solution (100 mL) and extracted with EA (3×80 mL). The combined organic layers were washed with brine and concentrated under reduced pressure. The crude was purified by silica gel column chromatography, eluting with PE and EA (100:0 to 90:10) to yield the product (9 g, 95% yield).

### Step 2: 1-aminobutan-2-one hydrochloride

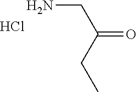

tert-butyl (2-oxobutyl)carbamate (9 g, 48.1 mmol) was added to HCl in dioxane (4 M, 60 mL) and the resulting solution was stirred at rt for 2 h. The reaction was concentrated under vacuum to afford the crude product (5 g, 84.7%).

### Step 3: 3-(methoxycarbonyl)cyclopentane-1-carboxylic acid

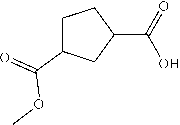

To a solution of dimethyl cyclopentane-1,3-dicarboxylate (20 g, 107.5 mmol) in MeOH (200 mL) was added NaOH (4.2 g, 107.5 mmol). The resulting solution was stirred at rt for 12 h. The mixture was acidified to pH<8 with aq. HCl (2M). The resulting mixture was extracted with EA (3×80 mL), and the organic layers were combined, dried over Na₂SO₄ then concentrated under vacuum to give the product (12.8 g, 68.8%). LCMS (ESI) m/z [M−H]—=171.

### Step 4: methyl 3-((2-oxobutyl)carbamoyl)cyclopentane-1-carboxylate

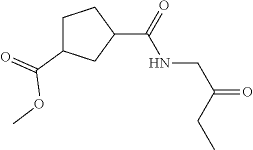 [SMILES: CCC(=O)CNC(=O)C1CCC(C(=O)OC)C1]

To a solution of 1-aminobutan-2-one hydrochloride (6.5 g, 52.8 mmol) in DMF (150 mL) were added 3-(methoxycarbonyl)cyclopentane-1-carboxylic acid (9.08 g, 52.8 mmol), HATU (24 g, 63.3 mmol) and DIEA (17 g, 132 mmol). The resulting solution was stirred at rt for overnight. The reaction was quenched with H₂O (300 mL) and extracted with EA (3×80 mL). The combined organic layers were washed with brine, dried over Na₂SO₄ then concentrated under vacuum. The residue was purified by silica gel column chromatography, eluting with PE:EA (100:0 to 60:40) to give the product (8 g, 63.5%). LCMS (ESI) m/z [M+H]⁺=242.

### Step 5: methyl 3-(5-ethyloxazol-2-yl)cyclopentane-1-carboxylate

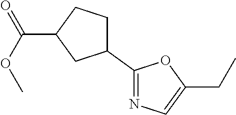 [SMILES: CCc1cnc(C2CCC(C(=O)OC)C2)o1]

methyl 3-((2-oxobutyl)carbamoyl)cyclopentane-1-carboxylate (6.9 g, 28.6 mmol) was dissolved in POCl₃ (30 mL) and stirred at 60° C. for 1 h. The resulting mixture was concentrated under vacuum. H₂O (100 mL) was added and the mixture was adjusted to Ph=8 with aq. Na₂CO₃ (2M). The resulting solution was extracted with DCM (2×80 mL). The combined organic layers were washed with brine, dried over Na₂SO₄ then concentrated under vacuum. The residue was purified by column chromatography on silica gel, eluting with PE:EA (100:0 to 62:38) to give the product (3.6 g, 57.1%). LCMS (ESI) m/z [M+H]⁺=224.

### Step 6: 3-(3-(5-ethyloxazol-2-yl)cyclopentyl)-3-oxopropanenitrile

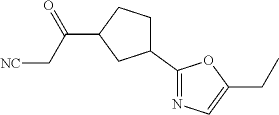 [SMILES: CCc1cnc(C2CCC(C(=O)CC#N)C2)o1]

To a solution of CH₃CN (1.2 g, 29.3 mmol) in THF (70 mL) was added n-Buli (2.5 M, 11.7 mL, 29.3 mmol) at −78° C. and stirred at this temperature for 1 h under N₂ atmosphere. The methyl 3-(5-ethyloxazol-2-yl)cyclopentane-1-carboxylate (3.3 g, 14.6 mmol) was added at −78° C. and further stirred for another hour. The reaction was quenched with NH₄Cl solution (80 mL) and extracted with EtOAc (3×80 mL). The combined organic layers were washed with brine, dried over Na₂SO₄ then concentrated under vacuum. The residue was purified by column chromatography on silica gel, eluting with PE:EA (100:0 to 60:40) to give the product (2 g, 58.3% yield). LCMS (ESI) m/z [M+H]⁺=233.

### Step 7: 5-(3-(5-ethyloxazol-2-yl)cyclopentyl)-1H-pyrazol-3-amine

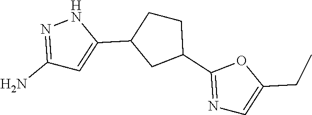 [SMILES: CCc1cnc(C2CCC(c3cc(N)n[nH]3)C2)o1]

To a solution of 3-(3-(5-ethyloxazol-2-yl)cyclopentyl)-3-oxopropanenitrile (1.6 g, 6.89 mmol) in EtOH (80 mL) were added hydrazine hydrate (379 mg, 7.58 mmol) and TsOH (50 mg). The resulting solution was stirred at 90° C. for 1 h. The reaction was concentrated under vacuum. The residue was purified by column chromatography on silica gel, eluting with DCM:MeOH (100:0 to 90:10) to give the product (769.3 mg, 45.5%). ¹H NMR (500 MHz, Chloroform-d) δ 6.63 (dt, J=18.1, 1.5 Hz, 1H), 5.49 (d, J=3.5 Hz, 1H), 4.74 (s, 2H), 3.43 (tdd, J=13.5, 7.6, 5.5 Hz, 1H), 3.33-3.23 (m, 1H), 2.66 (qd, J=7.6, 1.5 Hz, 2H), 2.54-2.38 (m, 1H), 2.30-2.18 (m, 1H), 2.17-2.08 (m, 1H), 2.11-2.00 (m, 2H), 1.85-1.73 (m, 1H), 1.26 (t, J=7.5 Hz, 3H). LCMS (ESI) m/z [M+H]⁺=247.

### Step 8: 5-((5-(3-(5-ethyloxazol-2-yl)cyclopentyl)-1H-pyrazol-3-yl)amino)-4-fluoro-2,3-dihydrobenzo[d]isothiazole 1,1-dioxide

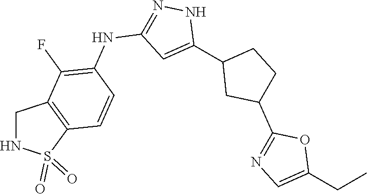 [SMILES: CCc1cnc(C2CCC(c3cc(Nc4ccc5c(c4F)CNS5(=O)=O)n[nH]3)C2)o1]

The titled compound was synthesized in the procedures similar to Example 6. ¹H NMR (500 MHz, DMSO-d₆) δ 12.04 (s, 1H), 8.73 (s, 1H), 8.22 (d, J=26.7 Hz, 1H), 7.69 (s, 1H), 7.48 (d, J=8.6 Hz, 1H), 6.71 (s, 1H), 5.86 (d, J=11.3 Hz, 1H), 4.41 (s, 2H), 3.29-3.15 (m, 2H), 2.67-2.61 (m, 2H), 2.48-2.41 (m, 1H), 2.22-2.06 (m, 2H), 2.02-1.85 (m, 2H), 1.79-1.68 (m, 1H), 1.17 (td, J=7.5, 4.2 Hz, 3H). LC-MS (ESI): m/z [M+H]⁺=432.2.

### Example 93: 4-fluoro-5-((5-(3-(5-(hydroxymethyl)oxazol-2-yl)cyclopentyl)-1H-pyrazol-3-yl)amino)-2,3-dihydrobenzo[d]isothiazole 1,1-dioxide

### Step 1: methyl 3-((2,3-dihydroxypropyl)carbamoyl)cyclopentane-1-carboxylate

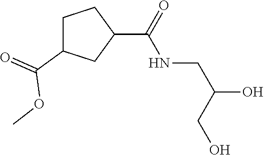 [SMILES: COC(=O)C1CCC(C(=O)NCC(O)CO)C1]

To a solution of 3-methoxycarbonyl)cyclopentane-1-carboxylic acid (11.3 g, 65.7 mmol) in DMF (60 mL) was added HATU (30 g, 78.9 mmol), 3-aminopropane-1,2-diol (6 g, 65.9 mmol) and DIEA (21 g, 162 mmol). The resulting mixture was stirred at rt for overnight. The reaction was quenched with H₂O (100 mL) and extracted with 3×80 mL of EA. The combined organic layers were washed with brine, concentrated under vacuum, and the residue was purified by silica gel column chromatography, eluting with PE:EA (100:0 to 60:40) to give the product (10 g, 95% yield). LCMS (ESI) m/z [M−H]—=246.

### Step 2: methyl 3-((3-((tert-butyldimethylsilyl)oxy)-2-hydroxypropyl)carbamoyl)cyclopentane-1-carboxylate

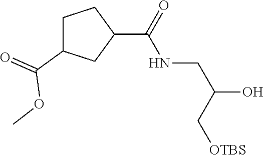 [SMILES: COC(=O)C1CCC(C(=O)NCC(O)CO[Si](C)(C)C(C)(C)C)C1]

To a solution of methyl 3-((2,3-dihydroxypropyl)carbamoyl)cyclopentane-1-carboxylate (10 g, 40.8 mmol) in DCM (100 mL) was added TBSCl (4.3 g, 28.6 mmol) and imidazole (8.3 g, 122 mmol). The resulting mixture was stirred at rt for 2 h. The reaction was concentrated under vacuum, and the residue was purified by silica gel column chromatography, eluting with PE:EA (100:0 to 70:30) to give the product (5 g, 36% yield). LCMS (ESI) m/z [M+H]⁺=360.

### Step 3: methyl 3-((3-((tert-butyldimethylsilyl)oxy)-2-oxopropyl)carbamoyl)cyclopentane-1-carboxylate

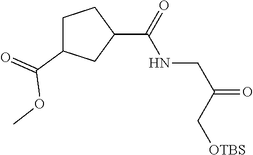 [SMILES: COC(=O)C1CCC(C(=O)NCC(=O)CO[Si](C)(C)C(C)(C)C)C1]

To a solution of methyl 3-((3-((tert-butyldimethylsilyl)oxy)-2-hydroxypropyl)carbamoyl)cyclopentane-1-carboxylate (5 g, 13.8 mmol) in DCM (60 mL) was added DMP (11.8 g, 27.8 mmol). The resulting mixture was stirred at rt for 2 h. The reaction was concentrated under vacuum, and the residue was purified by silica gel column chromatography, eluting with PE:EA (100:0 to 70:30) to give the product (3 g, 61%). LCMS (ESI) m/z [M+H]⁺=358.

### Step 3: methyl 3-(5-(((tert-butyldimethylsilyl)oxy)methyl)oxazol-2-yl)cyclopentane-1-carboxylate

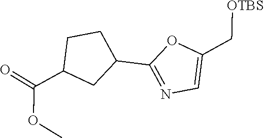 [SMILES: COC(=O)C1CCC(c2ncc(CO[Si](C)(C)C(C)(C)C)o2)C1]

To a solution of methyl 3-((3-((tert-butyldimethylsilyl)oxy)-2-oxopropyl)carbamoyl)cyclopentane-1-carboxylate (3 g, 8.3 mmol) in ACN (58 mL) were added a solution of C₂Cl₆ (5.75 g, 52.8 mmol) in ACN (45 mL), TEA (5 g, 49 mmol), and PPh₃ (6.37 g, 24.3 mmol) respectively. The resulting mixture was stirred at rt for 2 h. The reaction was quenched with H₂O (100 mL) and extracted with 3×80 mL of EA. The combined organic layers were washed with brine, dried over Na₂SO₄, and concentrated under vacuum. The residue was purified by silica gel column chromatography, eluting with PE:EA (100:0 to 60:40) to give the product (1.8 g, 65.5%). LCMS (ESI) m/z [M+1]⁺=340.

### Step 4: 3-(3-(5-(((tert-butyldimethylsilyl)oxy)methyl)oxazol-2-yl)cyclopentyl)-3-oxopropanenitrile

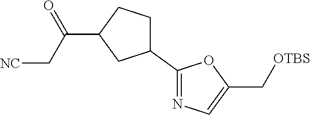 [SMILES: CC(C)(C)[Si](C)(C)OCc1cnc(C2CCC(C(=O)CC#N)C2)o1]

To a solution of CH₃CN (435 mg, 10.6 mmol) in THF (90 mL) was added n-BuLi (2.5 M) (4.23 mL, 10.6 mmol) at −78° C. and stirred for 1 h under N₂ atmosphere. A solution of methyl 3-(5-(((tert-butyldimethylsilyl)oxy)methyl)oxazol-2-yl)cyclopentane-1-carboxylate (1.8 g, 5.3 mmol) was added in it at −78° C. and stirred for another hour. The reaction was quenched with aq. NH₄Cl (80 mL), and extracted with 3×50 mL of EtOAc. The resulting mixture was concentrated under vacuum, and the residue was purified by silica gel column chromatography, eluting with PE:EA (100:0 to 60:40) to give the product (1.1 g, 61%). LCMS (ESI) m/z [M+H]⁺=349.

### Step 5: 5-(3-(5-(((tert-butyldimethylsilyl)oxy)methyl)oxazol-2-yl)cyclopentyl)-1H-pyrazol-3-amine

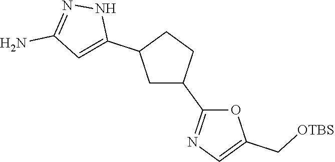 [SMILES: CC(C)(C)[Si](C)(C)OCc1cnc(C2CCC(c3cc(N)n[nH]3)C2)o1]

To a solution of 3-(3-(5-(((tert-butyldimethylsilyl)oxy)methyl)oxazol-2-yl)cyclopentyl)-3-oxopropanenitrile (1.1 g, 3.16 mmol) in EtOH (20 mL) was added NH₂NH₂H₂O (174 mg, 3.48 mmol) and TsOH (50 mg). The resulting mixture was stirred at 90° C. for 1 h. The reaction was concentrated under vacuum, and the residue was purified by silica gel column chromatography, eluting with DCM:MeOH (100:0 to 90:10) to give the product (723 mg, 65.7%). 1H NMR (300 MHz, DMSO-d₆) δ 11.12 (s, 1H), 6.96 (d, J=2.1 Hz, 1H), 5.20 (d, J=2.2 Hz, 1H), 4.65 (d, J=2.2 Hz, 2H), 3.34-3.25 (m, 2H), 3.14-2.92 (m, 1H), 2.38 (m, J=12.6, 7.0 Hz, 1H), 2.05 (m, J=13.3, 6.2 Hz, 2H), 1.97-1.89 (m, 1H), 1.87-1.76 (m, 1H), 1.76-1.61 (m, 1H), 1.31-1.09 (m, 1H), 0.86 (d, J=2.3 Hz, 9H), 0.06 (d, J=2.1 Hz, 6H). LCMS (ESI) m/z [M+H]⁺=363.

### Step 4: 5-((5-(3-(5-(((tert-butyldimethylsilyl)oxy)methyl)oxazol-2-yl)cyclopentyl)-1H-pyrazol-3-yl)amino)-4-fluoro-2,3-dihydrobenzo[d]isothiazole 1,1-dioxide

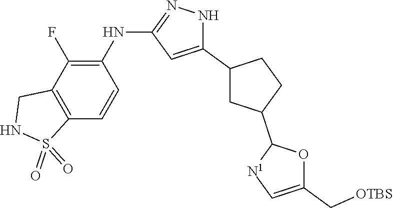

A mixture of 5-(3-(5-(((tert-butyldimethylsilyl)oxy)methyl)oxazol-2-yl)cyclopentyl)-1H-pyrazol-3-amine (50 mg, 0.138 mmol), 5-bromo-4-fluoro-2,3-dihydrobenzo[d]isothiazole 1,1-dioxide (44 mg, 0.165 mmol), Brettephos Pd G3 (13 mg, 0.014 mmol) and K₂CO₃ (57 mg, 0.414 mmol) in t-BuOH (10 mL) was stirred for 3 h at 110° C. in the sealed tube under N₂ atmosphere. The solution was concentrated under reduced pressure. The residue was purified by silica gel column chromatography, eluting with PE/EtOAc (1:4) to afford the product (40 mg, 53%). LC-MS (ESI): m/z [M+H]⁺=548.2.

### Step 5: 4-fluoro-5-((5-(3-(5-(hydroxymethyl)oxazol-2-yl)cyclopentyl)-1H-pyrazol-3-yl)amino)-2,3-dihydrobenzo[d]isothiazole 1,1-dioxide

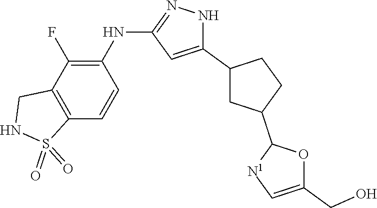

A solution of 5-((5-(3-(5-(((tert-butyldimethylsilyl)oxy)methyl)oxazol-2-yl)cyclopentyl)-1H-pyrazol-3-yl)amino)-4-fluoro-2,3-dihydrobenzo[d]isothiazole 1,1-dioxide (40 mg, 0.073 mmol) in DCM (4 mL) was added TFA (1 mL). The solution was stirred at room temperature for 4 h. The solution was concentrated under reduced pressure. The residue was dissolved in DCM (5 mL), then NH₃ in MeOH (7M, 1 mL) was added. After stirring for 2 h at room temperature, the mixture was concentrated under reduced pressure. The residue was purified by prep HPLC (Waters SunFire C18: RD-CO-095 column, eluting with 20%-60% of water (containing 0.1% FA) in acetonitrile to afford the product (15 mg, 47%).

¹H NMR (500 MHz, DMSO-d₆) δ 12.06 (s, 1H), 8.76 (s, 1H), 8.28 (t, J=8.0 Hz, 1H), 7.71 (s, 1H), 7.48 (d, J=8.6 Hz, 1H), 6.90 (s, 1H), 5.85 (s, 1H), 5.35-5.27 (m, 1H), 4.50-4.37 (m, 4H), 3.44-3.36 (m, 1H), 3.26-3.16 (m, 1H), 2.48-2.42 (m, 1H), 2.17-2.07 (m, 2H), 2.03-1.96 (m, 1H), 1.95-1.84 (m, 1H), 1.80-1.70 (m, 1H). LC-MS (ESI): m/z [M+H]⁺=434.38.

### Example 94: 5-((5-(3-(4-(tert-butyl)oxazol-2-yl)cyclopentyl)-1H-pyrazol-3-yl)amino)-4-fluoro-2,3-dihydrobenzo[d]isothiazole 1,1-dioxide

### Step 1: cis-1-(3,3-dimethyl-2-oxobutyl) 3-methyl cyclopentane-1,3-dicarboxylate

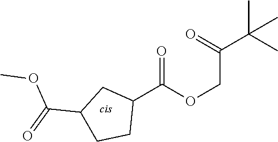 [SMILES: COC(=O)C1CCC(C(=O)OCC(=O)C(C)(C)C)C1]

To a solution of cis-3-(methoxycarbonyl)cyclopentane-1-carboxylic acid (5 g, 29 mmol) in DMF (60 mL) was added 1-chloro-3,3-dimethylbutan-2-one (5 g, 37.7 mmol) and K₂CO₃ (11.25 g, 81.2 mmol) and stirred at rt for 2 h. The reaction was quenched with H₂O (100 mL) and extracted with 3×80 mL of EA. The combined organic layers were washed with brine and concentrated under vacuum. The residue was purified by silica gel column chromatography, eluting with PE:EA (100:0 to 60:40) to give the product (6.2 g, 80%). LCMS (ESI): m/z [M+H]⁺=271.

### Step 2: cis-methyl 3-(4-(tert-butyl)oxazol-2-yl)cyclopentane-1-carboxylate

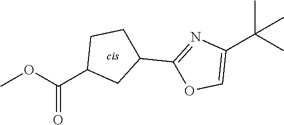 [SMILES: COC(=O)C1CCC(c2nc(C(C)(C)C)co2)C1]

To a solution of cis-1-(3,3-dimethyl-2-oxobutyl) 3-methyl cyclopentane-1,3-dicarboxylate (5 g, 18.5 mmol) in AcOH (200 mL) was added AcONH₄ (11 g, 183 mmol) at 100° C. for 1.5 h under microwave. The mixture was concentrated under vacuum and the residue was purified by silica gel column chromatography, eluting with PE:EA (100:0 to 60:40) to give the product (2 g, 44%). LCMS (ESI): m/z [M+H]⁺=252.

### Step 3: 3-(3-(4-(tert-butyl)oxazol-2-yl)cyclopentyl)-3-oxopropanenitrile

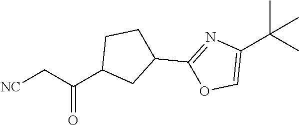 [SMILES: CC(C)(C)c1coc(C2CCC(C(=O)CC#N)C2)n1]

To a solution of CH₃CN (1.5 g, 36.5 mmol) in THF (70 mL) was added n-BuLi (2.5 M) (11 mL, 27.4 mmol) at −78° C. and stirred for 1 h under N₂ atmosphere. A solution of methyl 3-(4-(tert-butyl)oxazol-2-yl)cyclopentane-1-carboxylate (2.3 g, 9.2 mmol) in THF (30 ml) was added in it at −78° C. and stirred for another hour. The reaction was quenched with NH₄Cl solution (80 mL) and extracted with 3×50 mL of EtOAc. The resulting mixture was concentrated under vacuum and the residue was purified by silica gel column chromatography PE:EA (100:0 to 60:40) to give the product (1 g, 43.4%). LCMS (ESI): m/z [M+H]⁺=261.

### Step 4: 5-(3-(4-(tert-butyl)oxazol-2-yl)cyclopentyl)-1H-pyrazol-3-amine

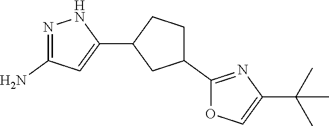 [SMILES: CC(C)(C)c1coc(C2CCC(c3cc(N)n[nH]3)C2)n1]

To a solution of 3-(3-(4-(tert-butyl)oxazol-2-yl)cyclopentyl)-3-oxopropanenitrile (1 g, 3.8 mmol) in EtOH (15 mL) was added hydrazine hydrate (211 mg, 4.2 mmol) and TsOH (50 mg). The mixture was stirred at 90° C. for 1 h. The reaction was concentrated under vacuum. The residue was purified by silica gel column chromatography, eluting with DCM:MeOH (100:0 to 90:10) to give the product (546 mg, 50%). ¹H NMR (300 MHz, DMSO-d₆) δ 11.17 (s, 1H), 7.62 (s, 1H), 5.22 (d, J=5.4 Hz, 1H), 4.45 (s, 2H), 3.33-3.25 (m, 1H), 3.15-2.87 (m, 1H), 2.36 (m, J=12.1, 7.2 Hz, 1H), 2.10-2.00 (m, 2H), 1.98-1.89 (m, 1H), 1.82 (m, J=12.3, 10.5 Hz, 1H), 1.74-1.62 (m, 1H), 1.20 (s, 9H). LCMS (ESI): m/z [M₋+H]⁺=275.

### Step 5: 5-((5-(3-(4-(tert-butyl)oxazol-2-yl)cyclopentyl)-1H-pyrazol-3-yl)amino)-4-fluoro-2,3-dihydrobenzo[d]isothiazole 1,1-dioxide

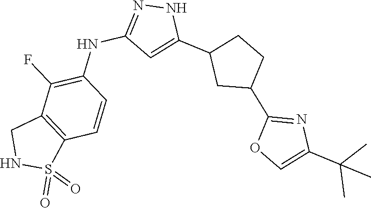 [SMILES: CC(C)(C)c1coc(C2CCC(c3cc(Nc4ccc5c(c4F)CNS5(=O)=O)n[nH]3)C2)n1]

To a mixture of 5-bromo-4-fluoro-2,3-dihydrobenzo[d]isothiazole 1,1-dioxide (26 mg, 0.1 mmol) and 5-(3-(4-(tert-butyl)oxazol-2-yl)cyclopentyl)-1H-pyrazol-3-amine (27 mg, 0.1 mmol) in t-BuOH (3 mL) was added BrettPhos Pd G3 (9 mg, 0.01 mmol) and K₂CO₃ (42 mg, 0.3 mmol). The reaction mixture was stirred at 110° C. under nitrogen atmosphere for 4 h. The mixture was concentrated under reduced pressure. The residue was purified by prep HPLC (Waters SunFire C18: RD-CO-058 column, eluting with 34%-49% of water (containing 0.1% FA) in acetonitrile to afford the product (11.14 mg, 24%). ¹H NMR (500 MHz, DMSO-d₆) δ 12.06 (s, 1H), 8.72 (s, 1H), 8.25 (s, 1H), 7.69-7.64 (m, 2H), 7.48 (d, J=8.6, 1H), 5.85 (s, 1H), 4.41 (s, 2H), 3.24-3.15 (m, 1H), 2.49-2.41 (m, 1H), 2.26-1.64 (m, 6H), 1.20-1.19 (m, 9H). LC-MS (ESI): m/z [M+H]⁺=460.3

### Example 95: 5-((5-(3-(5-(tert-butyl)thiazol-2-yl)cyclopentyl)-1H-pyrazol-3-yl)amino)-4-fluoro-2,3-dihydrobenzo[d]isothiazole 1,1-dioxide

### Step 1: cis-methyl-3-((3,3-dimethyl-2-oxobutyl) carbamoyl) cyclopentane-1-carboxylate

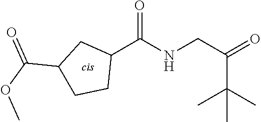 [SMILES: COC(=O)C1CCC(C(=O)NCC(=O)C(C)(C)C)C1]

To a stirred solution of cis-3-((3,3-dimethyl-2-oxobutyl) carbamoyl) cyclopentane-1-carboxylic acid (20 g, 7.839 mmol) in MeOH (50 mL) was added H₂SO₄ (15.2 g, 11.7578 mmol, 1 equiv) dropwise at room temperature. The resulting mixture was stirred for 16 h at room temperature under nitrogen atmosphere. The reaction was quenched by the addition of sat. NH₄Cl (aq.) (100 mL) at 0° C. The aqueous layer was extracted with EtOAc (3×150 mL). The residue was purified by silica gel column chromatography, eluted with DCM/MeOH (10:1) to afford the product (20 g, 98.8%). LC-MS (ESI): m/z [M+H]⁺=270.2.

### Step 2: cis-methyl-3-(5-(tert-butyl)thiazol-2-yl)cyclopentane-1-carboxylate

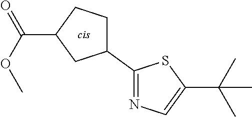 [SMILES: COC(=O)C1CCC(c2ncc(C(C)(C)C)s2)C1]

To a stirred mixture of cis-methyl-3-((3,3-dimethyl-2-oxobutyl) carbamoyl)cyclopentane-1-carboxylate in toluene (500 mL) was added Lawessons reagent (7.1 mg, 0.572 mmol, 1.5 equiv) in portions at room temperature. The resulting mixture was stirred for 16 h at 100° C. under nitrogen atmosphere. The reaction was quenched by the addition of sat. NH₄Cl (aq.) (100 mL) at 0° C. The aqueous layer was extracted with EtOAc (3×150 mL). The residue was purified by silica gel column chromatography, eluted with DCM/MeOH (10:1) to afford the crude product, which was further purified by reversed-phase flash chromatography with the following conditions (20 g, 0.3717 mmol, 97.2% yield): Column: XBridge Shield RP18 OBD Column 19*250 mm, 5 μm; Mobile Phase A: Water (10 mmol/L NH₄HCO₃+0.05% NH₃H₂O), Mobile Phase B: ACN; Flow rate: 20 mL/min mL/min; Gradient: 5% B to 30% B in 9 min; Wave Length: 254 nm/220 nm nm; RT1 (min): 8.9. LC-MS (ESI): m/z [M+H]⁺=268.39.

### Step 3: 3-(3-(5-(tert-butyl)thiazol-2-yl)cyclopentyl)-3-oxopropanenitrile

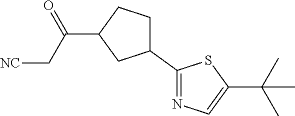 [SMILES: CC(C)(C)c1cnc(C2CCC(C(=O)CC#N)C2)s1]

To a stirred mixture of CH₃CN (5.68 g, 1.4972 mmol, 2.0 equiv) in THF (150 mL) was added n-BuLi (7.50 g, 1.4972 mmol, 1.5 equiv) dropwise at −78° C. The resulting mixture was stirred for 1 h at −78° C. under nitrogen atmosphere. To the above mixture was added cis-methyl-3-(5-(tert-butyl)thiazol-2-yl)cyclopentane-1-carboxylate (20 g, 0.7487 mmol, 1.0 equiv) in THF (150 mL) at −78° C. The resulting mixture was stirred for 1 h at −78° C. under nitrogen atmosphere. Then The reaction was stirred for 1 h at room temperature. The reaction was quenched by the addition of sat. NH₄Cl (aq.) (100 mL) at 0° C. The aqueous layer was extracted with EtOAc (3×150 mL). The residue was purified by silica gel column chromatography, eluted with DCM/MeOH (10:1) to afford the crude product, which was further purified by reversed-phase flash chromatography with the following conditions (15 g, 0.44 mmol, 81.0% yield): Column: XBridge Shield RP18 OBD Column 19*250 mm, 5 μm; Mobile Phase A: Water (10 mmol/L NH₄HCO₃+0.05% NH₃H₂O), Mobile Phase B: ACN; Flow rate: 20 mL/min mL/min; Gradient: 5% B to 30% B in 9 min; Wave Length: 254 nm/220 nm nm; RT1 (min): 8.9. LC-MS (ESI): m/z [M+H]⁺=277.40.

### Step 4: 5-(3-(5-(tert-butyl)thiazol-2-yl)cyclopentyl)-1H-pyrazol-3-amine

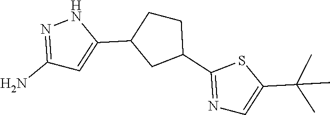 [SMILES: CC(C)(C)c1cnc(C2CCC(c3cc(N)n[nH]3)C2)s1]

To a stirred solution of 3-(3-(5-(tert-butyl)thiazol-2-yl)cyclopentyl)-3-oxopropanenitrile (10.0 g, 0.724 mmol, 1.0 equiv, 100%) and NH₂NH₂ H₂0 (2.5 g, 0796 mmol, 1.1 equiv) in EtOH (250 mL) was added PTSA (0.90 g, 0.071 mmol, 0.1 equiv, 100%) in portions at room temperature. The resulting mixture was stirred for 1 h at 90° C. under nitrogen atmosphere. The resulting mixture was concentrated under reduced pressure, and the residue was purified by reversed-phase flash chromatography with the following conditions: Column: Xselect CSH C18 OBD Column, 30*150 mm, 5 μm; Mobile Phase A: Water (0.1% FA), Mobile Phase B: ACN; Flow rate: 60 mL/min mL/min; Gradient: 10% B to 40% B in 10 min; Wave Length: 254 nm nm; RT1 (min): 9. This resulted in 1306.6 mg, 0.814 mmol, 91.9% yield. ¹H NMR (400 MHz, DMSO-d₆) δ 10.37 (s, 1H), 9.85-9.75 (m, 1H), 9.27-9.17 (m, 1H), 8.83 (s, 1H), 8.11-8.01 (m, 1H), 7.92 (s, 1H), 7.54-7.44 (m, 1H), 7.35-7.25 (m, 1H), 7.18-7.08 (m, 1H), 2.99-2.89 (m, 2H), 2.71-2.61 (m, 2H). LC-MS (ESI): m/z [M+H]⁺=291.43.

### Step 5: 5-((5-(3-(5-(tert-butyl)thiazol-2-yl)cyclopentyl)-1H-pyrazol-3-yl)amino)-4-fluoro-2,3-dihydrobenzo[d]isothiazole 1,1-dioxide

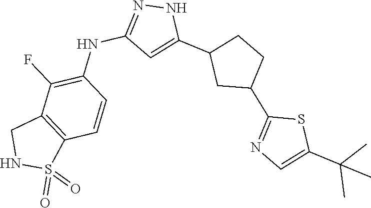 [SMILES: CC(C)(C)c1cnc(C2CCC(c3cc(Nc4ccc5c(c4F)CNS5(=O)=O)n[nH]3)C2)s1]

To a mixture of 5-bromo-4-fluoro-2,3-dihydrobenzo[d]isothiazole 1,1-dioxide (26 mg, 0.1 mmol) and 5-(3-(5-(tert-butyl)thiazol-2-yl)cyclopentyl)-1H-pyrazol-3-amine (29 mg, 0.1 mmol) in t-BuOH (3 mL) was added BrettPhos Pd G3 (9 mg, 0.01 mmol) and K₂CO₃ (42 mg, 0.3 mmol). The reaction mixture was stirred at 110° C. under nitrogen atmosphere for 4 h. The mixture was concentrated under reduced pressure. The residue was purified by prep HPLC (Waters SunFire C18: RD-CO-058 column, eluting with 34%-49% of water (containing 0.1% FA) in acetonitrile to afford the product (12.09 mg, 25%). ¹H NMR (500 MHz, DMSO-d₆) δ 12.00 (s, 1H), 8.74 (d, J=2.1 Hz, 1H), 8.27-8.19 (m, 1H), 7.70 (s, 1H), 7.48 (d, J=8.6 Hz, 1H), 7.39 (s, 1H), 5.87 (s, 1H), 4.41 (s, 2H), 3.66-3.60 (m, 1H), 3.37-3.27 (m, 1H), 2.33-2.06 (m, 4H), 1.97-1.70 (m, 2H), 1.34 (s, 9H). LC-MS (ESI): m/z [M+H]⁺=476.3.

### Example 96: cis-5-((5-(3-(5-(tert-butyl)-1,3,4-oxadiazol-2-yl)cyclopentyl)-1H-pyrazol-3-yl)amino)-4-fluoro-2,3-dihydrobenzo[d]isothiazole 1,1-dioxide

### Step 1: cis-3-(2-pivaloylhydrazine-1-carbonyl)cyclopentane-1-carboxylic acid

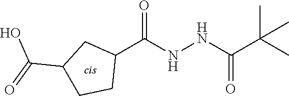 [SMILES: CC(C)(C)C(=O)NNC(=O)C1CCC(C(=O)O)C1]

Into a 500-mL round-bottom flask was added cis-3-oxa-bicyclo[3.2.1]octane-2,4-dione (15.0 g, 107.42 mmol), pivalohydrazide (24.82 g, 214.29 mmol), TEA (21.64 g, 214.29 mmol) and THF (300 mL) at room temperature. The resulting mixture was stirred 2 h at room temperature. The resulting mixture was concentrated under reduced pressure to afford cis-3-(2-pivaloylhydrazine-1-carbonyl)cyclopentane-1-carboxylic acid (23.0 g, 83.85%). LC-MS (ESI): m/z [M+H]⁺=257.2.

### Step 2: cis-methyl 3-(2-pivaloylhydrazine-1-carbonyl)cyclopentane-1-carboxylate

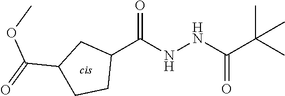 [SMILES: COC(=O)C1CCC(C(=O)NNC(=O)C(C)(C)C)C1]

Into a 250-mL round-bottom flask was added cis-3-(2-pivaloylhydrazine-1-carbonyl)cyclopentane-1-carboxylic acid (23.0 g, 89.84 mmol), H₂SO₄ (8.8 g, 89.84 mmol) and MeOH (100 mL) at room temperature. The resulting mixture was stirred overnight at room temperature. The mixture was neutralized to PH 7 with Na₂CO₃(aq.) aqueous solution and extracted with 300 mL of EtOAc. The combined organic layers were washed with brine (2×300 mL), dried over anhydrous Na₂SO₄. After filtration, the filtrate was concentrated under reduced pressure. The residue was purified by silica gel column chromatography, eluted with PE/EtOAc (100:0~50:50) to afford the product (15.1 g, 62.25%). LC-MS (ESI): m/z [M+H]⁺=271.2.

### Step 3: cis-methyl 3-(5-(tert-butyl)-1,3,4-oxadiazol-2-yl)cyclopentane-1-carboxylate

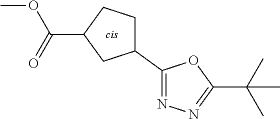 [SMILES: COC(=O)C1CCC(c2nnc(C(C)(C)C)o2)C1]

Into a 500-mL round-bottom flask was added cis-methyl 3-(2-pivaloylhydrazine-1-carbonyl)cyclopentane-1-carboxylate (15.0 g, 55.56 mmol), DIEA (21.50 g, 166.67 mmol), TsCl (31.83 g, 166.67 mmol) and CH₃CN (150 mL) at room temperature. The resulting mixture was stirred 2 h at 70° C. The resulting mixture was extracted with EtOAc (400 mL). The combined organic layers were washed with brine (2×300 mL), dried over anhydrous Na₂SO₄. After filtration, the filtrate was concentrated under reduced pressure. The residue was purified by silica gel column chromatography, eluted with PE/EtOAc (100:0~30:70) to afford cis-methyl 3-(5-(tert-butyl)-1,3,4-oxadiazol-2-yl)cyclopentane-1-carboxylate (6.0 g, 42.86%). LC-MS (ESI): m/z [M+H]⁺=253.2.

### Step 4: 3-(3-(5-(tert-butyl)-1,3,4-oxadiazol-2-yl)cyclopentyl)-3-oxopropanenitrile

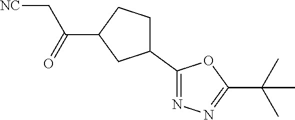 [SMILES: CC(C)(C)c1nnc(C2CCC(C(=O)CC#N)C2)o1]

Into CH₃CN (1.85 g, 45.24 mmol) was added n-BuLi (18.10 mL, 45.24 mmol) dropwise at −78° C. under nitrogen atmosphere. The resulting mixture was stirred for 30 min at −78° C. A solution of cis-methyl 3-(5-(tert-butyl)-1,3,4-oxadiazol-2-yl)cyclopentane-1-carboxylate (5.70 g, 22.62 mmol) in THF 20 mL was added dropwise at −78° C. The resulting mixture was stirred for 2 h at −78° C. The reaction was quenched with sat NH₄Cl (aq.) at 0° C. The resulting mixture was extracted with EtOAc (400 mL). The combined organic layers were washed with brine (2×200 mL), dried over anhydrous Na₂SO₄. After filtration, the filtrate was concentrated under reduced pressure. The residue was purified by silica gel column chromatography, eluted with PE/EA (100:0-70:30) to afford the product (5.0 g, 84.69%). LC-MS (ESI): m/z [M+H]⁺=262.2.

### Step 5: 5-(3-(5-(tert-butyl)-1,3,4-oxadiazol-2-yl)cyclopentyl)-1H-pyrazol-3-amine

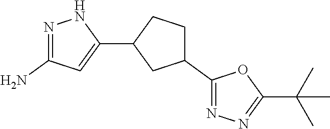 [SMILES: CC(C)(C)c1nnc(C2CCC(c3cc(N)n[nH]3)C2)o1]

To a solution of 3-(3-(5-(tert-butyl)-1,3,4-oxadiazol-2-yl)cyclopentyl)-3-oxopropanenitrile (4.0 g, 15.33 mmol) in EtOH (60 mL) was added NH₂NH₂·H₂O (0.84 g, 16.86 mmol) and PTSA (160.0 mg) at room temperature. The resulting mixture was stirred 1 h at 90° C. The resulting mixture was concentrated under reduced pressure and the residue was purified by silica gel column chromatography, eluted with DCM/MeOH (100:0~95:5) to afford the product (2.016 g, 47.83%). ¹H NMR (500 MHz, DMSO-d₆) δ 11.06 (s, 1H), 5.22 (d, J=17.6 Hz, 1H), 4.52 (s, 2H), 3.16-3.02 (m, 1H), 2.41 (dt, J=12.5, 1H), 2.25-1.80 (m, 5H), 1.78-1.64 (m, 1H), 1.34 (d, J=2.0 Hz, 9H). LC-MS (ESI): m/z [M+H]⁺=276.4.

### Step 6: cis-5-((5-(3-(5-(tert-butyl)-1,3,4-oxadiazol-2-yl)cyclopentyl)-1H-pyrazol-3-yl)amino)-4-fluoro-2,3-dihydrobenzo[d]isothiazole 1,1-dioxide

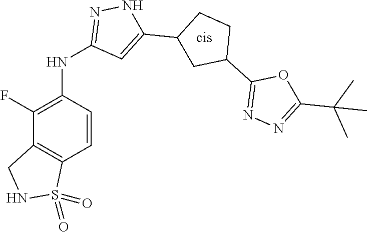 [SMILES: CC(C)(C)c1nnc(C2CCC(c3cc(Nc4ccc5c(c4F)CNS5(=O)=O)n[nH]3)C2)o1]

The titled compound was synthesized in the procedures similar to Example 6. ¹H NMR (500 MHz, DMSO-d₆) δ 11.69 (s, 1H), 8.04 (t, J=8.8 Hz, 1H), 7.92 (s, 1H), 6.85 (t, J=6.1 Hz, 1H), 6.68 (d, J=8.7 Hz, 1H), 5.69 (s, 1H), 5.00 (s, 1H), 4.72 (s, 2H), 4.33 (s, 1H), 3.03 (t, J=8.5 Hz, 1H), 2.99 (s, 3H), 2.92 (d, J=6.2 Hz, 2H), 2.44 (dd, J=14.0, 7.3 Hz, 1H), 2.06-1.97 (m, 1H), 1.92 (d, J=21.6 Hz, 1H), 1.72 (t, J=10.3 Hz, 2H), 1.60 (s, 1H), 1.02 (s, 6H). LC-MS (ESI): m/z [M+H]⁺=459.2.

### Example 97: trans-5-((5-(3-(5-(tert-butyl)-1,3,4-oxadiazol-2-yl)cyclopentyl)-1H-pyrazol-3-yl)amino)-4-fluoro-2,3-dihydrobenzo[d]isothiazole 1,1-dioxide

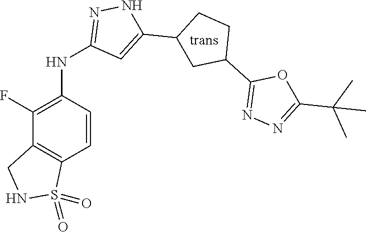 [SMILES: CC(C)(C)c1nnc(C2CCC(c3cc(Nc4ccc5c(c4F)CNS5(=O)=O)n[nH]3)C2)o1]

The titled compound was synthesized in the procedures similar to Example 96. ¹H NMR (500 MHz, DMSO-d₆) δ 12.05 (s, 1H), 8.74 (s, 1H), 8.26 (t, J=8.0 Hz, 1H), 7.69 (s, 1H), 7.48 (d, J=8.6 Hz, 1H), 5.88 (s, 1H), 4.41 (d, J=5.0 Hz, 2H), 3.58 (p, J=7.7 Hz, 1H), 3.31-3.22 (m, 1H), 2.33 (dt, J=13.3, 7.3 Hz, 1H), 2.27-2.14 (m, 1H), 2.14-2.06 (m, 1H), 1.99-1.91 (m, 1H), 1.79-1.71 (m, 1H), 1.35 (s, 6H). LC-MS (ESI): m/z [M+H]⁺=459.2.

### Example 98: 5-((5-(3-(1-(tert-butyl)-1H-pyrazol-3-yl)cyclopentyl)-1H-pyrazol-3-yl)amino)-4-fluoro-2,3-dihydrobenzo[d]isothiazole 1,1-dioxide

### Step 1: benzyl (3-(3-(4,4,5,5-tetramethyl-1,3,2-dioxaborolan-2-yl)cyclopent-2-en-1-yl)-1H-pyrazol-5-yl)carbamate

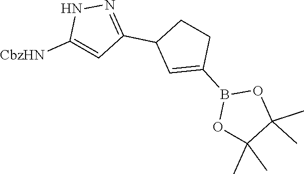 [SMILES: CC1(C)OB(C2=CC(c3cc(NC(=O)OCc4ccccc4)[nH]n3)CC2)OC1(C)C]

To a stirring solution of benzyl (1-(tert-butyl)-3-(3-(4,4,5,5-tetramethyl-1,3,2-dioxaborolan-2-yl)cyclopent-2-en-1-yl)-1H-pyrazol-5-yl)carbamate (lg, 0.086 mmol) was added FA (10 mL), The solution was stirred for 10 h at 75° C. The mixture was concentrated under reduced pressure. The residue was purified by silica gel column chromatography, eluting with PE/EA (1:1) to afford the product (500 mg, 56.8%). LC-MS (ESI): m/z [M+H]⁺=410.3.

### Step 2: benzyl (3-(3-(1-(tert-butyl)-1H-pyrazol-3-yl)cyclopent-2-en-1-yl)-1H-pyrazol-5-yl)carbamate

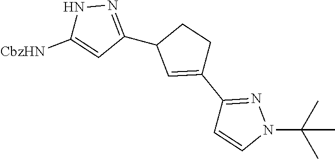 [SMILES: CC(C)(C)n1ccc(C2=CC(c3cc(NC(=O)OCc4ccccc4)[nH]n3)CC2)n1]

To a solution of benzyl (3-(3-(4,4,5,5-tetramethyl-1,3,2-dioxaborolan-2-yl)cyclopent-2-en-1-yl)-1H-pyrazol-5-yl)carbamate (500 mg, 1.22 mmol) and 3-bromo-1-(tert-butyl)-1H-pyrazole (369.7 mg, 1.83 mmol) in dioxane/H₂O (10:2 mL) was added Pd(dppf)Cl₂ (110.5 mg, 0.122 mmol), and K₃PO₄ (775.9 mg, 3.66 mmol). The reaction mixture was stirred for 6 hours at 100° C. under nitrogen. The resulting mixture was cooled to room temperature, concentrated under reduce pressure, purified by silica gel column chromatography, eluting with PE/EA (1:4) to afford the product (400 mg, 80.8%). LC-MS (ESI): m/z [M+H]⁺=406.3.

### Step 3: 5-(3-(1-(tert-butyl)-1H-pyrazol-3-yl)cyclopentyl)-1H-pyrazol-3-amine

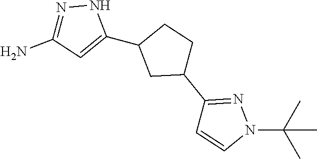 [SMILES: CC(C)(C)n1ccc(C2CCC(c3cc(N)n[nH]3)C2)n1]

benzyl (3-(3-(1-(tert-butyl)-1H-pyrazol-3-yl)cyclopent-2-en-1-yl)-1H-pyrazol-5-yl)carbamate (500 mg, 1.23 mmol) was dissolved in MeOH (10 mL), Pd/C (10% on carbon, 200 mg) was added, the reaction mixture was stirred for 2 hours at room temperature under 1 atm H₂. The resulting mixture was filtered, the filtrated was concentrated, dried over in vacuum, to afford the product (200 mg, 58.6%). LC-MS (ESI): m/z [M+H]⁺=274.3.

### Step 3: 5-((5-(3-(1-(tert-butyl)-1H-pyrazol-3-yl)cyclopentyl)-1H-pyrazol-3-yl)amino)-4-fluoro-2,3-dihydrobenzo[d]isothiazole 1,1-dioxide

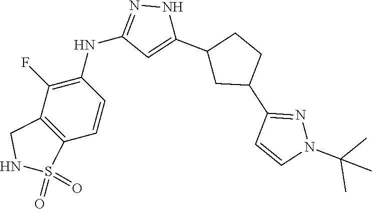 [SMILES: CC(C)(C)n1ccc(C2CCC(c3cc(Nc4ccc5c(c4F)CNS5(=O)=O)n[nH]3)C2)n1]

To a solution of 5-(3-(1-(tert-butyl)-1H-pyrazol-3-yl)cyclopentyl)-1H-pyrazol-3-amine (50 mg, 0.183 mmol) and 5-bromo-4-fluoro-2,3-dihydrobenzo[d]isothiazole 1,1-dioxide (48.5 mg, 0.183 mmol), Brettphos G3 Pd (16.3 mg, 0.018 mmol), and K₂CO₃ (75.8 mg, 0.549 mmol) in t-BuOH (10 mL) was stirred at 110° C. for 16 h under N₂ atmosphere. The mixture was concentrated under reduced pressure. The residue was purified by silica gel column chromatography, eluting with EA to afford the product 50 mg. The residue 50 mg was purified by prep HPLC (Waters XSelect C18: RD-CO-094 column, eluting with a gradient of acetonitrile/water containing 0.1% FA, 20%-40%) to afford the product (22.6 mg, 26.9%). 1H NMR (500 MHz, DMSO-d₆) δ=612.02 (s, 1H), 8.71 (d, J=2.3 Hz, 1H), 8.32-8.21 (m, 1H), 7.78-7.55 (m, 2H), 7.47 (d, J=8.6 Hz, 1H), 6.08-6.05 (m, 1H), 5.85 (d, J=4.0 Hz, 1H), 4.41 (d, J=5.0 Hz, 2H), 3.28-3.23 (m, 1H), 3.21-3.16 (m, 1H), 2.42-2.34 (m, 1H), 2.20-1.95 (m, 3H), 1.84-1.66 (m, 3H), 1.48 (s, 9H). LC-MS (ESI): m/z [M+H]⁺=459.2.

### Example 99: 5-((5-(3-(1-(tert-butyl)-1H-pyrazol-4-yl)cyclopentyl)-1H-pyrazol-3-yl)amino)-4-fluoro-2,3-dihydrobenzo[d]isothiazole 1-dioxide

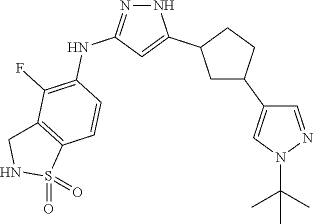 [SMILES: CC(C)(C)n1cc(C2CCC(c3cc(Nc4ccc5c(c4F)CNS5(=O)=O)n[nH]3)C2)cn1]

The titled compound was synthesized in the procedures similar to Example 98. ¹H NMR (500 MHz, DMSO-d₆) δ=11.99 (s, 1H), 8.71 (s, 1H), 8.25 (s, OH), 7.69 (t, J=5.1 Hz, OH), 7.61 (d, J=8.0 Hz, 1H), 7.47 (d, J=8.6 Hz, 1H), 7.31 (s, 1H), 5.84 (s, 1H), 4.40 (d, J=5.0 Hz, 2H), 3.28-2.99 (m, 1H), 2.42-2.34 (m, OH), 2.21-1.93 (m, 1H), 1.82-1.56 (m, 1H), 1.48 (d, J=2.2 Hz, 4H). LC-MS (ESI): m/z [M+H]⁺=459.2.

### Example 100: 5-((5-(3-(3-(tert-butyl)-1H-pyrazol-5-yl)cyclopentyl)-1H-pyrazol-3-yl)amino)-4-fluoro-2,3-dihydrobenzo[d]isothiazole 1,1-dioxide

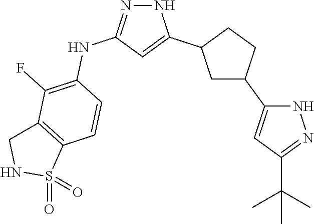 [SMILES: CC(C)(C)c1cc(C2CCC(c3cc(Nc4ccc5c(c4F)CNS5(=O)=O)n[nH]3)C2)[nH]n1]

The titled compound was synthesized in the procedures similar to Example 98. ¹H NMR (500 MHz, DMSO-d₆) δ 12.11 (brs, 1H), 12.03 (s, 1H), 8.72 (d, J=2.7 Hz, 2H), 8.26 (s, 1H), 7.69 (t, J=5.2 Hz, 1H), 7.48 (d, J=8.6 Hz, 1H), 5.85 (d, J=10.0 Hz, 2H), 4.40 (d, J=5.2 Hz, 2H), 3.19-3.13 (m, 1H), 2.41-2.33 (m, 2H), 2.17-2.08 (m, 2H), 1.83-1.64 (m, 3H), 1.23 (s, 9H). LC-MS (ESI): m/z [M+H]⁺=459.2.

### Example 101: 4-fluoro-5-((5-(3-(5-(2-hydroxypropan-2-yl)-1H-pyrazol-3-yl)cyclopentyl)-1H-pyrazol-3-yl)amino)-2,3-dihydrobenzo[d]isothiazole 1,1-dioxide

### Step 1: methyl 3-(3-(5-(((benzyloxy)carbonyl)amino)-1-(tert-butyl)-1H-pyrazol-3-yl)cyclopent-1-en-1-yl)-1H-pyrazole-5-carboxylate

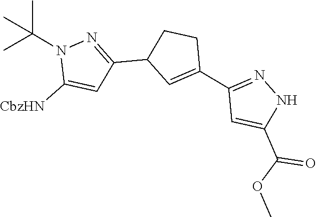 [SMILES: COC(=O)c1cc(C2=CC(c3cc(NC(=O)OCc4ccccc4)n(C(C)(C)C)n3)CC2)n[nH]1]

To a mixture of benzyl (1-(tert-butyl)-3-(3-(4,4,5,5-tetramethyl-1,3,2-dioxaborolan-2-yl)cyclopent-2-en-1-yl)-1H-pyrazol-5-yl)carbamate (930 mg, 2 mmol) and methyl 3-bromo-1H-pyrazole-5-carboxylate (410 mg, 2 mmol) in dioxane (20 mL) was added Pd(dppf)Cl₂ (146 mg, 0.2 mmol), K₂CO₃ (828 mg, 6 mmol) and H₂O (2 mL). The reaction mixture was stirred at 80° C. under nitrogen atmosphere for 4 h. The mixture was concentrated under reduced pressure and purified by silica gel column chromatography, eluting with PE/EA (4:1) to afford the product (740 mg, 80%). LC-MS (ESI): m/z [M+H]⁺=464.3

### Step 2: methyl 3-(3-(5-(((benzyloxy)carbonyl)amino)-1H-pyrazol-3-yl)cyclopent-1-en-1-yl)-1H-pyrazole-5-carboxylate

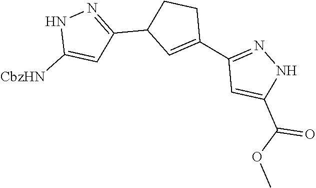 [SMILES: COC(=O)c1cc(C2=CC(c3cc(NC(=O)OCc4ccccc4)[nH]n3)CC2)n[nH]1]

A mixture of methyl 3-(3-(5-(((benzyloxy)carbonyl)amino)-1-(tert-butyl)-1H-pyrazol-3-yl)cyclopent-1-en-1-yl)-1H-pyrazole-5-carboxylate (740 mg, 1.6 mmol) in Formic Acid (7 mL) was stirred at 60° C. for 12 h. The mixture was concentrated under reduced pressure and purified by silica gel column chromatography, eluting with PE/EA (2:1) to afford the product (407 mg, 62%). LC-MS (ESI): m/z [M+H]⁺=408.3

### Step 3: benzyl (3-(3-(5-(2-hydroxypropan-2-yl)-1H-pyrazol-3-yl)cyclopent-2-en-1-yl)-1H-pyrazol-5-yl)carbamate

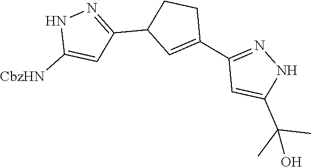 [SMILES: CC(C)(O)c1cc(C2=CC(c3cc(NC(=O)OCc4ccccc4)[nH]n3)CC2)n[nH]1]

To a mixture of methyl 3-(3-(5-(((benzyloxy)carbonyl)amino)-1H-pyrazol-3-yl)cyclopent-1-en-1-yl)-1H-pyrazole-5-carboxylate (407 mg, 1 mmol) in THF (10 mL) was added CH₃MgBr (5 mL, 1 N) at 0° C. slowly. The reaction mixture was stirred at 0° C. under nitrogen atmosphere for 1 h. The mixture was quenched by water and extracted with DCM, the organic phase was concentrated under reduced pressure and purified by silica gel column chromatography, eluting with DCM/MeOH (20:1) to afford the product (244 mg, 60%). LC-MS (ESI): m/z [M+H]⁺=408.3

### Step 4: 2-(3-(3-(5-amino-1H-pyrazol-3-yl)cyclopentyl)-1H-pyrazol-5-yl)propan-2-ol

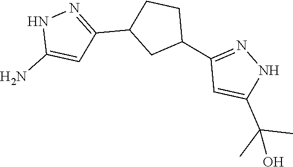 [SMILES: CC(C)(O)c1cc(C2CCC(c3cc(N)[nH]n3)C2)n[nH]1]

To a mixture of benzyl (3-(3-(5-(2-hydroxypropan-2-yl)-1H-pyrazol-3-yl)cyclopent-2-en-1-yl)-1H-pyrazol-5-yl)carbamate (244 mg, 0.6 mmol) and H₂O (1 mL) in THF (4 mL) was added Pd/C (120 mg, 0.5 M). The reaction was stirred at RT under H₂ for 12 h. The mixture was filtered through silica pad, the filtrate was concentrated under reduced pressure and purified by silica gel column chromatography, eluting with DCM/MeOH (10:1) to afford the product (130 mg, 79%). LC-MS (ESI): m/z [M+H]⁺=276.2

### Step 5: 4-fluoro-5-((5-(3-(5-(2-hydroxypropan-2-yl)-1H-pyrazol-3-yl)cyclopentyl)-1H-pyrazol-3-yl)amino)-2,3-dihydrobenzo[d]isothiazole 1,1-dioxide

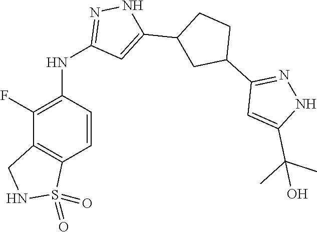 [SMILES: CC(C)(O)c1cc(C2CCC(c3cc(Nc4ccc5c(c4F)CNS5(=O)=O)n[nH]3)C2)n[nH]1]

To a mixture of 5-bromo-4-fluoro-2,3-dihydrobenzo[d]isothiazole 1,1-dioxide (26 mg, 0.1 mmol) and 2-(3-(3-(5-amino-1H-pyrazol-3-yl)cyclopentyl)-1H-pyrazol-5-yl)propan-2-ol (27 mg, 0.1 mmol) in t-BuOH (3 mL) was added BrettPhos Pd G3 (9 mg, 0.01 mmol) and K₂CO₃ (42 mg, 0.3 mmol). The reaction mixture was stirred at 110° C. under nitrogen atmosphere for 4 h. The mixture was concentrated under reduced pressure. The residue was purified by prep HPLC (Waters SunFire C18: RD-CO-058 column, eluting with 34%-49% of water (containing 0.1% FA) in acetonitrile to afford the product (10.09 mg, 10%). ¹H NMR (500 MHz, DMSO) δ 12.28-11.92 (m, 2H), 8.72 (s, 1H), 8.26 (s, 1H), 7.69 (s, 1H), 7.48 (d, J=8.6 Hz, 1H), 5.96 (s, 1H), 5.84 (s, 1H), 4.93 (s, 1H), 4.41 (d, J=3.5 Hz, 2H), 3.28-3.11 (m, 2H), 2.43-2.34 (m, 1H), 2.17-1.69 (m, 5H), 1.40 (s, 6H). LC-MS (ESI): m/z [M+H]⁺=461.3

### Example 102: 4-fluoro-5-((5-(3-(1-(2-hydroxy-2-methylpropyl)-1H-pyrazol-4-yl)cyclopentyl)-1H-pyrazol-3-yl)amino)-2,3-dihydrobenzo[d]isothiazole 1,1-dioxide

### Step 1:1-(4-bromo-1H-pyrazol-1-yl)-2-methylpropan-2-ol

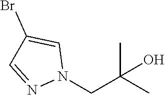

To a solution of 4-bromo-1H-pyrazole (5 g, 35.1 mmol) in MeCN (200 mL) was added Cs₂CO₃ (17 g, 53 mmol) follow with a solution of 2,2-dimethyloxirane (3.5 mL, 371 mmol) in MeCN (10 mL) dropwise to the solution in an ice bath. The mixture was stirred in a 80° C. for 16 hours. Cool the reaction mixture to room temperature and filter the mixture through Celite. The filtrate was concentrated and the residue was purified by flash column chromatography, eluting with 0-20% EtOAc/heptane to obtain the product (6.58 g, 86%). LC-MS (ESI): m/z [M+H]⁺=219.2.

### Step 2: 4-fluoro-5-((5-(3-(1-(2-hydroxy-2-methylpropyl)-1H-pyrazol-4-yl)cyclopentyl)-1H-pyrazol-3-yl)amino)-2,3-dihydrobenzo[d]isothiazole 1,1-dioxide

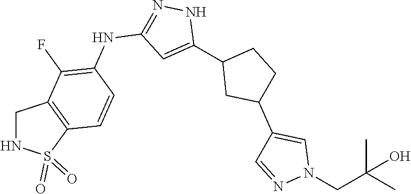 [SMILES: CC(C)(O)Cn1cc(C2CCC(c3cc(Nc4ccc5c(c4F)CNS5(=O)=O)n[nH]3)C2)cn1]

The titled compound was synthesized in the procedures similar to Example 98. 1H NMR (500 MHz, DMSO-d₆) δ=12.00 (s, 1H), 8.71 (s, 1H), 8.26 (s, 1H), 7.69 (s, 1H), 7.47 (d, J=7.8 Hz, 2H), 7.30 (s, 1H), 5.83 (s, 1H), 4.64 (s, 1H), 4.40 (d, J=4.8 Hz, 2H), 3.94 (s, 2H), 3.22-3.12 (m, 1H), 3.10-3.00 (m, 1H), 2.38 (dd, J=12.1, 6.4 Hz, 1H), 2.10 (s, 3H), 1.83-1.71 (m, 1H), 1.70-1.52 (m, 2H), 1.03 (s, 6H). LC-MS (ESI): m/z [M+H]⁺=475.2.

### Example 103: 5-((5-(3-(5-(tert-butyl)-1H-imidazol-2-yl)cyclopentyl)-1H-pyrazol-3-yl)amino)-4-fluoro-2,3-dihydrobenzo[d]isothiazole 1,1-dioxide

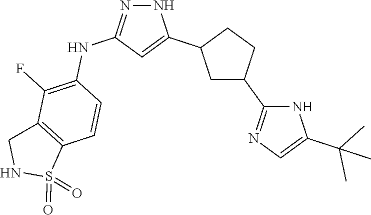 [SMILES: CC(C)(C)c1cnc(C2CCC(c3cc(Nc4ccc5c(c4F)CNS5(=O)=O)n[nH]3)C2)[nH]1]

### Step 1: 5-(tert-butyl)-1-((2-(trimethylsilyl)ethoxy)methyl)-1H-imidazole and 4-(tert-butyl)-1-((2-(trimethylsilyl)ethoxy)methyl)-1H-imidazole

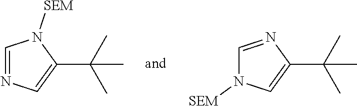 [SMILES: CC(C)(C)c1cncn1COCC[Si](C)(C)C ; CC(C)(C)c1cn(COCC[Si](C)(C)C)cn1]

To a solution of 5-(tert-butyl)-1H-imidazole (1.0 g, 8.05 mmol) in THF (15 mL) was added NaH (60% dispersion in mineral oil, 386 mg, 9.66 mmol) at 0° C., stirred for 30 minutes, SEMCl (1.41 g, 8.46 mmol) was added, the reaction mixture was stirred for 16 hours at room temperature. The resulting mixture was quenched with saturated NH₄Cl aqueous, extracted with EA, dried over Na₂SO₄, purified by column chromatography on silica gel (petroleum ether:ethyl acetate=1:1~1:9) to afford the product A (1.07 g, 52%) and product B (500 mg, 24%). LC-MS (ESI): m/z [M+H]⁺=439.1. Product A: ¹H NMR (500 MHz, DMSO-d₆) δ=7.66 (d, J=1.2, 1H), 6.93 (d, J=1.3, 1H), 5.28 (s, 2H), 3.55-3.47 (m, 2H), 1.24 (s, 9H), 0.92-0.81 (m, 2H), −0.00 (s, 9H). LC-MS (ESI): m/z [M+H]⁺=255.2. Product B: ¹H NMR (500 MHz, DMSO-d₆) δ=7.67 (s, 1H), 6.68 (s, 1H), 5.41 (s, 2H), 3.53-3.48 (m, 2H), 1.33 (s, 9H), 0.90-0.85 (m, 12H), −0.00 (s, 9H). LC-MS (ESI): m/z [M+H]⁺=255.2.

### Step 2: 2-bromo-5-(tert-butyl)-1-((2-(trimethylsilyl)ethoxy)methyl)-1H-imidazole

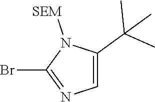 [SMILES: CC(C)(C)c1cnc(Br)n1COCC[Si](C)(C)C]

5-(tert-butyl)-1-((2-(trimethylsilyl)ethoxy)methyl)-1H-imidazole (255 mg, 1 mmol) was dissolved in THF (10 mL), cooled to −60° C. under nitrogen, n-BuLi (2.5 M in hexane, 0.44 mL, 1.1 mmol) was added dropwise, stirred for 1 hour at −60° C., CBr₄ (322 mg, 1 mmol) in THF (3 mL) was added, the reaction mixture was warmed to room temperature, stirred for 20 minutes. The resulting mixture was quenched with NH₄Cl aqueous, extracted with EA, dried over Na₂SO₄, purified by column chromatography on silica gel (petroleum ether:ethyl acetate=5:1) to afford the product (130 mg, 39%). ¹H NMR (500 MHz, DMSO-d₆) δ=7.19 (s, 1H), 5.24 (s, 2H), 3.56 (t, J=7.9, 2H), 1.22 (s, 9H), 0.87 (t, J=7.9, 2H), −0.00 (s, 9H). LC-MS (ESI): m/z [M+H]⁺=333.5.

### Step 3: 5-((5-(3-(5-(tert-butyl)-1H-imidazol-2-yl)cyclopentyl)-1H-pyrazol-3-yl)amino)-4-fluoro-2,3-dihydrobenzo[d]isothiazole 1,1-dioxide

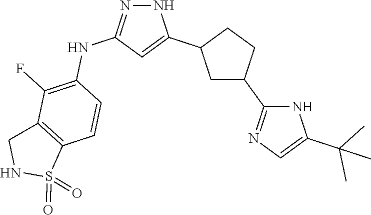 [SMILES: CC(C)(C)c1cnc(C2CCC(c3cc(Nc4ccc5c(c4F)CNS5(=O)=O)n[nH]3)C2)[nH]1]

The titled compound was synthesized in the procedures similar to Example 76. ¹H NMR (500 MHz, DMSO-d₆) δ=12.45 (s, 1H), 8.72 (s, 1H), 8.22 (t, J=7.8, 1H), 8.18 (s, 1H), 7.69 (s, 1H), 7.47 (d, J=8.6, 1H), 6.55 (s, 1H), 5.86 (s, 1H), 4.41 (s, 2H), 3.20 (m, 2H), 2.42-2.33 (m, 1H), 2.11-1.98 (m, 2H), 1.90 (m, 2H), 1.80-1.70 (m, 1H), 1.22 (s, 9H). LC-MS (ESI): m/z [M+H]⁺=459.35.

### Example 104: 5-((5-(3-(5-(tert-butyl)tetrahydrofuran-2-yl)cyclopentyl)-1H-pyrazol-3-yl)amino)-4-fluoro-2,3-dihydrobenzo[d]isothiazole 1,1-dioxide

### Step 1: 2-bromo-5-(tert-butyl)furan

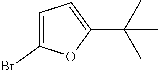

To a solution of 2-(tert-butyl)furan (1.24 g, 10 mmol) in DMF (15 mL) was added NBS (1.96 g, 11 mmol). The mixture was stirred at rt for 16 h. The mixture was stirred at rt for 16 h. The mixture was diluted with H₂O (50 mL), extracted with EA (3×50 mL). The combined organic layers were dried over Na₂SO₄, filtered and concentrated under vacuum. The residue was purified by silica gel column chromatography, eluting with EA/PE (0-30%) to afford the product (560 mg, 27.6%). LC-MS (ESI): m/z [M+H]⁺=203.

### Step 2: benzyl (1-(tert-butyl)-3-(3-(5-(tert-butyl)furan-2-yl)cyclopent-2-en-1-yl)-1H-pyrazol-5-yl)carbamate

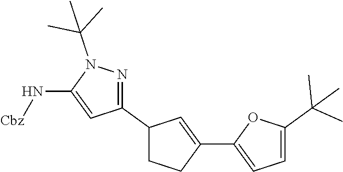 [SMILES: CC(C)(C)c1ccc(C2=CC(c3cc(NC(=O)OCc4ccccc4)n(C(C)(C)C)n3)CC2)o1]

To a mixture of 2-bromo-5-(tert-butyl)furan (110 mg, 0.54 mmol) in dioxane (10 mL) and H2O (2 mL) were added benzyl (1-(tert-butyl)-3-(3-(4,4,5,5-tetramethyl-1,3,2-dioxaborolan-2-yl)cyclopent-2-en-1-yl)-1H-pyrazol-5-yl)carbamate (278 mg, 0.6 mmol), Pd(dppf)Cl₂ (43.8 mg, 0.05 mmol), K₃PO₄ (286 mg, 1.35 mmol). The mixture was stirred at 90° C. for 16 h under N₂ atmosphere. The mixture was concentrated under vacuum. The residue was purified by silica gel column chromatography, eluting with EA/PE (0-30%) to afford the product (80 mg, 32%). LC-MS (ESI): m/z [M+H]⁺=462.3.

### Step 3: 1-(tert-butyl)-3-(3-(5-(tert-butyl)tetrahydrofuran-2-yl)cyclopentyl)-1H-pyrazol-5-amine

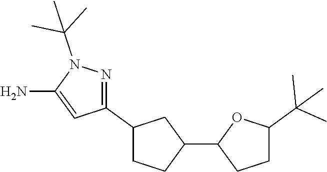 [SMILES: CC(C)(C)C1CCC(C2CCC(c3cc(N)n(C(C)(C)C)n3)C2)O1]

To a solution of benzyl (1-(tert-butyl)-3-(3-(5-(tert-butyl)furan-2-yl)cyclopent-2-en-1-yl)-1H-pyrazol-5-yl)carbamate (80 mg, 17.3 mmol) in MeOH (10 mL) was added Pd/C (10 mg). The mixture was stirred at rt for 5 h under a hydrogen atmosphere. The mixture was filtered and the filtrate was concentrated under vacuum to afford (53 mg, crude), which was used in the next step without further purification. LC-MS (ESI): m/z [M+H]⁺=334.3.

### Step 4: 5-((1-(tert-butyl)-3-(3-(5-(tert-butyl)tetrahydrofuran-2-yl)cyclopentyl)-1H-pyrazol-5-yl)amino)-4-fluoro-2-(4-methoxybenzyl)-2,3-dihydrobenzo[d]isothiazole 1,1-dioxide

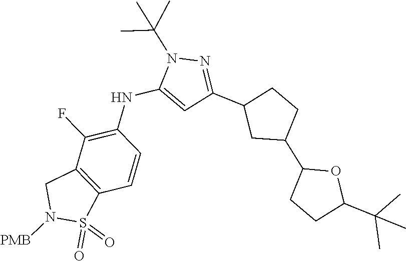

A mixture of 5-bromo-4-fluoro-2-(4-methoxybenzyl)-2,3-dihydrobenzo[d]isothiazole 1,1-dioxide (49 mg, 0.13 mmol), 1-(tert-butyl)-3-(3-(5-(tert-butyl)tetrahydrofuran-2-yl)cyclopentyl)-1H-pyrazol-5-amine (53 mg, 0.16 mmol), Pd₂dba₃ (14.6 mg, 0.016 mmol) and Xantphos (18.5 mg, 0.032 mmol) in dioxane (10 mL) was stirred at 100° C. for 16 h under N₂ atmosphere. The mixture was concentrated under vacuum. The residue was purified by silica gel column chromatography, eluting with EA/PE (0-80%) to afford the product (50 mg, 61.7%). LC-MS (ESI): m/z [M+H]⁺=639.3.

### Step 5: 5-((5-(3-(5-(tert-butyl)tetrahydrofuran-2-yl)cyclopentyl)-1H-pyrazol-3-yl)amino)-4-fluoro-2,3-dihydrobenzo[d]isothiazole 1,1-dioxide

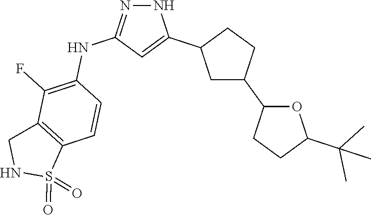 [SMILES: CC(C)(C)C1CCC(C2CCC(c3cc(Nc4ccc5c(c4F)CNS5(=O)=O)n[nH]3)C2)O1]

To a solution of 5-((1-(tert-butyl)-3-(3-(5-(tert-butyl)tetrahydrofuran-2-yl)cyclopentyl)-1H-pyrazol-5-yl)amino)-4-fluoro-2-(4-methoxybenzyl)-2,3-dihydrobenzo[d]isothiazole 1,1-dioxide (50 mg, 0.078 mmol) in DCM (10 mL) was added TfOH (0.5 mL). The mixture was stirred at rt for 2 h. The mixture was diluted with DCM (50 mL), washed with sat NaHCO₃ (3×20 mL). The combined organic layers were dried over Na₂SO₄, filtered and concentrated under vacuum. The residue was purified by Prep-HPLC to afford product (1.9 mg, 0.05%). LC-MS (ESI): m/z [M+H]⁺=463.2.

### Example 105: 4-fluoro-5-((5-(3-(2-isopropyloxazol-5-yl)cyclopentyl)-1H-pyrazol-3-yl)amino)-2,3-dihydrobenzo[d]isothiazole 1,1-dioxide

### Step 1: 2-bromo-5-iodooxazole

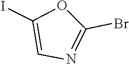

To a stirring solution of 2-bromooxazole (5 g, 34.03 mmol) in dry THF (60 mL) was dropwise added TMPMgCl·LiCl (1M solution in THF) (37.43 mL, 37.43 mmol) at −60° C. The reaction mixture was stirred at −60° C. for 1 h. Then I₂ (9.5, 37.43 mmol) in THF (20 mL) was added dropwise. The reaction mixture was slowly warmed to room temperature and subsequently quenched with satd. Na₂S₂O₈ solution (50 mL). The aqueous layer was extracted with EA (2×50 mL), and the combined organic layers were dried over Na₂SO₄. The solvent was evaporated under reduced pressure, and the remaining residue was purified by flash chromatography on silica gel column chromatography, eluting with PA/EA (0% to 20%) to afford the product (5 g, 53.9%). LC-MS (ESI): m/z [M+H]⁺=273.8.

### Step 2: benzyl (3-(3-(2-bromooxazol-5-yl)cyclopent-2-en-1-yl)-1-(tert-butyl)-1H-pyrazol-5-yl)carbamate

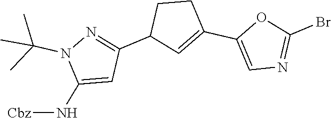 [SMILES: CC(C)(C)n1nc(C2C=C(c3cnc(Br)o3)CC2)cc1NC(=O)OCc1ccccc1]

To a solution of benzyl (1-(tert-butyl)-3-(3-(4,4,5,5-tetramethyl-1,3,2-dioxaborolan-2-yl)cyclopent-2-en-1-yl)-1H-pyrazol-5-yl)carbamate (450 mg, 0.97 mmol) and 2-bromo-5-iodooxazole (400 mg, 1.45 mmol) in dioxane (20 mL) and H₂O (2 mL) was added Pd(PPh₃)₄ (112 mg, 0.097 mmol) and Na₂CO₃ (310 mg, 2.91 mmol), the reaction mixture was stirred for 16 hours at 60° C. under nitrogen. The resulting mixture was cooled to room temperature, concentrated under reduce pressure, purified by column chromatography on silica gel (petroleum ether:ethyl acetate=3:1) to afford the product (276 mg, 59%). LC-MS (ESI): m/z [M+H]⁺=485.2.

### Step 3: benzyl (1-(tert-butyl)-3-(3-(2-(prop-1-en-2-yl)oxazol-5-yl)cyclopent-2-en-1-yl)-1H-pyrazol-5-yl)carbamate

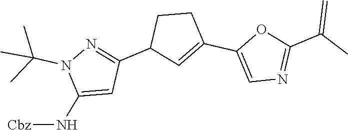 [SMILES: C=C(C)c1ncc(C2=CC(c3cc(NC(=O)OCc4ccccc4)n(C(C)(C)C)n3)CC2)o1]

To a solution of benzyl (3-(3-(2-bromooxazol-5-yl)cyclopent-2-en-1-yl)-1-(tert-butyl)-1H-pyrazol-5-yl)carbamate (100 mg, 0.207 mmol) and 4,4,5,5-tetramethyl-2-(prop-1-en-2-yl)-1,3,2-dioxaborolane (42 mg, 0.248 mmol) in dioxane (5 mL) and H₂O (0.5 mL) was added Pd(dppf)Cl₂ (15 mg, 0.02 mmol) and Na₂CO₃ (66 mg, 0.621 mmol), the reaction mixture was stirred for 2 hours at 100° C. under nitrogen. The resulting mixture was cooled to room temperature, concentrated under reduce pressure, purified by column chromatography on silica gel (petroleum ether:ethyl acetate=3:1) to afford the product (73 mg, 79%). LC-MS (ESI): m/z [M+H]⁺=447.0.

### Step 4: 1-(tert-butyl)-3-(3-(2-isopropyloxazol-5-yl)cyclopent-2-en-1-yl)-1H-pyrazol-5-amine

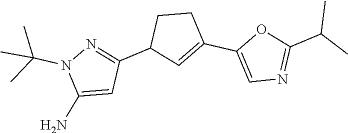 [SMILES: CC(C)c1ncc(C2=CC(c3cc(N)n(C(C)(C)C)n3)CC2)o1]

To a solution of benzyl (1-(tert-butyl)-3-(3-(2-(prop-1-en-2-yl)oxazol-5-yl)cyclopent-2-en-1-yl)-1H-pyrazol-5-yl)carbamate (73 mg, 0.164 mmol) in THF (10 mL) was added Pd/C (10% on carbon, 30 mg), the reaction mixture was stirred for 3 hours at room temperature under 1 atm hydrogen. The resulting mixture was filtered, the filtrated was concentrated under reduce pressure, dried over in vacuum, to afford the product (50 mg, 96%). LC-MS (ESI): m/z [M+H]⁺=316.6.

### Step 5: 4-fluoro-5-((5-(3-(2-isopropyloxazol-5-yl)cyclopentyl)-1H-pyrazol-3-yl)amino)-2,3-dihydrobenzo[d]isothiazole 1,1-dioxide

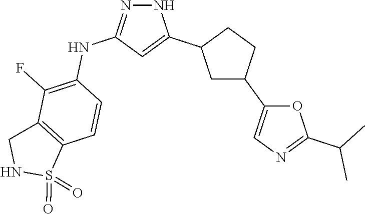 [SMILES: CC(C)c1ncc(C2CCC(c3cc(Nc4ccc5c(c4F)CNS5(=O)=O)n[nH]3)C2)o1]

The titled compound was synthesized in the procedures similar to Example 1. ¹H NMR (500 MHz, DMSO-d₆) δ=12.03 (s, 1H), 8.72 (d, J=1.8, 1H), 8.26 (t, J=7.6, 1H), 7.69 (s, 1H), 7.47 (d, J=8.6, 1H), 6.75 (d, J=0.8, 1H), 5.84 (s, 1H), 4.40 (d, J=4.3, 2H), 3.29-3.24 (m, 1H), 3.24-3.15 (m, 1H), 3.00 (m, 1H), 2.45-2.38 (m, 1H), 2.15-2.00 (m, 2H), 1.86-1.64 (m, 3H), 1.24 (d, J=6.9, 6H). LC-MS (ESI): m/z [M+H]⁺=446.27.

### Example 106: 5-((5-(3-(2-cyclopropyloxazol-5-yl)cyclopentyl)-1H-pyrazol-3-yl)amino)-4-fluoro-2,3-dihydrobenzo[d]isothiazole 1,1-dioxide

### Step 1: benzyl (1-(tert-butyl)-3-(3-(2-cyclopropyloxazol-5-yl)cyclopent-2-en-1-yl)-1H-pyrazol-5-yl)carbamate

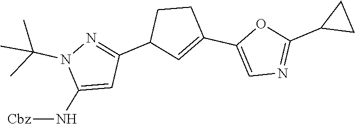 [SMILES: CC(C)(C)n1nc(C2C=C(c3cnc(C4CC4)o3)CC2)cc1NC(=O)OCc1ccccc1]

To a solution of benzyl (3-(3-(2-bromooxazol-5-yl)cyclopent-2-en-1-yl)-1-(tert-butyl)-1H-pyrazol-5-yl)carbamate (100 mg, 0.207 mmol) and cyclopropylboronic acid (89 mg, 1.035 mmol) in dioxane (5 mL) and H₂O (0.5 mL) was added Pd(dppf)Cl₂ (15 mg, 0.02 mmol) and Cs₂CO₃ (202 mg, 0.621 mmol), the reaction mixture was stirred for 16 hours at 100° C. under nitrogen. The resulting mixture was cooled to room temperature, concentrated under reduce pressure, purified by column chromatography on silica gel (petroleum ether:ethyl acetate=3:1) to afford the product (20 mg, 22%). LC-MS (ESI): m/z [M+H]⁺=447.0.

### Step 2: 5-((5-(3-(2-cyclopropyloxazol-5-yl)cyclopentyl)-1H-pyrazol-3-yl)amino)-4-fluoro-2,3-dihydrobenzo[d]isothiazole 1,1-dioxide

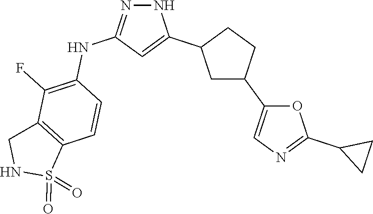 [SMILES: O=S1(=O)NCc2c1ccc(Nc1cc(C3CCC(c4cnc(C5CC5)o4)C3)[nH]n1)c2F]

The titled compound was synthesized in the procedures similar to Example 1. ¹H NMR (500 MHz, DMSO-d₆) δ=12.02 (s, 1H), 8.72 (s, 1H), 8.25 (s, 1H), 7.69 (t, J=5.0, 1H), 7.47 (d, J=8.6, 1H), 6.70 (s, 1H), 5.84 (s, 1H), 4.40 (d, J=4.9, 2H), 3.28-3.23 (m, 1H), 3.18 (m, 1H), 2.43-2.34 (m, 1H), 2.15-1.99 (m, 3H), 1.82-1.63 (m, 3H), 1.00-0.95 (m, 2H), 0.89-0.84 (m, 2H). LC-MS (ESI): m/z [M+H]⁺=444.33.

### Example 107: cis-5-((5-(3-(5-(tert-butyl)furan-2-yl)cyclopentyl)-1H-pyrazol-3-yl)amino)-4-fluoro-2,3-dihydrobenzo[d]isothiazole 1,1-dioxide

### Step 1:1-(tert-butyl)-3-(3-(5-(tert-butyl)furan-2-yl)cyclopentyl)-1H-pyrazol-5-amine

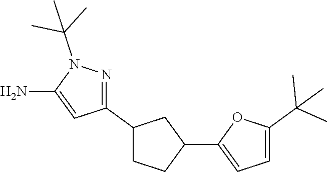 [SMILES: CC(C)(C)c1ccc(C2CCC(c3cc(N)n(C(C)(C)C)n3)C2)o1]

To a solution of benzyl (1-(tert-butyl)-3-(3-(5-(tert-butyl)furan-2-yl)cyclopent-2-en-1-yl)-1H-pyrazol-5-yl)carbamate (80 mg, 17.3 mmol) in THF/EA (5 mL/5 mL) was added Pd/C (10 mg). The mixture was stirred at rt for 1 h under a hydrogen atmosphere. The mixture was filtered and the filtrate was concentrated under vacuum to afford (62 mg, crude), which was used in the next step without further purification. LC-MS (ESI): m/z [M+H]⁺=330.3.

### Step 2: cis-5-((5-(3-(5-(tert-butyl)furan-2-yl)cyclopentyl)-1H-pyrazol-3-yl)amino)-4-fluoro-2,3-dihydrobenzo[d]isothiazole 1,1-dioxide

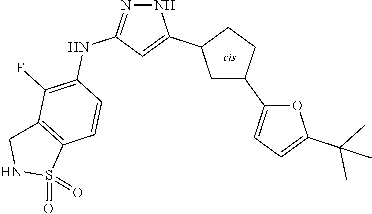 [SMILES: CC(C)(C)c1ccc(C2CCC(c3cc(Nc4ccc5c(c4F)CNS5(=O)=O)n[nH]3)C2)o1]

The titled compound was synthesized in the procedures similar to Example 104. ¹H NMR (500 MHz, DMSO-d₆) δ 12.02 (s, 1H), 8.71 (s, 1H), 8.30.-8.19 (m, 1H), 7.69 (t, J=5 Hz, 1H), 7.47 (d, J=10 Hz, 1H), 5.95 (d, J=5 Hz, 1H), 5.89 (d, J=5 Hz, 1H), 5.83 (s, 1H), 4.40 (d, J=5 Hz, 2H), 3.25-3.11 (m, 2H). 2.43-2.34 (m, 1H), 2.14-1.99 (m, 2H), 1.83-1.64 (m, 3H), 1.21 (s, 9H). LC-MS (ESI): m/z [M+H]⁺=459.2.

### Example 108: trans-5-((5-(3-(5-(tert-butyl)furan-2-yl)cyclopentyl)-1H-pyrazol-3-yl)amino)-4-fluoro-2,3-dihydrobenzo[d]isothiazole 1,1-dioxide

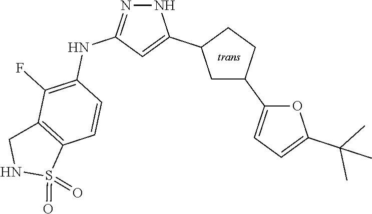 [SMILES: CC(C)(C)c1ccc(C2CCC(c3cc(Nc4ccc5c(c4F)CNS5(=O)=O)n[nH]3)C2)o1]

The titled compound was synthesized in the procedures similar to Example 107. ¹H NMR (500 MHz, DMSO-d₆) δ 12.00 (s, 1H), 8.72 (s, 1H), 8.32.-8.19 (m, 1H), 7.69 (t, J=5 Hz, 1H), 7.47 (d, J=10 Hz, 1H), 5.99 (d, J=5 Hz, 1H), 5.89 (d, J=5 Hz, 1H), 5.85 (s, 1H), 4.40 (d, J=5 Hz, 2H), 3.33-3.18 (m, 2H), 2.20-2.05 (m, 3H), 2.03-1.94 (m, 1H), 1.79-1.65 (m, 2H), 1.22 (s, 9H). LC-MS (ESI): m/z [M+H]⁺=459.2.

### Example 109: 4-fluoro-5-((5-(3-(5-isopropyloxazol-2-yl)cyclopentyl)-1H-pyrazol-3-yl)amino)-2,3-dihydrobenzo[d]isothiazole 1,1-dioxide

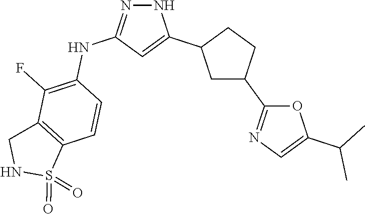 [SMILES: CC(C)c1cnc(C2CCC(c3cc(Nc4ccc5c(c4F)CNS5(=O)=O)n[nH]3)C2)o1]

The titled compound was synthesized in the procedures similar to Example 18 in a racemic form, which was separated by chiral HPLC to give:

Enantiomer 1 (Example 109a, 99.67% ee); Retention time: 4.06 min. ¹H NMR (500 MHz, DMSO-d₆) δ 12.04 (s, 1H), 8.73 (s, 1H), 8.26 (s, 1H), 7.69 (t, J=5.2 Hz, 1H), 7.48 (d, J=8.6 Hz, 1H), 6.69 (d, J=1.2 Hz, 1H), 5.85 (d, J=12.6 Hz, 1H), 4.40 (d, J=5.2 Hz, 2H), 3.24-3.13 (m, 1H), 2.98-2.89 (m, 1H), 2.48-2.41 (m, 1H), 2.18-1.85 (m, 5H), 1.78-1.69 (m, 1H), 1.19 (dd, J=6.8, 5.0 Hz, 6H). LC-MS (ESI): m/z [M+H]⁺=446.3.

Enantiomer 2 (Example 109b, 93.05% ee); Retention time: 4.20 min. ¹H NMR (500 MHz, DMSO-d₆) δ 12.04 (s, 1H), 8.73 (s, 1H), 8.26 (s, 1H), 7.69 (t, J=5.2 Hz, 1H), 7.48 (d, J=8.6 Hz, 1H), 6.69 (d, J=1.2 Hz, 1H), 5.85 (d, J=12.6 Hz, 1H), 4.40 (d, J=5.2 Hz, 2H), 3.24-3.13 (m, 1H), 2.98-2.89 (m, 1H), 2.48-2.41 (m, 1H), 2.18-1.85 (m, 5H), 1.78-1.69 (m, 1H), 1.19 (dd, J=6.8, 5.0 Hz, 6H). LC-MS (ESI): m/z [M+H]⁺=446.3.

Enantiomer 3 (Example 109c, 94.11% ee); Retention time: 4.89 min. ¹H NMR (500 MHz, DMSO-d₆) δ 12.04 (s, 1H), 8.73 (s, 1H), 8.26 (s, 1H), 7.69 (t, J=5.2 Hz, 1H), 7.48 (d, J=8.6 Hz, 1H), 6.69 (d, J=1.2 Hz, 1H), 5.85 (d, J=12.6 Hz, 1H), 4.40 (d, J=5.2 Hz, 2H), 3.24-3.13 (m, 1H), 2.98-2.89 (m, 1H), 2.48-2.41 (m, 1H), 2.18-1.85 (m, 5H), 1.78-1.69 (m, 1H), 1.19 (dd, J=6.8, 5.0 Hz, 6H). LC-MS (ESI): m/z [M+H]⁺=446.3.

Enantiomer 4 (Example 109d, 99.09% ee); Retention time: 6.12 min. ¹H NMR (500 MHz, DMSO-d₆) δ 12.04 (s, 1H), 8.73 (s, 1H), 8.26 (s, 1H), 7.69 (t, J=5.2 Hz, 1H), 7.48 (d, J=8.6 Hz, 1H), 6.69 (d, J=1.2 Hz, 1H), 5.85 (d, J=12.6 Hz, 1H), 4.40 (d, J=5.2 Hz, 2H), 3.24-3.13 (m, 1H), 2.98-2.89 (m, 1H), 2.48-2.41 (m, 1H), 2.18-1.85 (m, 5H), 1.78-1.69 (m, 1H), 1.19 (dd, J=6.8, 5.0 Hz, 6H). LC-MS (ESI): m/z [M+H]⁺=446.3.

Chiral analytical method: Column: CHIRALPAK IA 4.6*250 mm 5 μm; Mobile phase: A for MtBE (0.1% 2M NH₃ MeOH) and B for DCM:MeOH=50:50; Gradient: Mobile Phase A: Mobile Phase B=80:20 (v/v); HPLC Equipment: HPLC-Agilent; Column temperature: 35° C.

Chiral Prep-HPLC Condition: CHIRALPAK IA 20*250 mm 5 μm; Mobile phase: A for MtBE and B for MeOH:DCM=50:50; Gradient: Mobile Phase A: Mobile Phase B=80:20 (v/v); Flow Rate: 20 mL/min, Wavelength: UV 220 nm, Prep-HPLC Equipment: Prep-HPLC-Gilson; Column temperature: 25° C.

### Example 110: 4-fluoro-5-((5-(3-(5-(2-hydroxypropan-2-yl)oxazol-2-yl)cyclopentyl)-1H-pyrazol-3-yl)amino)-2,3-dihydrobenzo[d]isothiazole 1,1-dioxide

### Step 1: Benzyl (5-(3-oxocyclopentyl)-1H-pyrazol-3-yl)carbamate

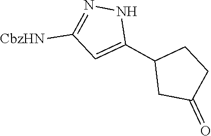 [SMILES: O=C1CCC(c2cc(NC(=O)OCc3ccccc3)n[nH]2)C1]

Into a 250-mL round-bottom flask was added Benzyl (1-(tert-butyl)-5-(3-oxocyclopentyl)-1H-pyrazol-3-yl)carbamate (10.00 g, 28.17 mmol) and formic acid (50 mL) at room temperature. The resulting mixture was stirred for 2 h at 75° C. The resulting mixture was concentrated under reduced pressure to afford Benzyl (5-(3-oxocyclopentyl)-1H-pyrazol-3-yl)carbamate (5.0 g, 59.36%). LC-MS (ESI): m/z [M+H]⁺=300.3.

### Step 2: tert-butyl 3-(((benzyloxy)carbonyl)amino)-5-(3-oxocyclopentyl)-1H-pyrazole-1-carboxylate

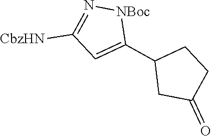 [SMILES: CC(C)(C)OC(=O)n1nc(NC(=O)OCc2ccccc2)cc1C1CCC(=O)C1]

Into a 500-mL round-bottom flask was added Benzyl (5-(3-oxocyclopentyl)-1H-pyrazol-3-yl)carbamate (6.00 g, 20.07 mmol), Boc₂O (6.56 g, 30.10 mmol), Et₃N (2.08 g, 60.20 mmol) and DCM (60 mL) at room temperature. The resulting mixture was stirred 5 h at room temperature. The resulting mixture was extracted with DCM (200 mL). The combined organic layers were washed with brine (2×200 mL), dried over anhydrous Na₂SO₄. After filtration, the filtrate was concentrated under reduced pressure. The residue was purified by silica gel column chromatography, eluted with PE/EA (100:0-90:10) to afford tert-butyl 3-(((benzyloxy)carbonyl)amino)-5-(3-oxocyclopentyl)-1H-pyrazole-1-carboxylate (7.00 g, 87.43%). LC-MS (ESI): m/z [M+H]⁺=400.2.

### Step 3: tert-butyl 3-(((benzyloxy)carbonyl)amino)-5-(3-(((trifluoromethyl)sulfonyl)oxy)cyclopent-2-en-1-yl)-1H-pyrazole-1-carboxylate

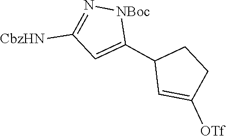 [SMILES: CC(C)(C)OC(=O)n1nc(NC(=O)OCc2ccccc2)cc1C1C=C(OS(=O)(=O)C(F)(F)F)CC1]

Into a 250-mL 3-necked round-bottom flask purged and maintained with an inert atmosphere of nitrogen, were placed tert-butyl 3-(((benzyloxy)carbonyl)amino)-5-(3-oxocyclopentyl)-1H-pyrazole-1-carboxylate (5.00 g, 12.53 mmol) in THF 100 mL, was added LiHMDS (18.80 mL, 18.80 mmol) dropwised at −78° C. under nitrogen atmosphere. The resulting mixture was stirred for 1 h at −78° C. To a solution of PhN(Tf)₂ (6.43 g, 18.80 mmol) in THF 20 mL dropwised at −78° C. The resulting mixture was stirred for 4 h at −78° C.—room temperature. The reaction was quenched with sat NH₄Cl(aq.) at 0° C. The resulting mixture was extracted with EtOAc (200 mL). The combined organic layers were washed with brine (2×200 mL), dried over anhydrous Na₂SO₄. After filtration, the filtrate was concentrated under reduced pressure. The residue was purified by silica gel column chromatography, eluted with PE/EA (100:0~95:5) to afford tert-butyl 3-(((benzyloxy)carbonyl)amino)-5-(3-(((trifluoromethyl)sulfonyl)oxy)cyclopent-2-en-1-yl)-1H-pyrazole-1-carboxylate (4.00 g, 60.11%). LC-MS (ESI): m/z [M+H]⁺=532.2.

### Step 4: tert-butyl 3-(((benzyloxy)carbonyl)amino)-5-(3-(4,4,5,5-tetramethyl-1,3,2-dioxaborolan-2-yl)cyclopent-2-en-1-yl)-1H-pyrazole-1-carboxylate

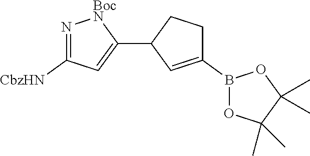 [SMILES: CC(C)(C)OC(=O)n1nc(NC(=O)OCc2ccccc2)cc1C1C=C(B2OC(C)(C)C(C)(C)O2)CC1]

Into a 250-mL round-bottom flask was added tert-butyl 3-(((benzyloxy)carbonyl)amino)-5-(3-(((trifluoromethyl)sulfonyl)oxy)cyclopent-2-en-1-yl)-1H-pyrazole-1-carboxylate (4.00 g, 7.53 mmol), B₂Pin₂ (3.84 g, 15.07 mmol), Pd(dppf)Cl₂ (607.5 mg, 0.75 mmol), KOAc (2.21 g, 22.59 mmol) and Dioxane (50 mL) at room temperature. The resulting mixture was stirred for 4 h at 80° C. under nitrogen atmosphere. The resulting mixture was concentrated under reduced pressure. The residue was purified by silica gel column chromatography, eluted with PE/EtOAc (100:0-97:3) to afford tert-butyl 3-(((benzyloxy)carbonyl)amino)-5-(3-(4,4,5,5-tetramethyl-1,3,2-dioxaborolan-2-yl)cyclopent-2-en-1-yl)-1H-pyrazole-1-carboxylate (3.4 g, 88.51%). LC-MS (ESI): m/z [M+H]⁺=510.4.

### Step 5: methyl 2-(3-(3-(((benzyloxy)carbonyl)amino)-1-(tert-butoxycarbonyl)-1H-pyrazol-5-yl)cyclopent-1-en-1-yl)oxazole-5-carboxylate

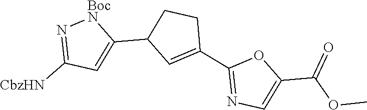 [SMILES: COC(=O)c1cnc(C2=CC(c3cc(NC(=O)OCc4ccccc4)nn3C(=O)OC(C)(C)C)CC2)o1]

Into a 100-mL round-bottom flask was added tert-butyl 3-(((benzyloxy)carbonyl)amino)-5-(3-(4,4,5,5-tetramethyl-1,3,2-dioxaborolan-2-yl)cyclopent-2-en-1-yl)-1H-pyrazole-1-carboxylate (2.40 g, 4.72 mmol), ethyl 2-bromooxazole-5-carboxylate (1.24 g, 5.66 mmol), Pd(dppf)Cl₂ (380.7 mg, 0.47 mmol), K₃PO₄ (3.0 Og, 14.16 mmol) and Dioxane/H₂O (5/1) (30 mL) at room temperature. The resulting mixture was stirred for 2 h at 90° C. under nitrogen atmosphere. The resulting mixture was concentrated under reduced pressure. The residue was purified by silica gel column chromatography, eluted with PE/EtOAc (100:0~90:10) to afford methyl 2-(3-(3-(((benzyloxy)carbonyl)amino)-1-(tert-butoxycarbonyl)-1H-pyrazol-5-yl)cyclopent-1-en-1-yl)oxazole-5-carboxylate (1.30 g, 54.27%). LC-MS (ESI): m/z [M+H]⁺=509.4.

### Step 6: tert-butyl 3-amino-S-(3-(5-(2-hydroxypropan-2-yl)oxazol-2-yl)cyclopent-2-en-1-yl)-1H-pyrazole-1-carboxylate

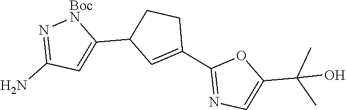 [SMILES: CC(C)(C)OC(=O)n1nc(N)cc1C1C=C(c2ncc(C(C)(C)O)o2)CC1]

Into a 100-mL 3-necked round-bottom flask purged and maintained with an inert atmosphere of nitrogen, were placed Methyl 2-(3-(3-(((benzyloxy)carbonyl)amino)-1-(tert-butoxycarbonyl)-1H-pyrazol-5-yl)cyclopent-1-en-1-yl)oxazole-5-carboxylate (600.0 mg, 1.18 mmol) in THF 10 mL, was added MeMgBr (1.97 mL, 5.91 mmol) dropwised at 0° C. under nitrogen atmosphere. The resulting mixture was stirred for 1 h at 0° C. The reaction was quenched with sat NH₄Cl(aq.) at 0° C. The resulting mixture was extracted with EtOAc (50 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EtOAc (100:0-50:50) to afford tert-butyl 3-amino-5-(3-(5-(2-hydroxypropan-2-yl)oxazol-2-yl)cyclopent-2-en-1-yl)-1H-pyrazole-1-carboxylate (300.0 mg, 67.91%). LC-MS (ESI): m/z [M+H]⁺=375.4.

### Step 7: tert-butyl 3-amino-5-(3-(5-(2-hydroxypropan-2-yl)oxazol-2-yl)cyclopentyl)-1H-pyrazole-1-carboxylate

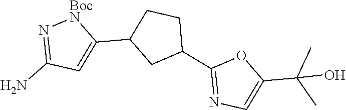 [SMILES: CC(C)(C)OC(=O)n1nc(N)cc1C1CCC(c2ncc(C(C)(C)O)o2)C1]

Into a 100-mL round-bottom flask was added tert-butyl 3-amino-5-(3-(5-(2-hydroxypropan-2-yl)oxazol-2-yl)cyclopent-2-en-1-yl)-1H-pyrazole-1-carboxylate (300.0 mg, 0.80 mmol), Pd/C (300.0 mg) and THF (10 mL) at room temperature. The resulting mixture was stirred for 2 h at room temperature under H₂ atmosphere. The resulting mixture was concentrated under reduced pressure. The residue was purified by silica gel column chromatography, eluted with PE/EtOAc (100:0-20:80) to afford tert-butyl 3-amino-5-(3-(5-(2-hydroxypropan-2-yl)oxazol-2-yl)cyclopentyl)-1H-pyrazole-1-carboxylate (150.0 mg, 49.73%). LC-MS (ESI): m/z [M+H]⁺=377.4.

### Step 8: 2-(2-(3-(3-amino-1H-pyrazol-5-yl)cyclopentyl)oxazol-5-yl)propan-2-ol

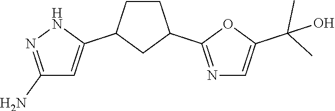 [SMILES: CC(C)(O)c1cnc(C2CCC(c3cc(N)n[nH]3)C2)o1]

Into a 100-mL round-bottom flask was added tert-butyl 3-amino-5-((1S,3R)-3-(5-(2-hydroxypropan-2-yl)oxazol-2-yl)cyclopentyl)-1H-pyrazole-1-carboxylate (220.0 mg, 0.59 mmol), SiO₂ (220.0 mg) and toluene (10 mL) at room temperature. The resulting mixture was stirred 15 h at 100° C. The resulting mixture was concentrated under reduced pressure. The residue was purified by silica gel column chromatography, eluted with DCM/MeOH (100:0~92:8) to afford the product (90.0 mg, 55.73%). ¹H NMR (300 MHz, Chloroform-d) δ 6.80 (d, J=9.9 Hz, 1H), 5.51 (s, 1H), 3.72 (s, 2H), 3.43 (d, J=6.6 Hz, 2H), 3.33 (s, 1H), 2.49 (dt, J=13.6 Hz, 1H), 2.14 (ddd, J=32.1 Hz, 3H), 1.82 (s, 1H), 1.46 (s, 6H). LC-MS (ESI): m/z [M+H]⁺=277.3.

### Step 9: 4-fluoro-5-((5-(3-(5-(2-hydroxypropan-2-yl)oxazol-2-yl)cyclopentyl)-1H-pyrazol-3-yl)amino)-2,3-dihydrobenzo[d]isothiazole 1,1-dioxide

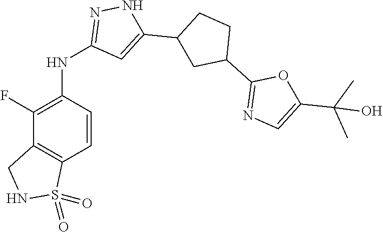 [SMILES: CC(C)(O)c1cnc(C2CCC(c3cc(Nc4ccc5c(c4F)CNS5(=O)=O)n[nH]3)C2)o1]

The titled compound was synthesized in the procedures similar to Example 6. ¹H NMR (500 MHz, DMSO-d₆) δ12.05 (s, 1H), 8.73 (d, J=2.6 Hz, 1H), 8.26 (s, 1H), 7.69 (t, J=5.1 Hz, 1H), 7.48 (d, J=8.6 Hz, 1H), 6.79 (s, 1H), 5.84 (s, 1H), 5.23 (s, 1H), 4.40 (d, J=5.0 Hz, 2H), 3.41-3.34 (m, 1H), 3.20 (ddd, J=17.5, 10.1, 7.3 Hz, 1H), 2.45 (dd, J=13.5, 6.2 Hz, 1H), 2.17-2.06 (m, 2H), 2.03-1.94 (m, 1H), 1.90 (dt, J=12.3, 10.2 Hz, 1H), 1.75 (ddd, J=12.7, 10.5, 5.6 Hz, 1H), 1.42 (s, 6H). LC-MS (ESI): m/z [M+H]⁺=462.3.

### Example 111: 5-((5-(3-(5-(tert-butyl)-4-methyloxazol-2-yl)cyclopentyl)-1H-pyrazol-3-yl)amino)-4-fluoro-2,3-dihydrobenzo[d]isothiazole 1,1-dioxide

### Step 1: tert-butyl (4,4-dimethyl-3-oxopentan-2-yl)carbamate

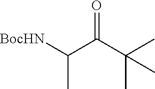 [SMILES: CC(NC(=O)OC(C)(C)C)C(=O)C(C)(C)C]

To a solution of tert-butyl (1-(methoxy(methyl)amino)-1-oxopropan-2-yl)carbamate (10.50 g, 45.26 mmol) in Et₂O (150 mL) was added n-BuLi (45.26 mL, 113.15 mmol) dropwised at −78° C. under nitrogen atmosphere. The resulting mixture was stirred for 2 h at −78° C. The reaction was quenched with sat NH₄Cl(aq.) at 0° C. and then extracted with EtOAc (3×300 mL). The combined organic layers were washed with brine, dried over anhydrous Na₂SO₄ and filtrated. The filtrate was concentrated under reduced pressure and the residue was purified by silica gel column chromatography, eluted with PE/EA (100:0~90:10) to afford the product (1.8 g, 17.37%). LC-MS (ESI): m/z [M+H]⁺=230.3.

### Step 2: 4-amino-2,2-dimethylpentan-3-one chloride

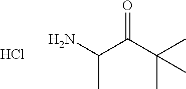

Into a 250-mL round-bottom flask were added tert-butyl (4,4-dimethyl-3-oxopentan-2-yl)carbamate (1.8 g, 13.95 mmol) and HCl-dioxane (4M, 20 mL) at room temperature. The resulting mixture was stirred for 2 h at room temperature. The resulting mixture was concentrated under reduced pressure to afford the product (1.0 g, 98.62% yield). LC-MS (ESI): m/z [M+H]⁺=130.3.

### Step 3: cis-3-((4,4-dimethyl-3-oxopentan-2-yl)carbamoyl)cyclopentane-1-carboxylic acid

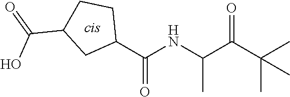 [SMILES: CC(NC(=O)C1CCC(C(=O)O)C1)C(=O)C(C)(C)C]

To a solution of (1R,5S)-3-oxabicyclo[3.2.1]octane-2,4-dione (1.09 g, 7.75 mmol) in THF (30 mL) was added 4-amino-2,2-dimethylpentan-3-one (1.0 g, 7.75 mmol) and TEA (1.57 g, 15.5 mmol). The resulting mixture was stirred 2 h at room temperature. The solution was concentrated under reduced pressure to afford the product (2.0 g, 95.91%). LC-MS (ESI): m/z [M+H]⁺=270.3.

### Step 4: cis-methyl 3-((4,4-dimethyl-3-oxopentan-2-yl)carbamoyl)cyclopentane-1-carboxylate

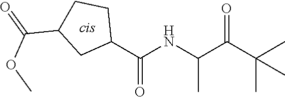 [SMILES: COC(=O)C1CCC(C(=O)NC(C)C(=O)C(C)(C)C)C1]

To a solution of cis-3-((4,4-dimethyl-3-oxopentan-2-yl)carbamoyl)cyclopentane-1-carboxylic acid (2.05 g, 7.62 mmol) in MeOH (50 mL) was added H₂SO₄ (1.49 g, 15.24 mmol). The resulting mixture was stirred for 3 h at room temperature. The mixture was neutralized to PH=7 with Na₂CO₃(aq.) aqueous solution and extracted with 150 mL of EtOAc. The combined organic layers were washed with brine, dried over anhydrous Na₂SO₄ and filtrated. The filtrate was concentrated under reduced pressure and the residue was purified by silica gel column chromatography, eluted with PE/EtOAc (100:0~70:30) to afford the product (1.90 g, 88% yield). LC-MS (ESI): m/z [M+H]⁺=284.3.

### Step 5: cis-methyl 3-(5-(tert-butyl)-4-methyloxazol-2-yl)cyclopentane-1-carboxylate

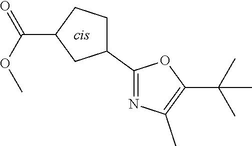 [SMILES: COC(=O)C1CCC(c2nc(C)c(C(C)(C)C)o2)C1]

cis-methyl 3-((4,4-dimethyl-3-oxopentan-2-yl)carbamoyl)cyclopentane-1-carboxylate (1.90 g, 6.71 mmol) was added to POCl₃ (40 mL). The resulting mixture was stirred for 5 h at 60° C. The resulting mixture was concentrated under reduced pressure. The residue was purified by silica gel column chromatography, eluted with PE/EtOAc (100:0-40:60) to afford the product (1.60 g, 89.93%). LC-MS (ESI): m/z [M+H]⁺=266.2.

### Step 6: 3-(3-(5-(tert-butyl)-4-methyloxazol-2-yl)cyclopentyl)-3-oxopropanenitrile

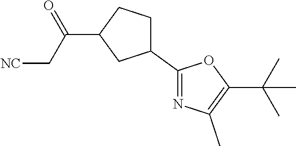 [SMILES: Cc1nc(C2CCC(C(=O)CC#N)C2)oc1C(C)(C)C]

Into a 100-mL 3-necked round-bottom flask purged with an inert atmosphere of nitrogen was placed CH₃CN (496.1 mg, 12.1 mmol) in THF 20 mL. Then n-BuLi (4.84 mL, 12.1 mmol) was added dropwise at −78° C. The resulting mixture was stirred for 30 min at −78° C., then added to a solution of cis-methyl 3-(5-(tert-butyl)-4-methyloxazol-2-yl)cyclopentane-1-carboxylate (1.60 g, 6.04 mmol) in THF (50 mL) dropwise at −78° C. The resulting mixture was stirred for 2 h at −78° C. The reaction was quenched with sat NH₄Cl(aq.) at 0° C. The resulting mixture was extracted with EtOAc (100 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EA (100:0-60:40) to afford the product (0.92 g, 55.61% yield). LC-MS (ESI): m/z [M+H]⁺=275.3.

### Step 7: 5-(3-(5-(tert-butyl)-4-methyloxazol-2-yl)cyclopentyl)-1H-pyrazol-3-amine

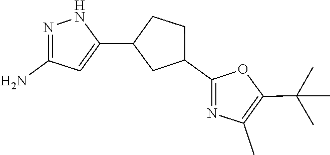 [SMILES: Cc1nc(C2CCC(c3cc(N)n[nH]3)C2)oc1C(C)(C)C]

To a solution of 3-(3-(5-(tert-butyl)-4-methyloxazol-2-yl)cyclopentyl)-3-oxopropanenitrile (900.0 mg, 3.28 mmol) in EtOH (20 mL) were added hydrazine hydrate (180.5 mg, 3.61 mmol)) and TsOH (50 mg). The resulting mixture was stirred at 90° C. for 1 h. The reaction was concentrated under vacuum and the residue was purified by silica gel column chromatography, eluting with PE:EA (100:0 to 80:20) to give the product (844.6 mg, 89.28% yield). ¹H NMR (300 MHz, DMSO-d₆) δ 5.19 (s, 1H), 3.21 (p, J=8.1 Hz, 1H), 3.10-2.94 (m, 1H), 2.39-2.24 (m, 1H), 2.10 (s, 4H), 2.08-1.85 (m, 3H), 1.88-1.59 (m, 1H), 1.27 (s, 9H). LC-MS (ESI): m/z [M+H]⁺=289.3.

### Step 8: 5-((5-(3-(5-(tert-butyl)-4-methyloxazol-2-yl)cyclopentyl)-1H-pyrazol-3-yl)amino)-4-fluoro-2,3-dihydrobenzo[d]isothiazole 1,1-dioxide

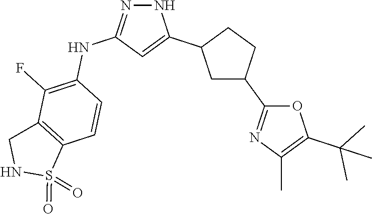 [SMILES: Cc1nc(C2CCC(c3cc(Nc4ccc5c(c4F)CNS5(=O)=O)n[nH]3)C2)oc1C(C)(C)C]

The titled compound was synthesized in the procedures similar to Example 6. ¹H NMR (500 MHz, DMSO-d₆) δ 12.03 (s, 1H), 8.72 (s, 1H), 8.26 (t, J=5 Hz, 1H), 7.69 (d, J=5 Hz, 1H), 7.47 (d, J=10 Hz, 1H), 5.84 (s, 1H), 4.40 (d, J=5 Hz, 2H), 3.30-3.12 (m, 2H), 2.46-2.38 (m, 1H), 2.18-2.03 (m, 5H), 1.99-1.82 (m, 2H), 1.77-1.69 (m, 1H), 1.27 (s, 9H). LC-MS (ESI): m/z [M+H]⁺=474.2.

### Example 112: 4-fluoro-5-((5-(3-(5-(1-methylcyclopropyl)oxazol-2-yl)cyclopentyl)-1H-pyrazol-3-yl)amino)-2,3-dihydrobenzo[d]isothiazole 1,1-dioxide

### Step 1: trimethyl((1-methylcyclopropyl)ethynyl)silane

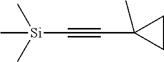

To a solution of (cyclopropylethynyl)trimethylsilane (10 g, 72.4 mmol) in diethyl ether (80 mL) was added n-BuLi (2.5M) (29 mL, 72.4 mmol) at r.t. and stirred for overnight. The dimethyl sulfate (24.5 g, 194 mmol) was added in it at −10° C. The reaction was stirred at rt for 1 h. The reaction was quenched with NH₄Cl:NH₃H₂O=1:3 (125 mL) and stirred at rt for 1 h. The resulting solution was extracted with 2×150 mL of Et₂O. The combined organic layers were washed with 5% HCl solution, NaHCO₃ solution and water. The reaction was concentrated under reduced pressure at 0° C. to give the crude product (11 g, 95%).

### Step 2: methyl 3-cyanocyclopentane-1-carboxylate

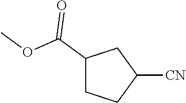

To a solution of methyl 3-carbamoylcyclopentane-1-carboxylate (6 g, 35 mmol) in THF (150 mL) were added TEA (10.6 g, 105 mmol) and TFAA (16.2 g, 77.2 mmol) and stirred at rt for 3 h. The reaction was concentrated under vacuum. The crude was purified by silica gel column chromatography, eluting with PE:EA (100:0 to 77:23) to give the product (4.6 g, 85.8%).

### Step 3: methyl 3-(5-(1-methylcyclopropyl)oxazol-2-yl)cyclopentane-1-carboxylate

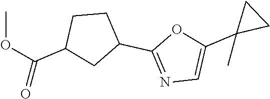 [SMILES: COC(=O)C1CCC(c2ncc(C3(C)CC3)o2)C1]

To a solution of methyl 3-cyanocyclopentane-1-carboxylate (3 g, 19.59 mmol) in trimethyl((1-methylcyclopropyl)ethynyl)silane (9 g, 58.77 mmol) was added Ph₃PAuNTf₂ (723 mg, 0.979 mmol) and 8-methylquinoline 1-oxide (4.05 g, 25.46 mmol). The resulting mixture was stirred at 60° C. for 4 h under N₂ atmosphere. The resulting mixture was concentrated under vacuum and the residue was purified by silica gel column chromatography, eluting with PE:EA (100:0 to 92:8) to give the product (5 g, 95%). LCMS (ESI) m/z [M+H]⁺=250.

### Step 4: 3-(3-(5-(1-methylcyclopropyl)oxazol-2-yl)cyclopentyl)-3-oxopropanenitrile

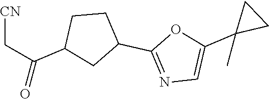 [SMILES: CC1(c2cnc(C3CCC(C(=O)CC#N)C3)o2)CC1]

To a solution of CH₃CN (330 mg, 1.6 mmol) in THF (30 mL) was added n-Buli (2.5 M) (3.2 mL, 1.6 mmol) at −78° C. and stirred for 1 h under N₂ atmosphere. The methyl 3-(5-(1-methylcyclopropyl)oxazol-2-yl)cyclopentane-1-carboxylate (1 g, 0.8 mmol) was added in it at −78° C. and stirred for another hour. The reaction was quenched with NH₄Cl solution (40 mL) and extracted with 3×30 mL of EtOAc. The combined organic layers were dried over Na₂SO₄ and concentrated under vacuum. The residue was purified by silica gel column chromatography, eluting with PE:EA (100:0 to 60:40) to give the product (540 mg, 52.4%). LCMS (ESI) m/z [M+H]⁺=259.

### Step 5: 5-(3-(5-(1-methylcyclopropyl)oxazol-2-yl)cyclopentyl)-1H-pyrazol-3-amine

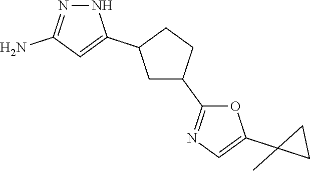 [SMILES: CC1(c2cnc(C3CCC(c4cc(N)n[nH]4)C3)o2)CC1]

To a solution of 3-(3-(5-(1-methylcyclopropyl)oxazol-2-yl)cyclopentyl)-3-oxopropanenitrile (500 mg, 1.93 mmol) in EtOH (10 mL) were added hydrazine dyrate (106.5 mg, 2.13 mmol) and TsOH (10 mg) and stirred at 90° C. for 1 h. The reaction was concentrated under vacuum, and the residue was purified by silica gel column chromatography, eluting with DCM:MeOH (100:0 to 94:6) to give the product (468 mg, 88.8%). H NMR (300 MHz, DMSO-d₆) δ 6.69 (s, 1H), 5.21 (d, J=7.8 Hz, 2H), 3.27-2.94 (m, 5H), 2.42-2.23 (m, 2H), 2.14-1.59 (m, 13H), 1.35 (s, 3H), 0.91 (q, J=4.0 Hz, 2H), 0.79-0.70 (m, 2H). LCMS (ESI) m/z [M+H]⁺=273.

### Step 6: 4-fluoro-5-((5-(3-(5-(1-methylcyclopropyl)oxazol-2-yl)cyclopentyl)-1H-pyrazol-3-yl)amino)-2,3-dihydrobenzo[d]isothiazole 1,1-dioxide

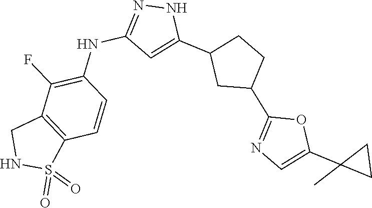 [SMILES: CC1(c2cnc(C3CCC(c4cc(Nc5ccc6c(c5F)CNS6(=O)=O)n[nH]4)C3)o2)CC1]

The titled compound was synthesized in the procedures similar to Example 6. ¹H NMR (500 MHz, DMSO-d₆) δ=12.04 (s, 1H), 8.73 (s, 1H), 8.26 (s, 1H), 7.69 (s, 1H), 7.48 (d, J=8.6 Hz, 1H), 6.70 (s, 1H), 5.83 (s, 1H), 4.41 (s, 2H), 3.24-3.14 (m, 2H), 2.46-2.38 (m, 1H), 2.16-2.02 (m, 2H), 2.01-1.83 (m, 2H), 1.77-1.67 (m, 1H), 1.34 (s, 3H), 0.91 (q, J=4.1 Hz, 2H), 0.75 (q, J=4.1 Hz, 2H). LC-MS (ESI): m/z [M+H]⁺=458.2.

### Example 113: methyl 5-(tert-butyl)-2-(3-(3-((4-fluoro-1,1-dioxido-2,3-dihydrobenzo[d]isothiazol-5-yl)amino)-1H-pyrazol-5-yl)cyclopentyl)oxazole-4-carboxylate

### Step 1: ethyl 2-bromo-5-(tert-butyl)oxazole-4-carboxylate

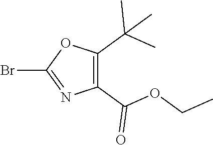

To a solution of ethyl 5-(tert-butyl)oxazole-4-carboxylate (2.0 g, 10.15 mmol) in THF (50 mL) was added LiHMDS (1.0 M in THF, 11.2 mL, 11.2 mmol) at −60° C. under nitrogen, stirred for 1 hour at −60° C., CBr₄ (3.37 g, 10.15 mmol) in THF (15 mL) was added at −60° C., the reaction mixture was warmed to room temperature, stirred another 30 minutes. The resulting mixture was quenched with saturated NH₄Cl aqueous, extracted with EA, dried over Na₂SO₄, and purified by column chromatography on silica gel (petroleum ether:ethyl acetate=5:1) to afford the product (1.4 g, 50%). LC-MS (ESI): m/z [M+H]⁺=276.0.

### Step 2: ethyl 5-(tert-butyl)-2-(3-(1-(tert-butyl)-5-((4-fluoro-1,1-dioxido-2,3-dihydrobenzo[d]isothiazol-5-yl)amino)-1H-pyrazol-3-yl)cyclopentyl)oxazole-4-carboxylate

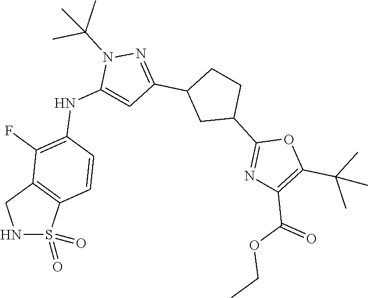 [SMILES: CCOC(=O)c1nc(C2CCC(c3cc(Nc4ccc5c(c4F)CNS5(=O)=O)n(C(C)(C)C)n3)C2)oc1C(C)(C)C]

The titled compound was synthesized in the procedures similar to Example 1. LC-MS (ESI): m/z [M+H]⁺=588.3.

### Step 3: ethyl 5-(tert-butyl)-2-(3-(5-((4-fluoro-2-formyl-1,1-dioxido-2,3-dihydrobenzo[d]isothiazol-5-yl)amino)-1H-pyrazol-3-yl)cyclopentyl)oxazole-4-carboxylate

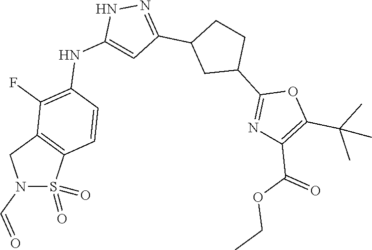 [SMILES: CCOC(=O)c1nc(C2CCC(c3cc(Nc4ccc5c(c4F)CN(C=O)S5(=O)=O)[nH]n3)C2)oc1C(C)(C)C]

ethyl 5-(tert-butyl)-2-(3-(1-(tert-butyl)-5-((4-fluoro-1,1-dioxido-2,3-dihydrobenzo[d]isothiazol-5-yl)amino)-1H-pyrazol-3-yl)cyclopentyl)oxazole-4-carboxylate (72 mg, 0.122 mmol) was dissolved in formic acid (10 mL) and stirred at 75° C. for 4 h. The solution was concentrated in vacuum to give the crude product which was directedly used for the next step without further purification. LC-MS (ESI): m/z [M+H]⁺=560.3.

### Step 4: methyl 5-(tert-butyl)-2-(3-(3-((4-fluoro-1,1-dioxido-2,3-dihydrobenzo[d]isothiazol-5-yl)amino)-1H-pyrazol-5-yl)cyclopentyl)oxazole-4-carboxylate

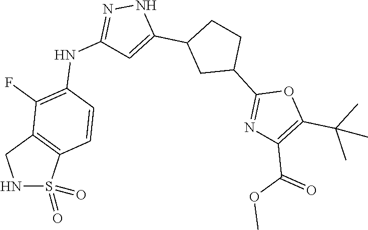 [SMILES: COC(=O)c1nc(C2CCC(c3cc(Nc4ccc5c(c4F)CNS5(=O)=O)n[nH]3)C2)oc1C(C)(C)C]

To a solution of ethyl 5-(tert-butyl)-2-(3-(5-((4-fluoro-2-formyl-1,1-dioxido-2,3-dihydrobenzo[d]isothiazol-5-yl)amino)-1H-pyrazol-3-yl)cyclopentyl)oxazole-4-carboxylate (70 mg, crude) in MeOH (10 mL) was added K₂CO₃ (34 mg, 0.25 mmol) and stirred at rt for 1 h. The mixture was filtrated and concentrated, and the residue was purified by column chromatography on silica gel (100% EA) to afford the product (20 mg, 32%). ¹H NMR (500 MHz, DMSO-d₆) δ=12.05 (d, J=8.4, 1H), 8.73 (d, J=5.7, 1H), 8.25 (d, J=7.7, 1H), 7.69 (s, 1H), 7.47 (d, J=8.6, 1H), 5.85 (d, J=16.4, 1H), 4.40 (d, J=2.4, 2H), 3.79 (d, J=5.3, 3H), 3.49-3.38 (m, 1H), 3.24-3.18 (m, 1H), 2.45 (m, 1H), 2.35-2.15 (m, 1H), 2.15-2.03 (m, 2H), 1.97-1.87 (m, 1H), 1.79-1.68 (m, 1H), 1.38 (d, J=7.1, 9H). LC-MS (ESI): m/z [M+H]⁺=518.41.

### Example 114: ethyl 5-(tert-butyl)-2-(3-(3-((4-fluoro-1,1-dioxido-2,3-dihydrobenzo[d]isothiazol-5-yl)amino)-1H-pyrazol-5-yl)cyclopentyl)oxazole-4-carboxylate

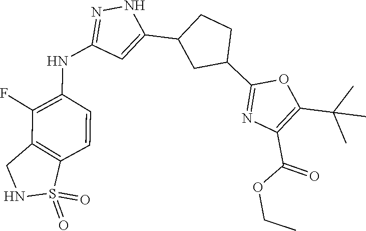 [SMILES: CCOC(=O)c1nc(C2CCC(c3cc(Nc4ccc5c(c4F)CNS5(=O)=O)n[nH]3)C2)oc1C(C)(C)C]

The titled compound was isolated in the Example 113, step 4 (column chromatography on silica gel, eluting with 100% EA). ¹H NMR (500 MHz, DMSO-d₆) δ=12.05 (d, J=10.3, 1H), 8.73 (d, J=5.9, 1H), 8.26 (s, 1H), 7.69 (s, 1H), 7.47 (m, 1H), 5.86 (d, J=13.6, 1H), 4.41 (s, 2H), 4.27 (m, 2H), 3.49-3.38 (m, 1H), 3.25-3.16 (m, 1H), 2.48-2.43 (m, 1H), 2.35-2.16 (m, 1H), 2.15-2.01 (m, 2H), 2.00-1.86 (m, 1H), 1.75 (m, 1H), 1.38 (d, J=6.8, 9H), 1.28 (m, 3H). LC-MS (ESI): m/z [M+H]⁺=532.40.

### Example 115: ethyl 2-(3-(3-((4-fluoro-1,1-dioxido-2,3-dihydrobenzo[d]isothiazol-5-yl)amino)-1H-pyrazol-5-yl)cyclopentyl)-5-isopropyloxazole-4-carboxylate

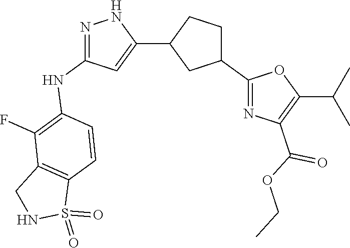 [SMILES: CCOC(=O)c1nc(C2CCC(c3cc(Nc4ccc5c(c4F)CNS5(=O)=O)n[nH]3)C2)oc1C(C)C]

The titled compound was synthesized in the procedures similar to Example 114. ¹H NMR (500 MHz, DMSO-d₆) δ 12.06 (s, 1H), 8.73 (d, J=6.0 Hz, 1H), 8.30 (d, J=46.7 Hz, 1H), 7.69 (s, 1H), 7.47 (dd, J=8.6, 3.2 Hz, 1H), 5.86 (d, J=15.3 Hz, 1H), 4.41 (s, 2H), 4.31-4.18 (m, 2H), 3.66 (tt, J=13.9, 6.9 Hz, 1H), 3.54-3.43 (m, 1H), 3.28-3.17 (m, 1H), 2.49-2.43 (m, 1H), 2.25-2.09 (m, 2H), 2.08-1.87 (m, 2H), 1.81-1.65 (m, 1H), 1.28 (td, J=7.1, 3.8 Hz, 3H), 1.23 (dd, J=10.3, 4.3 Hz, 6H). LC-MS (ESI): m/z [M+H]⁺=519.2.

### Example 116: 5-((5-(3-(5-cyclopropyloxazol-2-yl)cyclopentyl)-1H-pyrazol-3-yl)amino)-4-fluoro-2,3-dihydrobenzo[d]isothiazole 1,1-dioxide

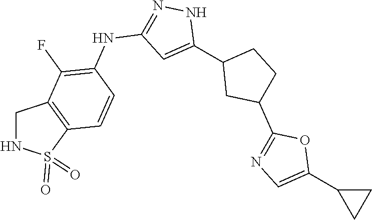 [SMILES: O=S1(=O)NCc2c1ccc(Nc1cc(C3CCC(c4ncc(C5CC5)o4)C3)[nH]n1)c2F]

The titled compound was synthesized in the procedures similar to Example 9. ¹H NMR (500 MHz, DMSO) δ 12.03 (s, 1H), 8.73 (s, 1H), 8.26 (s, 1H), 7.69 (t, J=5.0 Hz, 1H), 7.47 (d, J=8.6 Hz, 1H), 6.70 (d, J=0.6 Hz, 1H), 5.83 (s, 1H), 4.40 (d, J=5.1 Hz, 2H), 3.29 (s, 1H), 3.23-3.14 (m, 1H), 2.46-2.38 (m, 1H), 2.15-2.05 (m, 2H), 2.00-1.84 (m, 3H), 1.77-1.69 (m, 1H), 0.92-0.86 (m, 2H), 0.71-0.66 (m, 2H). LC-MS (ESI): m/z [M+H]⁺=444.3

### Example 117: 5-((3-(3-(4-(tert-butyl)pyridin-2-yl)cyclopentyl)-1H-pyrazol-5-yl)amino)-4-fluoro-2,3-dihydrobenzo[d]isothiazole 1,1-dioxide

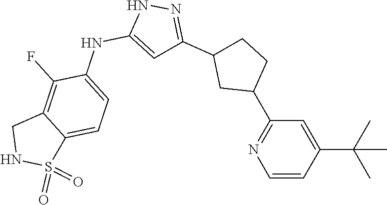 [SMILES: CC(C)(C)c1ccnc(C2CCC(c3cc(Nc4ccc5c(c4F)CNS5(=O)=O)[nH]n3)C2)c1]

The titled compound was synthesized in the procedures similar to Example 1. ¹H NMR (500 MHz, DMSO-d₆) δ 12.08 (s, 1H), 8.73 (s, 1H), 8.50-8.41 (m, 1H), 8.32-8.22 (m, 1H), 7.69 (d, J=5 Hz, 1H), 7.48 (d, J=10 Hz, 1H), 7.42-7.24 (m, 2H), 5.88 (s, 1H), 4.40 (d, J=5 Hz, 2H), 3.48-3.38 (m, 1H), 3.27-3.19 (m, 1H), 2.45-2.35 (m, 1H), 2.29-2.06 (m, 2H), 2.01-1.80 (m, 3H), 1.28 (s, 9H). LC-MS (ESI): m/z [M+H]⁺=470.2.

### Example 118: 5-((3-(3-(6-(tert-butyl)pyridin-2-yl)cyclopentyl)-1H-pyrazol-5-yl)amino)-4-fluoro-2,3-dihydrobenzo[d]isothiazole 1,1-dioxide

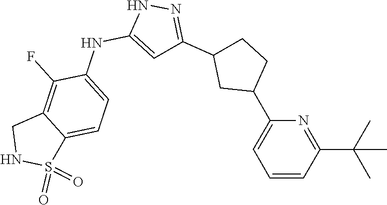 [SMILES: CC(C)(C)c1cccc(C2CCC(c3cc(Nc4ccc5c(c4F)CNS5(=O)=O)[nH]n3)C2)n1]

The titled compound was synthesized in the procedures similar to Example 1. ¹H NMR (500 MHz, DMSO-d₆) δ 12.08 (s, 1H), 8.74 (s, 1H), 8.31-8.21 (m, 1H), 7.77-7.56 (m, 2H), 7.47 (d, J=10 Hz, 1H), 7.32-7.03 (m, 2H), 5.87 (s, 1H), 4.41 (s, 2H), 3.44-3.17 (m, 2H), 2.45-2.38 (m, 1H), 2.30-2.03 (m, 2H), 2.01-1.78 (m, 3H), 1.33 (s, 9H). LC-MS (ESI): m/z [M+H]⁺=470.2.

### Example 119: 5-((3-(3-(5-(tert-butyl)pyridin-2-yl)cyclopentyl)-1H-pyrazol-5-yl)amino)-4-fluoro-2,3-dihydrobenzo[d]isothiazole 1,1-dioxide

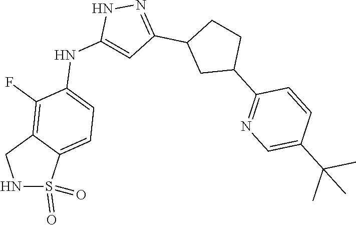 [SMILES: CC(C)(C)c1ccc(C2CCC(c3cc(Nc4ccc5c(c4F)CNS5(=O)=O)[nH]n3)C2)nc1]

The titled compound was synthesized in the procedures similar to Example 1. ¹H NMR (500 MHz, DMSO-d₆) δ 12.06 (s, 1H), 8.72 (d, J=5 Hz, 1H), 8.56 (d, J=5 Hz, 1H), 8.33-8.22 (m, 1H), 7.75-7.66 (m, 2H), 7.48 (d, J=10 Hz, 1H), 7.23 (d, J=10 Hz, 1H), 5.87 (s, 1H), 4.41 (d, J=5 Hz, 2H), 3.30-3.18 (m, 2H), 2.42-2.33 (m, 1H), 2.22-2.02 (m, 2H), 1.98-1.76 (m, 3H), 1.30 (s, 9H). LC-MS (ESI): m/z [M+H]⁺=470.2.

### Example 120: 4-fluoro-5-((5-(3-(6-methylpyridin-2-yl)cyclopentyl)-1H-pyrazol-3-yl)amino)-2,3-dihydrobenzo[d]isothiazole 1,1-dioxide

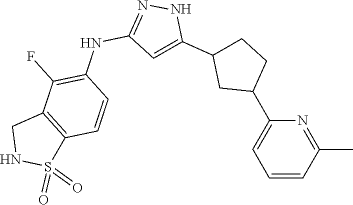 [SMILES: Cc1cccc(C2CCC(c3cc(Nc4ccc5c(c4F)CNS5(=O)=O)n[nH]3)C2)n1]

The titled compound was synthesized in the procedures similar to Example 1. ¹H NMR (500 MHz, DMSO-d₆) δ 12.11 (s, 1H), 8.75 (s, 1H), 8.47-8.16 (m, 2H), 7.94-7.62 (m, 3H), 7.48 (d, J=10 Hz, 1H), 5.91 (s, 1H), 4.41 (s, 2H), 3.49-3.25 (m, 2H), 2.69 (s, 3H), 2.57-2.54 (m, 1H), 2.33-2.15 (m, 2H), 2.02-1.84 (m, 3H). LC-MS (ESI): m/z [M+H]⁺=427.1.

### Example 121: 4-fluoro-5-((5-(3-(6-methoxypyridin-2-yl)cyclopentyl)-1H-pyrazol-3-yl)amino)-2,3-dihydrobenzo[d]isothiazole 1,1-dioxide

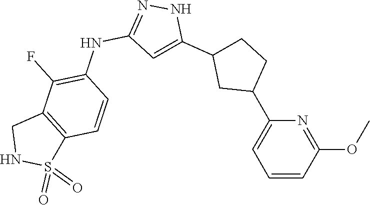 [SMILES: COc1cccc(C2CCC(c3cc(Nc4ccc5c(c4F)CNS5(=O)=O)n[nH]3)C2)n1]

The titled compound was synthesized in the procedures similar to Example 1. ¹H NMR (500 MHz, DMSO-d₆) δ 12.05 (s, 1H), 8.73 (s, 1H), 8.25 (t, J=5 Hz, 1H), 7.74-7.65 (m, 1H), 7.60 (t, J=10 Hz, 1H), 7.48 (d, J=10 Hz, 1H), 6.88 (d, J=10 Hz, 1H), 6.62 (d, J=10 Hz, 1H), 5.87 (s, 1H), 4.41 (s, 2H), 3.84 (s, 3H), 3.44-3.16 (m, 2H), 2.44-2.34 (m, 1H), 2.27-2.01 (m, 2H), 1.99-1.72 (m, 3H). LC-MS (ESI): m/z [M+H]⁺=444.1.

### Example 122: 4-fluoro-5-((5-(3-(4-isopropylpyridin-2-yl)cyclopentyl)-1H-pyrazol-3-yl)amino)-2,3-dihydrobenzo[d]isothiazole 1,1-dioxide

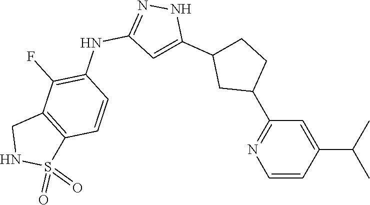 [SMILES: CC(C)c1ccnc(C2CCC(c3cc(Nc4ccc5c(c4F)CNS5(=O)=O)n[nH]3)C2)c1]

The titled compound was synthesized in the procedures similar to Example 4 (90 mg). ¹H NMR (500 MHz, DMSO-d₆) δ 12.07 (s, 1H), 8.73 (s, 1H), 8.41 (d, J=7.7 Hz, 1H), 8.28 (t, J=7.7 Hz, 1H), 7.69 (s, 1H), 7.48 (d, J=8.6 Hz, 1H), 7.17 (s, 1H), 7.09 (dd, J=5.1, 1.4 Hz, 1H), 5.87 (s, 1H), 4.41 (d, J=4.4 Hz, 2H), 3.25-3.16 (m, 1H), 2.90-2.82 (m, 1H), 2.57-2.54 (m, 1H), 2.40-2.35 (m, 1H), 2.20-2.05 (m, 2H), 2.00-1.90 (m, 2H), 1.89-1.77 (m, 1H), 1.20 (d, J=6.9 Hz, 6H). LC-MS (ESI): m/z [M+H]⁺=456.34.

### Example 123: 5-((5-(3-(4-(tert-butyl)-6-methylpyridin-2-yl)cyclopentyl)-1H-pyrazol-3-yl)amino)-4-fluoro-2,3-dihydrobenzo[d]isothiazole 1,1-dioxide

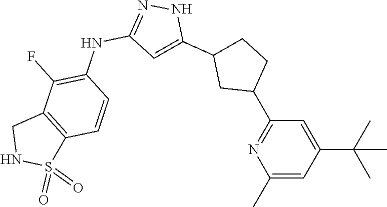 [SMILES: Cc1cc(C(C)(C)C)cc(C2CCC(c3cc(Nc4ccc5c(c4F)CNS5(=O)=O)n[nH]3)C2)n1]

The titled compound was synthesized in the procedures similar to Example 2. ¹H NMR (500 MHz, DMSO-d₆) δ 12.18 (s, 1H), 8.72 (s, 1H), 8.33-8.22 (m, 1H), 7.69 (t, J=5 Hz, 1H), 7.48 (d, J=10 Hz, 1H), 7.07 (s, 2H), 5.88 (s, 1H), 4.41 (d, J=5 Hz, 2H), 3.30-3.18 (m, 2H), 2.44 (s, 3H), 2.40-2.32 (m, 1H), 2.18-2.02 (m, 2H), 1.98-1.78 (m, 3H), 1.25 (s, 9H). LC-MS (ESI): m/z [M+H]⁺=484.2.

### Example 124: 4-fluoro-5-((5-(3-(4-methylpyrimidin-2-yl)cyclopentyl)-1H-pyrazol-3-yl)amino)-2,3-dihydrobenzo[d]isothiazole 1,1-dioxide

### Step 1: benzyl (1-(tert-butyl)-3-(3-(4-methylpyrimidin-2-yl)cyclopent-2-en-1-yl)-1H-pyrazol-5-yl)carbamate

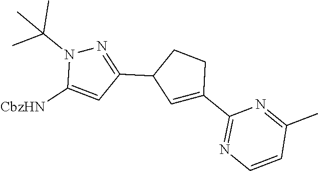 [SMILES: Cc1ccnc(C2=CC(c3cc(NC(=O)OCc4ccccc4)n(C(C)(C)C)n3)CC2)n1]

To a mixture of benzyl (1-(tert-butyl)-3-(3-(4,4,5,5-tetramethyl-1,3,2-dioxaborolan-2-yl)cyclopent-2-en-1-yl)-1H-pyrazol-5-yl)carbamate (930 mg, 2 mmol) and 2-chloro-4-methylpyrimidine (256 mg, 2 mmol) in dioxane (20 mL) was added Pd(dppf)Cl₂ (146 mg, 0.2 mmol), K₂CO₃ (828 mg, 6 mmol) and H₂O (2 mL). The reaction mixture was stirred at 80° C. under nitrogen atmosphere for 4 h. The mixture was concentrated under reduced pressure and purified by silica gel column chromatography, eluting with PE/EA (4:1) to afford the product (265 mg, 25%). LC-MS (ESI): m/z [M+H]⁺=432.3

### Step 2: benzyl (5-(3-(4-methylpyrimidin-2-yl)cyclopent-2-en-1-yl)-1H-pyrazol-3-yl)carbamate

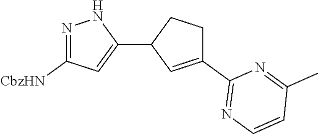 [SMILES: Cc1ccnc(C2=CC(c3cc(NC(=O)OCc4ccccc4)n[nH]3)CC2)n1]

A mixture of benzyl (1-(tert-butyl)-3-(3-(4-methylpyrimidin-2-yl)cyclopent-2-en-1-yl)-1H-pyrazol-5-yl)carbamate (265 mg, 0.5 mmol) in Formic Acid (3 mL) was stirred at 60° C. for 12 h. The mixture was concentrated under reduced pressure and purified by silica gel column chromatography, eluting with PE/EA (2:1) to afford the product (125 mg, 66%). LC-MS (ESI): m/z [M+H]⁺=376.2

### Step 3: 5-(3-(4-methylpyrimidin-2-yl)cyclopentyl)-1H-pyrazol-3-amine

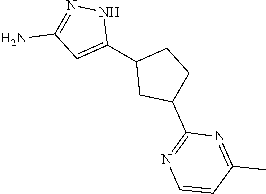 [SMILES: Cc1ccnc(C2CCC(c3cc(N)n[nH]3)C2)n1]

To a mixture of benzyl (5-(3-(4-methylpyrimidin-2-yl)cyclopent-2-en-1-yl)-1H-pyrazol-3-yl)carbamate (125 mg, 0.33 mmol) and H₂O (1 mL) in THF (4 mL) was added Pd/C (63 mg, 0.5 M). The reaction was stirred at RT under H₂ for 12 h. The mixture was filtered through silica pad, the filtrate was concentrated under reduced pressure and purified by silica gel column chromatography, eluting with DCM/MeOH (10:1) to afford the product (36 mg, 44%). LC-MS (ESI): m/z [M+H]⁺=244.2

### Step 4: 4-fluoro-5-((5-(3-(4-methylpyrimidin-2-yl)cyclopentyl)-1H-pyrazol-3-yl)amino)-2,3-dihydrobenzo[d]isothiazole 1,1-dioxide

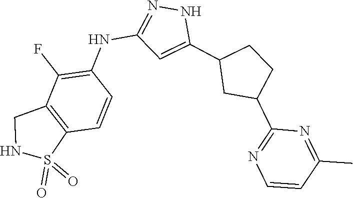 [SMILES: Cc1ccnc(C2CCC(c3cc(Nc4ccc5c(c4F)CNS5(=O)=O)n[nH]3)C2)n1]

To a mixture of 5-bromo-4-fluoro-2,3-dihydrobenzo[d]isothiazole 1,1-dioxide (26 mg, 0.1 mmol) and 5-(3-(4-methylpyrimidin-2-yl)cyclopentyl)-1H-pyrazol-3-amine (24 mg, 0.1 mmol) in t-BuOH (3 mL) was added BrettPhos Pd G3 (9 mg, 0.01 mmol) and K₂CO₃ (42 mg, 0.3 mmol). The reaction mixture was stirred at 110° C. under nitrogen atmosphere for 4 h. The mixture was concentrated under reduced pressure. The residue was purified by prep HPLC (Waters SunFire C18: RD-CO-058 column, eluting with 34%-49% of water (containing 0.1% FA) in acetonitrile to afford the product (6.79 mg, 16%). ¹H NMR (500 MHz, DMSO-d₆) δ 12.04 (s, 1H), 8.72 (s, 1H), 8.58 (d, J=5.1 Hz, 1H), 8.28 (t, J=7.8 Hz, 1H), 7.69 (s, 1H), 7.48 (d, J=8.6 Hz, 1H), 7.20 (d, J=5.1 Hz, 1H), 5.86 (s, 1H), 4.41 (s, 2H), 3.44 (dt, J=16.1, 8.2 Hz, 1H), 3.27-3.19 (m, 1H), 2.48-2.38 (m, 4H), 2.11-1.95 (m, 4H), 1.87-1.76 (m, 1H). LC-MS (ESI): m/z [M+H]⁺=429.3.

### Example 125: 5-((5-(3-(4-ethylpyrimidin-2-yl)cyclopentyl)-1H-pyrazol-3-yl)amino)-4-fluoro-2,3-dihydrobenzo[d]isothiazole 1,1-dioxide

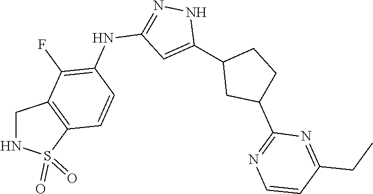 [SMILES: CCc1ccnc(C2CCC(c3cc(Nc4ccc5c(c4F)CNS5(=O)=O)n[nH]3)C2)n1]

The titled compound was synthesized in the procedures similar to Example 1. ¹H NMR (500 MHz, DMSO-d₆) δ=12.04 (s, 1H), 8.72 (d, J=4.7, 1H), 8.61 (d, J=5.1, 1H), 8.27 (s, 1H), 7.69 (t, J=5.0, 1H), 7.48 (d, J=8.5, 1H), 7.21 (d, J=5.1, 1H), 5.87 (d, J=8.6, 1H), 4.41 (d, J=5.0, 2H), 3.58-3.41 (m, 1H), 3.28-3.18 (m, 1H), 2.71 (m, 2H), 2.47-2.31 (m, 1H), 2.24-2.09 (m, 2H), 2.09-1.91 (m, 2H), 1.87-1.73 (m, 1H), 1.22 (m, 3H). LC-MS (ESI): m/z [M+H]⁺=443.3.

### Example 126: 4-fluoro-5-((5-(3-(4-isopropylpyrimidin-2-yl)cyclopentyl)-1H-pyrazol-3-yl)amino)-2,3-dihydrobenzo[d]isothiazole 1,1-dioxide

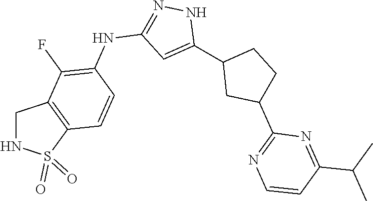 [SMILES: CC(C)c1ccnc(C2CCC(c3cc(Nc4ccc5c(c4F)CNS5(=O)=O)n[nH]3)C2)n1]

The titled compound was synthesized in the procedures similar to Example 1. ¹H NMR (500 MHz, DMSO-d₆) δ 12.04 (s, 1H), 8.72 (s, 1H), 8.62 (d, J=4.9 Hz, 1H), 8.27 (s, 1H), 7.69 (s, 1H), 7.48 (d, J=8.5 Hz, 1H), 7.22 (d, J=5.0 Hz, 1H), 5.87 (d, J=10.0 Hz, 1H), 4.41 (s, 2H), 3.59-3.43 (m, 1H), 3.27-3.15 (m, 1H), 3.00-2.88 (m, 1H), 2.45 (dd, J=12.7, 6.6 Hz, 1H), 2.25-1.95 (m, 4H), 1.82 (dd, J=26.1, 16.8 Hz, 1H), 1.23 (d, J=6.7 Hz, 6H). LC-MS (ESI): m/z [M+H]⁺=457.2.

### Example 127: cis-5-((5-(3-(4-(tert-butyl)pyrimidin-2-yl)cyclopentyl)-1H-pyrazol-3-yl)amino)-4-fluoro-2,3-dihydrobenzo[d]isothiazole 1,1-dioxide

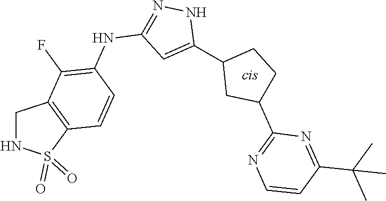 [SMILES: CC(C)(C)c1ccnc(C2CCC(c3cc(Nc4ccc5c(c4F)CNS5(=O)=O)n[nH]3)C2)n1]

The titled compound was synthesized in the procedures similar to Example 1. 1H NMR (500 MHz, DMSO-d₆) δ=12.04 (s, 1H), 8.72 (d, J=2.1 Hz, 1H), 8.63 (d, J=5.3 Hz, 1H), 8.26 (s, 1H), 7.69 (t, J=5.2 Hz, 1H), 7.47 (d, J=8.6 Hz, 1H), 7.33 (d, J=5.3 Hz, 1H), 5.85 (s, 1H), 4.40 (d, J=5.1 Hz, 2H), 3.51-3.43 (m, 2H), 3.25-3.19 (m, 1H), 2.47-2.42 (m, 1H), 2.20-1.94 (m, 5H), 1.88-1.76 (m, 1H), 1.30 (s, 9H). LC-MS (ESI): m/z [M+H]⁺=471.2.

### Example 128: 5-((5-(3-(5-(tert-butyl)pyridin-3-yl)cyclopentyl)-1H-pyrazol-3-yl)amino)-4-fluoro-2,3-dihydrobenzo[d]isothiazole 1,1-dioxide

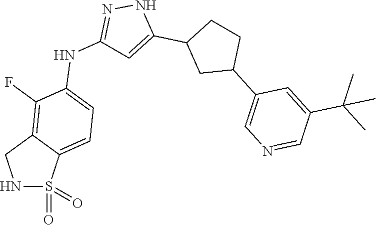 [SMILES: CC(C)(C)c1cncc(C2CCC(c3cc(Nc4ccc5c(c4F)CNS5(=O)=O)n[nH]3)C2)c1]

The titled compound was synthesized in the procedures similar to Example 1. ¹H NMR (500 MHz, DMSO-d₆) δ 12.06 (s, 1H), 8.74 (s, 1H), 8.38 (s, 1H), 8.27 (s, 1H), 8.31-8.22 (m, 1H), 7.73 (s, 1H), 7.69 (t, J=5 Hz, 1H), 7.48 (d, J=10 Hz, 1H), 5.89 (s, 1H), 4.41 (d, J=5 Hz, 2H), 3.29-3.15 (m, 2H), 2.46-2.39 (m, 1H), 2.24-2.12 (m, 2H), 1.95-1.71 (m, 3H), 1.31 (s, 9H). LC-MS (ESI): m/z [M+H]⁺=470.2.

### Example 129: 5-((5-(3-(2-(tert-butyl)pyridin-4-yl)cyclopentyl)-1H-pyrazol-3-yl)amino)-4-fluoro-2,3-dihydrobenzo[d]isothiazole 1,1-dioxide

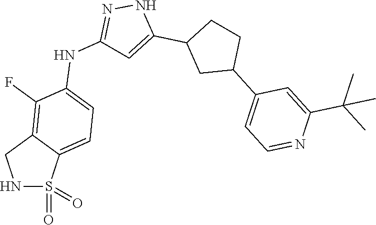 [SMILES: CC(C)(C)c1cc(C2CCC(c3cc(Nc4ccc5c(c4F)CNS5(=O)=O)n[nH]3)C2)ccn1]

The titled compound was synthesized in the procedures similar to Example 1. ¹H NMR (500 MHz, DMSO-d₆) δ 12.08 (s, 1H), 8.74 (s, 1H), 8.53 (s, 1H), 8.13 (s, 1H), 7.38-7.32 (m, 4H), 5.90 (s, 1H), 4.41 (s, 2H), 3.30-3.21 (m, 2H), 2.48-2.42 (m, 1H), 2.26-2.14 (m, 2H), 1.95-1.72 (m, 3H), 1.36 (s, 9H). LC-MS (ESI): m/z [M+H]⁺=470.2.

### Example 130: 5-((5-(3-(6-(tert-butyl)pyrimidin-4-yl)cyclopentyl)-1H-pyrazol-3-yl)amino)-4-fluoro-2,3-dihydrobenzo[d]isothiazole 1,1-dioxide

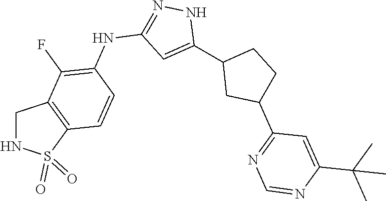 [SMILES: CC(C)(C)c1cc(C2CCC(c3cc(Nc4ccc5c(c4F)CNS5(=O)=O)n[nH]3)C2)ncn1]

The titled compound was synthesized in the procedures similar to Example 1. ¹H NMR (500 MHz, DMSO-d₆) δ=12.04 (d, J=8.9, 1H), 9.03 (m, 1H), 8.73 (s, 1H), 8.28 (d, J=7.0, 1H), 7.69 (s, 1H), 7.48 (d, J=8.6, 1H), 7.43 (s, 1H), 5.88 (s, 1H), 4.41 (d, J=4.4, 2H), 3.43 (m, 1H), 3.23 (m, 1H), 2.45-2.23 (m, 1H), 2.23-2.08 (m, 2H), 2.08-1.72 (m, 3H), 1.31 (d, J=5.1, 9H). LC-MS (ESI): m/z [M+H]⁺=471.35.

### Example 131: 5-((5-(3-(5-(tert-butyl)pyridazin-3-yl)cyclopentyl)-1H-pyrazol-3-yl)amino)-4-fluoro-2,3-dihydrobenzo[d]isothiazole 1,1-dioxide

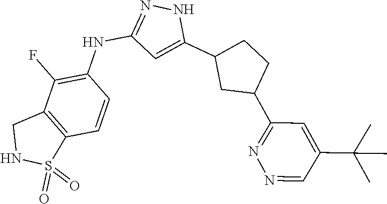 [SMILES: CC(C)(C)c1cnnc(C2CCC(c3cc(Nc4ccc5c(c4F)CNS5(=O)=O)n[nH]3)C2)c1]

The titled compound was synthesized in the procedures similar to Example 1. ¹H NMR (500 MHz, DMSO-d₆) δ 12.08 (s, 1H), 9.20 (s, 1H), 8.74 (s, 1H), 8.28 (t, J=5 Hz, 1H), 7.69 (t, J=5 Hz, 1H), 7.54-7.45 (m, 2H), 5.89 (s, 1H), 4.41 (d, J=5 Hz, 2H), 3.62-3.47 (m, 1H), 3.30-3.24 (m, 1H), 2.49-2.44 (m, 1H), 2.34-2.13 (m, 2H), 2.07-1.83 (m, 3H), 1.31 (s, 9H). LC-MS (ESI): m/z [M+H]⁺=470.2.

### Example 132: 5-((5-(3-(3-(tert-butyl)phenyl)cyclopentyl)-1H-pyrazol-3-yl)amino)-4-fluoro-2,3-dihydrobenzo[d]isothiazole 1,1-dioxide

### Step 1: benzyl (1-(tert-butyl)-3-(3-(3-(tert-butyl)phenyl)cyclopent-2-en-1-yl)-1H-pyrazol-5-yl)carbamate

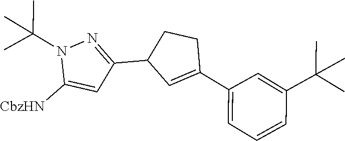 [SMILES: CC(C)(C)c1cccc(C2=CC(c3cc(NC(=O)OCc4ccccc4)n(C(C)(C)C)n3)CC2)c1]

To a mixture of benzyl (1-(tert-butyl)-3-(3-(4,4,5,5-tetramethyl-1,3,2-dioxaborolan-2-yl)cyclopent-2-en-1-yl)-1H-pyrazol-5-yl)carbamate (930 mg, 2 mmol) and 1-bromo-3-(tert-butyl)benzene (426 mg, 2 mmol) in dioxane (20 mL) was added Pd(dppf)Cl₂ (146 mg, 0.2 mmol), K₂CO₃ (828 mg, 6 mmol) and H₂O (2 mL). The reaction mixture was stirred at 80° C. under nitrogen atmosphere for 4 h. The mixture was concentrated under reduced pressure and purified by silica gel column chromatography, eluting with PE/EA (4:1) to afford the product (472 mg, 50%). LC-MS (ESI): m/z [M+H]⁺=472.3

### Step 2: benzyl (3-(3-(3-(tert-butyl)phenyl)cyclopent-2-en-1-yl)-1H-pyrazol-5-yl)carbamate

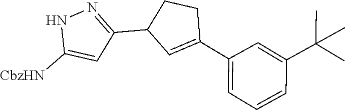 [SMILES: CC(C)(C)c1cccc(C2=CC(c3cc(NC(=O)OCc4ccccc4)[nH]n3)CC2)c1]

A mixture of benzyl (1-(tert-butyl)-3-(3-(3-(tert-butyl)phenyl)cyclopent-2-en-1-yl)-1H-pyrazol-5-yl)carbamate (472 mg, 1 mmol) in Formic Acid (4 mL) was stirred at 60° C. for 12 h. The mixture was concentrated under reduced pressure and purified by silica gel column chromatography, eluting with PE/EA (2:1) to afford the product (332 mg, 80%). LC-MS (ESI): m/z [M+H]⁺=416.3

### Step 3: 5-(3-(3-(tert-butyl)phenyl)cyclopentyl)-1H-pyrazol-3-amine

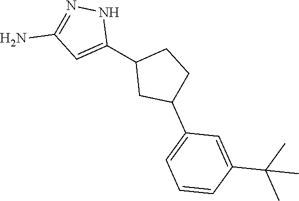 [SMILES: CC(C)(C)c1cccc(C2CCC(c3cc(N)n[nH]3)C2)c1]

To a mixture of benzyl (3-(3-(3-(tert-butyl)phenyl)cyclopent-2-en-1-yl)-1H-pyrazol-5-yl)carbamate (104 mg, 0.25 mmol) and H₂O (1 mL) in THF (4 mL) was added Pd/C (104 mg, 0.5 M). The reaction was stirred at RT under H₂ for 12 h. The mixture was filtered through silica pad, the filtrate was concentrated under reduced pressure and purified by silica gel column chromatography, eluting with DCM/MeOH (10:1) to afford the product (28 mg, 39%). LC-MS (ESI): m/z [M+H]⁺=284.2

### Step 4: 5-((5-(3-(3-(tert-butyl)phenyl)cyclopentyl)-1H-pyrazol-3-yl)amino)-4-fluoro-2,3-dihydrobenzo[d]isothiazole 1,1-dioxide

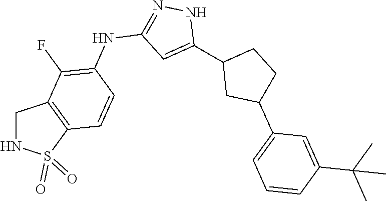 [SMILES: CC(C)(C)c1cccc(C2CCC(c3cc(Nc4ccc5c(c4F)CNS5(=O)=O)n[nH]3)C2)c1]

To a mixture of 5-bromo-4-fluoro-2,3-dihydrobenzo[d]isothiazole 1,1-dioxide (26 mg, 0.1 mmol) and 5-(3-(3-(tert-butyl)phenyl)cyclopentyl)-1H-pyrazol-3-amine (28 mg, 0.1 mmol) in t-BuOH (3 mL) was added BrettPhos Pd G3 (9 mg, 0.01 mmol) and K₂CO₃ (42 mg, 0.3 mmol). The reaction mixture was stirred at 110° C. under nitrogen atmosphere for 4 h. The mixture was concentrated under reduced pressure. The residue was purified by prep HPLC (Waters SunFire C18: RD-CO-058 column, eluting with 34%-49% of water (containing 0.1% FA) in acetonitrile to afford the product (25.9 mg, 55%). ¹H NMR (500 MHz, DMSO) δ 12.04 (s, 1H), 8.73 (s, 1H), 8.27 (s, 1H), 7.69 (t, J=4.6 Hz, 1H), 7.48 (d, J=8.6 Hz, 1H), 7.28 (s, 1H), 7.25-7.19 (m, 2H), 7.10 (d, J=6.0 Hz, 1H), 5.88 (s, 1H), 4.41 (d, J=4.4 Hz, 2H), 3.25-3.12 (m, 2H), 2.44-2.11 (m, 3H), 2.09-1.66 (m, 3H), 1.28-1.27 (m, 9H). LC-MS (ESI): m/z [M+H]⁺=469.3.

### Example 133: 5-((5-(3-(5-ethyloxazol-2-yl)cyclopentyl)-1H-pyrazol-3-yl)amino)-1,3-dihydrobenzo[c]isothiazole 2,2-dioxide

### Step 1: 5-bromo-1-(4-methoxybenzyl)-1,3-dihydrobenzo[c]isothiazole 2,2-dioxide

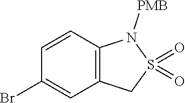

A mixture of 5-bromo-1,3-dihydrobenzo[c]isothiazole 2,2-dioxide (1 g, 3.76 mmol), 4-Methoxybenzyl bromide (1.2 g, 6 mmol) and K₂CO₃ (1.5 g, 11 mmol) in DMF (20 mL) was stirred at 20° C. for 3 h. The solution was concentrated under reduced pressure. The residue was purified by silica gel column chromatography, eluting with PE/EtOAc (3:1) to afford the product (530 mg, 41%), LC-MS (ESI): m/z [M+H]⁺=368.2.

### Step 2: 5-((5-(3-(5-ethyloxazol-2-yl)cyclopentyl)-1H-pyrazol-3-yl)amino)-1-(4-methoxybenzyl)-1,3-dihydrobenzo[c]isothiazole 2,2-dioxide

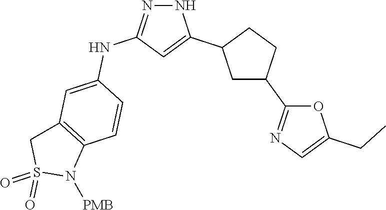

The titled compound was synthesized in the procedures similar to Example 92. LC-MS (ESI): m/z [M+H]⁺=534.2.

### Step 3: 5-((5-(3-(5-ethyloxazol-2-yl)cyclopentyl)-1H-pyrazol-3-yl)amino)-1,3-dihydrobenzo[c]isothiazole 2,2-dioxide

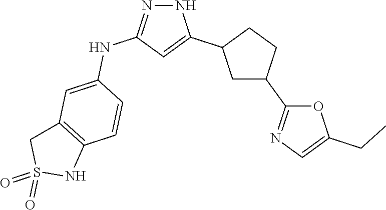 [SMILES: CCc1cnc(C2CCC(c3cc(Nc4ccc5c(c4)CS(=O)(=O)N5)n[nH]3)C2)o1]

5-((5-(3-(5-ethyloxazol-2-yl)cyclopentyl)-1H-pyrazol-3-yl)amino)-1-(4-methoxybenzyl)-1,3-dihydrobenzo[c]isothiazole 2,2-dioxide (64 mg, 0.12 mmol) was dissolved in formic acid (20 mL). The mixture was stirred at 75° C. for 5 h. The resulting mixture was concentrated, and the residue was purified with silica gel column chromatography, eluting with 100% EtOAc to afford the product (35 mg, 72%). ¹H NMR (500 MHz, DMSO-d₆) δ 11.67 (s, 1H), 9.75 (s, 1H), 8.18 (s, 1H), 7.37 (s, 1H), 7.15 (t, J=10 Hz, 1H), 6.73-6.68 (m, 2H), 5.63-5.56 (m, 1H), 4.42 (s, 2H), 3.44-3.35 (m, 1H), 3.24-3.10 (m, 1H), 2.67-2.58 (m, 2H), 2.45-2.38 (m, 1H), 2.20-2.03 (m, 2H), 2.02-1.83 (m, 2H), 1.78-1.66 (m, 1H), 1.20-1.13 (m, 3H). LC-MS (ESI): m/z [M+H]⁺=414.2.

### Example 134: 5-((5-(3-(5-(hydroxymethyl)oxazol-2-yl)cyclopentyl)-1H-pyrazol-3-yl)amino)-1,3-dihydrobenzo[c]isothiazole 2,2-dioxide

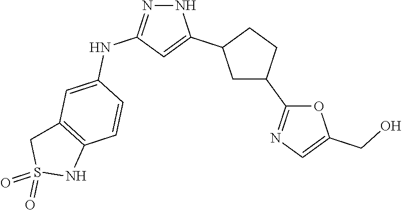 [SMILES: O=S1(=O)Cc2cc(Nc3cc(C4CCC(c5ncc(CO)o5)C4)[nH]n3)ccc2N1]

The titled compound was synthesized in the procedures similar to Example 133. ¹H NMR (500 MHz, DMSO-d₆) δ 11.70 (s, 1H), 9.75 (s, 1H), 8.22 (s, 1H), 7.37 (s, 1H), 7.15 (d, J=7.9 Hz, 1H), 6.89 (d, J=5.9 Hz, 1H), 6.71 (d, J=8.6 Hz, 1H), 5.60 (s, 1H), 5.38-5.25 (m, 1H), 4.48-4.39 (m, 4H), 3.42-3.37 (m, 1H), 3.23-3.12 (m, 1H), 2.47-2.40 (m, 1H), 2.15-2.06 (m, 2H), 2.03-1.95 (m, 1H), 1.93-1.85 (m, 1H), 1.78-1.68 (m, 1H). LC-MS (ESI): m/z [M+H]⁺=416.28.

### Example 135: cis-5-((5-(3-(5-(tert-butyl)oxazol-2-yl)cyclopentyl)-1H-pyrazol-3-yl)amino)-1,3-dihydrobenzo[c]isothiazole 2,2-dioxide

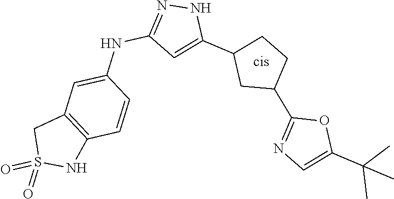 [SMILES: CC(C)(C)c1cnc(C2CCC(c3cc(Nc4ccc5c(c4)CS(=O)(=O)N5)n[nH]3)C2)o1]

The titled compound was synthesized in the procedures similar to Example 133. 1H NMR (500 MHz, DMSO-d₆) δ=11.68 (s, 1H), 9.73 (s, 1H), 8.17 (d, J=15.4 Hz, 1H), 7.36 (s, 1H), 7.15 (d, J=8.1 Hz, 1H), 6.71 (d, J=8.6 Hz, 1H), 6.66 (s, 1H), 5.58 (s, 1H), 4.43 (s, 2H), 3.44-3.34 (dd, J=22.7, 14.4 Hz, 1H), 3.23-3.09 (m, 1H), 2.54 (s, 2H), 2.46-2.37 (m, 1H), 2.15-2.04 (m, 2H), 2.01-1.82 (m, 2H), 1.77-1.67 (m, 1H), 1.21 (s, 9H). LC-MS (ESI): m/z [M+H]⁺=442.2.

### Example 136: 5-((5-(3-(4-(tert-butyl)oxazol-2-yl)cyclopentyl)-1H-pyrazol-3-yl)amino)-1,3-dihydrobenzo[c]isothiazole 2,2-dioxide

### Step 1: 5-((5-(3-(4-(tert-butyl)oxazol-2-yl)cyclopentyl)-1H-pyrazol-3-yl)amino)-1-(4-methoxybenzyl)-1,3-dihydrobenzo[c]isothiazole 2,2-dioxide

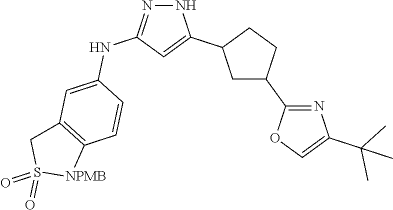

To a mixture of 5-bromo-1-(4-methoxybenzyl)-1,3-dihydrobenzo[c]isothiazole 2,2-dioxide (37 mg, 0.1 mmol) and 5-(3-(4-(tert-butyl)oxazol-2-yl)cyclopentyl)-1H-pyrazol-3-amine (27 mg, 0.1 mmol) in t-BuOH (3 mL) was added BrettPhos Pd G3 (9 mg, 0.01 mmol) and K₂CO₃ (42 mg, 0.3 mmol). The reaction mixture was stirred at 110° C. under nitrogen atmosphere for 4 h. The mixture was concentrated under reduced pressure and purified by silica gel column chromatography, eluting with PE/EA (1:1) to afford the product (28 mg, 50%). LC-MS (ESI): m/z [M+H]⁺=562.3.

### Step 2: 5-((5-(3-(4-(tert-butyl)oxazol-2-yl)cyclopentyl)-1H-pyrazol-3-yl)amino)-1,3-dihydrobenzo[c]isothiazole 2,2-dioxide

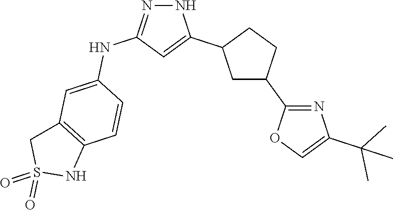 [SMILES: CC(C)(C)c1coc(C2CCC(c3cc(Nc4ccc5c(c4)CS(=O)(=O)N5)n[nH]3)C2)n1]

A mixture of 5-((5-(3-(4-(tert-butyl)oxazol-2-yl)cyclopentyl)-1H-pyrazol-3-yl)amino)-1-(4-methoxybenzyl)-1,3-dihydrobenzo[c]isothiazole 2,2-dioxide (28 mg, 0.5 mmol) and TFA (2 mL) in DCM (2 mL) was stirred at RT for 12 h. The mixture was concentrated under reduced pressure. The residue was purified by prep HPLC (Waters SunFire C18: RD-CO-058 column, eluting with 34%-49% of water (containing 0.1% FA) in acetonitrile to afford the product (2.18 mg, 10%). ¹H NMR (500 MHz, DMSO) δ 7.47 (s, 1H), 7.21 (s, 1H), 7.08 (dd, J=8.5, 2.2 Hz, 1H), 6.83 (d, J=8.5 Hz, 1H), 4.38 (s, 2H), 3.46-3.42 (m, 1H), 3.30-3.23 (m, 1H), 2.59-2.45 (m, 1H), 2.25-1.88 (m, 5H), 1.24 (s, 9H). LC-MS (ESI): m/z [M+H]⁺=442.3.

### Example 137: 5-((5-(3-(5-(tert-butyl)thiazol-2-yl)cyclopentyl)-1H-pyrazol-3-yl)amino)-1,3-dihydrobenzo[c]isothiazole 2,2-dioxide

### Step 1: 5-((5-(3-(5-(tert-butyl)thiazol-2-yl)cyclopentyl)-1H-pyrazol-3-yl)amino)-1-(4-methoxybenzyl)-1,3-dihydrobenzo[c]isothiazole 2,2-dioxide

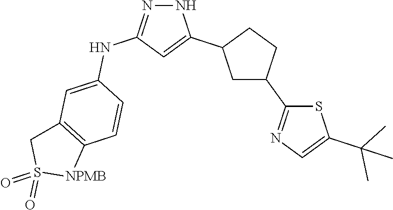

To a mixture of 5-bromo-1-(4-methoxybenzyl)-1,3-dihydrobenzo[c]isothiazole 2,2-dioxide (37 mg, 0.1 mmol) and 5-(3-(5-(tert-butyl)thiazol-2-yl)cyclopentyl)-1H-pyrazol-3-amine (29 mg, 0.1 mmol) in t-BuOH (3 mL) was added BrettPhos Pd G3 (9 mg, 0.01 mmol) and K₂CO₃ (42 mg, 0.3 mmol). The reaction mixture was stirred at 110° C. under nitrogen atmosphere for 4 h. The mixture was concentrated under reduced pressure and purified by silica gel column chromatography, eluting with PE/EA (1:1) to afford the product (15 mg, 25%). LC-MS (ESI): m/z [M+H]⁺=578.3

### Step 2: 5-((5-(3-(5-(tert-butyl)thiazol-2-yl)cyclopentyl)-1H-pyrazol-3-yl)amino)-1,3-dihydrobenzo[c]isothiazole 2,2-dioxide

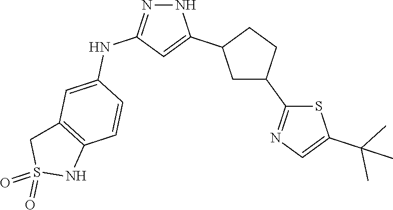 [SMILES: CC(C)(C)c1cnc(C2CCC(c3cc(Nc4ccc5c(c4)CS(=O)(=O)N5)n[nH]3)C2)s1]

A mixture of 5-((5-(3-(5-(tert-butyl)thiazol-2-yl)cyclopentyl)-1H-pyrazol-3-yl)amino)-1-(4-methoxybenzyl)-1,3-dihydrobenzo[c]isothiazole 2,2-dioxide (15 mg, 0.025 mmol) and TFA (2 mL) in DCM (2 mL) was stirred at RT for 12 h. The mixture was concentrated under reduced pressure. The residue was purified by prep HPLC (Waters SunFire C18: RD-CO-058 column, eluting with 34%-49% of water (containing 0.1% FA) in acetonitrile to afford the product (1.02 mg, 9%). ¹H NMR (500 MHz, DMSO) δ 7.36 (s, 1H), 7.22 (s, 1H), 7.06 (dd, J=8.5, 2.4 Hz, 1H), 6.79 (d, J=8.5 Hz, 1H), 4.36 (s, 2H), 3.71-3.62 (m, 1H), 3.41-3.36 (m, 1H), 2.40-2.24 (m, 4H), 2.00-1.84 (m, 2H), 1.39 (s, 10H). LC-MS (ESI): m/z [M+H]⁺=458.3

### Example 138: 5-((5-(3-(5-isopropyloxazol-2-yl)cyclopentyl)-1H-pyrazol-3-yl)amino)-1,3-dihydrobenzo[c]isothiazole 2,2-dioxide

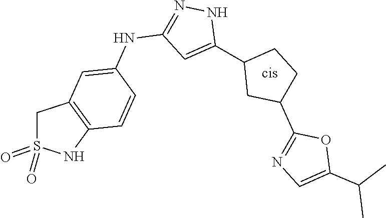 [SMILES: CC(C)c1cnc(C2CCC(c3cc(Nc4ccc5c(c4)CS(=O)(=O)N5)n[nH]3)C2)o1]

The titled compound was synthesized in the procedures similar to Example 133. ¹H NMR (500 MHz, DMSO-d₆) δ 11.69 (s, 1H), 9.74 (s, 1H), 8.21 (s, 1H), 7.41-7.34 (m, 1H), 7.15 (d, J=10 Hz, 1H), 7.74-7.66 (m, 3H), 5.59 (s, 1H), 4.44 (s, 2H), 3.20-3.10 (m, 1H), 2.97-2.88 (m, 1H), 2.46-2.38 (m, 1H), 2.14-2.03 (m, 2H), 2.01-1.92 (m, 1H), 1.92-1.83 (m, 1H), 1.77-1.68 (m, 1H), 1.22-1.16 (m, 6H). LC-MS (ESI): m/z [M+H]⁺=428.2.

### Example 138a and 138b: cis-5-((5-(3-(5-isopropyloxazol-2-yl)cyclopentyl)-1H-pyrazol-3-yl)amino)-1,3-dihydrobenzo[c]isothiazole 2,2-dioxide

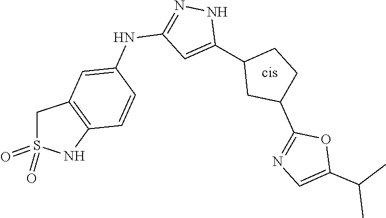 [SMILES: CC(C)c1cnc(C2CCC(c3cc(Nc4ccc5c(c4)CS(=O)(=O)N5)n[nH]3)C2)o1]

The titled compound was synthesized in the procedures similar to Example 133 in a racemic form, which was further separated by Chiral Prep-HPLC to give:

Enantiomer 1 (Example 138a, 90.3% ee); Retention time: 5.324 min. ¹H NMR (500 MHz, DMSO-d₆) δ 11.69 (s, 1H), 9.74 (s, 1H), 8.21 (s, 1H), 7.41-7.34 (m, 1H), 7.15 (d, J=10 Hz, 1H), 7.74-7.66 (m, 3H), 5.59 (s, 1H), 4.44 (s, 2H), 3.20-3.10 (m, 1H), 2.97-2.88 (m, 1H), 2.46-2.38 (m, 1H), 2.14-2.03 (m, 2H), 2.01-1.92 (m, 1H), 1.92-1.83 (m, 1H), 1.77-1.68 (m, 1H), 1.22-1.16 (m, 6H). LC-MS (ESI): m/z [M+H]⁺=428.2.

Enantiomer 2 (Example 138b, 100% ee); Retention time: 6.284 min. ¹H NMR (500 MHz, DMSO-d₆) δ 11.69 (s, 1H), 9.74 (s, 1H), 8.21 (s, 1H), 7.41-7.34 (m, 1H), 7.15 (d, J=10 Hz, 1H), 7.74-7.66 (m, 3H), 5.59 (s, 1H), 4.44 (s, 2H), 3.20-3.10 (m, 1H), 2.97-2.88 (m, 1H), 2.46-2.38 (m, 1H), 2.14-2.03 (m, 2H), 2.01-1.92 (m, 1H), 1.92-1.83 (m, 1H), 1.77-1.68 (m, 1H), 1.22-1.16 (m, 6H). LC-MS (ESI): m/z [M+H]⁺=428.2.

Chiral analytical method: Column: Cellulose-C, 4.6*250 mm 5 μm; Mobile phase: A for Hex, and B for EtOH (0.1% 2M NH₃). Gradient: Mobile Phase A: Mobile Phase B=50:50 (v/v); HPLC Equipment: HPLC-Agilent, Back pressure: 100 bar; Column temperature: 35° C.

Chiral Prep-HPLC Condition: Cellulose-C, 4.6*250 mm 5 μm; Mobile phase: A for Hex, and B for EtOH (0.2% 2M NH₃). Gradient: Mobile Phase A: Mobile Phase B=50:50 (v/v); HPLC Equipment: HPLC-Agilent, Back pressure: 100 bar; Column temperature: 35° C.

### Example 139: cis-5-((5-(3-((2-(tert-butyl)pyridin-3-yl)oxy)cyclopentyl)-1H-pyrazol-3-yl)amino)-1,3-dihydrobenzo[c]isothiazole 2,2-dioxide

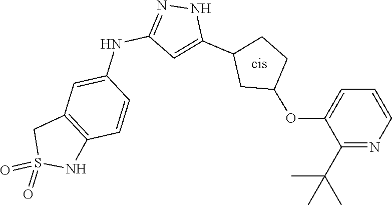 [SMILES: CC(C)(C)c1ncccc1OC1CCC(c2cc(Nc3ccc4c(c3)CS(=O)(=O)N4)n[nH]2)C1]

The titled compound was synthesized in the procedures similar to Example 133. ¹H NMR (500 MHz, DMSO-d₆) δ=11.71 (s, 1H), 9.73 (s, 1H), 8.18 (s, 1H), 8.01 (m, 1H), 7.35 (s, 1H), 7.33-7.25 (m, 1H), 7.18 (m, 1H), 7.14 (m, 1H), 6.70 (d, J=8.6, 1H), 5.54 (s, 1H), 5.02-4.87 (m, 1H), 4.43 (s, 2H), 3.13 (m, 1H), 2.75-2.65 (m, 1H), 2.15-2.08 (m, 1H), 2.04 (m, 1H), 1.97-1.90 (m, 1H), 1.82 (m, 1H), 1.75 (m, 1H), 1.34 (s, 9H). LC-MS (ESI): m/z [M+H]⁺=468.34.

### Example 140: 5-((5-(3-(5-(tert-butyl)oxazol-2-yl)cyclopentyl)-1H-pyrazol-3-yl)amino)-1-methyl-1,3-dihydrobenzo[c]isothiazole 2,2-dioxide

### Step 1: 5-bromo-1-methyl-1,3-dihydrobenzo[c]isothiazole 2,2-dioxide

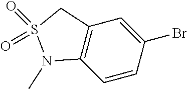

To a stirring solution of 5-bromo-1,3-dihydrobenzo[c]isothiazole 2,2-dioxide (2.5 g, 0.01 mol) in DMF (30 mL) was added K₂CO₃ (2.76 g, 0.02 mol), CH₃I (2.1 g, 0.015 mol). The solution was stirred for 3 h at RT. The solution was diluted with H₂O (50 mL), extracted with EA (50 mL×2). The organic phase was concentrated under reduced pressure. The residue was purified by silica gel column chromatography, eluting with PE/EtOAc (5:1) to afford the product (2.3 g, 94.6%), LC-MS (ESI): m/z [M+Na]⁺=283.9.

### Step 2: 5-((5-(3-(5-(tert-butyl)oxazol-2-yl)cyclopentyl)-1H-pyrazol-3-yl)amino)-1-methyl-1,3-dihydrobenzo[c]isothiazole 2,2-dioxide

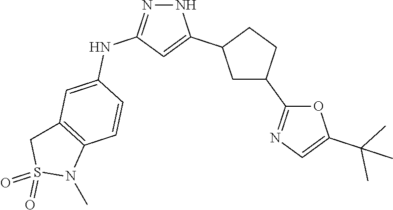 [SMILES: CN1c2ccc(Nc3cc(C4CCC(c5ncc(C(C)(C)C)o5)C4)[nH]n3)cc2CS1(=O)=O]

The titled compound was synthesized in the procedures similar to Example 6. ¹H NMR (500 MHz, DMSO-d₆) δ 11.70 (s, 1H), 8.31-8.20 (m, 1H), 7.40 (s, 1H), 7.25 (t, J=10 Hz, 1H), 6.78 (t, J=10 Hz, 1H), 6.66 (s, 1H), 5.64-5.55 (m, 1H), 4.56 (s, 2H), 3.45-3.34 (m, 1H), 3.24-3.10 (m, 1H), 2.95 (s, 3H), 2.46-2.38 (m, 1H), 2.30-2.02 (m, 2H), 2.01-1.83 (m, 2H), 1.79-1.66 (m, 1H), 1.28-1.20 (m, 9H). LC-MS (ESI): m/z [M+H]⁺=456.2.

### Example 141: 5-((5-((1S,3R)-3-(5-(tert-butyl)oxazol-2-yl)cyclopentyl)-1H-pyrazol-3-yl)amino)-1-methyl-1,3-dihydrobenzo[c]isothiazole 2,2-dioxide

### Step 1: 5-((1S,3R)-3-(5-(tert-butyl)oxazol-2-yl)cyclopentyl)-1H-pyrazol-3-amine

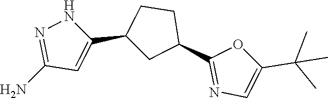 [SMILES: CC(C)(C)c1cnc([C@@H]2CC[C@H](c3cc(N)n[nH]3)C2)o1]

cis-5-(3-(5-(tert-butyl)oxazol-2-yl)cyclopentyl)-1H-pyrazol-3-amine (15.00 g, 54.74 mmol) was purified by Chiral-HPLC with the following conditions: Column: CHIRALPAK IG-3 4.6*100 mm 3 um; Mobile Phase A: H₂O (5 mM NH₄HCO₃)-HPLC, Mobile Phase B: ACN; Flow rate: 1.0 mL/min; Gradient: 60% A to 40% B in 12 min; Wave Length: 220/254 nm; RT (min): 4.787; Sample Solvent: ACN-HPLC; Injection Volume: 3.0 μL; Number Of Runs: 4. This resulted in 3-((1S,3R)-3-(5-(tert-butyl)oxazol-2-yl)cyclopentyl)-1H-pyrazol-5-amine (3.15 g, 21.02%). LC-MS (ES, m/z) [M+H]⁺=275.15, Retention time: 0.618 min. ¹H NMR (300 MHz, DMSO-d₆) δ 11.11 (s, 1H), 6.65 (s, 1H), 5.19 (s, 1H), 4.85-4.13 (s, 2H), 3.34 (s, 1H), 3.11-2.94 (m, 1H), 2.35 (dt, J=13.9 Hz, 1H), 2.13-2.00 (m, 2H), 2.05-1.80 (m, 2H), 1.81-1.60 (m, 1H), 1.23 (s, 9H).

### Step 2: 5-((5-((1S,3R)-3-(5-(tert-butyl)oxazol-2-yl)cyclopentyl)-1H-pyrazol-3-yl)amino)-1-methyl-1,3-dihydrobenzo[c]isothiazole 2,2-dioxide

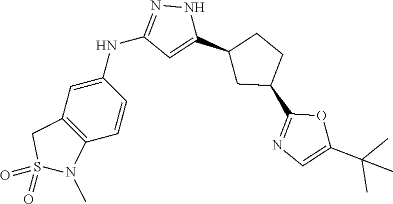 [SMILES: CN1c2ccc(Nc3cc([C@H]4CC[C@@H](c5ncc(C(C)(C)C)o5)C4)[nH]n3)cc2CS1(=O)=O]

The titled compound was synthesized in the procedures similar to Example 6. ¹H NMR (500 MHz, DMSO-d₆) δ=11.69 (s, 1H), 8.22 (s, 1H), 7.41 (s, 1H), 7.25 (d, J=7.1, 1H), 6.78 (d, J=8.6, 1H), 6.66 (s, 1H), 5.58 (s, 1H), 4.56 (s, 2H), 3.39-3.33 (m, 1H), 3.20-3.11 (m, 1H), 2.95 (s, 3H), 2.47-2.38 (m, 1H), 2.13-2.03 (m, 2H), 1.96 (m, 1H), 1.88 (m, 1H), 1.73 (m, 1H), 1.23 (s, 9H). LC-MS (ESI): m/z [M+H]⁺=456.37.

### Example 142: 5-((5-((1R,3S)-3-(5-(tert-butyl)oxazol-2-yl)cyclopentyl)-1H-pyrazol-3-yl)amino)-1-methyl-1,3-dihydrobenzo[c]isothiazole 2,2-dioxide

### Step 1: 5-((1R,3S)-3-(5-(tert-butyl)oxazol-2-yl)cyclopentyl)-1H-pyrazol-3-amine

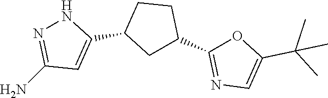

cis-3-(3-(5-(tert-butyl)oxazol-2-yl)cyclopentyl)-1H-pyrazol-5-amine (6.70 g, 20.80 mmol) was purified by Chiral-SFC with the following conditions: CHIRALPAK IC-3 4.6*50 mm 3 um; Mobile Phase A: MTBE (0.1% DEA)-SFC, Mobile Phase B: EtOH-SFC; Flow rate: 1.0 mL/min; Gradient: 90% A to 10% B in 6 min; Wave Length: 220/254 nm; RT1 (min): 3.24; Sample Solvent: EtOH-SFC; Injection Volume: 2.0 L; Number Of Runs: 4. This resulted in 3-((1R,3S)-3-(5-(tert-butyl)oxazol-2-yl)cyclopentyl)-1H-pyrazol-5-amine (1.91 g, 28.51%). LC-MS (ES, m/z) [M+H]⁺=275.15, Retention time: 6.558 min. ¹H NMR (300 MHz, DMSO-d₆) δ 11.11 (s, 1H), 6.65 (s, 1H), 5.19 (s, 1H), 4.85-4.13 (s, 2H), 3.34 (s, 1H), 3.11-2.94 (m, 1H), 2.35 (dt, J=13.9 Hz, 1H), 2.13-2.00 (m, 2H), 2.05-1.80 (m, 2H), 1.81-1.60 (m, 1H), 1.23 (s, 9H).

### Step 2: 5-((5-((1R,3S)-3-(5-(tert-butyl)oxazol-2-yl)cyclopentyl)-1H-pyrazol-3-yl)amino)-1-methyl-1,3-dihydrobenzo[c]isothiazole 2,2-dioxide

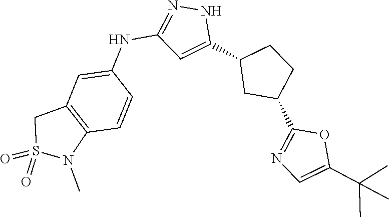 [SMILES: CN1c2ccc(Nc3cc([C@@H]4CC[C@H](c5ncc(C(C)(C)C)o5)C4)[nH]n3)cc2CS1(=O)=O]

The titled compound was synthesized in the procedures similar to Example 6. ¹H NMR (500 MHz, DMSO-d₆) δ=11.69 (s, 1H), 8.22 (s, 1H), 7.41 (s, 1H), 7.25 (d, J=7.1, 1H), 6.78 (d, J=8.7, 1H), 6.66 (s, 1H), 5.58 (s, 1H), 4.56 (s, 2H), 3.38 (m, 1H), 3.20-3.10 (m, 1H), 2.95 (s, 3H), 2.47-2.38 (m, 1H), 2.14-2.03 (m, 2H), 2.02-1.92 (m, 1H), 1.88 (m, 1H), 1.74 (m, 1H), 1.24 (s, 9H). LC-MS (ESI): m/z [M+H]⁺=456.36.

### Example 143: 5-((5-(3-(5-isopropyloxazol-2-yl)cyclopentyl)-1H-pyrazol-3-yl)amino)-1-methyl-1,3-dihydrobenzo[c]isothiazole 2,2-dioxide

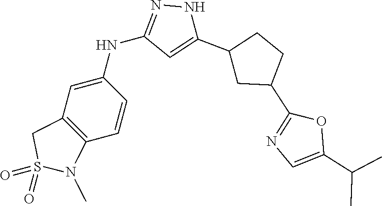 [SMILES: CC(C)c1cnc(C2CCC(c3cc(Nc4ccc5c(c4)CS(=O)(=O)N5C)n[nH]3)C2)o1]

To a mixture of 5-bromo-1-methyl-1,3-dihydrobenzo[c]isothiazole 2,2-dioxide (26 mg, 0.1 mmol) and 5-(3-(5-isopropyloxazol-2-yl)cyclopentyl)-1H-pyrazol-3-amine (26 mg, 0.1 mmol) in t-BuOH (3 mL) was added BrettPhos Pd G3 (9 mg, 0.01 mmol) and K₂CO₃ (42 mg, 0.3 mmol). The reaction was stirred at 110° C. under nitrogen atmosphere for 4 h. The mixture was concentrated under reduced pressure. The residue was purified by prep HPLC (Waters SunFire C18: RD-CO-058 column, eluting with 34%-49% of water (containing 0.1% FA) in acetonitrile to afford the product (6.93 mg, 16%). ¹H NMR (500 MHz, DMSO) δ 8.29-8.10 (m, 1H), 7.40 (d, J=1.4 Hz, 1H), 7.25 (dd, J=8.6, 2.0 Hz, 1H), 6.78 (d, J=8.6 Hz, 1H), 6.69 (s, 1H), 5.59 (s, 1H), 4.56 (s, 2H), 3.23-3.11 (m, 2H), 2.99-2.89 (m, 4H), 2.46-2.39 (m, 1H), 2.10-2.04 (m, 2H), 2.01-1.84 (m, 2H), 1.75-1.71 (m, 1H), 1.19 (d, J=6.9 Hz, 6H). LC-MS (ESI): m/z [M+H]⁺=442.3.

### Example 144: 5-((5-(3-(5-ethyloxazol-2-yl)cyclopentyl)-1H-pyrazol-3-yl)amino)-4-fluoro-1,3-dihydrobenzo[c]isothiazole 2,2-dioxide

### Step 1: S-(2-chloro-6-fluorobenzyl) ethanethioate

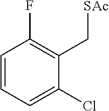

To the solution of 1-chloro-2-(chloromethyl)-3-fluorobenzene (100.0 g, 561.8 mmol, leg) in DMF (1.0 L), was added Potassium thioacetate (128.3 g, 1123.6 mmol, 2.0 eq) at 0° C. Then the reaction was warmed to room temperature and stirred for 1 h. Diluted with water (1 L), extracted with EtOAc (600 mL×3), washed with brine (600 mL×5), dried over Na₂SO₄, concentrated to give product (120.0 g, yield 97.8%). ¹H NMR (300 MHz, DMSO-d₆) δ 7.42-7.33 (m, 2H), 7.27-7.21 (m, 1H), 4.26 (s, 2H), 2.36 (s, 3H).

### Step 2: (2-chloro-6-fluorophenyl)methanesulfonyl chloride

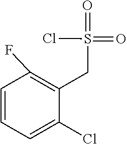 [SMILES: O=S(=O)(Cl)Cc1c(F)cccc1Cl]

To the solution of NCS (244.3 g, 1829.2 mmol, 4.0 eq) in 2N HCl (150 mL) and MeCN (500 mL), a solution of S-(2-chloro-6-fluorobenzyl) ethanethioate (100.0 g, 457.2 mmol, 1.0 eq) in MeCN (500 mL) was added dropwise below 20° C. The mixture was stirred at room temperature for 2 h. Concentrated and diluted with water (200 mL), extracted with EtOAc (300 mL×3), washed with brine (300 mL×2), dried over Na₂SO₄ and concentrated to give product (130.0 g, crude).

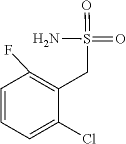 [SMILES: NS(=O)(=O)Cc1c(F)cccc1Cl]

### Step 3: (2-chloro-6-fluorophenyl)methanesulfonamide

Dissolved the (2-chloro-6-fluorophenyl)methanesulfonyl chloride (130.0 g, 534.8 mmol, 1.0 eq) in THF (1.3 L), the mixture was added dropwise to Ammonium hydroxide (28%) (240 mL) at 0° C., then the mixture was stirred at room temperature for 2 h. Then diluted with water (600 mL), extracted with EtOAc (500 mL×3), dried over Na₂SO₄, concentrated to get residue. The residue was triturated with MeCN (200 mL), filtered and the cake was dried under vacuum to give product (72.0 g, yield 68.9% over 2 steps). ¹H NMR (300 MHz, DMSO-d₆) δ 7.48-7.37 (m, 2H), 7.30-7.25 (m, 1H), 7.19 (brs, 2H), 4.48 (s, 2H). LC-MS (ESI): m/z [M+NH₄]⁺=241.0.

### Step 4: 4-fluoro-1,3-dihydrobenzo[c]isothiazole 2,2-dioxide

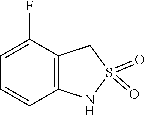 [SMILES: O=S1(=O)Cc2c(F)cccc2N1]

Under N₂ atmosphere, to the solution of (2-chloro-6-fluorophenyl)methanesulfonamide (80.0 g, 357.7 mmol, 1.0 eq) in DMF (800 mL), was added CuI (20.4 g, 107.3 mmol, 0.3 eq), K₃PO₄ (379.6 g, 1788.5 mmol, 4.0 eq) and Sarcosine (159.3 g, 1788 mmol, 5.0 eq). The mixture was stirred at 100° C. for 8 h. Cooled and diluted with water (800 mL), adjusted pH to 2 with HCl (2N), extracted with EtOAc (800 mL×5), washed with brine (800 mL×5), dried over Na₂SO₄, concentrated to get a residue. The residue was triturated with DCM (300 mL), filtered and the cake was dried under vacuum to give product (52.0 g, yield 77.7%). ¹H NMR (400 MHz, CDCl₃) δ 10.88 (brs, 1H), 7.34-7.27 (m, 1H), 6.83 (t, J=8.7 Hz, 1H), 6.67 (d, J=8.1 Hz, 1H), 4.63 (s, 2H). LC-MS (ESI): m/z [M−H]—=186.0.

### Step 5: 5-bromo-4-fluoro-1,3-dihydrobenzo[c]isothiazole 2,2-dioxide

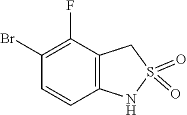 [SMILES: O=S1(=O)Cc2c(ccc(Br)c2F)N1]

Under N₂ atmosphere, to a solution of 4-fluoro-1,3-dihydrobenzo[c]isothiazole 2,2-dioxide (40.0 g, 213.7 mmol, 1.0 eq) in AcOH (200 mL), was added dropwise a solution of Br₂ (37.1 g, 235.0 mmol, 1.1 eq) in AcOH (200 mL). Then the mixture was stirred at 15° C. for 3 h. Quenched with 10% Na₂S₂O₃ (400 mL), and ice water (400 mL) was added, the mixture was filtered and the cake was washed with ice water (200 mL×2), the cake was triturated with MeCN (100 mL), and DCM (100 mL), filtered and the cake was dried under vacuum to give 5-bromo-4-fluoro-1,3-dihydrobenzo[c]isothiazole 2,2-dioxide (29.0 g, yield 51.0%). ¹H NMR (400 MHz, DMSO-d₆): δ 11.08 (brs, 1H), 7.58 (t, J=8.0 Hz, 1H), 6.65 (d, J=8.8 Hz, 1H), 4.74 (s, 2H). LC-MS (ESI): m/z [M+H]⁺=265.9.

### Step 6: 5-bromo-4-fluoro-1-(4-methoxybenzyl)-1,3-dihydrobenzo[c]isothiazole 2,2-dioxide

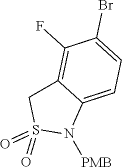

To a solution of 5-bromo-4-fluoro-1,3-dihydrobenzo[c]isothiazole 2,2-dioxide (500 mg, 1.89 mmol) in DMF (10 mL) was added K₂CO₃ (522 mg, 3.78 mmol) and 4-methoxybenzylchloride (444 mg, 2.8 mmol) at 0° C. The result mixture was stirred at room temperature for 2 h. The solution was poured into 20 mL water and extracted with EA. EA layers were combined, dried and concentrated under vacuum. The residue was applied to a silica gel column chromatography, eluting with PE/EtOAc (4:1) to afford the product (512 mg, 70%), LC-MS (ESI): m/z [M+Na]⁺=408.2.

### Step 7: 5-((5-(3-(5-ethyloxazol-2-yl)cyclopentyl)-1H-pyrazol-3-yl)amino)-4-fluoro-1,3-dihydrobenzo[c]isothiazole 2,2-dioxide

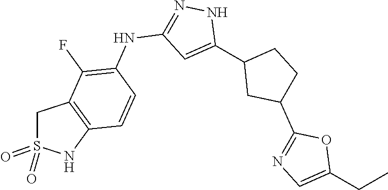 [SMILES: CCc1cnc(C2CCC(c3cc(Nc4ccc5c(c4F)CS(=O)(=O)N5)n[nH]3)C2)o1]

The titled compound was synthesized in the procedures similar to Example 133. ¹H NMR (500 MHz, DMSO-d₆) δ 12.1 (s, 1H), 10.26 (s, 1H), 7.99 (s, 1H), 7.86 (s, 1H), 6.70 (s, 1H), 6.60 (d, J=8.6 Hz, 1H), 5.69 (s, 1H), 4.60 (s, 2H), 3.33 (d, J=8.8 Hz, 1H), 3.21-3.13 (m, 1H), 2.63 (dd, J=5.2, 2.3 Hz, 2H), 2.46-2.39 (m, 1H), 2.21-2.03 (m, 2H), 2.02-1.82 (m, 2H), 1.76-1.63 (m, 1H), 1.16 (td, J=7.5, 3.9 Hz, 3H). LC-MS (ESI): m/z [M+H]⁺=432.2.

### Example 145: cis-5-((5-(3-(5-(tert-butyl)oxazol-2-yl)cyclopentyl)-1H-pyrazol-3-yl)amino)-4-fluoro-1,3-dihydrobenzo[c]isothiazole 2,2-dioxide

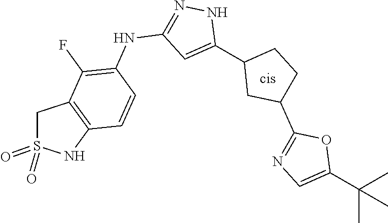 [SMILES: CC(C)(C)c1cnc(C2CCC(c3cc(Nc4ccc5c(c4F)CS(=O)(=O)N5)n[nH]3)C2)o1]

The titled compound was synthesized in the procedures similar to Example 144. 1H NMR (500 MHz, DMSO-d₆) δ=11.73 (s, 1H), 10.20 (s, 1H), 7.93 (s, 1H), 7.86 (s, 1H), 6.66 (s, 1H), 6.58 (d, J=8.6 Hz, 1H), 5.67 (s, 1H), 4.58 (s, 2H), 3.38-3.33 (m, 1H), 3.19-3.10 (m, 1H), 2.46-2.37 (m, 1H), 2.15-2.03 (m, 2H), 2.02-1.92 (m, 1H), 1.90-1.84 (m, 1H), 1.76-1.68 (m, 1H), 1.23 (s, 9H). LC-MS (ESI): m/z [M+H]⁺=460.2.

### Example 146: cis-5-((5-(3-(5-(tert-butyl)-1,3,4-oxadiazol-2-yl)cyclopentyl)-1H-pyrazol-3-yl)amino)-4-fluoro-1,3-dihydrobenzo[c]isothiazole 2,2-dioxide

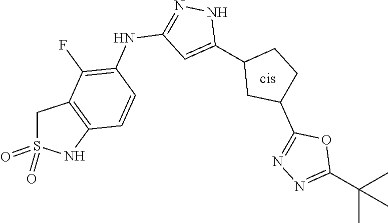 [SMILES: CC(C)(C)c1nnc(C2CCC(c3cc(Nc4ccc5c(c4F)CS(=O)(=O)N5)n[nH]3)C2)o1]

The titled compound was synthesized in the procedures similar to Example 144. ¹H NMR (500 MHz, DMSO-d₆) δ 11.75 (brs, 1H), 10.42 (brs, 1H), 7.88 (d, J=15.0 Hz, 2H), 6.58 (d, J=8.6 Hz, 1H), 5.68 (s, 1H), 4.57 (s, 2H), 3.53-3.45 (m, 1H), 3.24-3.15 (m, 1H), 2.49-2.43 (m, 1H), 2.19-2.07 (m, 2H), 2.04-1.96 (m, 1H), 1.94-1.85 (m, 1H), 1.80-1.70 (m, 1H), 1.33 (s, 9H). LC-MS (ESI): m/z [M+H]⁺=461.32.

### Example 147: trans-5-((5-(3-(5-(tert-butyl)-1,3,4-oxadiazol-2-yl)cyclopentyl)-1H-pyrazol-3-yl)amino)-4-fluoro-1,3-dihydrobenzo[c]isothiazole 2,2-dioxide

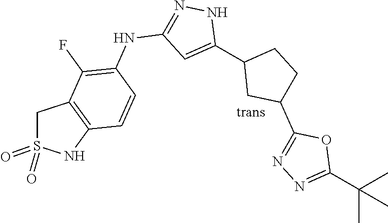 [SMILES: CC(C)(C)c1nnc(C2CCC(c3cc(Nc4ccc5c(c4F)CS(=O)(=O)N5)n[nH]3)C2)o1]

The titled compound was synthesized in the procedures similar to Example 144. ¹H NMR (500 MHz, DMSO-d₆) δ 11.72 (brs, 1H), 10.18 (brs, 1H), 7.86 (s, 2H), 6.58 (d, J=8.6 Hz, 1H), 5.71 (s, 1H), 4.57 (s, 2H), 3.59-3.52 (m, 1H), 3.26-3.17 (m, 1H), 2.33-2.26 (m, 1H), 2.25-2.18 (m, 1H), 2.17-2.13 (m, 1H), 2.12-2.04 (m, 1H), 1.97-1.88 (m, 1H), 1.80-1.69 (m, 1H), 1.35 (s, 9H). LC-MS (ESI): m/z [M+H]⁺=461.30.

### Example 148: 5-((5-(3-(4-(tert-butyl)oxazol-2-yl)cyclopentyl)-1H-pyrazol-3-yl)amino)-4-fluoro-1,3-dihydrobenzo[c]isothiazole 2,2-dioxide

### Step 1: 5-((5-(3-(4-(tert-butyl)oxazol-2-yl)cyclopentyl)-1H-pyrazol-3-yl)amino)-4-fluoro-1-(4-methoxybenzyl)-1,3-dihydrobenzo[c]isothiazole 2,2-dioxide

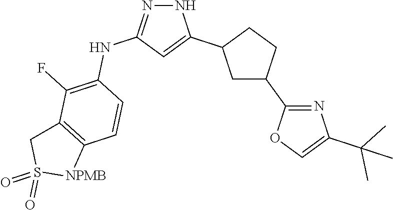

To a mixture of 5-bromo-4-fluoro-1-(4-methoxybenzyl)-1,3-dihydrobenzo[c]isothiazole 2,2-dioxide (38 mg, 0.1 mmol) and 5-(3-(4-(tert-butyl)oxazol-2-yl)cyclopentyl)-1H-pyrazol-3-amine (27 mg, 0.1 mmol) in t-BuOH (3 mL) was added BrettPhos Pd G3 (9 mg, 0.01 mmol) and K₂CO₃ (42 mg, 0.3 mmol). The reaction mixture was stirred at 110° C. under nitrogen atmosphere for 4 h. The mixture was concentrated under reduced pressure and purified by silica gel column chromatography, eluting with PE/EA (1:1) to afford the product (29 mg, 50%). LC-MS (ESI): m/z [M+H]⁺=580.3.

### Step 2: 5-((5-(3-(4-(tert-butyl)oxazol-2-yl)cyclopentyl)-1H-pyrazol-3-yl)amino)-4-fluoro-1,3-dihydrobenzo[c]isothiazole 2,2-dioxide

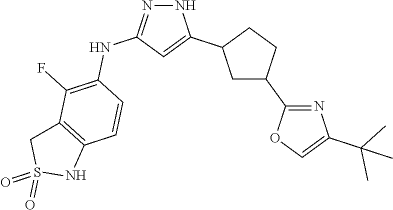 [SMILES: CC(C)(C)c1coc(C2CCC(c3cc(Nc4ccc5c(c4F)CS(=O)(=O)N5)n[nH]3)C2)n1]

A mixture of 5-((5-(3-(4-(tert-butyl)oxazol-2-yl)cyclopentyl)-1H-pyrazol-3-yl)amino)-4-fluoro-1-(4-methoxybenzyl)-1,3-dihydrobenzo[c]isothiazole 2,2-dioxide (29 mg, 0.5 mmol) and TFA (2 mL) in DCM (2 mL) was stirred at RT for 12 h. The mixture was concentrated under reduced pressure. The residue was purified by prep HPLC (Waters SunFire C18: RD-CO-058 column, eluting with 34%-49% of water (containing 0.1% FA) in acetonitrile to afford the product (2.33 mg, 8%). ¹H NMR (500 MHz, DMSO) δ 7.53 (t, J=8.7 Hz, 1H), 7.36 (s, 1H), 6.61 (d, J=8.6 Hz, 1H), 5.78 (s, 1H), 4.44 (s, 2H), 3.74-3.61 (m, 1H), 3.39-3.36 (m, 1H), 2.42-2.24 (m, 4H), 2.00-1.83 (m, 2H), 1.39 (s, 9H). LC-MS (ESI): m/z [M+H]⁺=460.2.

### Example 149: cis-5-((5-(3-(5-(tert-butyl)thiazol-2-yl)cyclopentyl)-1H-pyrazol-3-yl)amino)-4-fluoro-1,3-dihydrobenzo[c]isothiazole 2,2-dioxide

### Step 1: 5-((5-(3-(5-(tert-butyl)thiazol-2-yl)cyclopentyl)-1H-pyrazol-3-yl)amino)-4-fluoro-1-(4-methoxybenzyl)-1,3-dihydrobenzo[c]isothiazole 2,2-dioxide

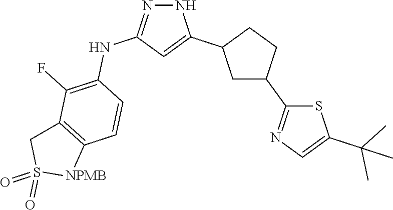

To a mixture of 5-bromo-4-fluoro-1-(4-methoxybenzyl)-1,3-dihydrobenzo[c]isothiazole 2,2-dioxide (38 mg, 0.1 mmol) and 5-(3-(5-(tert-butyl)thiazol-2-yl)cyclopentyl)-1H-pyrazol-3-amine (29 mg, 0.1 mmol) in t-BuOH (3 mL) was added BrettPhos Pd G3 (9 mg, 0.01 mmol) and K₂CO₃ (42 mg, 0.3 mmol). The reaction mixture was stirred at 110° C. under nitrogen atmosphere for 4 h. The mixture was concentrated under reduced pressure and purified by silica gel column chromatography, eluting with PE/EA (1:1) to afford the product (32 mg, 54%). LC-MS (ESI): m/z [M+H]⁺=596.3.

### Step 2: cis-5-((5-(3-(5-(tert-butyl)thiazol-2-yl)cyclopentyl)-1H-pyrazol-3-yl)amino)-4-fluoro-1,3-dihydrobenzo[c]isothiazole 2,2-dioxide

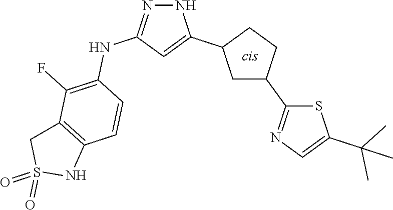 [SMILES: CC(C)(C)c1cnc(C2CCC(c3cc(Nc4ccc5c(c4F)CS(=O)(=O)N5)n[nH]3)C2)s1]

A mixture of 5-((5-(3-(5-(tert-butyl)thiazol-2-yl)cyclopentyl)-1H-pyrazol-3-yl)amino)-4-fluoro-1-(4-methoxybenzyl)-1,3-dihydrobenzo[c]isothiazole 2,2-dioxide (32 mg, 0.54 mmol) and TFA (2 mL) in DCM (2 mL) was stirred at RT for 12 h. The mixture was concentrated under reduced pressure. The residue was purified by prep HPLC (Waters SunFire C18: RD-CO-058 column, eluting with 34%-49% of water (containing 0.1% FA) in acetonitrile to afford the product (2.41 mg, 9%). ¹H NMR (500 MHz, DMSO-d₆) δ 7.52 (t, J=8.7 Hz, 1H), 7.36 (s, 1H), 6.61 (d, J=8.6 Hz, 1H), 5.77 (s, 1H), 4.44 (s, 2H), 3.66-3.56 (m, 1H), 3.27-3.24 (m, 1H), 2.63-2.52 (m, 1H), 2.32-2.21 (m, 2H), 2.06-1.88 (m, 3H), 1.38 (s, 9H). LC-MS (ESI): m/z [M+H]⁺=476.3.

### Example 150: 4-fluoro-5-((5-(3-(5-(2-hydroxypropan-2-yl)-1H-pyrazol-3-yl)cyclopentyl)-1H-pyrazol-3-yl)amino)-1,3-dihydrobenzo[c]isothiazole 2,2-dioxide

### Step 1: 4-fluoro-5-((5-(3-(5-(2-hydroxypropan-2-yl)-1H-pyrazol-3-yl)cyclopentyl)-1H-pyrazol-3-yl)amino)-1-(4-methoxybenzyl)-1,3-dihydrobenzo[c]isothiazole 2,2-dioxide

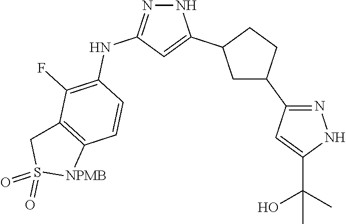

To a mixture of 5-bromo-4-fluoro-1-(4-methoxybenzyl)-1,3-dihydrobenzo[c]isothiazole 2,2-dioxide (76 mg, 0.2 mmol) and 2-(3-(3-(5-amino-1H-pyrazol-3-yl)cyclopentyl)-1H-pyrazol-5-yl)propan-2-ol (55 mg, 0.2 mmol) in t-BuOH (3 mL) was added BrettPhos Pd G3 (18 mg, 0.02 mmol) and K₂CO₃ (84 mg, 0.6 mmol). The reaction mixture was stirred at 110° C. under nitrogen atmosphere for 4 h. The mixture was concentrated under reduced pressure and purified by silica gel column chromatography, eluting with DCM/MeOH (20/1) to afford the product (29 mg, 25%). LC-MS (ESI): m/z [M+H]⁺=581.3.

### Step 2: 4-fluoro-5-((5-(3-(5-(2-hydroxypropan-2-yl)-1H-pyrazol-3-yl)cyclopentyl)-1H-pyrazol-3-yl)amino)-1,3-dihydrobenzo[c]isothiazole 2,2-dioxide

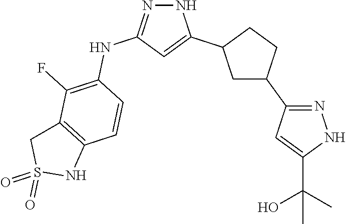 [SMILES: CC(C)(O)c1cc(C2CCC(c3cc(Nc4ccc5c(c4F)CS(=O)(=O)N5)n[nH]3)C2)n[nH]1]

A mixture of 4-fluoro-5-((5-(3-(5-(2-hydroxypropan-2-yl)-1H-pyrazol-3-yl)cyclopentyl)-1H-pyrazol-3-yl)amino)-1-(4-methoxybenzyl)-1,3-dihydrobenzo[c]isothiazole 2,2-dioxide (29 mg, 0.05 mmol) and TFA (2 mL) in DCM (2 mL) was stirred at RT for 12 h. The mixture was concentrated under reduced pressure. The residue was purified by prep HPLC (Waters SunFire C18: RD-CO-058 column, eluting with 34%-49% of water (containing 0.1% FA) in acetonitrile to afford the product (1.7 mg, 7%). H NMR (500 MHz, DMSO) δ 8.67 (s, 1H), 8.49 (s, 2H), 8.36 (s, 1H), 8.24 (s, 1H), 8.13-8.04 (m, 2H), 7.62 (d, J=8.5 Hz, 1H), 7.19-7.09 (m, 2H), 4.59-4.76 (m, 4H), 3.85 (s, 2H), 1.77-1.76 (m, OH), 1.49-1.45 (m, 3H), 1.09-1.06 (m, 2H), 1.00-0.86 (m, 4H). LC-MS (ESI): m/z [M+H]⁺=461.3.

### Example 151: 4-fluoro-5-((5-(3-(5-(prop-1-en-2-yl)-1H-pyrazol-3-yl)cyclopentyl)-1H-pyrazol-3-yl)amino)-1,3-dihydrobenzo[c]isothiazole 2,2-dioxide

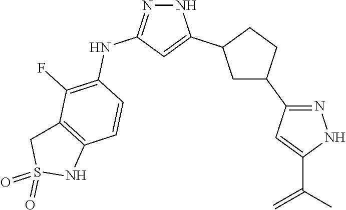 [SMILES: C=C(C)c1cc(C2CCC(c3cc(Nc4ccc5c(c4F)CS(=O)(=O)N5)n[nH]3)C2)n[nH]1]

A mixture of 4-fluoro-5-((5-(3-(5-(2-hydroxypropan-2-yl)-1H-pyrazol-3-yl)cyclopentyl)-1H-pyrazol-3-yl)amino)-1-(4-methoxybenzyl)-1,3-dihydrobenzo[c]isothiazole 2,2-dioxide (29 mg, 0.05 mmol) and TFA (2 mL) in DCM (2 mL) was stirred at RT for 12 h. The mixture was concentrated under reduced pressure. The residue was purified by prep HPLC (Waters SunFire C18: RD-CO-058 column, eluting with 34%-49% of water (containing 0.1% FA) in acetonitrile to afford the product (0.5 mg, 2%). LC-MS (ESI): m/z [M+H]⁺=443.3.

### Example 152: cis-4-fluoro-5-((5-(3-(5-isopropyloxazol-2-yl)cyclopentyl)-1H-pyrazol-3-yl)amino)-1,3-dihydrobenzo[c]isothiazole 2,2-dioxide

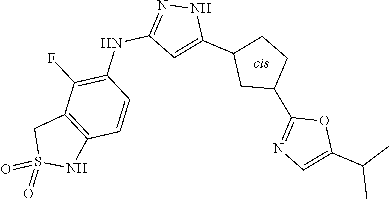 [SMILES: CC(C)c1cnc(C2CCC(c3cc(Nc4ccc5c(c4F)CS(=O)(=O)N5)n[nH]3)C2)o1]

The titled compound was synthesized in the procedures similar to Example 144 in a racemic form, which was separated by chiral HPLC to give:

Enantiomer 1 (Example 152a, 100% ee); Retention time: 2.596 min. ¹H NMR (500 MHz, DMSO-d₆) δ 11.73 (s, 1H), 10.19 (s, 1H), 7.94 (s, 1H), 7.86 (s, 1H), 6.68 (d, J=0.9 Hz, 1H), 6.58 (d, J=8.6 Hz, 1H), 5.68 (s, 1H), 4.58 (s, 2H), 3.37-3.32 (m, 1H), 3.20-3.11 (m, 1H), 2.98-2.88 (m, 1H), 2.46-2.39 (m, 1H), 2.14-2.02 (m, 2H), 2.01-1.92 (m, 1H), 1.87 (dd, J=22.5, 10.4 Hz, 1H), 1.76-1.67 (m, 1H), 1.19 (d, J=6.9 Hz, 6H). LC-MS (ESI): m/z [M+H]⁺=446.3

Enantiomer 2 (Example 152b, 99.76% ee); Retention time: 3.329 min. ¹H NMR (500 MHz, DMSO-d₆) δ 11.73 (s, 1H), 10.19 (s, 1H), 7.94 (s, 1H), 7.86 (s, 1H), 6.68 (d, J=1.1 Hz, 1H), 6.58 (d, J=8.7 Hz, 1H), 5.68 (s, 1H), 4.58 (s, 2H), 3.37-3.32 (m, 1H), 3.20-3.11 (m, 1H), 2.98-2.88 (m, 1H), 2.46-2.39 (m, 1H), 2.14-2.02 (m, 2H), 2.01-1.92 (m, 1H), 1.91-1.82 (m, 1H), 1.76-1.67 (m, 1H), 1.19 (d, J=6.9 Hz, 6H). LC-MS (ESI): m/z [M+H]⁺=446.3

Chiral analytical method: Column: CHIRALPAK IA-3 4.6 mm×50 mm, 3 μm; Mobile phase: A for MTBE (0.1% DEA) and B for MEOH (80/20); Gradient: Mobile Phase A: Mobile Phase B=50:50 (v/v); HPLC Equipmen: HPLC-Agilent, Back pressure: 100 bar; Column temperature: 40° C.

Chiral Prep-HPLC Condition: CHIRALPAK IA 20 mm×250 mm, 5 μm; Mobile phase: A for MTBE and B for MEOH (80/20); Flow Rate: 20 mL/min, Wave Length: UV 220 nm, Prep-HPLC Equipment: Prep-HPLC-Gilson, Back pressure: 100 bar; Column temperature: 25° C.

### Example 153: 5-((5-(3-(5-(tert-butyl)furan-2-yl)cyclopentyl)-1H-pyrazol-3-yl)amino)-4-fluoro-1,3-dihydrobenzo[c]isothiazole 2,2-dioxide

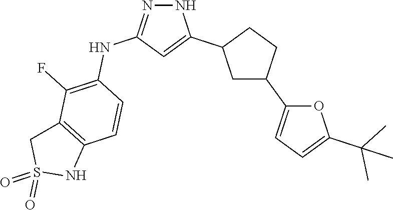 [SMILES: CC(C)(C)c1ccc(C2CCC(c3cc(Nc4ccc5c(c4F)CS(=O)(=O)N5)n[nH]3)C2)o1]

The titled compound was synthesized in the procedures similar to Example 144. LC-MS (ESI): m/z [M+H]⁺=459.2.

### Example 154: 5-((5-(3-(5-(tert-butyl)-4-methyloxazol-2-yl)cyclopentyl)-1H-pyrazol-3-yl)amino)-4-fluoro-1,3-dihydrobenzo[c]isothiazole 2,2-dioxide

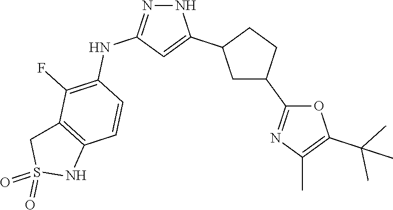 [SMILES: Cc1nc(C2CCC(c3cc(Nc4ccc5c(c4F)CS(=O)(=O)N5)n[nH]3)C2)oc1C(C)(C)C]

The titled compound was synthesized in the procedures similar to Example 144. LC-MS (ESI): m/z [M+H]⁺=474.2.

### Example 155: 5-((5-(3-(4-(tert-butyl)pyridin-2-yl)cyclopentyl)-1H-pyrazol-3-yl)amino)-4-fluoro-1,3-dihydrobenzo[c]isothiazole 2,2-dioxide

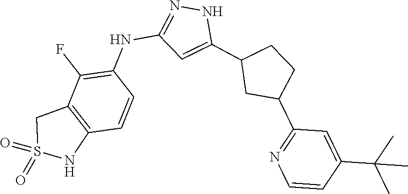 [SMILES: CC(C)(C)c1ccnc(C2CCC(c3cc(Nc4ccc5c(c4F)CS(=O)(=O)N5)n[nH]3)C2)c1]

The titled compound was synthesized in the procedures similar to Example 144. ¹H NMR (500 MHz, DMSO-d₆) δ 11.80 (brs, 1H), 10.20 (s, 1H), 8.44 (d, J=5.2 Hz, 1H), 7.94 (s, 1H), 7.88 (s, 1H), 7.31 (d, J=29.1 Hz, 2H), 6.65-6.53 (m, 1H), 5.72 (d, J=5.0 Hz, 1H), 4.59 (s, 2H), 3.50-3.42 (m, 1H), 3.25-3.15 (m, 1H), 2.42-2.30 (m, 1H), 2.22-2.04 (m, 2H), 2.01-1.73 (m, 3H), 1.28 (d, J=5.1 Hz, 9H). LC-MS (ESI): m/z [M+H]⁺=470.35.

### Example 156: 5-((5-(3-(5-(tert-butyl)pyridin-2-yl)cyclopentyl)-1H-pyrazol-3-yl)amino)-4-fluoro-1,3-dihydrobenzo[c]isothiazole 2,2-dioxide

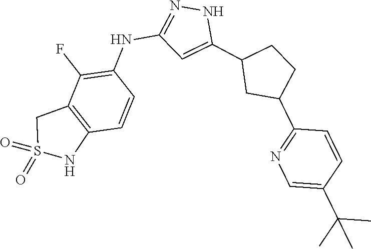 [SMILES: CC(C)(C)c1ccc(C2CCC(c3cc(Nc4ccc5c(c4F)CS(=O)(=O)N5)n[nH]3)C2)nc1]

The titled compound was synthesized in the procedures similar to Example 144. LC-MS (ESI): m/z [M+H]⁺=470.35.

### Example 157: 5-((5-(3-(6-(tert-butyl)pyridin-2-yl)cyclopent-2-en-1-yl)-1H-pyrazol-3-yl)amino)-4-fluoro-1,3-dihydrobenzo[c]isothiazole 2,2-dioxide

### Step 1: benzyl (1-(tert-butyl)-3-(3-(6-(tert-butyl)pyridin-2-yl)cyclopent-2-en-1-yl)-1H-pyrazol-5-yl)carbamate

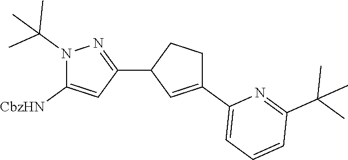 [SMILES: CC(C)(C)c1cccc(C2=CC(c3cc(NC(=O)OCc4ccccc4)n(C(C)(C)C)n3)CC2)n1]

A solution of benzyl (1-(tert-butyl)-3-(3-(4,4,5,5-tetramethyl-1,3,2-dioxaborolan-2-yl)cyclopent-2-en-1-yl)-1H-pyrazol-5-yl)carbamate (930 mg, 2 mmol) in a mixture of dioxane and H₂O (10:1) was added 2-(tert-butyl)-6-chloropyridine (507 mg, 3 mmol), Pd(dppf)Cl₂ (146 mg, 0.2 mmol) and K₂CO₃ (828 mg, 6 mmol). The mixture was heated to 100° C. and stirred for 3 h under nitrogen. The solution was removed under vacuum and the residue was purified by silica gel chromatography, eluting with PE/EA (1:1) to afford the product (613 mg, 65% yield). LC-MS (ESI): m/z [M+H]⁺=473.4.

### Step 2: 5-(3-(6-(tert-butyl)pyridin-2-yl)cyclopent-2-en-1-yl)-1H-pyrazol-3-amine

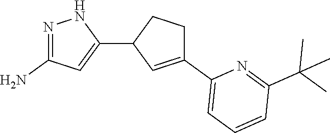 [SMILES: CC(C)(C)c1cccc(C2=CC(c3cc(N)n[nH]3)CC2)n1]

To a solution of benzyl (1-(tert-butyl)-3-(3-(6-(tert-butyl)pyridin-2-yl)cyclopent-2-en-1-yl)-1H-pyrazol-5-yl)carbamate (613 mg, 1.3 mmol) in DCM (15 mL) was added TfOH (3 mL). The mixture was stirred at rt for 2 h. The mixture was quenched by aq NaHCO₃ (50 mL) at 0° C. and extracted with DCM (3×50 mL). The combined organic layers were combined, dried over Na₂SO₄, filtered and concentrated under vacuum. The residue was purified by silica gel chromatography, eluting with DCM/MeOH (0% to 10%) to afford the product (235 mg, 64% yield). LC-MS (ESI): m/z [M+H]⁺=283.3.

### Step 3: 5-((5-(3-(6-(tert-butyl)pyridin-2-yl)cyclopent-2-en-1-yl)-1H-pyrazol-3-yl)amino)-4-fluoro-1,3-dihydrobenzo[c]isothiazole 2,2-dioxide

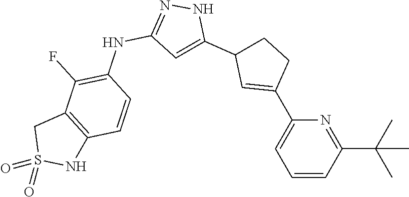 [SMILES: CC(C)(C)c1cccc(C2=CC(c3cc(Nc4ccc5c(c4F)CS(=O)(=O)N5)n[nH]3)CC2)n1]

The titled compound was synthesized in the procedures similar to Example 144. ¹H NMR (500 MHz, DMSO-d₆) δ 11.76 (brs, 1H), 7.94 (s, 1H), 7.86 (s, 1H), 7.68 (dd, J=15.2, 7.4 Hz, 1H), 7.36 (t, J=7.6 Hz, 1H), 7.28 (dd, J=11.6, 7.9 Hz, 1H), 6.68-6.49 (m, 2H), 5.70 (s, 1H), 4.57 (s, 2H), 3.65-3.55 (m, 1H), 3.25-3.15 (m, 1H), 3.00-2.90 (m, 1H), 2.88-2.78 (m, 1H), 2.68-2.58 (m, 2H), 1.31 (d, J=5.2 Hz, 9H). LC-MS (ESI): m/z [M+H]⁺=468.31.

### Example 158: 5-((5-(3-(6-(tert-butyl)pyridin-2-yl)cyclopentyl)-1H-pyrazol-3-yl)amino)-4-fluoro-1,3-dihydrobenzo[c]isothiazole 2,2-dioxide

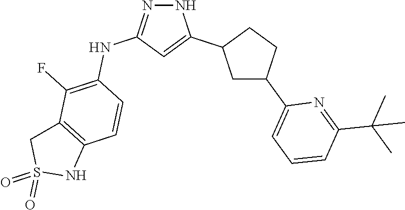 [SMILES: CC(C)(C)c1cccc(C2CCC(c3cc(Nc4ccc5c(c4F)CS(=O)(=O)N5)n[nH]3)C2)n1]

To a solution of 5-((5-(3-(6-(tert-butyl)pyridin-2-yl)cyclopent-2-en-1-yl)-1H-pyrazol-3-yl)amino)-4-fluoro-1,3-dihydrobenzo[c]isothiazole 2,2-dioxide (15 mg, 0.032 mmol) in THF (10 mL) was added Pd/C (10%, wet, 10 mg). The suspension was degassed and purged with hydrogen 3 times. The resulting mixture was stirred at room temperature under a hydrogen balloon for 3 h. The mixture was filtered, and the filter cake was washed with EA (10 mL×3). The filtrate was concentrated under reduced pressure and purified by silica gel column chromatography, eluting with 100% EA to give the product (10.8 mg, 72% yield). ¹H NMR (500 MHz, DMSO-d₆) δ 11.73 (brs, 1H), 10.31 (brs, 1H), 7.93 (s, 1H), 7.86 (s, 1H), 7.61 (t, J=7.7 Hz, 1H), 7.21 (d, J=7.9 Hz, 1H), 7.16-7.03 (m, 1H), 6.58 (d, J=8.5 Hz, 1H), 5.70 (s, 1H), 4.58 (s, 2H), 3.50-3.38 (m, 1H), 3.23-3.12 (m, 1H), 2.45-2.33 (m, 1H), 2.21-2.03 (m, 2H), 1.99-1.71 (m, 3H), 1.30 (s, 9H). LC-MS (ESI): m/z [M+H]⁺=470.36.

### Example 159: 4-fluoro-5-((5-(3-(6-methylpyridin-2-yl)cyclopentyl)-1H-pyrazol-3-yl)amino)-1,3-dihydrobenzo[c]isothiazole 2,2-dioxide

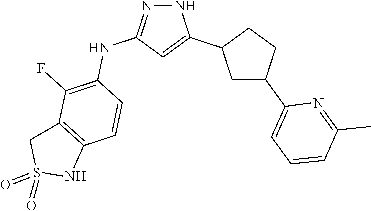 [SMILES: Cc1cccc(C2CCC(c3cc(Nc4ccc5c(c4F)CS(=O)(=O)N5)n[nH]3)C2)n1]

The titled compound was synthesized in the procedures similar to Example 144. ¹H NMR (500 MHz, DMSO-d₆) δ 11.87 (s, 1H), 10.20 (s, 1H), 7.98-7.83 (m, 2H), 7.76-7.55 (m, 1H), 7.31-7.03 (m, 2H), 6.59 (t, J=10 Hz, 1H), 5.72 (s, 1H), 4.56 (s, 2H), 3.25-3.14 (m, 1H), 2.48 (s, 3H), 2.42-2.33 (m, 1H), 2.24-2.06 (m, 3H), 1.98-1.78 (m, 3H). LC-MS (ESI): m/z [M+H]⁺=428.2.

### Example 160: 4-fluoro-5-((5-(3-(6-methoxypyridin-2-yl)cyclopentyl)-1H-pyrazol-3-yl)amino)-1,3-dihydrobenzo[c]isothiazole 2,2-dioxide

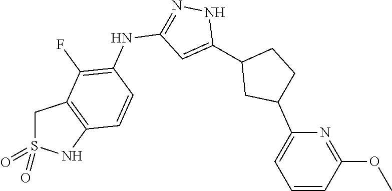 [SMILES: COc1cccc(C2CCC(c3cc(Nc4ccc5c(c4F)CS(=O)(=O)N5)n[nH]3)C2)n1]

The titled compound was synthesized in the procedures similar to Example 144. ¹H NMR (500 MHz, DMSO-d₆) δ 11.76 (s, 1H), 10.50 (s, 1H), 7.97-7.88 (m, 1H), 7.84 (s, 1H), 7.59 (t, J=10 Hz, 1H), 6.91-6.84 (m, 1H), 6.65-6.55 (m, 2H), 5.70 (s, 1H), 4.57 (s, 2H), 3.90-3.80 (m, 3H), 3.29-3.22 (m, 1H), 3.21-3.13 (m, 1H), 2.41-2.32 (m, 1H), 2.25-1.99 (m, 2H), 1.98-1.71 (m, 3H). LC-MS (ESI): m/z [M+H]⁺=444.1.

### Example 161: 5-((5-(3-(2-(tert-butyl)pyridin-4-yl)cyclopentyl)-1H-pyrazol-3-yl)amino)-4-fluoro-1,3-dihydrobenzo[c]isothiazole 2,2-dioxide

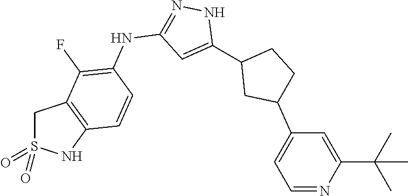 [SMILES: CC(C)(C)c1cc(C2CCC(c3cc(Nc4ccc5c(c4F)CS(=O)(=O)N5)n[nH]3)C2)ccn1]

The titled compound was synthesized in the procedures similar to Example 144. ¹H NMR (500 MHz, DMSO-d₆) δ 11.75 (brs, 1H), 10.18 (brs, 1H), 8.40 (d, J=5.0 Hz, 1H), 7.91 (s, 1H), 7.86 (s, 1H), 7.29 (s, 1H), 7.12 (d, J=4.0 Hz, 1H), 6.58 (d, J=8.6 Hz, 1H), 5.72 (s, 1H), 4.57 (s, 2H), 3.25-3.13 (m, 2H), 2.43-2.35 (m, 1H), 2.20-2.10 (m, 2H), 1.87-1.69 (m, 3H), 1.30 (s, 9H). LC-MS (ESI): m/z [M+H]⁺=470.33.

### Example 162: 5-((5-(3-(5-(tert-butyl)pyridin-3-yl)cyclopentyl)-1H-pyrazol-3-yl)amino)-4-fluoro-1,3-dihydrobenzo[c]isothiazole 2,2-dioxide

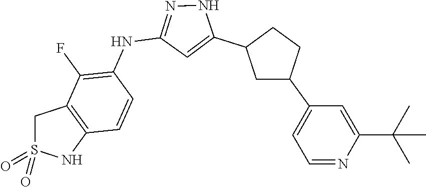 [SMILES: CC(C)(C)c1cc(C2CCC(c3cc(Nc4ccc5c(c4F)CS(=O)(=O)N5)n[nH]3)C2)ccn1]

The titled compound was synthesized in the procedures similar to Example 144. ¹H NMR (500 MHz, DMSO-d₆) δ 11.78 (brs, 1H), 10.20 (s, 1H), 8.47 (d, J=2.3 Hz, 1H), 8.35 (d, J=1.9 Hz, 1H), 7.93 (s, 1H), 7.88 (s, 1H), 7.66 (t, J=2.1 Hz, 1H), 6.59 (d, J=8.6 Hz, 1H), 5.74 (s, 1H), 4.59 (s, 2H), 3.25-3.12 (m, 2H), 2.45-2.35 (m, 1H), 2.21-2.10 (m, 2H), 1.91-1.82 (m, 1H), 1.81-1.70 (m, 2H), 1.31 (d, J=3.9 Hz, 9H). LC-MS (ESI): m/z [M+H]⁺=470.34.

### Example 163: 5-((5-(3-(5-(tert-butyl)pyridazin-3-yl)cyclopentyl)-1H-pyrazol-3-yl)amino)-4-fluoro-1,3-dihydrobenzo[c]isothiazole 2,2-dioxide

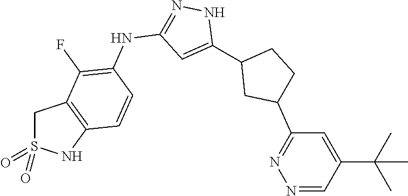 [SMILES: CC(C)(C)c1cnnc(C2CCC(c3cc(Nc4ccc5c(c4F)CS(=O)(=O)N5)n[nH]3)C2)c1]

The titled compound was synthesized in the procedures similar to Example 144. ¹H NMR (500 MHz, DMSO-d₆) δ 11.81 (s, 1H), 10.21 (s, 1H), 9.20 (s, 1H), 7.92 (s, 2H), 7.52 (d, J=5 Hz, 1H), 6.59 (d, J=5 Hz, 1H), 5.74 (s, 1H), 4.59 (s, 2H), 3.58-3.47 (m, 1H), 3.29-3.20 (m, 1H), 2.48-2.42 (m, 1H), 2.25-2.12 (m, 2H), 2.06-1.83 (m, 3H), 1.31 (s, 9H). LC-MS (ESI): m/z [M+H]⁺=471.2.

### Example 164: 5-((5-(3-(4-(tert-butyl)-6-methylpyridin-2-yl)cyclopentyl)-1H-pyrazol-3-yl)amino)-4-fluoro-1,3-dihydrobenzo[c]isothiazole 2,2-dioxide

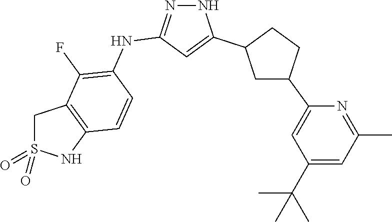 [SMILES: Cc1cc(C(C)(C)C)cc(C2CCC(c3cc(Nc4ccc5c(c4F)CS(=O)(=O)N5)n[nH]3)C2)n1]

The titled compound was synthesized in the procedures similar to Example 144. ¹H NMR (500 MHz, DMSO-d₆) δ 11.85 (s, 1H), 10.38 (s, 1H), 7.92 (d, J=10 Hz, 1H), 7.85 (s, 1H), 7.07 (s, 2H), 6.58 (d, J=10 Hz, 1H), 5.71 (s, 1H), 4.57 (s, 2H), 3.30-3.24 (m, 1H), 3.23-3.14 (m, 1H), 2.43 (s, 3H), 2.38-2.30 (m, 1H), 2.16-2.00 (m, 2H), 1.96-1.76 (m, 3H), 1.25 (s, 9H). LC-MS (ESI): m/z [M+H]⁺=484.2.

### Example 165: 4-fluoro-5-((5-(3-(4-isopropylpyrimidin-2-yl)cyclopentyl)-1H-pyrazol-3-yl)amino)-1,3-dihydrobenzo[c]isothiazole 2,2-dioxide

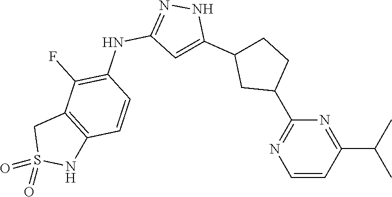 [SMILES: CC(C)c1ccnc(C2CCC(c3cc(Nc4ccc5c(c4F)CS(=O)(=O)N5)n[nH]3)C2)n1]

The titled compound was synthesized in the procedures similar to Example 144. ¹H NMR (500 MHz, DMSO-d₆) δ 11.73 (s, 1H), 10.20 (s, 1H), 8.61 (dd, J=5.2, 1.8 Hz, 1H), 7.95 (s, 1H), 7.87 (d, J=6.0 Hz, 1H), 7.22 (d, J=5.2 Hz, 1H), 6.59 (d, J=8.7 Hz, 1H), 5.71 (d, J=10.6 Hz, 1H), 4.59 (s, 2H), 3.55-3.43 (m, 1H), 3.23-3.13 (m, 1H), 2.99-2.90 (m, 1H), 2.42 (dd, J=12.9, 6.4 Hz, 1H), 2.22-2.14 (m, 1H), 2.11 (dd, J=10.3, 8.0 Hz, 1H), 2.07-2.02 (m, 1H), 2.00-1.92 (m, 1H), 1.86-1.72 (m, 1H), 1.23 (dd, J=6.9, 3.7 Hz, 6H). LC-MS (ESI): m/z [M+H]⁺=457.2.

### Example 166: 6-((5-(3-(5-(tert-butyl)oxazol-2-yl)cyclopentyl)-1H-pyrazol-3-yl)amino)-5-fluoro-3,4-dihydro-1H-benzo[c][1,2]thiazine 2,2-dioxide

### Step 1: 1,4-dibromo-2-fluoro-3-(2-methoxyvinyl)benzene

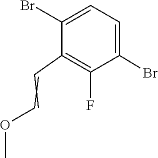

To a solution of (methoxymethyl)triphenylphosphonium chloride (20.7 g, 60.4 mmol) in THF (150 mL) was added KHMDS (71 mL, 71 mmol, 1M) at 0° C. The resulting solution was stirred for 30 min at 0° C. Then the solution of 3,6-dibromo-2-fluorobenzaldehyde (10 g, 35.5 mmol) in THF (20 mL) was added to the reaction at 0° C. The resulting solution was stirred for 2 h at rt under nitrogen atmosphere. The reaction was quenched by the addition of sat.aq. NH₄Cl solution. The resulting solution was extracted with 3×150 mL of EtOAc. The resulting mixture was concentrated under vacuum. The residue was purified by silica gel column chromatography PE to give the product (7.7 g, 70%). LCMS (ESI) m/z [M+H]⁺=311.

### Step 2: 2-(3,6-dibromo-2-fluorophenyl)acetaldehyde

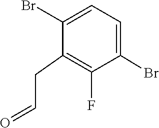 [SMILES: O=CCc1c(Br)ccc(Br)c1F]

The solution of 1,4-dibromo-2-fluoro-3-(2-methoxyvinyl) benzene (7.7 g, 24.8 mmol) in HCl-dioxane (70 mL) was stirred for 1 h at rt. The resulting mixture was concentrated under vacuum. The residue was purified by silica gel column chromatography (EtOAc/PE=4%) to give the product (5.6 g, 76.7%). LCMS (ESI) m/z [M+H]⁺=297.

### Step 3: 2-(3,6-dibromo-2-fluorophenyl)ethan-1-ol

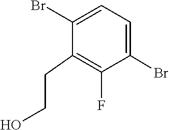 [SMILES: OCCc1c(Br)ccc(Br)c1F]

To a solution of 2-(3,6-dibromo-2-fluorophenyl) acetaldehyde (5.6 g, 19 mmol) in THF (80 mL) was added NaBH₄ (1.4 g, 38 mmol) at 0° C. The resulting solution was stirred for 2 h at rt. The reaction was then quenched by the addition of MeOH. The resulting mixture was concentrated under vacuum. The residue was purified by silica gel column chromatography (EtOAc/PE=12%-40%) to give the product (3.1 g, 54.4%%). LCMS (ESI) m/z [M+H]⁺=299.

### Step 4: 1,4-dibromo-2-(2-bromoethyl)-3-fluorobenzene

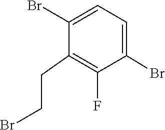 [SMILES: Fc1c(Br)ccc(Br)c1CCBr]

To a solution of 2-(3,6-dibromo-2-fluorophenyl) ethan-1-ol (3.1, 10.4 mmol) in DCM (40 mL) was added PPh₃ (3.5 g, 13.5 mmol). Then the solution of CBr₄ (5.8 g, 17.6 mmol) in DCM (10 mL) was added to the reaction at 0° C. The resulting solution was stirred for 3 h at rt under nitrogen atmosphere. The resulting solution was extracted with 3×30 mL of DCM. The resulting mixture was concentrated under vacuum. The residue was purified by silica gel column chromatography PE to give the product (3.7 g, 97.4%). LCMS (ESI) m/z [M+H]⁺=361.

### Step 5: S-(3,6-dibromo-2-fluorophenethyl) ethanethioate

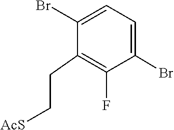

To a solution of 1,4-dibromo-2-(2-bromoethyl)-3-fluorobenzene (3.7 g, 10.2 mmol) in DMF (35 mL) was added potassium ethanethioate (2.3 g, 20.4 mmol) at 0° C. The resulting solution was stirred for 2 h at rt. The resulting solution was extracted with 3×50 mL of EtOAc. The resulting mixture was concentrated under vacuum. The residue was the crude product (3 g, 83.3%). LCMS (ESI) m/z [M+H]⁺=357.

### Step 6: 2-(3,6-dibromo-2-fluorophenyl)ethane-1-sulfonyl chloride

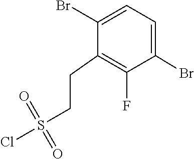 [SMILES: O=S(=O)(Cl)CCc1c(Br)ccc(Br)c1F]

To a solution of NCS (4.5 g, 33.6 mmol) in MeCN (30 mL) and HCl (8 mL, 2M) was added the solution of S-(3,6-dibromo-2-fluorophenethyl) ethanethioate (3 g, 8.4 mmol) in MeCN (10 mL) at 0° C. The resulting solution was stirred for 30 min at 0° C. The resulting solution was extracted with 3×50 mL of EtOAc. The resulting mixture was concentrated under vacuum. The residue was the crude product (3.2 g, 100%). LCMS (ESI) m/z [M+H]⁺=381.

### Step 7: 2-(3,6-dibromo-2-fluorophenyl)ethane-1-sulfonamide

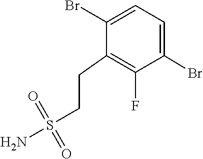 [SMILES: NS(=O)(=O)CCc1c(Br)ccc(Br)c1F]

To a solution of 2-(3,6-dibromo-2-fluorophenyl) ethane-1-sulfonyl chloride (3.2 g, 8.4 mmol) in THF (40 mL) was added NH₃·H₂O (10 mL) at 0° C. The resulting solution was stirred for 1 h at rt. The resulting mixture was concentrated under vacuum. The residue was purified by silica gel column chromatography (MeOH/DCM=31%) to give the product (1.5 g, 46.9%). LCMS (ESI) m/z [M+H]⁺=362.

### Step 8: 6-bromo-5-fluoro-3,4-dihydro-1H-benzo[c][1,2]thiazine 2,2-dioxide

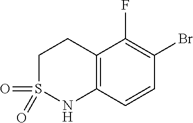 [SMILES: O=S1(=O)CCc2c(ccc(Br)c2F)N1]

To a solution of 2-(3,6-dibromo-2-fluorophenyl)ethane-1-sulfonamide (1.5 g, 4.2 mmol) in DMF (15 mL) were added CuI (223 mg, 2.5 mmol), methylglycine (27 mg, 1.3 mmol), K₃PO₄ (4.5 g, 21 mmol). The resulting solution was stirred for 1 h at 80° C. under nitrogen atmosphere. The resulting solution was extracted with 3×50 mL of EtOAc. The resulting mixture was concentrated under vacuum. The residue was purified by silica gel column chromatography (MeOH/DCM=0%-11%) to give the product (315 mg, 24.2%). LC-MS (ESI): m/z [M+H]⁺=279.9.

### Step 9: 6-bromo-5-fluoro-1-(4-methoxybenzyl)-3,4-dihydro-1H-benzo[c][1,2]thiazine 2,2-dioxide

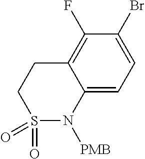

To a stirring mixture of 6-bromo-5-fluoro-3,4-dihydro-1H-benzo[c][1,2]thiazine 2,2-dioxide (280 mg, 1 mmol) and K₂CO₃ (276 mg, 2 mmol) in DMF (5 mL) was added PMBBr (211 mg, 1.05 mmol) dropwise at 0° C. The solution was warmed to room temperature and stirred under nitrogen atmosphere for 1 h. The mixture was concentrated under reduced pressure, and the residue was purified by silica gel column chromatography, eluting with PE/EA (10:1) to afford the product (320 mg, 80%). LC-MS (ESI): m/z [M+Na]⁺=422.1.

### Step 10: 6-((5-(3-(5-(tert-butyl)oxazol-2-yl)cyclopentyl)-1H-pyrazol-3-yl)amino)-5-fluoro-1-(4-methoxybenzyl)-3,4-dihydro-1H-benzo[c][1,2]thiazine 2,2-dioxide

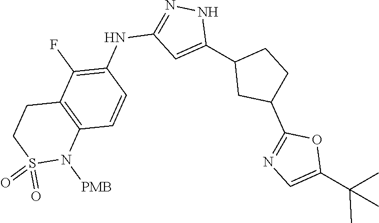

To a mixture of 6-bromo-5-fluoro-1-(4-methoxybenzyl)-3,4-dihydro-1H-benzo[c][1,2]thiazine 2,2-dioxide (84 mg, 0.2 mmol) and 5-(3-(5-(tert-butyl)oxazol-2-yl)cyclopentyl)-1H-pyrazol-3-amine (54 mg, 0.2 mmol) in t-BuOH (3 mL) was added BrettPhos Pd G3 (18 mg, 0.02 mmol) and K₂CO₃ (84 mg, 0.6 mmol). The reaction mixture was stirred at 110° C. under nitrogen atmosphere for 4 h. The mixture was concentrated under reduced pressure and purified by silica gel column chromatography, eluting with 100% EA to afford the product (37 mg, 31%). LC-MS (ESI): m/z [M+H]⁺=594.3.

### Step 11: 6-((5-(3-(5-(tert-butyl)oxazol-2-yl)cyclopentyl)-1H-pyrazol-3-yl)amino)-5-fluoro-3,4-dihydro-1H-benzo[c][1,2]thiazine 2,2-dioxide

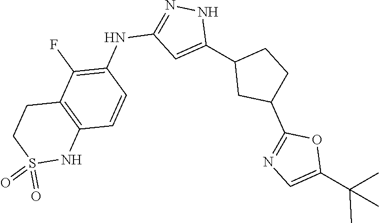 [SMILES: CC(C)(C)c1cnc(C2CCC(c3cc(Nc4ccc5c(c4F)CCS(=O)(=O)N5)n[nH]3)C2)o1]

A mixture of 6-((5-(3-(5-(tert-butyl)oxazol-2-yl)cyclopentyl)-1H-pyrazol-3-yl)amino)-5-fluoro-1-(4-methoxybenzyl)-3,4-dihydro-1H-benzo[c][1,2]thiazine 2,2-dioxide (37 mg, 0.06 mmol) and TFA (2 mL) in DCM (2 mL) was stirred at RT for 12 h. The mixture was concentrated under reduced pressure. The residue was purified by prep HPLC (Waters SunFire C18: RD-CO-058 column, eluting with 30%-41% of water (containing 0.1% FA) in acetonitrile to afford the product (4.8 mg, 17%). ¹H NMR (500 MHz, DMSO-d₆) δ 11.76 (s, 1H), 9.76 (s, 1H), 7.82 (s, 2H), 6.76-6.64 (m, 1H), 6.50 (d, J=8.9 Hz, 1H), 5.70 (d, J=14.3 Hz, 1H), 3.31 (s, 2H), 3.26 (d, J=6.2 Hz, 2H), 3.21-3.11 (m, 1H), 2.46-2.39 (m, 1H), 2.30-2.04 (m, 3H), 2.01-1.82 (m, 2H), 1.71 (ddd, J=22.2, 14.0, 7.1 Hz, 1H), 1.31-1.17 (m, 9H). LC-MS (ESI): m/z [M+H]⁺=474.2.

### Example 167: 5-fluoro-6-((5-(3-(5-isopropyloxazol-2-yl)cyclopentyl)-1H-pyrazol-3-yl)amino)-3,4-dihydro-1H-benzo[c][1,2]thiazine 2,2-dioxide

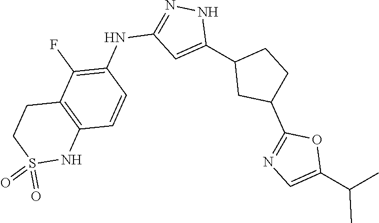 [SMILES: CC(C)c1cnc(C2CCC(c3cc(Nc4ccc5c(c4F)CCS(=O)(=O)N5)n[nH]3)C2)o1]

The titled compound was synthesized in the procedures similar to Example 166. ¹H NMR (500 MHz, DMSO-d₆) δ 11.74 (s, 1H), 9.76 (s, 1H), 7.83 (t, J=11.2 Hz, 2H), 6.68 (t, J=1.8 Hz, 1H), 6.51 (t, J=9.5 Hz, 1H), 5.77-5.64 (m, 1H), 3.44-3.35 (m, 2H), 3.25 (t, J=6.5 Hz, 2H), 3.14 (ddd, J=17.6, 10.0, 7.5 Hz, 1H), 2.97-2.88 (m, 1H), 2.53 (d, J=8.8 Hz, 1H), 2.41 (dd, J=13.3, 6.2 Hz, 1H), 2.13-2.04 (m, 2H), 2.02-1.92 (m, 1H), 1.87 (dt, J=12.2, 10.3 Hz, 1H), 1.76-1.67 (m, 1H), 1.23-1.15 (m, 6H). LC-MS (ESI): m/z [M+H]⁺=460.2.

### Example 168: 5-((5-(3-(5-isopropyloxazol-2-yl)cyclopentyl)-1H-pyrazol-3-yl)amino)-1-methyl-3H-1λ⁴-benzo[d]isothiazole 1-oxide

### Step 1: (4-bromo-2-(bromomethyl)phenyl)(methyl)sulfane

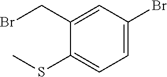

Into a 500-mL round-bottom flask was added (4-bromo-2-methylphenyl)(methyl)sulfane (7.00 g, 32.41 mmol), NBS (11.54 g, 64.82 mmol), AIBN (1.06 g, 6.48 mmol) and DCE (100 mL) at room temperature. The resulting mixture was stirred overnight at 80° C. under nitrogen atmosphere. The resulting mixture was extracted with DCM (2×300 mL). The combined organic layers were washed with brine (2×200 mL) and dried over anhydrous Na₂SO₄. After filtration, the filtrate was concentrated under reduced pressure. The residue was purified by silica gel column chromatography, eluted with PE/EA (100:0-90:10) to afford (4-bromo-2-(bromomethyl)phenyl)(methyl)sulfane (2.0 g, 21% yield).

### Step 2: (2-(azidomethyl)-4-bromophenyl)(methyl)sulfane

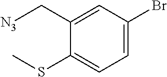

To a solution of (4-bromo-2-(bromomethyl)phenyl)(methyl)sulfane (2.0 g, 6.80 mmol) in the mixture of H₂O (25 mL) and acetone (100 mL) was added NaN₃ (0.53 g, 8.16 mmol) at room temperature. The resulting mixture was stirred at room temperature for 2 h. The mixture was extracted with EtOAc (2×200 mL). The combined organic layers were washed with brine and dried over anhydrous Na₂SO₄. After filtration, the filtrate was concentrated under reduced pressure. The residue was purified by silica gel column chromatography, eluted with PE/EA (100:0-80:20) to afford the product (1.60 g, 91.52% yield).

### Step 3: 2-(azidomethyl)-4-bromo-1-(methylsulfinyl)benzene

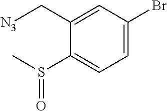

To a solution of (2-(azidomethyl)-4-bromophenyl)(methyl)sulfane (1.50 g, 5.84 mmol) in MeOH (50 mL) was added H₂O₂ (7.94 g, 70.04 mmol) at room temperature. The resulting mixture was stirred 2 h at 60° C. under nitrogen atmosphere. The resulting mixture was extracted with EtOAc (2×100 mL). The combined organic layers were washed with brine and dried over anhydrous Na₂SO₄. After filtration, the filtrate was concentrated under reduced pressure. The residue was purified by silica gel column chromatography, eluted with PE/EA (100:0-60:40) to afford the product (0.86 g, 54% yield). LC-MS (ESI): m/z [M+H]⁺=273.9.

### Step 4: 5-bromo-1-methyl-3H-1λ⁴-benzo[d]isothiazole 1-oxide

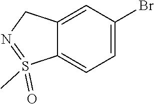

To a solution of 2-(azidomethyl)-4-bromo-1-(methylsulfinyl)benzene (800.0 mg, 2.93 mmol) in toluene (15 mL) was added phthalocyaninato)iron(II) (335.1 mg, 0.59 mmol). The resulting mixture was stirred 5 h at 100° C. under nitrogen atmosphere. The resulting mixture was extracted with EtOAc (2×50 mL). The combined organic layers were washed with brine, dried over anhydrous Na₂SO₄ and filtrated. The filtrate was concentrated under reduced pressure, and the residue was purified by silica gel column chromatography, eluted with PE/EA (100:0-65:35) to afford 5-bromo-1-methyl-3H-114-benzo[d]isothiazole 1-oxide (447.2 mg, 62.3% yield). ¹H NMR (500 MHz, DMSO-d₆) δ 8.04 (d, J=8.2 Hz, 1H), 7.90 (s, 1H), 7.83-7.78 (m, 1H), 4.70 (d, J=17.7 Hz, 1H), 4.56 (d, J=17.7 Hz, 1H), 3.43 (s, 3H). LC-MS (ESI): m/z [M+H]⁺=245.9.

### Step 5: 5-((5-(3-(5-isopropyloxazol-2-yl)cyclopentyl)-1H-pyrazol-3-yl)amino)-1-methyl-3H-1λ⁴-benzo[d]isothiazole 1-oxide

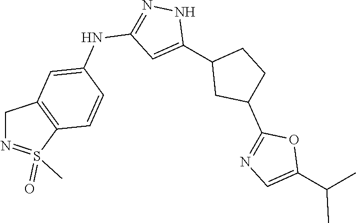 [SMILES: CC(C)c1cnc(C2CCC(c3cc(Nc4ccc5c(c4)CN=S5(C)=O)n[nH]3)C2)o1]

The titled compound was synthesized in the procedures similar to Example 7. ¹H NMR (500 MHz, DMSO-d₆) δ=11.95 (s, 1H), 9.00 (s, 1H), 7.74 (d, J=8.6, 1H), 7.53 (s, 1H), 7.34 (d, J=8.5, 1H), 6.69 (d, J=1.2, 1H), 5.72 (s, 1H), 4.60 (d, J=16.9, 1H), 4.44 (d, J=16.9, 1H), 3.37 (m, 1H), 3.27 (s, 3H), 3.19 (m, 1H), 2.93 (m, 1H), 2.48-2.41 (m, 1H), 2.15-2.05 (m, 2H), 1.98 (m, 1H), 1.90 (m, 1H), 1.80-1.69 (m, 1H), 1.19 (m, 6H). LC-MS (ESI): m/z [M+H]⁺=426.36.

### Example 169: 5-((5-(3-(5-(tert-butyl)oxazol-2-yl)cyclopentyl)-1H-pyrazol-3-yl)amino)-1-methyl-3H-1λ⁴-benzo[d]isothiazole 1-oxide

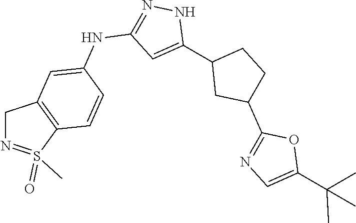 [SMILES: CC(C)(C)c1cnc(C2CCC(c3cc(Nc4ccc5c(c4)CN=S5(C)=O)n[nH]3)C2)o1]

The titled compound was synthesized in the procedures similar to Example 6 in a racemic form, which was further separated by Chiral Prep-HPLC to give:

Enantiomer 1 (Example 169a, 100% ee); Retention time: 8.50 min. ¹H NMR (500 MHz, DMSO-d₆) δ=11.95 (s, 1H), 9.00 (s, 1H), 7.74 (d, J=8.6 Hz, 1H), 7.53 (s, 1H), 7.33 (d, J=7.8 Hz, 1H), 6.67 (s, 1H), 5.71 (s, 1H), 4.60 (d, J=16.9 Hz, 1H), 4.44 (d, J=16.9 Hz, 1H), 3.40-3.34 (m, 1H), 3.27 (s, 3H), 3.22-3.14 (m, 1H), 2.46-2.37 (m, 1H), 2.15-2.03 (m, 2H), 2.02-1.83 (m, 2H), 1.81-1.68 (m, 1H), 1.24 s, 9H) LC-MS (ESI): m/z [M+H]⁺=440.36.

Enantiomer 2 (Example 169b, 61% ee); Retention time: 9.63 min. ¹H NMR (500 MHz, DMSO-d₆) δ=1H NMR (500 MHz, DMSO-d₆) δ1.95 (s, 1H), 9.00 (s, 1H), 7.74 (d, J=8.6 Hz, 1H), 7.53 (s, 1H), 7.33 (d, J=7.8 Hz, 1H), 6.67 (s, 1H), 5.71 (s, 1H), 4.60 (d, J=16.9 Hz, 1H), 4.44 (d, J=16.9 Hz, 1H), 3.40-3.34 (m, 1H), 3.27 (s, 3H), 3.22-3.14 (m, 1H), 2.46-2.37 (m, 1H), 2.15-2.03 (m, 2H), 2.02-1.83 (m, 2H), 1.81-1.68 (m, 1H), 1.24 s, 9H). LC-MS (ESI): m/z [M+H]⁺=440.36;

Enantiomer 3 (Example 169c, 100% ee); Retention time: 11.39 min. ¹H NMR (500 MHz, DMSO-d₆) δ=12.00 (s, 1H), 8.64 (s, 1H), 8.19 (t, J=7.8, 1H), 7.51 (s, 1H), 7.49 (s, 1H), 7.18 (s, 2H), 6.67 (s, 1H), 5.86 (s, 1H), 3.44 (m, 1H), 3.27-3.22 (m, 1H), 2.28 (m, 1H), 2.22-2.11 (m, 2H), 2.08-1.99 (m, 1H), 1.98-1.88 (m, 1H), 1.77-1.66 (m, 1H), 1.24 (s, 9H). LC-MS (ESI): m/z [M+H]⁺=448.30.

Enantiomer 4 (Example 169d, 100% ee); Retention time: 12.06 min¹H NMR (500 MHz, DMSO-d₆) δ=11.95 (s, 1H), 9.00 (s, 1H), 7.74 (d, J=8.6 Hz, 1H), 7.53 (s, 1H), 7.33 (d, J=7.8 Hz, 1H), 6.67 (s, 1H), 5.71 (s, 1H), 4.60 (d, J=16.9 Hz, 1H), 4.44 (d, J=16.9 Hz, 1H), 3.40-3.34 (m, 1H), 3.27 (s, 3H), 3.22-3.14 (m, 1H), 2.46-2.37 (m, 1H), 2.15-2.03 (m, 2H), 2.02-1.83 (m, 2H), 1.81-1.68 (m, 1H), 1.24 s, 9H). LC-MS (ESI): m/z [M+H]⁺=440.33;

Chiral analytical method: Column: CHIRALPAK SA 4.6*250 mm 5 μm; Mobile phase: A for MtBE (0.1% 2M NH₃ MeOH) and B for DCM:MeOH=50:50; Gradient: Mobile Phase A: Mobile Phase B=80:20 (v/v); HPLC Equipment: HPLC-Agilent; Column temperature: 35° C.

Chiral Prep-HPLC Condition: CHIRALPAK SA 20*250 mm 5 μm; Mobile phase: A for MtBE and B for MeOH:DCM=50:50; Gradient: Mobile Phase A: Mobile Phase B=80:20 (v/v); Flow Rate: 20 mL/min, Wavelength: UV 220 nm, Prep-HPLC Equipment: Prep-HPLC-Gilson; Column temperature: 25° C.

### Example 171: 5-((5-(3-(5-(tert-butyl)oxazol-2-yl)cyclopentyl)-1H-pyrazol-3-yl)amino)-4-fluoro-1-methyl-3H-1λ⁴-benzo[d]isothiazole 1-oxide

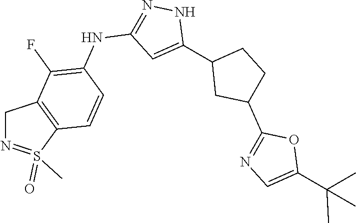 [SMILES: CC(C)(C)c1cnc(C2CCC(c3cc(Nc4ccc5c(c4F)CN=S5(C)=O)n[nH]3)C2)o1]

The titled compound was synthesized in the procedures similar to Example 6. ¹H NMR (500 MHz, DMSO-d₆) δ=12.04 (s, 1H), 8.71 (d, J=2.4, 1H), 8.28 (s, 1H), 7.69 (d, J=8.6, 1H), 6.69-6.65 (m, 1H), 5.86 (d, J=15.3, 1H), 4.73 (d, J=16.8, 1H), 4.57 (d, J=16.8, 1H), 3.46-3.38 (m, 1H), 3.34 (s, 3H), 3.19 (m, 1H), 2.45 (m, 1H), 2.22-2.13 (m, 1H), 2.09 (m, 1H), 2.06-1.96 (m, 1H), 1.91 (m, 1H), 1.80-1.67 (m, 1H), 1.24 (d, J=6.0, 9H). LC-MS (ESI): m/z [M+H]⁺=458.36.

### Example 172: 5-((5-(3-(4-(tert-butyl)pyrimidin-2-yl)cyclopentyl)-1H-pyrazol-3-yl)amino)-4-fluoro-1-methyl-3H-1λ⁴-benzo[d]isothiazole 1-oxide

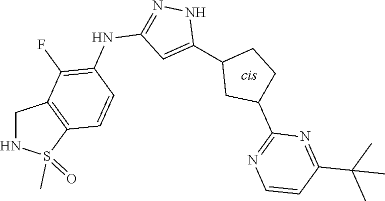

The titled compound was synthesized in the procedures similar to Example 4. ¹H NMR (500 MHz, DMSO-d₆) δ=11.96 (s, 1H), 8.73 (s, 1H), 8.15 (d, J=7.0 Hz, 1H), 7.72-7.49 (m, 2H), 6.65 (s, 1H), 5.83 (s, 1H), 4.94 (s, 1H), 4.30 (d, J=4.7 Hz, 2H), 4.24 (s, 1H), 3.13-2.96 (m, 1H), 2.48-2.39 (m, 1H), 2.06-1.97 (m, 1H), 1.95-1.85 (m, 1H), 1.84-1.75 (m, 6H), 1.73-1.64 (m, 2H), 1.59-1.49 (m, 7H). LC-MS (ESI): m/z [M+H]⁺=467.2.

### Example 173: 5-((5-(3-(5-(tert-butyl)oxazol-2-yl)cyclopentyl)-1H-pyrazol-3-yl)amino)-4-fluoro-1,3-dihydrobenzo[c]thiophene 2,2-dioxide

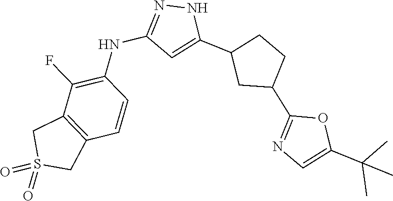 [SMILES: CC(C)(C)c1cnc(C2CCC(c3cc(Nc4ccc5c(c4F)CS(=O)(=O)C5)n[nH]3)C2)o1]

The titled compound was synthesized in the procedures similar to Example 6. ¹H NMR (500 MHz, DMSO-d₆) δ=11.87 (s, 1H), 8.21 (d, J=2.2, 1H), 8.07 (s, 1H), 7.05 (d, J=8.5, 1H), 6.67 (d, J=3.0, 1H), 5.77 (d, J=14.0, 1H), 4.51 (s, 2H), 4.42 (s, 2H), 3.47-3.35 (m, 1H), 3.25-3.12 (m, 1H), 2.43 (m, 1H), 2.21-2.10 (m, 1H), 2.10-2.04 (m, 1H), 1.99 (m, 1H), 1.94-1.84 (m, 1H), 1.78-1.63 (m, 1H), 1.24 (d, J=5.3, 9H). LC-MS (ESI): m/z [M+H]⁺=459.34.

### Example 174: 4-fluoro-5-((5-(3-(5-isopropyloxazol-2-yl)cyclopentyl)-1H-pyrazol-3-yl)amino)-1,3-dihydrobenzo[c]thiophene 2,2-dioxide

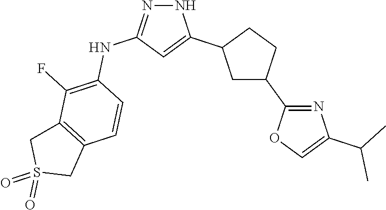 [SMILES: CC(C)c1coc(C2CCC(c3cc(Nc4ccc5c(c4F)CS(=O)(=O)C5)n[nH]3)C2)n1]

To a mixture of 5-bromo-4-fluoro-1,3-dihydrobenzo[c]thiophene 2,2-dioxide (26.5 mg, 0.1 mmol) and 5-(3-(5-isopropyloxazol-2-yl)cyclopentyl)-1H-pyrazol-3-amine (26 mg, 0.1 mmol) in t-BuOH (3 mL) was added BrettPhos Pd G3 (9 mg, 0.01 mmol) and K₂CO₃ (42 mg, 0.3 mmol). The reaction mixture was stirred at 110° C. under nitrogen atmosphere for 4 h. The mixture was concentrated under reduced pressure. The residue was purified by prep HPLC (Waters SunFire C18: RD-CO-058 column, eluting with 34%-49% of water (containing 0.1% FA) in acetonitrile to afford the product (13.68 mg, 31%). ¹H NMR (500 MHz, DMSO) δ 8.22 (s, 1H), 8.06 (t, J=8.3 Hz, 1H), 7.05 (d, J=8.4 Hz, 1H), 6.69 (s, 1H), 5.77 (s, 1H), 4.51 (s, 2H), 4.42 (s, 2H), 3.23-3.13 (m, 2H), 2.96-2.90 (m, 1H), 2.47-2.40 (m, 1H), 2.16-1.84 (m, 4H), 1.79-1.66 (m, 1H), 1.19 (d, J=6.8 Hz, 6H). LC-MS (ESI): m/z [M+H]⁺=445.3.

### Example 175: 5-((5-(3-(4-(tert-butyl)-6-methylpyridin-2-yl)cyclopentyl)-1H-pyrazol-3-yl)amino)-4-fluoro-1,3-dihydrobenzo[c]thiophene 2,2-dioxide

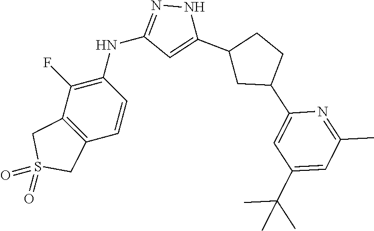 [SMILES: Cc1cc(C(C)(C)C)cc(C2CCC(c3cc(Nc4ccc5c(c4F)CS(=O)(=O)C5)n[nH]3)C2)n1]

The titled compound was synthesized in the procedures similar to Example 2. ¹H NMR (500 MHz, DMSO-d₆) δ 12.01 (s, 1H), 8.25-8.19 (m, 1H), 8.14-8.03 (m, 1H), 7.11-7.03 (m, 3H), 5.80 (s, 1H), 4.51 (s, 2H), 4.42 (s, 2H), 3.30-3.24 (m, 1H), 3.23-3.17 (m, 1H), 2.44 (s, 3H), 2.39-2.31 (m, 1H), 2.18-2.02 (m, 2H), 1.97-1.77 (m, 3H), 1.25 (s, 9H). LC-MS (ESI): m/z [M+H]⁺=483.2.

### Example 176: 5-((5-(3-(5-(tert-butyl)furan-2-yl)cyclopentyl)-1H-pyrazol-3-yl)amino)-4-fluoro-1,3-dihydrobenzo[c]thiophene 2,2-dioxide

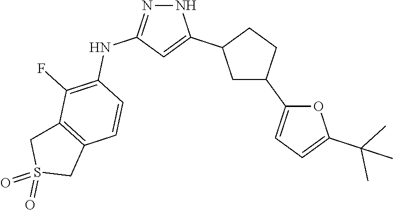 [SMILES: CC(C)(C)c1ccc(C2CCC(c3cc(Nc4ccc5c(c4F)CS(=O)(=O)C5)n[nH]3)C2)o1]

The titled compound was synthesized in the procedures similar to Example 107. ¹H NMR (500 MHz, DMSO-d₆) δ 11.84 (s, 1H), 8.20 (s, 1H), 8.12-8.01 (m, 1H), 7.05 (d, J=10 Hz, 1H), 5.97-5.93 (m, 1H), 5.90-5.87 (m, 1H), 5.76 (s, 1H), 4.51 (s, 2H), 4.42 (s, 2H), 3.25-3.10 (m, 2H), 2.42-2.33 (m, 1H), 2.13-1.98 (m, 2H), 1.83-1.62 (m, 3H), 1.21 (s, 9H). LC-MS (ESI): m/z [M+H]⁺=458.2.

### Example 177: 5-((5-(3-(5-(tert-butyl)-4-methyloxazol-2-yl)cyclopentyl)-1H-pyrazol-3-yl)amino)-4-fluoro-1,3-dihydrobenzo[c]thiophene 2,2-dioxide

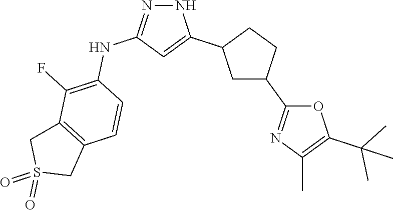 [SMILES: Cc1nc(C2CCC(c3cc(Nc4ccc5c(c4F)CS(=O)(=O)C5)n[nH]3)C2)oc1C(C)(C)C]

The titled compound was synthesized in the procedures similar to Example 111. ¹H NMR (500 MHz, DMSO-d₆) δ 11.88 (s, 1H), 8.24-8.16 (m, 1H), 8.10-8.01 (m, 1H), 7.05 (d, J=10 Hz, 1H), 5.76 (s, 1H), 4.51 (s, 2H), 4.42 (s, 2H), 3.30-3.10 (m, 2H), 2.44-2.35 (m, 1H), 2.17-2.00 (m, 5H), 1.99-1.79 (m, 2H), 1.76-1.66 (m, 1H), 1.27 (s, 9H). LC-MS (ESI): m/z [M+H]⁺=473.2.

### Example 178: 4-fluoro-5-((5-(3-(4-isopropylpyridin-2-yl)cyclopentyl)-1H-pyrazol-3-yl)amino)-1,3-dihydrobenzo[c]thiophene 2,2-dioxide

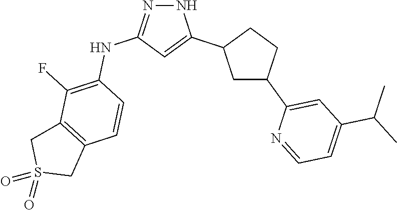 [SMILES: CC(C)c1ccnc(C2CCC(c3cc(Nc4ccc5c(c4F)CS(=O)(=O)C5)n[nH]3)C2)c1]

The titled compound was synthesized in the procedures similar to Example 1. ¹H NMR (500 MHz, DMSO-d₆) δ 11.90 (s, 1H), 8.41 (d, J=5.0 Hz, 1H), 8.21 (s, 1H), 8.09 (s, 1H), 7.20 (s, 1H), 7.12 (s, 1H), 7.05 (d, J=8.5 Hz, 1H), 5.80 (s, 1H), 4.51 (s, 2H), 4.42 (s, 2H), 3.50-3.40 (m, 1H), 3.25-3.16 (m, 1H), 2.92-2.83 (m, 1H), 2.41-2.33 (m, 1H), 2.18-2.05 (m, 2H), 2.00-1.88 (m, 2H), 1.88-1.78 (m, 1H), 1.20 (d, J=6.9 Hz, 6H). LC-MS (ESI): m/z [M+H]⁺=455.34.

### Example 179: 4-fluoro-5-((5-(3-(5-(2-hydroxypropan-2-yl)oxazol-2-yl)cyclopentyl)-1H-pyrazol-3-yl)amino)-1,3-dihydrobenzo[c]thiophene 2,2-dioxide

### Step 1: Benzyl (5-(3-oxocyclopentyl)-1H-pyrazol-3-yl)carbamate

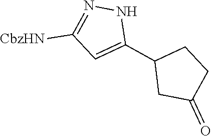 [SMILES: O=C1CCC(c2cc(NC(=O)OCc3ccccc3)n[nH]2)C1]

Into a 250-mL round-bottom flask was added Benzyl (1-(tert-butyl)-5-(3-oxocyclopentyl)-1H-pyrazol-3-yl)carbamate (10.00 g, 28.17 mmol) and formic acid (50 mL) at room temperature. The resulting mixture was stirred for 2 h at 75° C. The resulting mixture was concentrated under reduced pressure to afford Benzyl (5-(3-oxocyclopentyl)-1H-pyrazol-3-yl)carbamate (5.0 g, 59.36%). LC-MS (ESI): m/z [M+H]⁺=300.3.

Step 2: tert-butyl 3-(((benzyloxy)carbonyl)amino)-5-(3-oxocyclopentyl)-1H-pyrazole-1-carboxylate

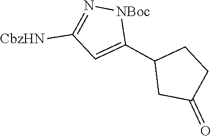 [SMILES: CC(C)(C)OC(=O)n1nc(NC(=O)OCc2ccccc2)cc1C1CCC(=O)C1]

Into a 500-mL round-bottom flask was added Benzyl (5-(3-oxocyclopentyl)-1H-pyrazol-3-yl)carbamate (6.00 g, 20.07 mmol), Boc₂O (6.56 g, 30.10 mmol), Et₃N (2.08 g, 60.20 mmol) and DCM (60 mL) at room temperature. The resulting mixture was stirred 5 h at room temperature. The resulting mixture was extracted with DCM (200 mL). The combined organic layers were washed with brine (2×200 mL), dried over anhydrous Na₂SO₄. After filtration, the filtrate was concentrated under reduced pressure. The residue was purified by silica gel column chromatography, eluted with PE/EA (100:0-90:10) to afford tert-butyl 3-(((benzyloxy)carbonyl)amino)-5-(3-oxocyclopentyl)-1H-pyrazole-1-carboxylate (7.00 g, 87.43%). LC-MS (ESI): m/z [M+H]⁺=400.2.

### Step 3: tert-butyl 3-(((benzyloxy)carbonyl)amino)-5-(3-(((trifluoromethyl)sulfonyl)oxy)cyclopent-2-en-1-yl)-1H-pyrazole-1-carboxylate

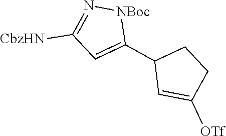 [SMILES: CC(C)(C)OC(=O)n1nc(NC(=O)OCc2ccccc2)cc1C1C=C(OS(=O)(=O)C(F)(F)F)CC1]

Into a 250-mL 3-necked round-bottom flask purged and maintained with an inert atmosphere of nitrogen, were placed tert-butyl 3-(((benzyloxy)carbonyl)amino)-5-(3-oxocyclopentyl)-1H-pyrazole-1-carboxylate (5.00 g, 12.53 mmol) in THF 100 mL, was added LiHMDS (18.80 mL, 18.80 mmol) dropwised at −78° C. under nitrogen atmosphere. The resulting mixture was stirred for 1 h at −78° C. To a solution of PhN(Tf)₂ (6.43 g, 18.80 mmol) in THF 20 mL dropwised at −78° C. The resulting mixture was stirred for 4 h at −78° C.—room temperature. The reaction was quenched with sat NH₄Cl(aq.) at 0° C. The resulting mixture was extracted with EtOAc (200 mL). The combined organic layers were washed with brine (2×200 mL), dried over anhydrous Na₂SO₄. After filtration, the filtrate was concentrated under reduced pressure. The residue was purified by silica gel column chromatography, eluted with PE/EA (100:0~95:5) to afford tert-butyl 3-(((benzyloxy)carbonyl)amino)-5-(3-(((trifluoromethyl)sulfonyl)oxy)cyclopent-2-en-1-yl)-1H-pyrazole-1-carboxylate (4.00 g, 60.11%). LC-MS (ESI): m/z [M+H]⁺=532.2.

### Step 4: tert-butyl 3-(((benzyloxy)carbonyl)amino)-5-(3-(4,4,5,5-tetramethyl-1,3,2-dioxaborolan-2-yl)cyclopent-2-en-1-yl)-1H-pyrazole-1-carboxylate

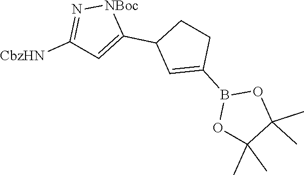 [SMILES: CC(C)(C)OC(=O)n1nc(NC(=O)OCc2ccccc2)cc1C1C=C(B2OC(C)(C)C(C)(C)O2)CC1]

Into a 250-mL round-bottom flask was added tert-butyl 3-(((benzyloxy)carbonyl)amino)-5-(3-(((trifluoromethyl)sulfonyl)oxy)cyclopent-2-en-1-yl)-1H-pyrazole-1-carboxylate (4.00 g, 7.53 mmol), B₂Pin₂ (3.84 g, 15.07 mmol), Pd(dppf)Cl₂ (607.5 mg, 0.75 mmol), KOAc (2.21 g, 22.59 mmol) and Dioxane (50 mL) at room temperature. The resulting mixture was stirred for 4 h at 80° C. under nitrogen atmosphere. The resulting mixture was concentrated under reduced pressure. The residue was purified by silica gel column chromatography, eluted with PE/EtOAc (100:0-97:3) to afford tert-butyl 3-(((benzyloxy)carbonyl)amino)-5-(3-(4,4,5,5-tetramethyl-1,3,2-dioxaborolan-2-yl)cyclopent-2-en-1-yl)-1H-pyrazole-1-carboxylate (3.4 g, 88.51%). LC-MS (ESI): m/z [M+H]⁺=510.4.

### Step 5: methyl 2-(3-(3-(((benzyloxy)carbonyl)amino)-1-(tert-butoxycarbonyl)-1H-pyrazol-5-yl)cyclopent-1-en-1-yl)oxazole-5-carboxylate

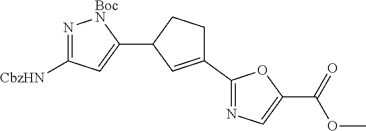 [SMILES: COC(=O)c1cnc(C2=CC(c3cc(NC(=O)OCc4ccccc4)nn3C(=O)OC(C)(C)C)CC2)o1]

Into a 100-mL round-bottom flask was added tert-butyl 3-(((benzyloxy)carbonyl)amino)-5-(3-(4,4,5,5-tetramethyl-1,3,2-dioxaborolan-2-yl)cyclopent-2-en-1-yl)-1H-pyrazole-1-carboxylate (2.40 g, 4.72 mmol), Ethyl 2-bromooxazole-5-carboxylate (1.24 g, 5.66 mmol), Pd(dppf)Cl₂ (380.7 mg, 0.47 mmol), K₃PO₄ (3.0 Og, 14.16 mmol) and Dioxane/H₂O (5/1) (30 mL) at room temperature. The resulting mixture was stirred for 2 h at 90° C. under nitrogen atmosphere. The resulting mixture was concentrated under reduced pressure. The residue was purified by silica gel column chromatography, eluted with PE/EtOAc (100:0~90:10) to afford methyl 2-(3-(3-(((benzyloxy)carbonyl)amino)-1-(tert-butoxycarbonyl)-1H-pyrazol-5-yl)cyclopent-1-en-1-yl)oxazole-5-carboxylate (1.30 g, 54.27%). LC-MS (ESI): m/z [M+H]⁺=509.4.

### Step 6: tert-butyl 3-amino-5-(3-(5-(2-hydroxypropan-2-yl)oxazol-2-yl)cyclopent-2-en-1-yl)-1H-pyrazole-1-carboxylate

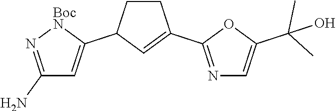 [SMILES: CC(C)(C)OC(=O)n1nc(N)cc1C1C=C(c2ncc(C(C)(C)O)o2)CC1]

Into a 100-mL 3-necked round-bottom flask purged and maintained with an inert atmosphere of nitrogen, were placed Methyl 2-(3-(3-(((benzyloxy)carbonyl)amino)-1-(tert-butoxycarbonyl)-1H-pyrazol-5-yl)cyclopent-1-en-1-yl)oxazole-5-carboxylate (600.0 mg, 1.18 mmol) in THF 10 mL, was added MeMgBr (1.97 mL, 5.91 mmol) dropwised at 0° C. under nitrogen atmosphere. The resulting mixture was stirred for 1 h at 0° C. The reaction was quenched with sat NH₄Cl(aq.) at 0° C. The resulting mixture was extracted with EtOAc (50 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EtOAc (100:0-50:50) to afford tert-butyl 3-amino-5-(3-(5-(2-hydroxypropan-2-yl)oxazol-2-yl)cyclopent-2-en-1-yl)-1H-pyrazole-1-carboxylate (300.0 mg, 67.91%). LC-MS (ESI): m/z [M+H]⁺=375.4.

### Step 7: tert-butyl 3-amino-5-(3-(5-(2-hydroxypropan-2-yl)oxazol-2-yl)cyclopentyl)-1H-pyrazole-1-carboxylate

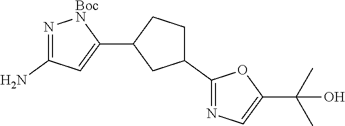 [SMILES: CC(C)(C)OC(=O)n1nc(N)cc1C1CCC(c2ncc(C(C)(C)O)o2)C1]

Into a 100-mL round-bottom flask was added tert-butyl 3-amino-5-(3-(5-(2-hydroxypropan-2-yl)oxazol-2-yl)cyclopent-2-en-1-yl)-1H-pyrazole-1-carboxylate (300.0 mg, 0.80 mmol), Pd/C (300.0 mg) and THF (10 mL) at room temperature. The resulting mixture was stirred for 2 h at room temperature under H₂ atmosphere. The resulting mixture was concentrated under reduced pressure. The residue was purified by silica gel column chromatography, eluted with PE/EtOAc (100:0-20:80) to afford tert-butyl 3-amino-5-(3-(5-(2-hydroxypropan-2-yl)oxazol-2-yl)cyclopentyl)-1H-pyrazole-1-carboxylate (150.0 mg, 49.73%). LC-MS (ESI): m/z [M+H]⁺=377.4.

### Step 8: 2-(2-(3-(3-amino-1H-pyrazol-5-yl)cyclopentyl)oxazol-5-yl)propan-2-ol

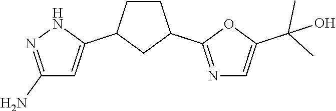 [SMILES: CC(C)(O)c1cnc(C2CCC(c3cc(N)n[nH]3)C2)o1]

Into a 100-mL round-bottom flask was added tert-butyl 3-amino-5-((1S,3R)-3-(5-(2-hydroxypropan-2-yl)oxazol-2-yl)cyclopentyl)-1H-pyrazole-1-carboxylate (220.0 mg, 0.59 mmol), SiO₂ (220.0 mg) and toluene (10 mL) at room temperature. The resulting mixture was stirred 15 h at 100° C. The resulting mixture was concentrated under reduced pressure. The residue was purified by silica gel column chromatography, eluted with DCM/MeOH (100:0-92:8) to afford 2-(2-(3-(3-amino-1H-pyrazol-5-yl)cyclopentyl)oxazol-5-yl)propan-2-ol (90.0 mg, 55.73%). ¹H NMR (300 MHz, Chloroform-d) δ 6.80 (d, J=9.9 Hz, 1H), 5.51 (s, 1H), 3.72 (s, 2H), 3.43 (d, J=6.6 Hz, 2H), 3.33 (s, 1H), 2.49 (dt, J=13.6 Hz, 1H), 2.14 (ddd, J=32.1 Hz, 3H), 1.82 (s, 1H), 1.46 (s, 6H). LC-MS (ESI): m/z [M+H]⁺=277.3.

### Step 9: 4-fluoro-5-((5-(3-(5-(2-hydroxypropan-2-yl)oxazol-2-yl)cyclopentyl)-1H-pyrazol-3-yl)amino)-1,3-dihydrobenzo[c]thiophene 2,2-dioxide

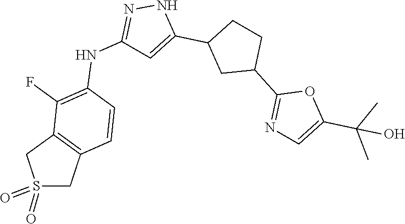 [SMILES: CC(C)(O)c1cnc(C2CCC(c3cc(Nc4ccc5c(c4F)CS(=O)(=O)C5)n[nH]3)C2)o1]

The titled compound was synthesized in the procedures similar to Example 110. ¹H NMR (500 MHz, DMSO-d₆) δ 11.87 (s, 1H), 8.22 (d, J=2.5 Hz, 1H), 8.08 (s, 1H), 7.05 (d, J=8.5 Hz, 1H), 6.78 (s, 1H), 5.77 (s, 1H), 5.23 (s, 1H), 4.51 (s, 2H), 4.42 (s, 2H), 3.39 (d, J=9.5 Hz, 1H), 3.17 (td, J=10.0, 5.0 Hz, 1H), 2.45 (dt, J=12.4, 7.5 Hz, 1H), 2.13-2.091 (m, 2H), 2.02-1.94 (m, 1H), 1.89 (dt, J=12.3, 10.2 Hz, 1H), 1.77-1.69 (m, 1H), 1.42 (s, 6H). LC-MS (ESI): m/z [M+H]⁺=461.3.

### CDK2/Cyclin E1 and CDK1/Cyclin B1 Biochemical Assays

Compounds disclosed herein were tested for inhibition of CDK2/Cyclin E1 or CDK1/Cyclin B1 kinase in an assay based on the time-resolved fluorescence-resonance energy transfer (TR-FRET) methodology. The assay was carried out in 384-well low volume black plates in a reaction mixture containing CDK2/Cyclin E1 or CDK1/Cyclin B1, 0.060 mM ATP for CDK2/Cyclin E1 or 0.015 mM ATP for CDK1/Cyclin B1, 0.15 μM Rb (Ser780)-biotin substrate and 0-10 μM compound in buffer containing 50 mM HEPES pH7.0, 0.02% NaN₃, 0.01% BSA, 0.1 mM Orthovanadate, 50 mM MgCl₂, 1 mM DTT and 0.005% Tween-20. The kinase was incubated with compound for 60 minutes at room temperature and the reaction was initiated by the addition of ATP and Rb (Ser780)-biotin substrate. After reaction at room temperature for 120 minutes, an equal volume of stop/detection solution was added according to the manufacture's instruction (Cisbio Bioassays). The stop/detection solution contained Streptavidin-XL665 and Anti-pRb (Ser780) mAb-Eu Cryptate in Detection buffer (Cisbio Bioassays). Plates were incubated at room temperature for 60 minutes, and the TR-FRET signals (ex337 nm, em665 nm/620 nm) were recorded on a PHERAstar FSX plate reader (BMG Labtech). The inhibition percentage of CDK2/Cyclin E1 or CDK1/Cyclin B1 kinase activity in presence of increasing concentrations of compounds was calculated based on the ratio of fluorescence at 665 nm to that at 620 nm. The IC₅₀ for each compound was derived from fitting the data to the four-parameter logistic equation by Dotmatics. The IC₅₀ data as measured for the Examples are shown in Table 1.

TABLE 1

| | CDK2/Cyclin | CDK1/Cyclin |
|---|---|---|
| Example | E1 IC₅₀ (nM) | B1 IC₅₀ (nM) |
| 21 | 14 | 142 |
| 22 | 43 | 239 |
| 23 | 132 | 526 |
| 24 | 5.7 | 49 |
| 25 | 1.4 | 11 |
| 26 | 140 | 1692 |
| 27 | 0.7 | 3.7 |
| 28 | 130 | 1243 |
| 29 | 2.9 | 35 |
| 30 | 2080 | >10000 |
| 31 | 8.5 | 73 |
| 32 | 48 | 3572 |
| 33 | 7.5 | 703 |
| 34 | 63 | 1551 |
| 35 | 2.6 | 35 |
| 39 | 55 | 2331 |
| 40 | 19 | 552 |
| 41 | 33 | 474 |
| 42 | 171 | 531 |
| 43 | 4.3 | 58 |
| 44 | 1404 | 8336 |
| 45 | 764 | 7123 |
| 46 | 419 | 7449 |
| 47 | 3435 | >10000 |
| 48 | 38 | 425 |
| 49 | 478 | 2562 |
| 50 | 33 | 1322 |
| 51 | 214 | 1768 |
| 52 | 7.1 | 121 |
| 53 | 4.1 | 118 |
| 54 | 49 | 418 |
| 55 | 14 | 486 |
| 57 | 341 | 8295 |
| 58 | 269 | 7198 |
| 59 | 7.6 | 556 |
| 60 | 12 | 732 |
| 61 | 1 | 56 |
| 62 | 24 | 571 |
| 63 | 90 | 3121 |
| 64 | 296 | 1904 |
| 65 | 70 | 1362 |
| 66 | 2532 | >10000 |
| 67 | 54 | 2146 |
| 68 | 12 | 198 |
| 69 | 829 | >10000 |
| 70 | >10000 | >10000 |
| 71 | 391 | 7809 |
| 73 | 12 | 445 |
| 74a | 4.9 | 301 |
| 74b | 18 | 671 |
| 75 | 53 | 1072 |
| 76 | 53 | 1613 |
| 77 | 530 | >10000 |
| 78 | 306 | >10000 |
| 79 | 10 | 486 |
| 81 | 1185 | >10000 |
| 82 | 602 | >10000 |
| 83 | 710 | >10000 |
| 84a | 7.6 | 237 |
| 84b | 14 | 142 |
| 85 | 31 | 289 |
| 86 | 73 | 1343 |
| 87 | 229 | 3253 |
| 91a | 1.3 | 53 |
| 91b | 23 | 2572 |
| 92 | 21 | 509 |
| 93 | 70 | 1759 |
| 133 | 6.2 | 82 |
| 134 | 41 | 1113 |
| 135 | 4.6 | 11 |
| 144 | 16 | 334 |
| 145 | 3.3 | 11 |

### CDK2/Cyclin E1 and CDK1/Cyclin B1 Biochemical Assays

Compounds disclosed herein were tested for inhibition of CDK2/Cyclin E1 or CDK1/Cyclin B kinase in an assay based on the time-resolved fluorescence-resonance energy transfer (TR-FRET) methodology. The assay was carried out in 384-well low volume black plates in a reaction mixture containing CDK2/Cyclin E1 or CDK1/Cyclin B1, 1 mM ATP for CDK2/Cyclin E1 and CDK1/Cyclin B1, 0.15 μM Rb (Ser780)-biotin substrate and 0-10 μM compound in buffer containing 50 mM HEPES pH7.0, 0.02% NaN3, 0.01% BSA, 0.1 mM Orthovanadate, 50 mM MgCl2, 1 mM DTT and 0.005% Tween-20. The kinase was incubated with compound for 60 minutes at room temperature and the reaction was initiated by the addition of ATP and Rb (Ser780)-biotin substrate. After reaction at room temperature for 90 minutes, an equal volume of stop/detection solution was added according to the manufacture's instruction (Cisbio Bioassays). The stop/detection solution contained Streptavidin-XL665 and Anti-pRb (Ser780) mAb-Eu Cryptate in Detection buffer (Cisbio Bioassays). Plates were incubated at room temperature for 60 minutes, and the TR-FRET signals (ex337 nm, em665 nm/620 nm) were recorded on a PHERAstar FSX plate reader (BMG Labtech). The inhibition percentage of CDK2/Cyclin E1 or CDK1/Cyclin B1 kinase activity in presence of increasing concentrations of compounds was calculated based on the ratio of fluorescence at 665 nm to that at 620 nm. The IC50 for each compound was derived from fitting the data to the four-parameter logistic equation by Dotmatics. The IC50 data as measured for the Examples are shown in Table 2.

TABLE 2

| | CDK2/Cyclin | CDK1/Cyclin |
|---|---|---|
| Example | E1 IC₅₀ (nM) | B1 IC₅₀ (nM) |
| 1 | 100 | >10000 |
| 2 | 5677 | >10000 |
| 3 | 147 | >10000 |
| 4 | 7.1 | 848 |
| 5 | 4.6 | 656 |
| 6a | 3 | 2001 |
| 6b | 88 | >10000 |
| 6c | 1907 | >10000 |
| 6d | 503 | >10000 |
| 7 | 93 | >10000 |
| 8 | 79 | >10000 |
| 9 | 131 | >10000 |
| 10 | >10000 | >10000 |
| 11 | 2758 | >10000 |
| 12 | 3990 | >10000 |
| 13 | 462 | >10000 |
| 14 | 369 | >10000 |
| 15 | 3.7 | 1062 |
| 16 | 12 | 2494 |
| 17a | 3.9 | 2086 |
| 17b | 7711 | >10000 |
| 17c | 102 | >10000 |
| 17d | 2868 | >10000 |
| 18 | 42 | >10000 |
| 19 | 43 | >10000 |
| 20 | 73 | >10000 |
| 36 | >10000 | >10000 |
| 37 | 0.5 | 11 |
| 38 | 2368 | >10000 |
| 56 | 4.3 | 3497 |
| 72 | 5909 | >10000 |
| 80 | 1121 | >10000 |
| 88 | >10000 | >10000 |
| 89 | 351 | >10000 |
| 90 | 3313 | >10000 |
| 94 | 417 | >10000 |
| 95 | 7198 | >10000 |
| 96 | 314 | >10000 |
| 97 | 3181 | >10000 |
| 98 | 453 | >10000 |
| 99 | 3294 | >10000 |
| 100 | 173 | >10000 |
| 101 | 564 | >10000 |
| 102 | 813 | >10000 |
| 103 | >10000 | >10000 |
| 104 | 1792 | >10000 |
| 105 | 812 | >10000 |
| 106 | 1046 | >10000 |
| 107 | 2437 | >10000 |
| 108 | 8508 | >10000 |
| 109a | 8.6 | 2867 |
| 109b | 172 | >10000 |
| 109c | 1805 | >10000 |
| 109d | 158 | >10000 |
| 110 | 32 | 7244 |
| 111 | 17 | 4471 |
| 112 | 16 | 6852 |
| 113 | 235 | >10000 |
| 114 | 429 | >10000 |
| 115 | 264 | >10000 |
| 116 | 98 | >10000 |
| 117 | 34 | 6770 |
| 118 | 725 | >10000 |
| 119 | >10000 | >10000 |
| 120 | 4414 | >10000 |
| 121 | 3872 | >10000 |
| 122 | 72 | >10000 |
| 123 | 1740 | >10000 |
| 124 | 107 | 8475 |
| 125 | 40 | 2764 |
| 126 | 3.9 | 669 |
| 127 | 1.5 | 142 |
| 128 | 1250 | >10000 |
| 129 | 8019 | >10000 |
| 130 | 2119 | >10000 |
| 131 | 167 | >10000 |
| 132 | >10000 | >10000 |
| 136 | 60 | 4955 |
| 137 | 2030 | >10000 |
| 138a | 1.8 | 160 |
| 138b | 12 | 2424 |
| 139 | 54 | 5711 |
| 140 | 6 | 624 |
| 141 | 0.6 | 135 |
| 142 | 12 | 2532 |
| 143 | 14 | 1319 |
| 146 | 74 | >10000 |
| 147 | 851 | >10000 |
| 148 | 78 | 7405 |
| 149 | 6117 | >10000 |
| 150 | 189 | >10000 |
| 151 | 1404 | >10000 |
| 152a | 2.8 | 238 |
| 152b | 19 | 4577 |
| 153 | 1018 | >10000 |
| 154 | 9.6 | 540 |
| 155 | 4.6 | 685 |
| 156 | 2015 | >10000 |
| 157 | >10000 | >10000 |
| 158 | 84 | 3926 |
| 159 | 1041 | >10000 |
| 160 | 868 | >10000 |
| 161 | 1057 | >10000 |
| 162 | 308 | >10000 |
| 163 | 16 | 2020 |
| 164 | 285 | >10000 |
| 165 | 1.7 | 126 |
| 166 | 0.9 | 202 |
| 167 | 8 | 1102 |
| 168 | 15 | 2348 |
| 169a | 0.6 | 189 |
| 169b | 21 | 2354 |
| 169c | 57 | 6714 |
| 169d | 173 | >10000 |
| 170 | 5.4 | 1436 |
| 171 | 2 | 348 |
| 172 | 1.1 | 24 |
| 173 | 2.8 | 198 |
| 174 | 1.8 | 366 |
| 175 | 439 | >10000 |
| 176 | 438 | >10000 |
| 177 | 3 | 336 |
| 178 | 7 | 1516 |
| 179 | 1.5 | 537 |

What is claimed is:

1. A compound of Formula (I) 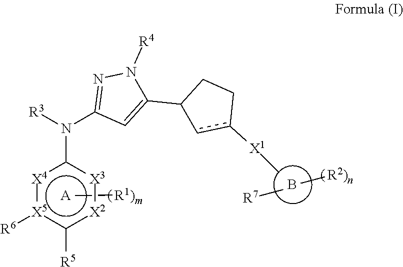
or a N-oxide thereof, or a pharmaceutically acceptable salt thereof, or a stereoisomer thereof, or a tautomer, or a deuterated analog thereof, or a prodrug thereof, wherein:
m is 0, 1, 2, 3, or 4, provided that the valency theory has been met;
n is 0, 1, 2, 3, 4, or 5, provided that the valency theory has been met; [symbol]
is independently a single bond or double bond;
ring A is selected from a heteroaryl ring and an aryl ring;
X¹ is a single bond or 0;
X², X³, X⁴, and X⁵ are each independently selected from N and C;
R¹ is each independently selected from halogen, —C₁-C₈alkyl, —C₃-C₅cycloalkyl, 3- to 8-membered heterocyclyl, —CN, and —OH, wherein each of said —C₁-C₅alkyl, C₃-C₅cycloalkyl, or 3- to 8-membered heterocyclyl is optionally substituted with at least one substituent independently selected from halogen, —C₁-C₅alkyl, —C₂-C₅alkenyl, —C₂-C₅alkynyl, —C₃-C₅cycloalkyl, 3- to 8-membered heterocyclyl, —OH, —C₆-C₁₂aryl, 5- to 12-membered heteroaryl, and oxo (═O);
R⁵ is selected from —SO₂R^{5a} and —SO₂NR^{5a}R^{5b}, wherein R^{5a} and R^{5b} are each independently selected from hydrogen and —C₁-C₃alkyl;
R⁶ is selected from hydrogen, —C₁-C₃alkyl, and —C₃-C₅cycloalkyl; or
R⁵ and R⁶, together with the atoms to which they are attached, form a 5- to 12-membered ring, said ring comprising S(═O)₂ or S(═O), and additional 0-2 heteroatom(s) independently selected from nitrogen and oxygen as ring member(s); said ring is optionally substituted with at least one substituent R^{5c};
R^{5c} is each independently selected from halogen, —C₁-C₅alkyl, —C₃-C₅cycloalkyl, 3- to 8-membered heterocyclyl, —C₆-C₁₂aryl, 5- to 12-membered heteroaryl, and oxo (═O);
ring B is selected from 5- to 7-membered heterocyclyl, phenyl, and 5- to 7-membered heteroaryl;
R² is each independently selected from halogen, —C₁-C₅alkyl, —C₂-C₅alkenyl, —C₂-C₅alkynyl, —C₃-C₈cycloalkyl, 3- to 8-membered heterocyclyl, —C₆-C₁₂aryl, 5- to 12-membered heteroaryl, oxo (═O), —OR^{2a}, —CN, —C(O)R^{2a}, —CO₂R^{2a}, —NR^{2a}R^{2b}, —NR^{2a}COR^{2b}, and —NR^{2a}CO₂R^{2b}, wherein each of said —C₁-C₈alkyl, —C₂-C₅alkenyl, —C₂-C₅alkynyl, —C₃-C₅cycloalkyl, 3- to 8-membered heterocyclyl, —C₆-C₁₂aryl or 5- to 12-membered heteroaryl is optionally substituted with at least one substituent independently selected from halogen, —C₁-C₅alkyl, —C₂-C₅alkenyl, —C₂-C₅alkynyl, —C₃-C₅cycloalkyl, 3- to 8-membered heterocyclyl, C₆-C₁₂aryl, 5- to 12-membered heteroaryl, oxo (═O), —CN, —OR^{2c}, —C₁-C₅alkyl-OR^{2c}, —C(O)R^{2c}, —CO₂R^{2c}, —NR^{2c}R^{2d}, —NR^{2c}COR^{2d}, and —NR^{2c}CO₂R^{2d};
R^{2a}, R^{2b}, R^{2c} and R^{2d} are each independently selected from hydrogen, —C₁-C₅alkyl, —C₂-C₅alkenyl, —C₂-C₅alkynyl, C₃-C₅cycloalkyl, 3- to 8-membered heterocyclyl, C₆-C₁₂aryl, and 5- to 12-membered heteroaryl, wherein each of said —C₁-C₅alkyl, —C₂-C₅alkenyl, —C₂-C₅alkynyl, C₃-C₅cycloalkyl, 3- to 8-membered heterocyclyl, C₆-C₁₂aryl, or 5- to 12-membered heteroaryl is optionally substituted with at least one substituent selected from halogen, —C₁-C₅alkyl, —C₂-C₅alkenyl, —C₂-C₅alkynyl, C₃-C₅cycloalkyl, 3- to 8-membered heterocyclyl, C₆-C₁₂aryl, 5- to 12-membered heteroaryl, oxo (═O), —CN, —OH, and —C₁-C₈alkoxyl;
R⁷ is selected from hydrogen, —C₁-C₃alkyl, and —C₃-C₅cycloalkyl; or
adjacent R² and R⁷, together with the atoms to which they are attached, form a 3- to 12-membered ring, said ring comprising 0-3 heteroatoms independently selected from nitrogen, oxygen and optionally oxidized sulfur as ring member(s); said ring is optionally substituted with at least one substituent R^{7a};
R^{7a} is each independently selected from halogen, —C₁-C₈alkyl, —C₃-C₅cycloalkyl, 3- to 8-membered heterocyclyl, —C₆-C₁₂aryl, 5- to 12-membered heteroaryl, and oxo (═O);
R³ and R⁴ are each independently selected from hydrogen, —C₁-C₈alkyl, C₃-C₅cycloalkyl, and —C(O)R^{3a}; wherein each of said —C₁-C₈alkyl and C₃-C₅cycloalkyl is optionally substituted with at least one substituent independently selected from halogen, oxo (═O), —CN, —OR^{3b} and —NR^{3b}R^{3c}; and
R^{3a}, R^{3b} and R^{3c} are each independently selected from hydrogen, —C₁-C₅alkyl, and C₃-C₅cycloalkyl;
wherein each of said —C₁-C₅alkyl and C₃-C₅cycloalkyl is optionally substituted with at least one substituent independently selected from halogen, —C₁-C₈alkyl, C₃-C₅cycloalkyl, oxo (═O), —CN, —OH, and —C₁-C₅alkoxyl.

2. The compound of claim 1, wherein the ring A is a 6-membered heteroaryl ring and three of X², X³, X⁴, and X⁵ are N; preferably, ring A is a 6-membered heteroaryl ring and two of X², X³, X⁴, and X⁵ are N; more preferably, ring A is a 6-membered heteroaryl ring and one of X², X³, X⁴, and X⁵ is N; even more preferably, ring A is a 6-membered heteroaryl ring and X² is N.

3. The compound of claim 1, wherein the compound is selected from (IIa), (IIb), (IIc), and (IId): 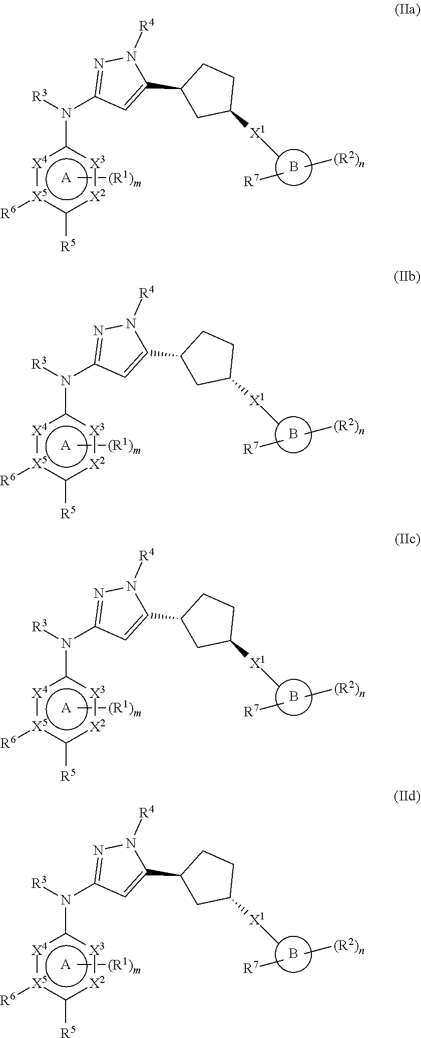
preferably, the compound is selected from (IIIa), (IIIb), (IIIc), and (IIId):

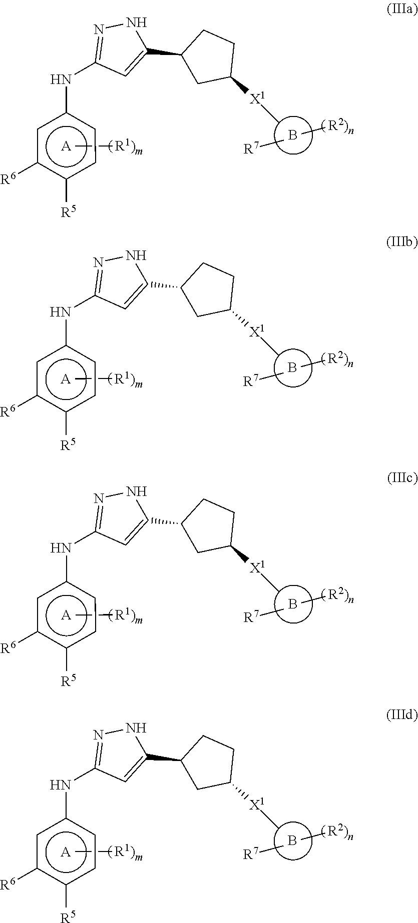

4. The compound of any one of the preceding claims, wherein
m is 0, 1, 2, 3, or 4, provided that the valency theory has been met;
R¹ is each independently selected from —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, —CN, and —OH, wherein each of said methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, or 3- to 8-membered heterocyclyl is optionally substituted with at least one substituent independently selected from —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, —C₂-C₅alkenyl, —C₂-C₈alkynyl, cyclopropyl, cyclobutyl, cyclopentyl, cyclohexyl, cycloheptyl, cyclooctyl, 3- to 8-membered heterocyclyl, —OH, —C₆-C₁₂aryl, 5- to 12-membered heteroaryl, and oxo(═O);
preferably, m is 0, 1, 2, 3, or 4, provided that the valency theory has been met;
R¹ is each independently selected from —F, —Cl, methyl, ethyl, propyl (n-propyl or isopropyl), cyclopropyl, cyclobutyl, cyclopentyl, 3- to 8-membered heterocyclyl, —CN, and —OH, wherein each of said methyl, ethyl, propyl (n-propyl or isopropyl), cyclopropyl, cyclobutyl, cyclopentyl, or 3- to 8-membered heterocyclyl is optionally substituted with at least one substituent independently selected from —F, —Cl, —Br, —I, methyl, ethyl, propyl (n-propyl or isopropyl), —C₂-C₅alkenyl, —C₂-C₅alkynyl, cyclopropyl, cyclobutyl, 3- to 8-membered heterocyclyl, —OH, —C₆-C₁₂aryl, 5- to 12-membered heteroaryl, and oxo (═O);
more preferably, m is 0 or 1, provided that the valency theory has been met;
R¹ is each independently selected from —F, —Cl, methyl, ethyl, propyl (n-propyl or isopropyl), cyclopropyl, cyclobutyl, cyclopentyl, —CN, and —OH; wherein said methyl is substituted with —OH;
even more preferably, m is 0 or 1, provided that the valency theory has been met; R¹ is —F or CH₂OH.

5. The compound of any one of the preceding claims, wherein
R⁵ is selected from —SO₂R^{5a} and —SO₂NR^{5a}R^{5b}, wherein R^{5a} and R^{5b} are each independently selected from hydrogen, methyl, ethyl, and propyl (n-propyl or isopropyl);
preferably, R⁵ is selected from —SO₂R^{5a} and —SO₂NR^{5a}R^{5b}, wherein R^{5a} and R^{5b} are each independently selected from hydrogen and methyl;
more preferably, R⁵ is selected from —SO₂R^{5a} and —SO₂NR^{5a}R^{5b}, wherein R^{5a} and R^{5b} are each hydrogen;
even more preferably, R⁵ is —SO₂NH₂.

6. The compound of any one of the preceding claims, wherein
R⁶ is selected from hydrogen, methyl, ethyl, propyl (n-propyl or isopropyl), cyclopropyl, cyclobutyl, cyclopentyl, cyclohexyl, cycloheptyl, and cyclooctyl;
preferably, R⁶ is selected from hydrogen, methyl, ethyl, cyclopropyl, cyclobutyl, and cyclopentyl;
more preferably, R⁶ is selected from hydrogen, methyl, ethyl, and cyclopropyl;
even more preferably, R⁶ is hydrogen.

7. The compound of any one of claims 1-4, wherein
adjacent R⁵ and R⁶, together with the atoms to which they are attached, form a 3- to 12-membered ring, said ring comprising S(═O)₂ or S(═O), and additional 0-2 heteroatom(s) independently selected from nitrogen and oxygen as ring member(s); said ring is optionally substituted with at least one substituent R^{5c};
R^{5c} is each independently selected from —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, —C₆-C₁₂aryl, 5- to 12-membered heteroaryl, and oxo (═O);
preferably, adjacent R⁵ and R⁶, together with the atoms to which they are attached, form a 4-, 5-, 6-, or 7-membered ring, said ring comprising S(═O)₂ or S(═O), and additional 0-2 heteroatom(s) independently selected from nitrogen and oxygen as ring member(s); said ring is optionally substituted with at least one substituent R^{5c};
R^{5c} is each independently selected from —F, —Cl, methyl, ethyl, propyl (n-propyl or isopropyl), butyl (n-butyl, sec-butyl, iso-butyl or tert-butyl), cyclopropyl, cyclobutyl, cyclopentyl, 3- to 8-membered heterocyclyl, —C₆-C₁₂aryl, 5- to 12-membered heteroaryl, and oxo (═O);
more preferably, adjacent R⁵ and R⁶, together with the atoms to which they are attached, form a 5- or 6-membered ring, said ring comprising S(═O)₂ or S(═O), and additional 0-1 nitrogen atom as ring member; said ring is optionally substituted with at least one substituent R^{5c};
R^{5c} is each independently selected from —F, methyl, ethyl, cyclopropyl, cyclobutyl, cyclopentyl, 3- to 8-membered heterocyclyl, 5- to 12-membered heteroaryl, and oxo (═O);
even more preferably, adjacent R⁵ and R⁶, together with the atoms to which they are attached, form a 5- or 6-membered ring, said ring comprising S(═O)₂ or S(═O), and additional 0-1 nitrogen atom as ring member; said ring is optionally substituted with methyl.

8. The compound of any one of the preceding claims, wherein
ring B is selected from 5- or 6-membered heterocyclyl, phenyl, and 5- or 6-membered heteroaryl;
preferably, ring B is selected from 5-membered heterocyclyl, 5-membered heteroaryl, 6-membered heteroaryl, and phenyl; wherein said 5-membered heterocyclyl, 5-membered heteroaryl, or 6-membered heteroaryl comprising 1-3 heteroatoms independently selected from nitrogen, oxygen, and optionally oxidized sulfur as ring member(s);
more preferably, ring B is selected from 5-membered heterocyclyl comprising 1-2 heteroatoms independently selected from oxygen and nitrogen as ring member(s), 5-membered heteroaryl comprising 1-3 heteroatoms independently selected from nitrogen, oxygen, and optionally oxidized sulfur as ring member(s), 6-membered heteroaryl comprising 1-2 nitrogen atoms as ring member(s), and phenyl;
even more preferably, ring B is selected from furanyl, pyridinyl, oxazolyl, pyrimidinyl, pyridazinyl, pyrazolyl, thiazolyl, oxadiazolyl, phenyl, tetrahydrofuranyl, imidazolyl, triazolyl, pyrrolyl, pyrrolidinyl, imidazolidinyl, isothiazolyl, and isoxazolyl.

9. The compound of any one of the preceding claims, wherein
n is 0, 1, 2, 3, 4, or 5, provided that the valency theory has been met;
R² is each independently selected from —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, —C₂-C₅alkenyl, —C₂-C₅alkynyl, cyclopropyl, cyclobutyl, cyclopentyl, cyclohexyl, cycloheptyl, cyclooctyl, 3- to 8-membered heterocyclyl, —C₆-C₁₂aryl, 5- to 12-membered heteroaryl, oxo (═O), —OR^{2a}, —CN, —C(O)R^{2a}, —CO₂R^{2a}, —NR^{2a}R^{2b}, —NR^{2a}COR^{2b}, and —NR^{2a}CO₂R^{2b}, wherein each of said methyl, ethyl, propyl (n-propyl or isopropyl), butyl (n-butyl, sec-butyl, iso-butyl or tert-butyl), pentyl, hexyl, heptyl, octyl, —C₂-C₅alkenyl, —C₂-C₅alkynyl, cyclopropyl, cyclobutyl, cyclopentyl, cyclohexyl, cycloheptyl, cyclooctyl, 3- to 8-membered heterocyclyl, —C₆-C₁₂aryl or 5- to 12-membered heteroaryl is optionally substituted with at least one substituent independently selected from —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, —C₂-C₅alkenyl, —C₂-C₅alkynyl, cyclopropyl, cyclobutyl, cyclopentyl, cyclohexyl, cycloheptyl, cyclooctyl, 3- to 8-membered heterocyclyl, C₆-C₁₂aryl, 5- to 12-membered heteroaryl, oxo (═O), —CN, —OR^{2c}, —C₁-C₈alkyl-OR^{2c}, —C(O)R^{2c}, —CO₂R^{2c}, —NR^{2c}R^{2d}, —NR^{2c}COR^{2d}, and —NR^{2c}CO₂R^{2d};
R^{2a}, R^{2b}, R^{2c} and R^{2d} are each independently selected from hydrogen, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₅alkynyl, cyclopropyl, cyclobutyl, cyclopentyl, cyclohexyl, cycloheptyl, cyclooctyl, 3- to 8-membered heterocyclyl, C₆-C₁₂aryl, and 5- to 12-membered heteroaryl, wherein each of said methyl, ethyl, propyl (n-propyl or isopropyl), butyl (n-butyl, sec-butyl, iso-butyl or tert-butyl), pentyl, hexyl, heptyl, octyl, —C₂-C₅alkenyl, —C₂-C₅alkynyl, cyclopropyl, cyclobutyl, cyclopentyl, cyclohexyl, cycloheptyl, cyclooctyl, 3- to 8-membered heterocyclyl, C₆-C₁₂aryl, or 5- to 12-membered heteroaryl is optionally substituted with at least one substituent independently selected from —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, —C₂-C₅alkenyl, —C₂-C₅alkynyl, cyclopropyl, cyclobutyl, cyclopentyl, cyclohexyl, cycloheptyl, cyclooctyl, 3- to 8-membered heterocyclyl, C₆-C₁₂aryl, 5- to 12-membered heteroaryl, oxo (═O), —CN, —OH, methoxy, ethoxy, propoxy, butoxy, pentoxy, hexoxy, hepthoxy, and octyloxy;
preferably, n is 0, 1, 2, 3, 4, or 5, provided that the valency theory has been met;
R² is each independently selected from —F, —Cl, methyl, ethyl, propyl (n-propyl or isopropyl), butyl (n-butyl, sec-butyl, iso-butyl or tert-butyl), —C₂-C₅alkenyl, cyclopropyl, cyclobutyl, cyclopentyl, cyclohexyl, 3- to 8-membered heterocyclyl, —C₆-C₁₂aryl, 5- to 12-membered heteroaryl, oxo (═O), —OR^{2a}, —CN, —C(O)R^{2a}, —CO₂R^{2a}, —NR^{2a}R^{2b}, —NR^{2a}COR^{2b}, and —NR^{2a}CO₂R^{2b}, wherein each of said methyl, ethyl, propyl (n-propyl or isopropyl), butyl (n-butyl, sec-butyl, iso-butyl or tert-butyl), —C₂-C₅alkenyl, cyclopropyl, cyclobutyl, cyclopentyl, cyclohexyl, 3- to 8-membered heterocyclyl, —C₆-C₁₂aryl or 5- to 12-membered heteroaryl is optionally substituted with at least one substituent independently selected from —F, —Cl, methyl, ethyl, propyl (n-propyl or isopropyl), butyl (n-butyl, sec-butyl, iso-butyl or tert-butyl), pentyl, hexyl, heptyl, octyl, —C₂-C₅alkenyl, —C₂-C₅alkynyl, cyclopropyl, cyclobutyl, 3- to 8-membered heterocyclyl, C₆-C₁₂aryl, 5- to 12-membered heteroaryl, oxo (═O), —CN, —OR^{2c}, —C₁-C₅alkyl-OR^{2c}, —C(O)R^{2c}, —CO₂R^{2c}, —NR^{2c}R^{2d}, —NR^{2c}COR^{2d}, and —NR^{2c}CO₂R^{2d};
R^{2a}, R^{2b}, R^{2c} and R^{2d} are each independently selected from hydrogen, methyl, ethyl, propyl (n-propyl or isopropyl), cyclopropyl, cyclobutyl, cyclopentyl, 3- to 8-membered heterocyclyl, C₆-C₁₂aryl, and 5- to 12-membered heteroaryl, wherein each of said methyl, ethyl, propyl (n-propyl or isopropyl), cyclopropyl, cyclobutyl, cyclopentyl, 3- to 8-membered heterocyclyl, C₆-C₁₂aryl, or 5- to 12-membered heteroaryl is optionally substituted with at least one substituent independently selected from —F, —Cl, methyl, ethyl, propyl (n-propyl or isopropyl), cyclopropyl, cyclobutyl, cyclopentyl, C₆-C₁₂aryl, oxo (═O), —CN, —OH, methoxy, and ethoxy;
more preferably, n is 0, 1, 2, 3, 4, or 5, provided that the valency theory has been met;
R² is each independently selected from —Cl, methyl, ethyl, isopropyl, tert-butyl, vinyl, cyclopropyl, cyclobutyl, phenyl, oxo (═O), —OR^{2a}, and —CO₂R^{2a}, wherein each of said methyl, ethyl, isopropyl, tert-butyl, vinyl, cyclopropyl, cyclobutyl, or phenyl is optionally substituted with at least one substituent independently selected from —F, methyl, —C₁-C₈alkyl-OR^{2c}, and —OR^{2c};
R^{2a} and R^{2c} are each independently selected from hydrogen, methyl, and ethyl;
even more preferably, n is 0, 1, 2, 3, 4, or 5, provided that the valency theory has been met;
R² is each independently selected from —Cl, methyl, ethyl, isopropyl, tert-butyl, vinyl, cyclopropyl, cyclobutyl, phenyl, oxo (═O), CF₃, methoxy, —OH, —CH₂OCH₃, CH₂OH, —CO₂CH₃, and —CO₂CH₂CH₃, wherein each of said ethyl, isopropyl, tert-butyl, vinyl, or cyclopropyl is optionally substituted with at least one substituent independently selected from methyl, —CH₂OH, and —OH.

10. The compound of any one of the preceding claims, wherein
R⁷ is selected from hydrogen, methyl, ethyl, propyl (n-propyl or isopropyl), cyclopropyl, cyclobutyl, cyclopentyl, cyclohexyl, cycloheptyl, and cyclooctyl;
preferably, R⁷ is selected from hydrogen, methyl, ethyl, cyclopropyl, cyclobutyl, and cyclopentyl;
more preferably, R⁷ is selected from hydrogen, methyl, ethyl, and cyclopropyl;
even more preferably, R⁷ is hydrogen.

11. The compound of any one of claims 1-9, wherein
n is 2, 3, 4, or 5, provided that the valency theory has been met; adjacent R² and R⁷, together with the atoms to which they are attached, form a 5-, 6-, 7-, 8-, or 9-membered ring, said ring comprising 0-3 heteroatoms independently selected from nitrogen, oxygen or optionally oxidized sulfur as ring member(s); said ring is optionally substituted with at least one substituent R^{7a};
R^{7a} is each independently selected from —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, —C₆-C₁₂aryl, 5- to 12-membered heteroaryl, and oxo (═O);
preferably, n is 2, 3, 4, or 5, provided that the valency theory has been met; adjacent R² and R⁷, together with the atoms to which they are attached, form a 5-, 6-, 7-, 8-, or 9-membered ring, said ring comprising 0-3 heteroatoms independently selected from nitrogen and oxygen as ring member(s); said ring is optionally substituted with at least one substituent R^{7a};
R^{7a} is each independently selected from —F, —Cl, methyl, ethyl, cyclopropyl, cyclobutyl, cyclopentyl, cyclohexyl, 3- to 8-membered heterocyclyl, —C₆-C₁₂aryl, and oxo (═O);
more preferably, n is 2, 3, or 4, provided that the valency theory has been met; adjacent R² and R⁷, together with the atoms to which they are attached, form a 5- or 6-membered ring, said ring comprising 0-1 heteroatom selected from nitrogen and oxygen atom as ring member; said ring is optionally substituted with at least one substituent R^{7a};
R^{7a} is each independently selected from —F, methyl, ethyl, cyclopropyl, cyclobutyl, —C₆-C₁₂aryl, and oxo (═O);
even more preferably, n is 2, provided that the valency theory has been met; adjacent R² and R⁷, together with the atoms to which they are attached, form cyclopentyl, cyclohexyl, oxanyl, tetrahydrofuranyl, phenyl or pyridinyl; said cyclopentyl, cyclohexyl, oxanyl, tetrahydrofuranyl is optionally substituted with two methyl substituents.

12. The compound of any one of the preceding claims, wherein
R³ and R⁴ are each independently selected from hydrogen, methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, or —C(O)R^{3a}; wherein each of said methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, or cyclooctyl is optionally substituted at least one substituent independently selected from with —F, —Cl, —Br, —I, —CN, —OR^{3b} and —NR^{3b}R^{3c};
R^{3a}, R^{3b} and R^{3c} are each independently selected from hydrogen, methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, or cyclooctyl; wherein each of said methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, or cyclooctyl is optionally substituted with at least one substituent independently selected from —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, oxo (═O), —CN, —OH, methoxy, ethoxy, propoxy, butoxy, pentoxy, hexoxy, hepthoxy and octyloxy;
preferably, R³ and R⁴ are each independently selected from hydrogen, methyl, ethyl, propyl (n-propyl or isopropyl), butyl (n-butyl, sec-butyl, iso-butyl or tert-butyl), cyclopropyl, cyclobutyl, cyclopentyl, and —C(O)R^{3a}; wherein each of said methyl, ethyl, propyl (n-propyl or isopropyl), butyl (n-butyl, sec-butyl, iso-butyl or tert-butyl), cyclopropyl, cyclobutyl, or cyclopentyl is optionally substituted with at least one substituent independently selected from —F and —NR^{3b}R^{3c};
R^{3a}, R^{3b} and R^{3c} are each independently selected from hydrogen, methyl, ethyl, propyl (n-propyl or isopropyl), butyl (n-butyl, sec-butyl, iso-butyl or tert-butyl), cyclopropyl, cyclobutyl, cyclopentyl and cyclohexyl;
more preferably, R³ and R⁴ are each independently selected from hydrogen, methyl, and —C(O)R^{3a}; R^{3a} is selected from hydrogen, methyl, and ethyl;
even more preferably, R³ and R⁴ are each independently hydrogen.

13. The compound of any one of the preceding claims, wherein the 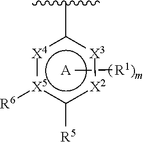
moiety is selected from

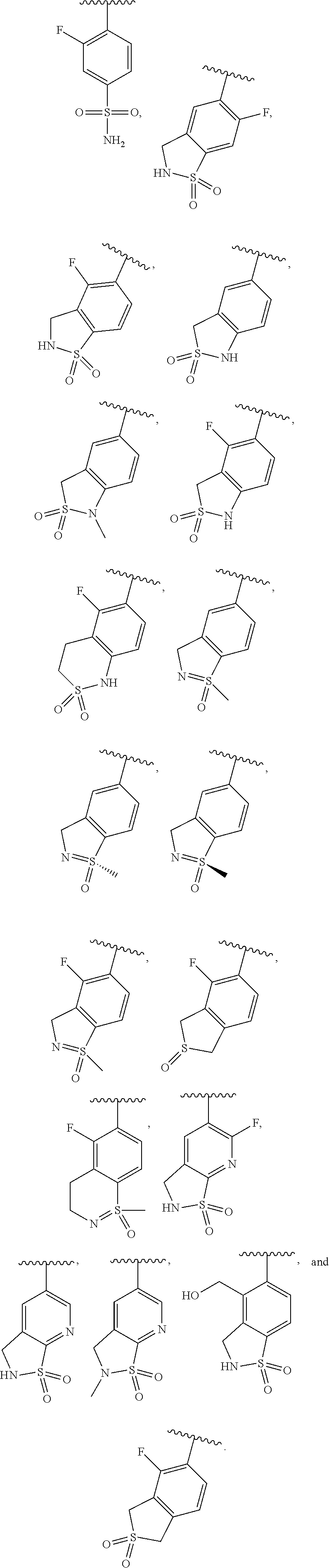

14. The compound of any one of the preceding claims, wherein the 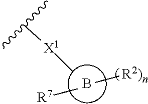
moiety is selected from

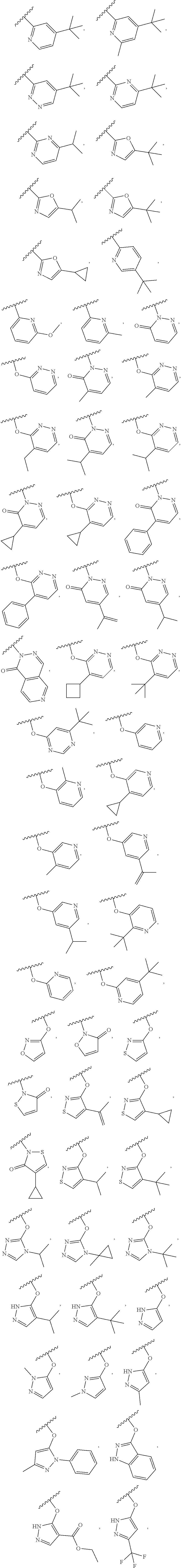

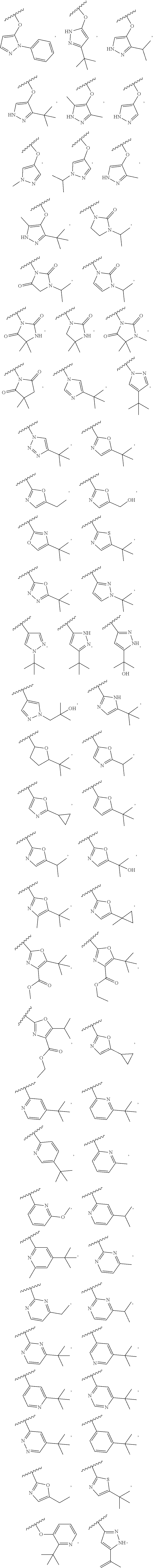

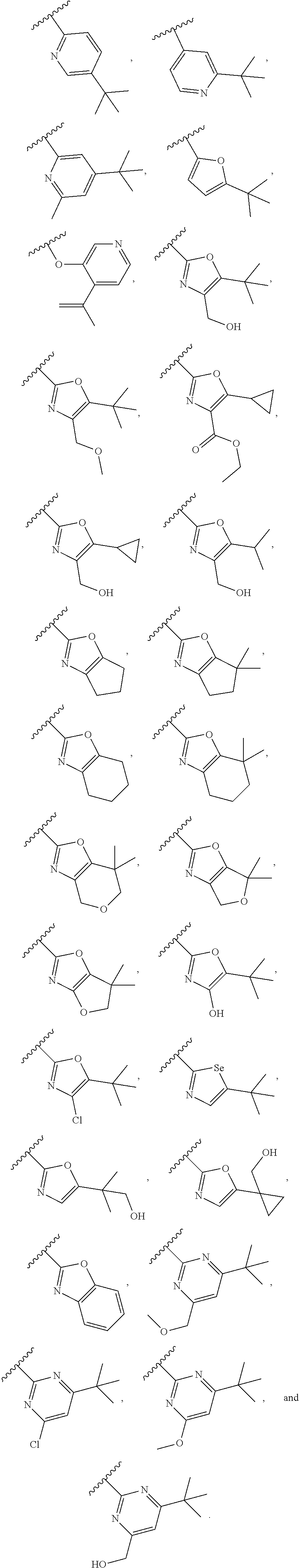

15. The compound of any one of the preceding claims, wherein the compound is selected from

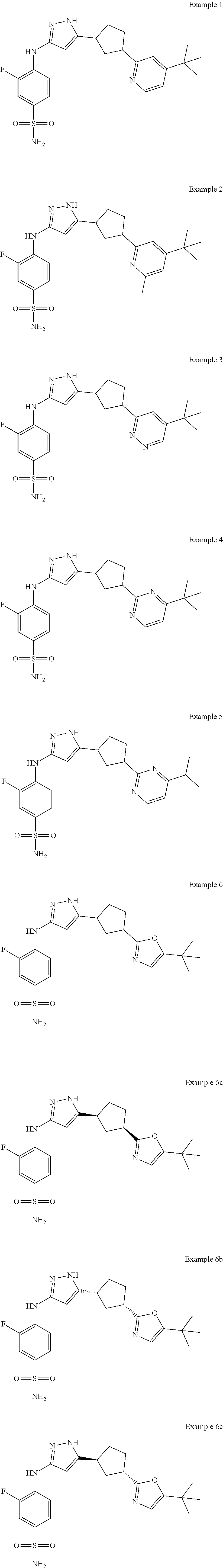

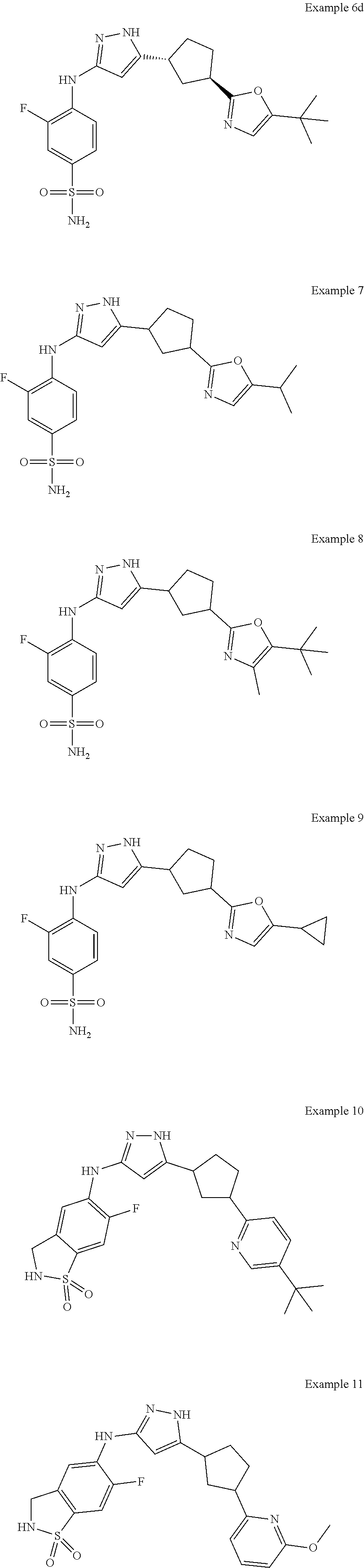 [SMILES: CC(C)(C)c1cnc([C@@H]2CC[C@@H](c3cc(Nc4ccc(S(N)(=O)=O)cc4F)n[nH]3)C2)o1 ; CC(C)c1cnc(C2CCC(c3cc(Nc4ccc(S(N)(=O)=O)cc4F)n[nH]3)C2)o1 ; Cc1nc(C2CCC(c3cc(Nc4ccc(S(N)(=O)=O)cc4F)n[nH]3)C2)oc1C(C)(C)C ; NS(=O)(=O)c1ccc(Nc2cc(C3CCC(c4ncc(C5CC5)o4)C3)[nH]n2)c(F)c1 ; CC(C)(C)c1ccc(C2CCC(c3cc(Nc4cc5c(cc4F)S(=O)(=O)NC5)n[nH]3)C2)nc1 ; COc1cccc(C2CCC(c3cc(Nc4cc5c(cc4F)S(=O)(=O)NC5)n[nH]3)C2)n1]

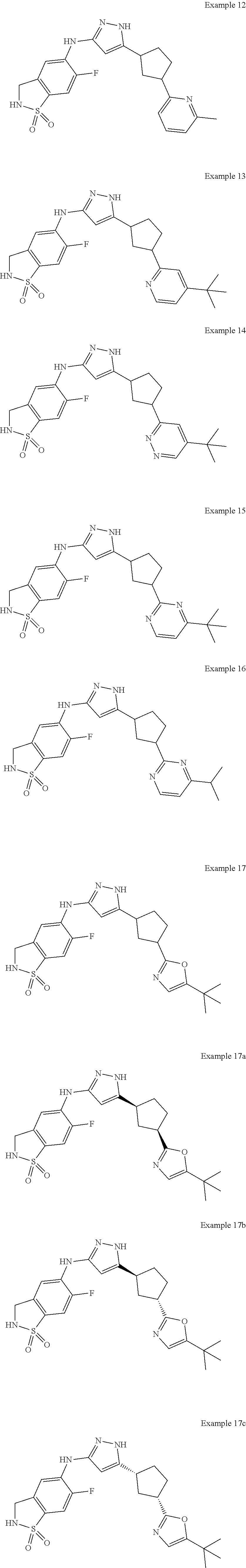

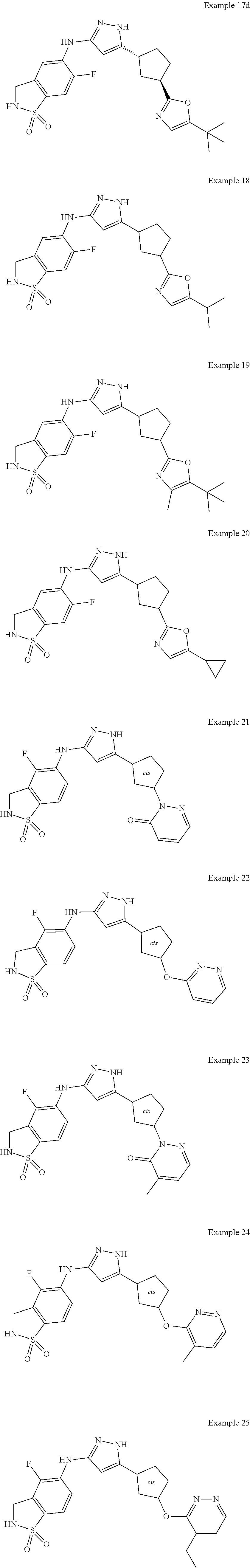

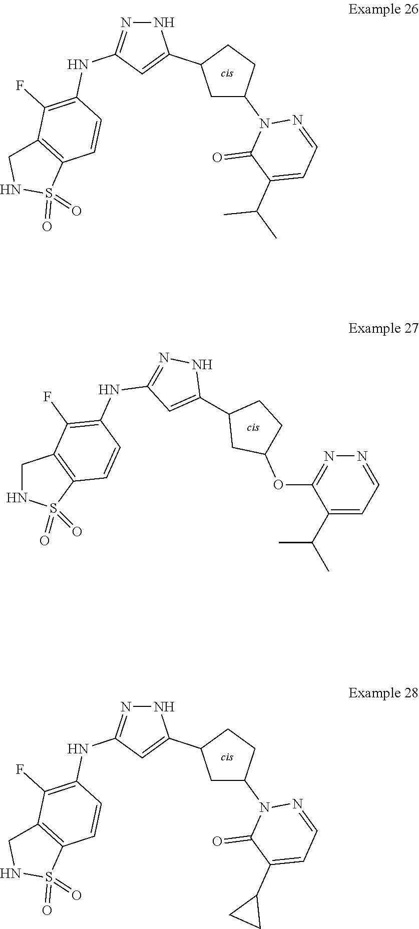 [SMILES: CC(C)c1ccnn(C2CCC(c3cc(Nc4ccc5c(c4F)CNS5(=O)=O)n[nH]3)C2)c1=O ; CC(C)c1ccnnc1OC1CCC(c2cc(Nc3ccc4c(c3F)CNS4(=O)=O)n[nH]2)C1 ; O=c1c(C2CC2)ccnn1C1CCC(c2cc(Nc3ccc4c(c3F)CNS4(=O)=O)n[nH]2)C1]

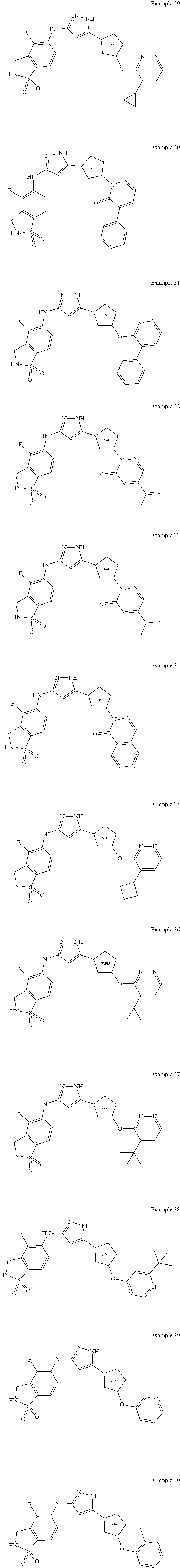

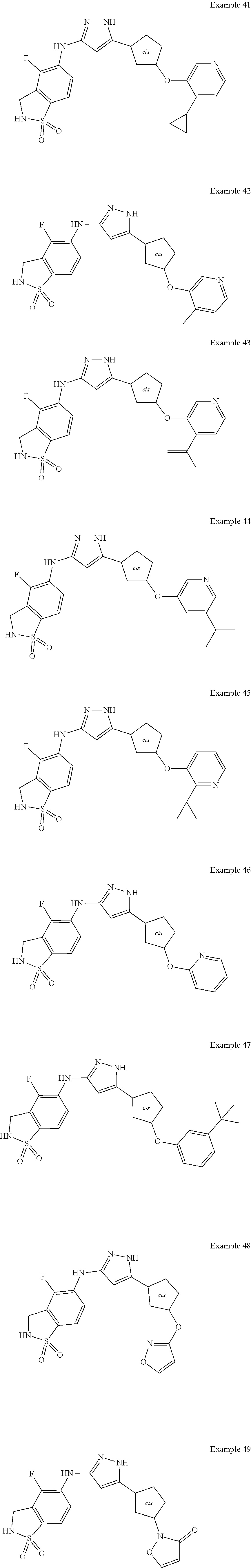

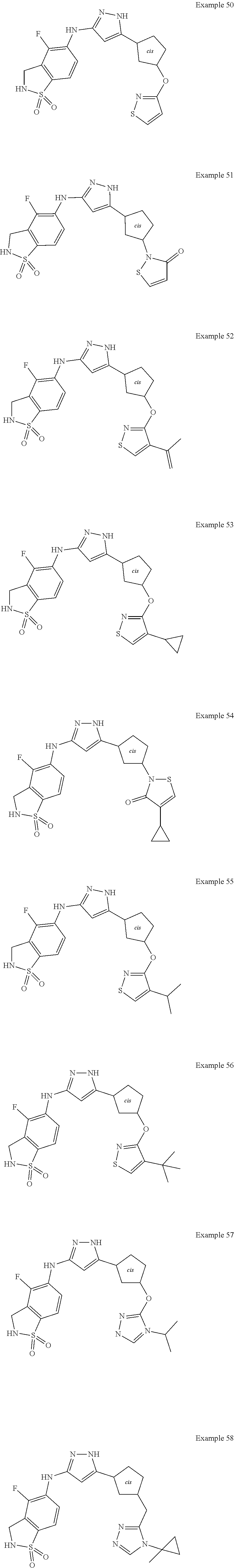

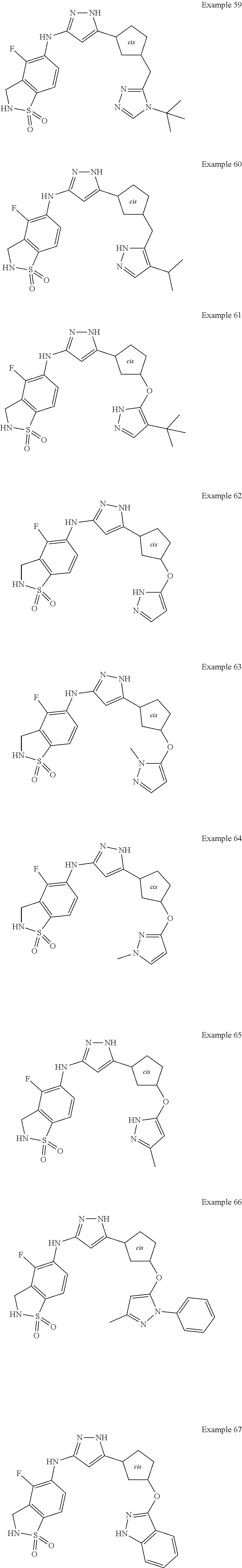

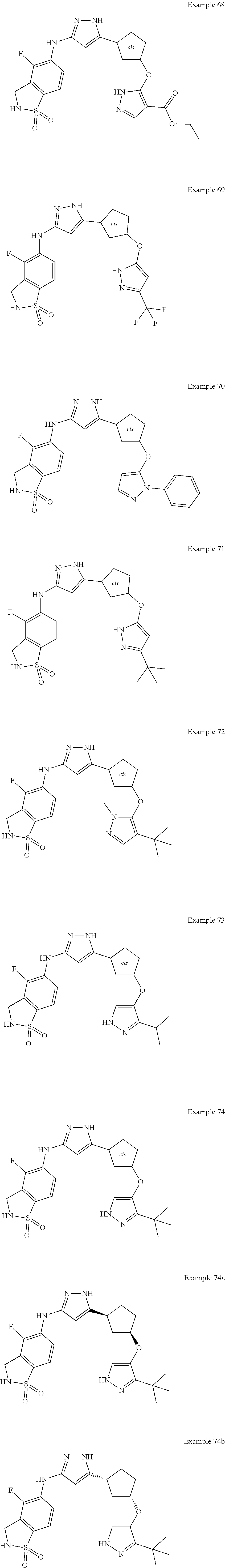

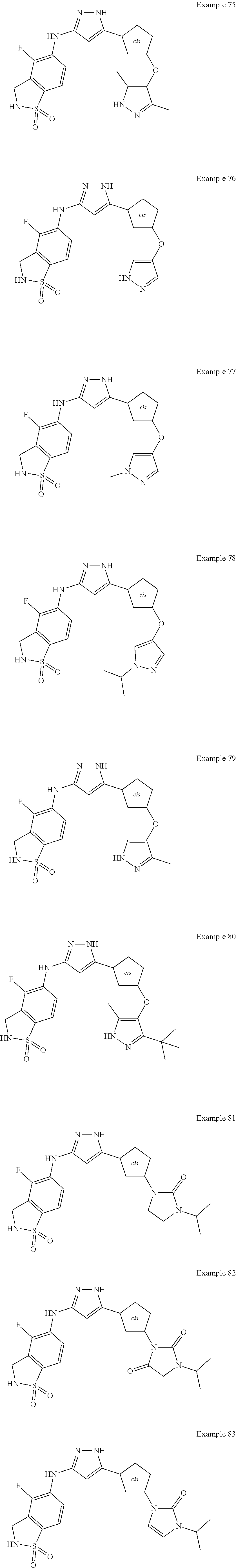

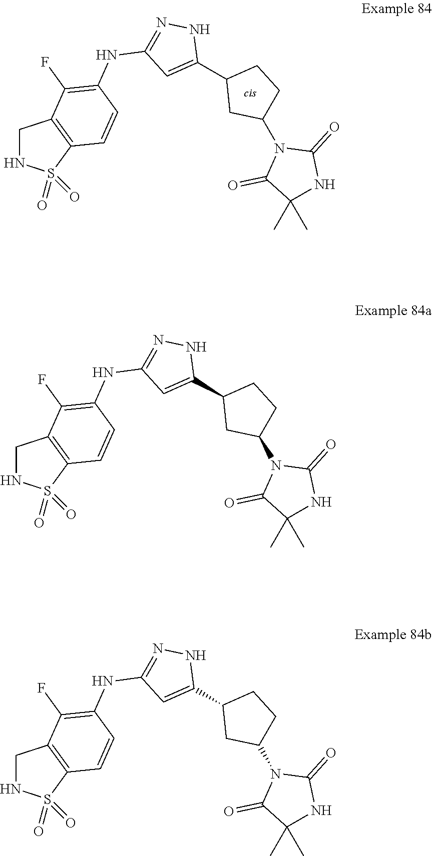

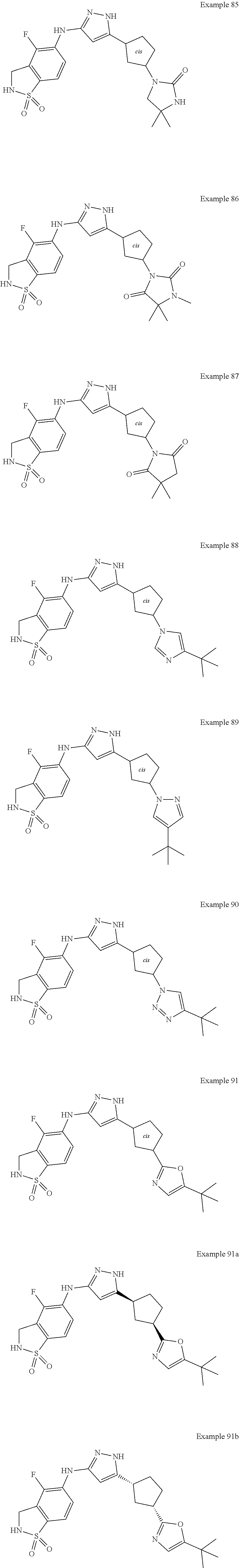

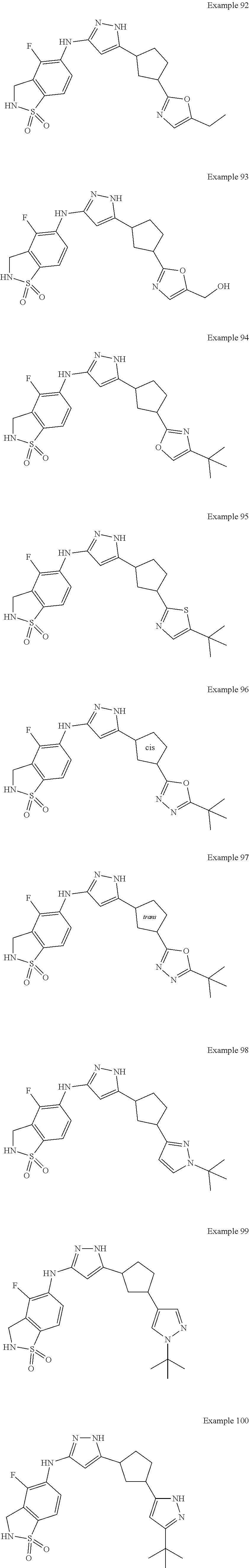

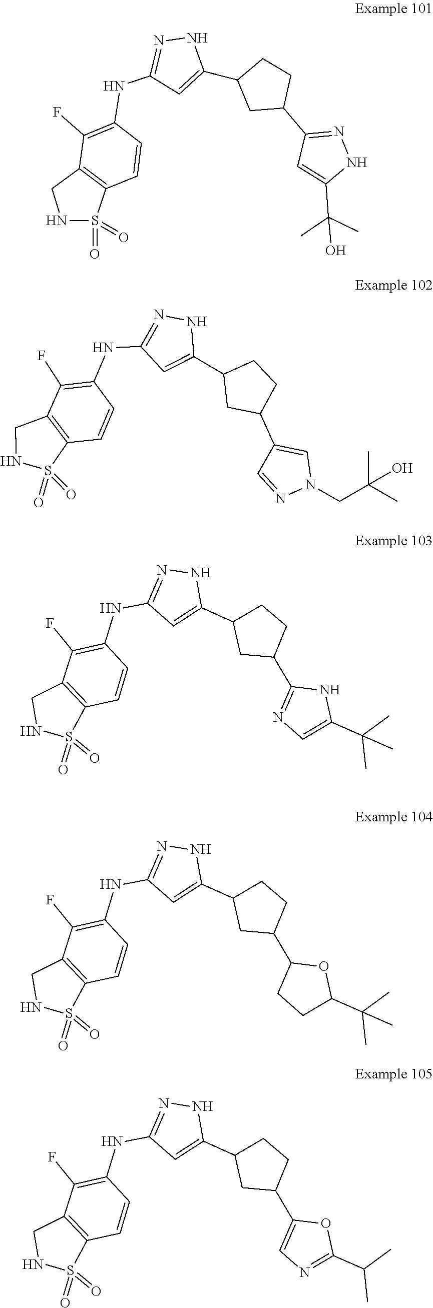 [SMILES: CC(C)(O)c1cc(C2CCC(c3cc(Nc4ccc5c(c4F)CNS5(=O)=O)n[nH]3)C2)n[nH]1 ; CC(C)(O)Cn1cc(C2CCC(c3cc(Nc4ccc5c(c4F)CNS5(=O)=O)n[nH]3)C2)cn1 ; CC(C)(C)c1cnc(C2CCC(c3cc(Nc4ccc5c(c4F)CNS5(=O)=O)n[nH]3)C2)[nH]1 ; CC(C)(C)C1CCC(C2CCC(c3cc(Nc4ccc5c(c4F)CNS5(=O)=O)n[nH]3)C2)O1 ; CC(C)c1ncc(C2CCC(c3cc(Nc4ccc5c(c4F)CNS5(=O)=O)n[nH]3)C2)o1]

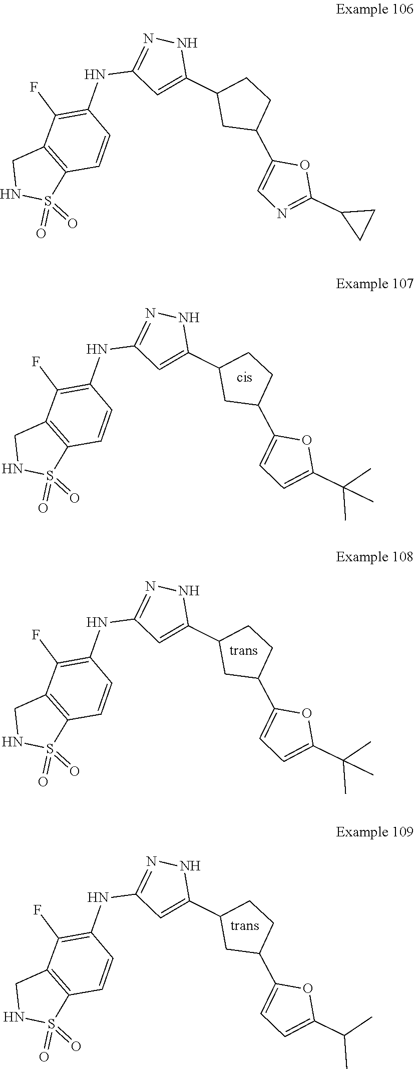 [SMILES: O=S1(=O)NCc2c1ccc(Nc1cc(C3CCC(c4cnc(C5CC5)o4)C3)[nH]n1)c2F ; CC(C)(C)c1ccc(C2CCC(c3cc(Nc4ccc5c(c4F)CNS5(=O)=O)n[nH]3)C2)o1 ; CC(C)(C)c1ccc(C2CCC(c3cc(Nc4ccc5c(c4F)CNS5(=O)=O)n[nH]3)C2)o1 ; CC(C)c1ccc(C2CCC(c3cc(Nc4ccc5c(c4F)CNS5(=O)=O)n[nH]3)C2)o1]

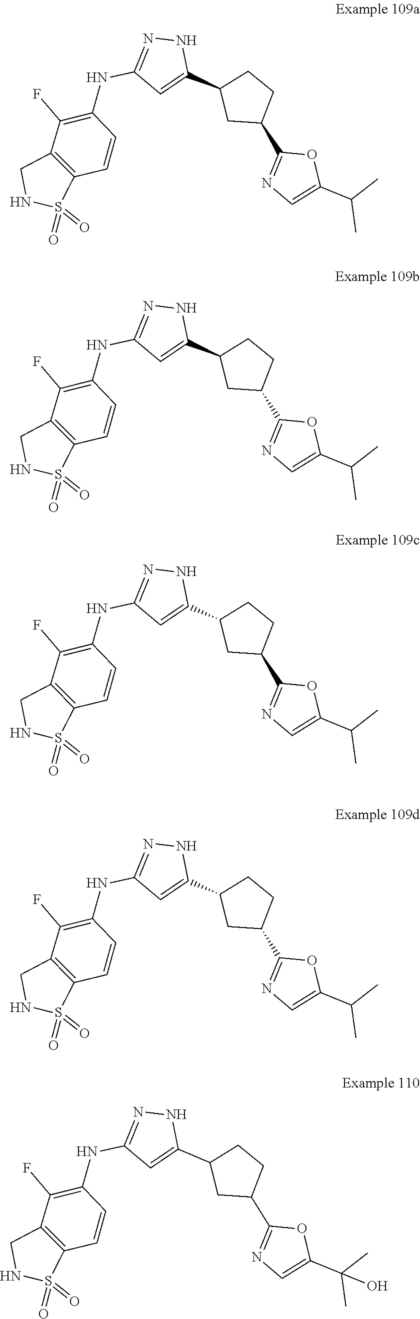 [SMILES: CC(C)c1cnc([C@@H]2CC[C@H](c3cc(Nc4ccc5c(c4F)CNS5(=O)=O)n[nH]3)C2)o1 ; CC(C)c1cnc([C@H]2CC[C@H](c3cc(Nc4ccc5c(c4F)CNS5(=O)=O)n[nH]3)C2)o1 ; CC(C)c1cnc([C@@H]2CC[C@@H](c3cc(Nc4ccc5c(c4F)CNS5(=O)=O)n[nH]3)C2)o1 ; CC(C)c1cnc([C@H]2CC[C@@H](c3cc(Nc4ccc5c(c4F)CNS5(=O)=O)n[nH]3)C2)o1 ; CC(C)(O)c1cnc(C2CCC(c3cc(Nc4ccc5c(c4F)CNS5(=O)=O)n[nH]3)C2)o1]

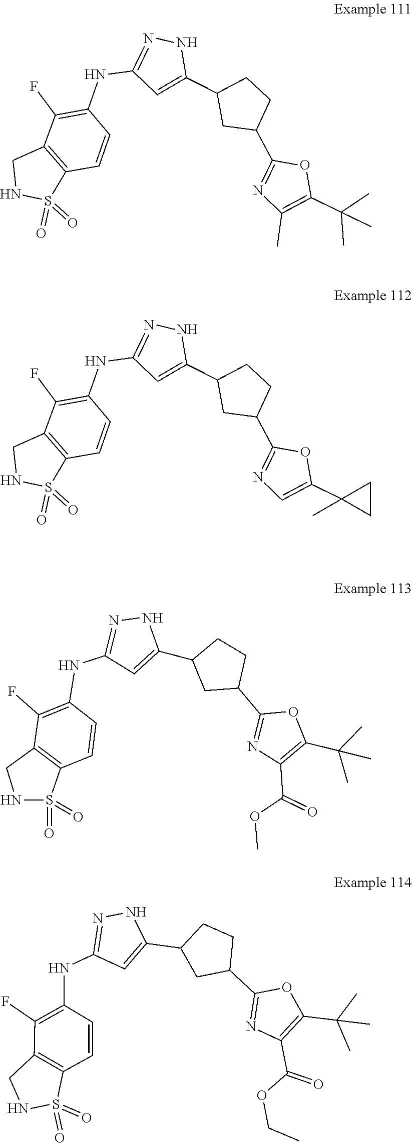 [SMILES: Cc1nc(C2CCC(c3cc(Nc4ccc5c(c4F)CNS5(=O)=O)n[nH]3)C2)oc1C(C)(C)C ; CC1(c2cnc(C3CCC(c4cc(Nc5ccc6c(c5F)CNS6(=O)=O)n[nH]4)C3)o2)CC1 ; COC(=O)c1nc(C2CCC(c3cc(Nc4ccc5c(c4F)CNS5(=O)=O)n[nH]3)C2)oc1C(C)(C)C ; CCOC(=O)c1nc(C2CCC(c3cc(Nc4ccc5c(c4F)CNS5(=O)=O)n[nH]3)C2)oc1C(C)(C)C]

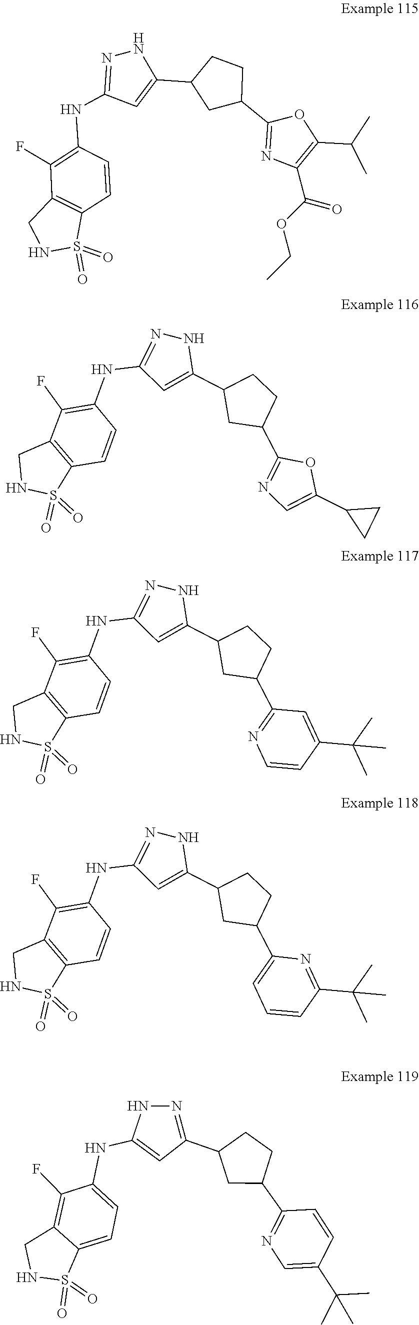 [SMILES: CCOC(=O)c1nc(C2CCC(c3cc(Nc4ccc5c(c4F)CNS5(=O)=O)n[nH]3)C2)oc1C(C)C ; O=S1(=O)NCc2c1ccc(Nc1cc(C3CCC(c4ncc(C5CC5)o4)C3)[nH]n1)c2F ; CC(C)(C)c1ccnc(C2CCC(c3cc(Nc4ccc5c(c4F)CNS5(=O)=O)n[nH]3)C2)c1 ; CC(C)(C)c1cccc(C2CCC(c3cc(Nc4ccc5c(c4F)CNS5(=O)=O)n[nH]3)C2)n1 ; CC(C)(C)c1ccc(C2CCC(c3cc(Nc4ccc5c(c4F)CNS5(=O)=O)[nH]n3)C2)nc1]

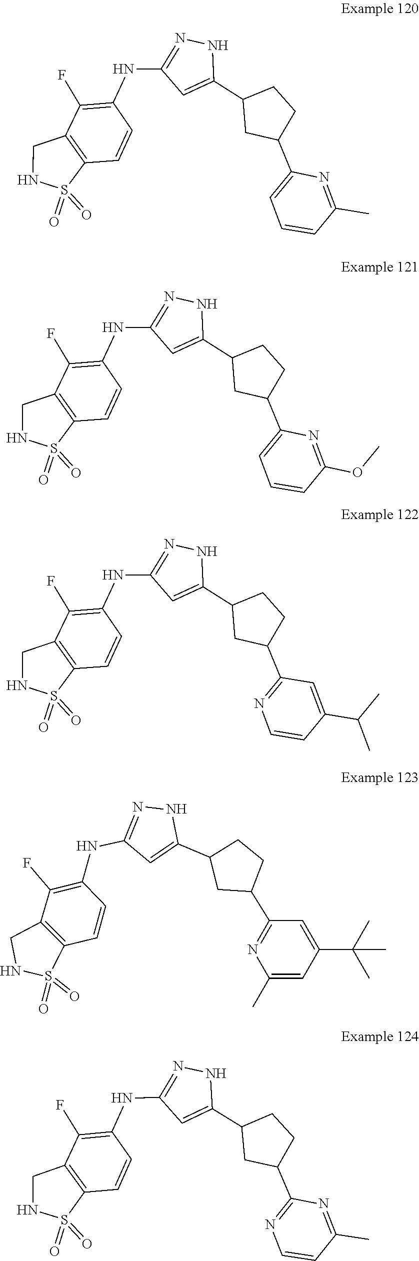 [SMILES: Cc1cccc(C2CCC(c3cc(Nc4ccc5c(c4F)CNS5(=O)=O)n[nH]3)C2)n1 ; COc1cccc(C2CCC(c3cc(Nc4ccc5c(c4F)CNS5(=O)=O)n[nH]3)C2)n1 ; CC(C)c1ccnc(C2CCC(c3cc(Nc4ccc5c(c4F)CNS5(=O)=O)n[nH]3)C2)c1 ; Cc1cc(C(C)(C)C)cc(C2CCC(c3cc(Nc4ccc5c(c4F)CNS5(=O)=O)n[nH]3)C2)n1 ; Cc1ccnc(C2CCC(c3cc(Nc4ccc5c(c4F)CNS5(=O)=O)n[nH]3)C2)n1]

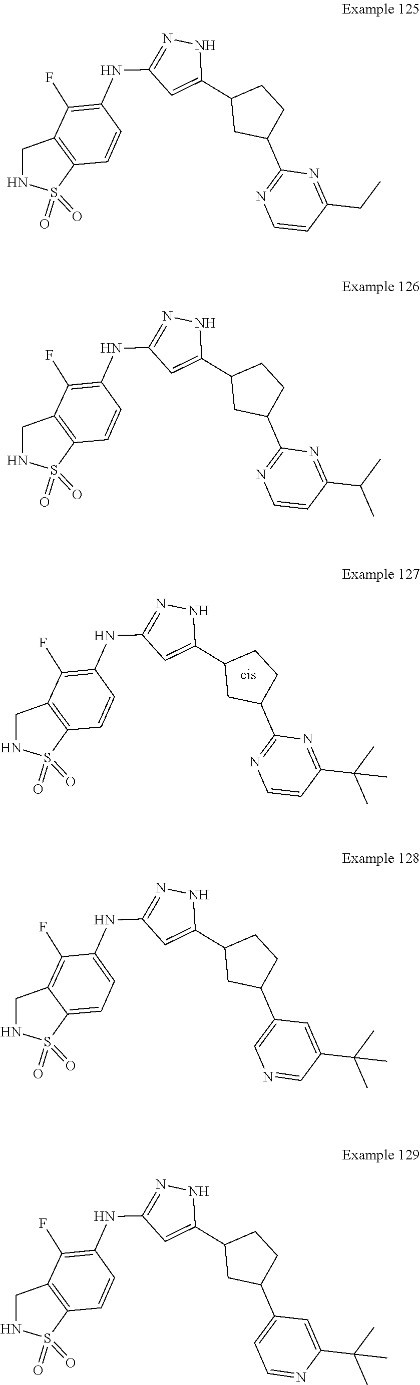 [SMILES: CCc1ccnc(C2CCC(c3cc(Nc4ccc5c(c4F)CNS5(=O)=O)n[nH]3)C2)n1 ; CC(C)c1ccnc(C2CCC(c3cc(Nc4ccc5c(c4F)CNS5(=O)=O)n[nH]3)C2)n1 ; CC(C)(C)c1ccnc(C2CCC(c3cc(Nc4ccc5c(c4F)CNS5(=O)=O)n[nH]3)C2)n1 ; CC(C)(C)c1cncc(C2CCC(c3cc(Nc4ccc5c(c4F)CNS5(=O)=O)n[nH]3)C2)c1 ; CC(C)(C)c1cc(C2CCC(c3cc(Nc4ccc5c(c4F)CNS5(=O)=O)n[nH]3)C2)ccn1]

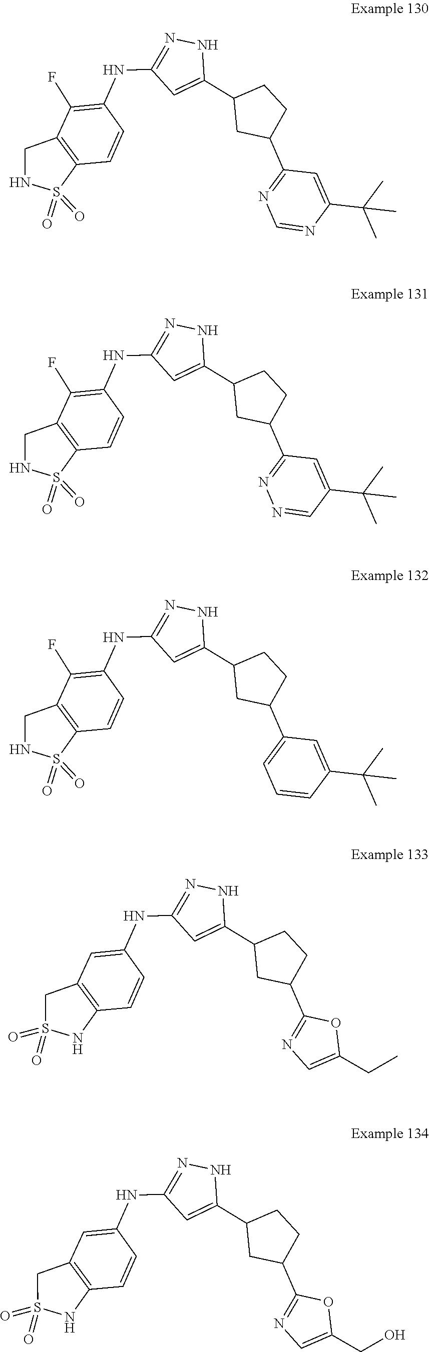 [SMILES: CC(C)(C)c1cc(C2CCC(c3cc(Nc4ccc5c(c4F)CNS5(=O)=O)n[nH]3)C2)ncn1 ; CC(C)(C)c1cnnc(C2CCC(c3cc(Nc4ccc5c(c4F)CNS5(=O)=O)n[nH]3)C2)c1 ; CC(C)(C)c1cccc(C2CCC(c3cc(Nc4ccc5c(c4F)CNS5(=O)=O)n[nH]3)C2)c1 ; CCc1cnc(C2CCC(c3cc(Nc4ccc5c(c4)CS(=O)(=O)N5)n[nH]3)C2)o1 ; O=S1(=O)Cc2cc(Nc3cc(C4CCC(c5ncc(CO)o5)C4)[nH]n3)ccc2N1]

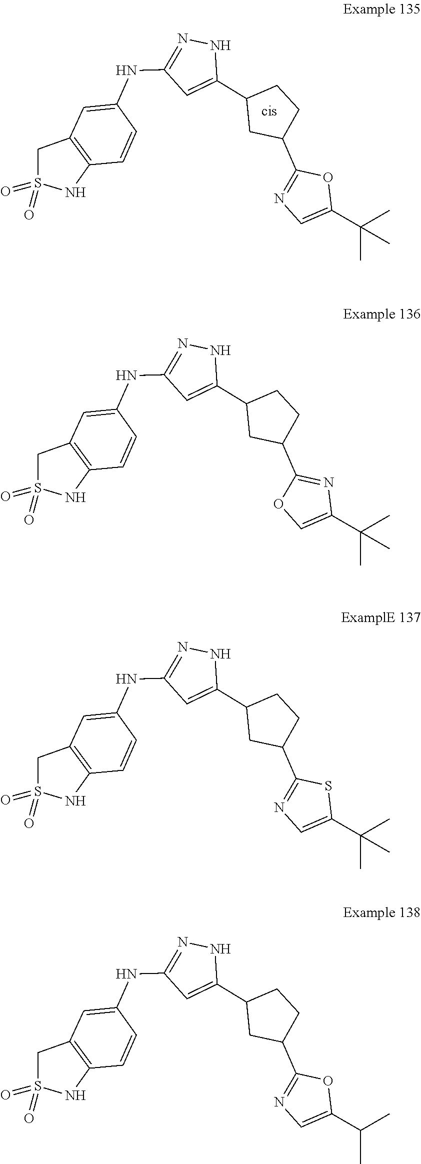 [SMILES: CC(C)(C)c1cnc(C2CCC(c3cc(Nc4ccc5c(c4)CS(=O)(=O)N5)n[nH]3)C2)o1 ; CC(C)(C)c1coc(C2CCC(c3cc(Nc4ccc5c(c4)CS(=O)(=O)N5)n[nH]3)C2)n1 ; CC(C)(C)c1cnc(C2CCC(c3cc(Nc4ccc5c(c4)CS(=O)(=O)N5)n[nH]3)C2)s1 ; CC(C)c1cnc(C2CCC(c3cc(Nc4ccc5c(c4)CS(=O)(=O)N5)n[nH]3)C2)o1]

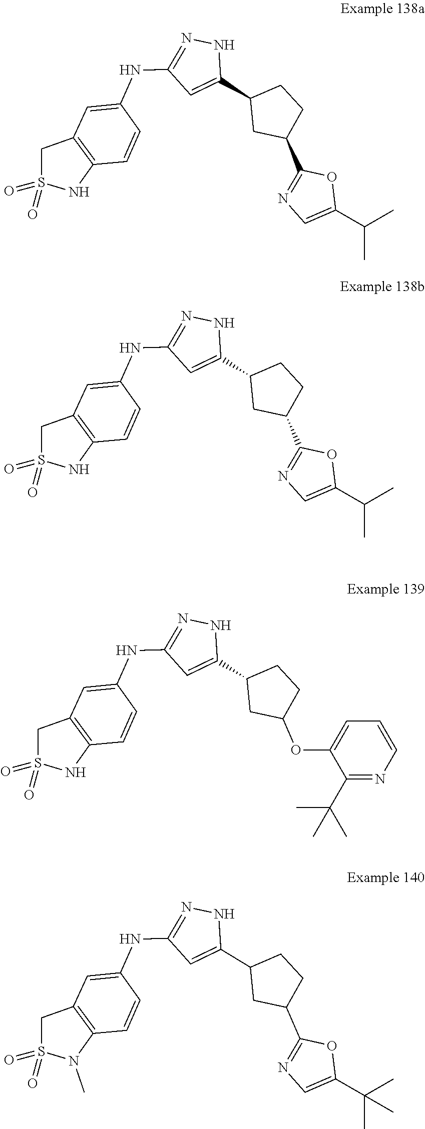 [SMILES: CC(C)c1cnc([C@@H]2CC[C@H](c3cc(Nc4ccc5c(c4)CS(=O)(=O)N5)n[nH]3)C2)o1 ; CC(C)c1cnc([C@H]2CC[C@@H](c3cc(Nc4ccc5c(c4)CS(=O)(=O)N5)n[nH]3)C2)o1 ; CC(C)(C)c1ncccc1OC1CC[C@@H](c2cc(Nc3ccc4c(c3)CS(=O)(=O)N4)n[nH]2)C1 ; CN1c2ccc(Nc3cc(C4CCC(c5ncc(C(C)(C)C)o5)C4)[nH]n3)cc2CS1(=O)=O]

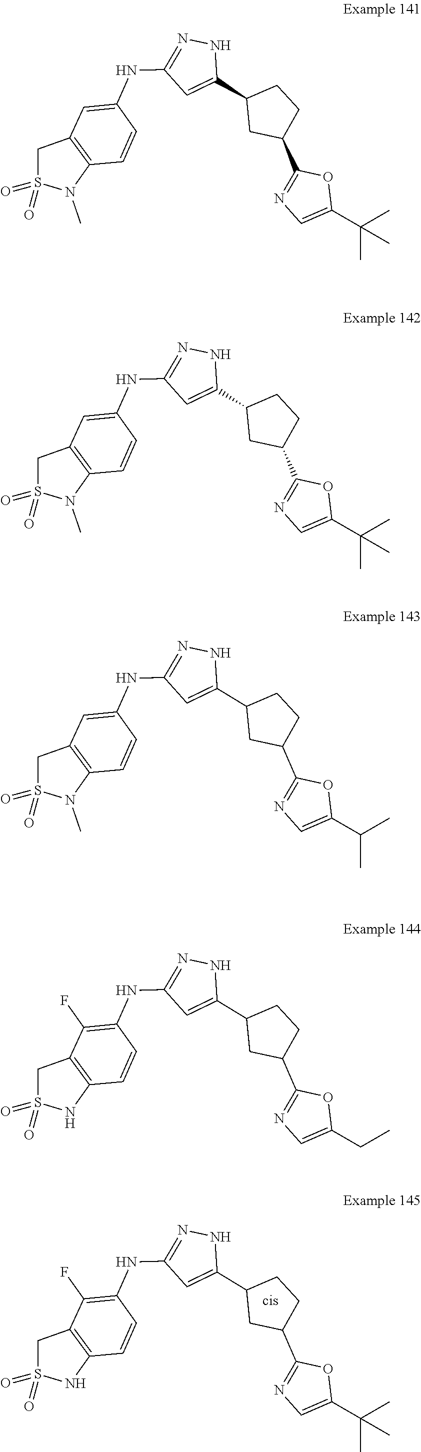 [SMILES: CN1c2ccc(Nc3cc([C@H]4CC[C@@H](c5ncc(C(C)(C)C)o5)C4)[nH]n3)cc2CS1(=O)=O ; CN1c2ccc(Nc3cc([C@@H]4CC[C@H](c5ncc(C(C)(C)C)o5)C4)[nH]n3)cc2CS1(=O)=O ; CC(C)c1cnc(C2CCC(c3cc(Nc4ccc5c(c4)CS(=O)(=O)N5C)n[nH]3)C2)o1 ; CCc1cnc(C2CCC(c3cc(Nc4ccc5c(c4F)CS(=O)(=O)N5)n[nH]3)C2)o1 ; CC(C)(C)c1cnc(C2CCC(c3cc(Nc4ccc5c(c4F)CS(=O)(=O)N5)n[nH]3)C2)o1]

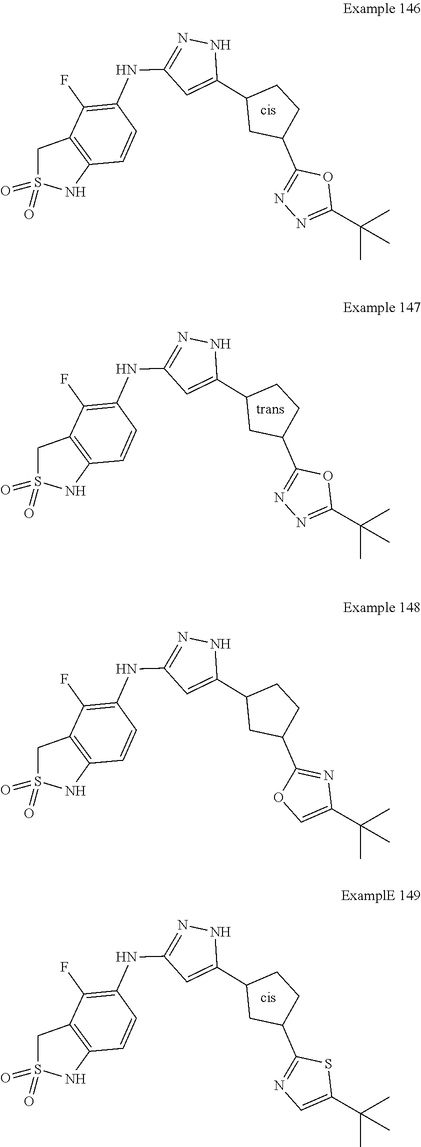 [SMILES: CC(C)(C)c1nnc(C2CCC(c3cc(Nc4ccc5c(c4F)CS(=O)(=O)N5)n[nH]3)C2)o1 ; CC(C)(C)c1nnc(C2CCC(c3cc(Nc4ccc5c(c4F)CS(=O)(=O)N5)n[nH]3)C2)o1 ; CC(C)(C)c1coc(C2CCC(c3cc(Nc4ccc5c(c4F)CS(=O)(=O)N5)n[nH]3)C2)n1 ; CC(C)(C)c1cnc(C2CCC(c3cc(Nc4ccc5c(c4F)CS(=O)(=O)N5)n[nH]3)C2)s1]

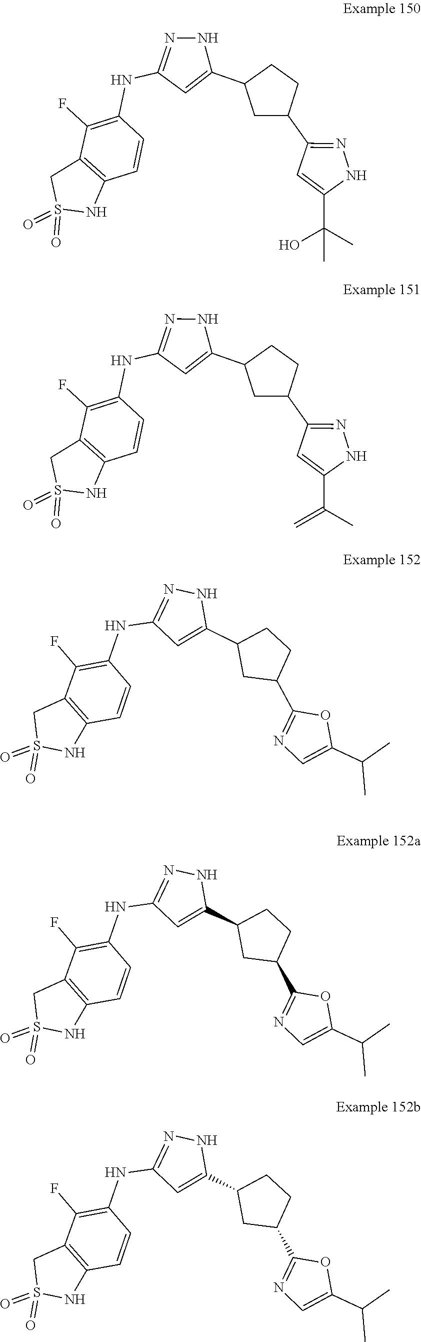 [SMILES: CC(C)(O)c1cc(C2CCC(c3cc(Nc4ccc5c(c4F)CS(=O)(=O)N5)n[nH]3)C2)n[nH]1 ; C=C(C)c1cc(C2CCC(c3cc(Nc4ccc5c(c4F)CS(=O)(=O)N5)n[nH]3)C2)n[nH]1 ; CC(C)c1cnc(C2CCC(c3cc(Nc4ccc5c(c4F)CS(=O)(=O)N5)n[nH]3)C2)o1 ; CC(C)c1cnc([C@@H]2CC[C@H](c3cc(Nc4ccc5c(c4F)CS(=O)(=O)N5)n[nH]3)C2)o1 ; CC(C)c1cnc([C@H]2CC[C@@H](c3cc(Nc4ccc5c(c4F)CS(=O)(=O)N5)n[nH]3)C2)o1]

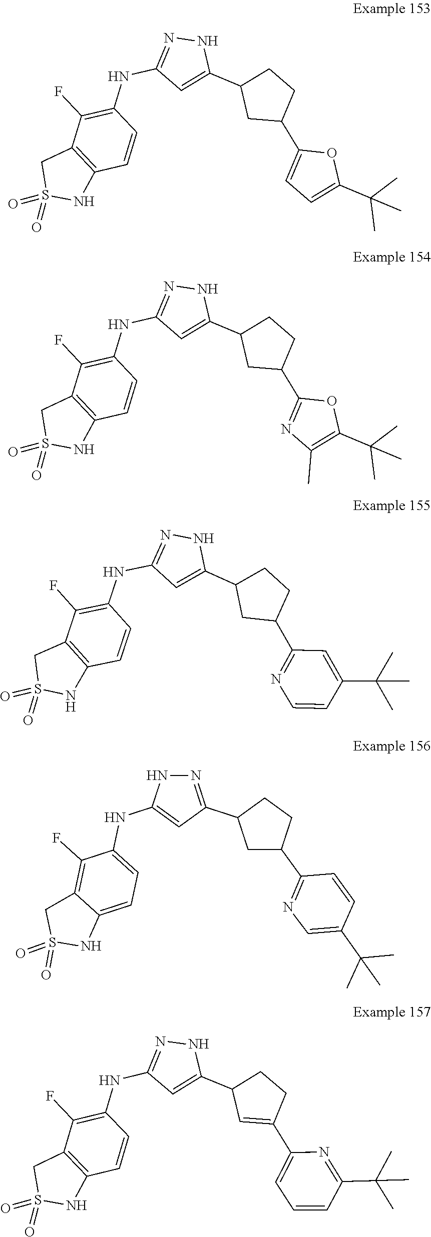 [SMILES: CC(C)(C)c1ccc(C2CCC(c3cc(Nc4ccc5c(c4F)CS(=O)(=O)N5)n[nH]3)C2)o1 ; Cc1nc(C2CCC(c3cc(Nc4ccc5c(c4F)CS(=O)(=O)N5)n[nH]3)C2)oc1C(C)(C)C ; CC(C)(C)c1ccnc(C2CCC(c3cc(Nc4ccc5c(c4F)CS(=O)(=O)N5)n[nH]3)C2)c1 ; CC(C)(C)c1ccc(C2CCC(c3cc(Nc4ccc5c(c4F)CS(=O)(=O)N5)[nH]n3)C2)nc1 ; CC(C)(C)c1cccc(C2=CC(c3cc(Nc4ccc5c(c4F)CS(=O)(=O)N5)n[nH]3)CC2)n1]

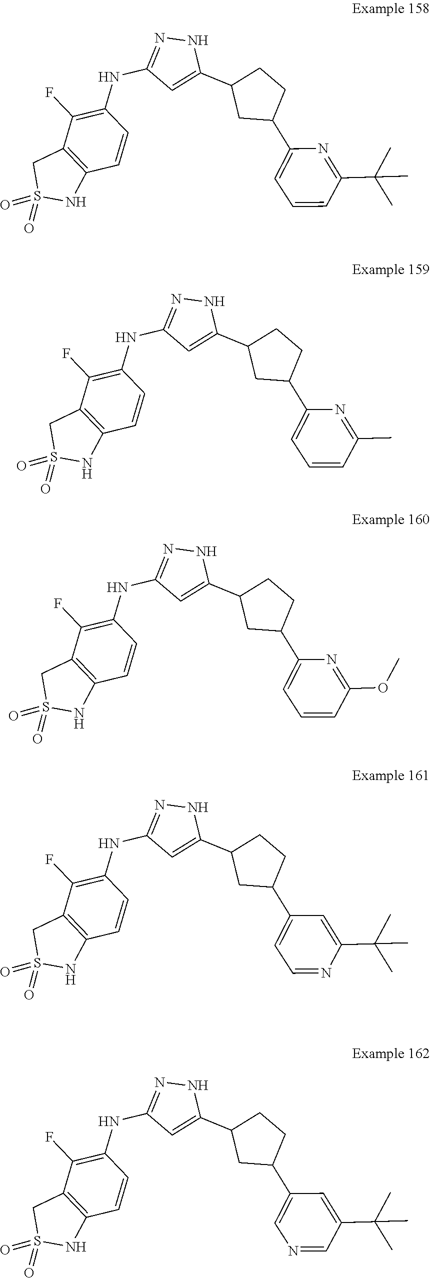 [SMILES: CC(C)(C)c1cccc(C2CCC(c3cc(Nc4ccc5c(c4F)CS(=O)(=O)N5)n[nH]3)C2)n1 ; Cc1cccc(C2CCC(c3cc(Nc4ccc5c(c4F)CS(=O)(=O)N5)n[nH]3)C2)n1 ; COc1cccc(C2CCC(c3cc(Nc4ccc5c(c4F)CS(=O)(=O)N5)n[nH]3)C2)n1 ; CC(C)(C)c1cc(C2CCC(c3cc(Nc4ccc5c(c4F)CS(=O)(=O)N5)n[nH]3)C2)ccn1 ; CC(C)(C)c1cncc(C2CCC(c3cc(Nc4ccc5c(c4F)CS(=O)(=O)N5)n[nH]3)C2)c1]

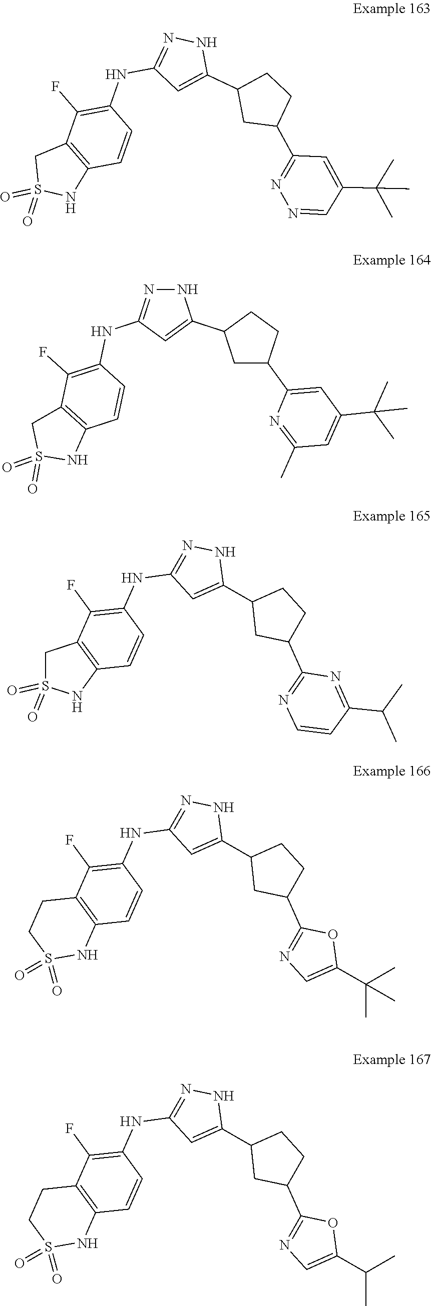 [SMILES: CC(C)(C)c1cnnc(C2CCC(c3cc(Nc4ccc5c(c4F)CS(=O)(=O)N5)n[nH]3)C2)c1 ; Cc1cc(C(C)(C)C)cc(C2CCC(c3cc(Nc4ccc5c(c4F)CS(=O)(=O)N5)n[nH]3)C2)n1 ; CC(C)c1ccnc(C2CCC(c3cc(Nc4ccc5c(c4F)CS(=O)(=O)N5)n[nH]3)C2)n1 ; CC(C)(C)c1cnc(C2CCC(c3cc(Nc4ccc5c(c4F)CCS(=O)(=O)N5)n[nH]3)C2)o1 ; CC(C)c1cnc(C2CCC(c3cc(Nc4ccc5c(c4F)CCS(=O)(=O)N5)n[nH]3)C2)o1]

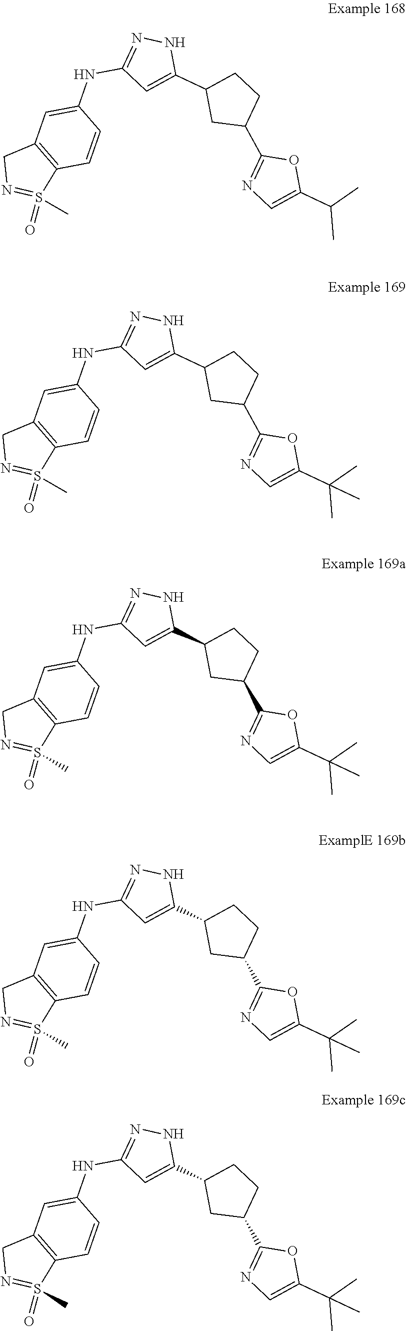 [SMILES: CC(C)c1cnc(C2CCC(c3cc(Nc4ccc5c(c4)CN=S5(C)=O)n[nH]3)C2)o1 ; CC(C)(C)c1cnc(C2CCC(c3cc(Nc4ccc5c(c4)CN=S5(C)=O)n[nH]3)C2)o1 ; CC(C)(C)c1cnc([C@@H]2CC[C@H](c3cc(Nc4ccc5c(c4)CN=[S@]5(C)=O)n[nH]3)C2)o1 ; CC(C)(C)c1cnc([C@H]2CC[C@@H](c3cc(Nc4ccc5c(c4)CN=[S@]5(C)=O)n[nH]3)C2)o1 ; CC(C)(C)c1cnc([C@H]2CC[C@@H](c3cc(Nc4ccc5c(c4)CN=[S@@]5(C)=O)n[nH]3)C2)o1]

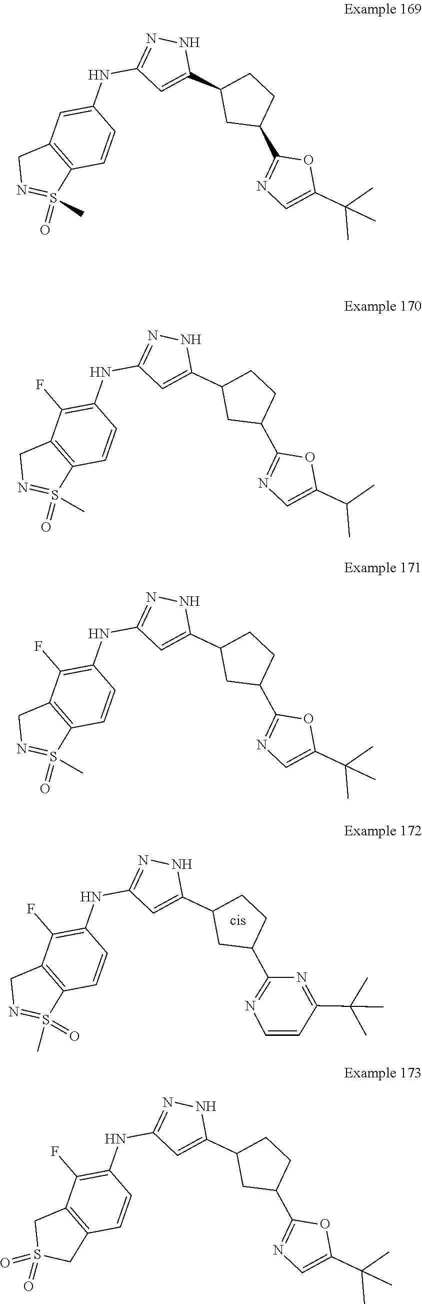 [SMILES: CC(C)(C)c1cnc([C@@H]2CC[C@H](c3cc(Nc4ccc5c(c4)CN=[S@@]5(C)=O)n[nH]3)C2)o1 ; CC(C)c1cnc(C2CCC(c3cc(Nc4ccc5c(c4F)CN=S5(C)=O)n[nH]3)C2)o1 ; CC(C)(C)c1cnc(C2CCC(c3cc(Nc4ccc5c(c4F)CN=S5(C)=O)n[nH]3)C2)o1 ; CC(C)(C)c1ccnc(C2CCC(c3cc(Nc4ccc5c(c4F)CN=S5(C)=O)n[nH]3)C2)n1 ; CC(C)(C)c1cnc(C2CCC(c3cc(Nc4ccc5c(c4F)CS(=O)(=O)C5)n[nH]3)C2)o1]

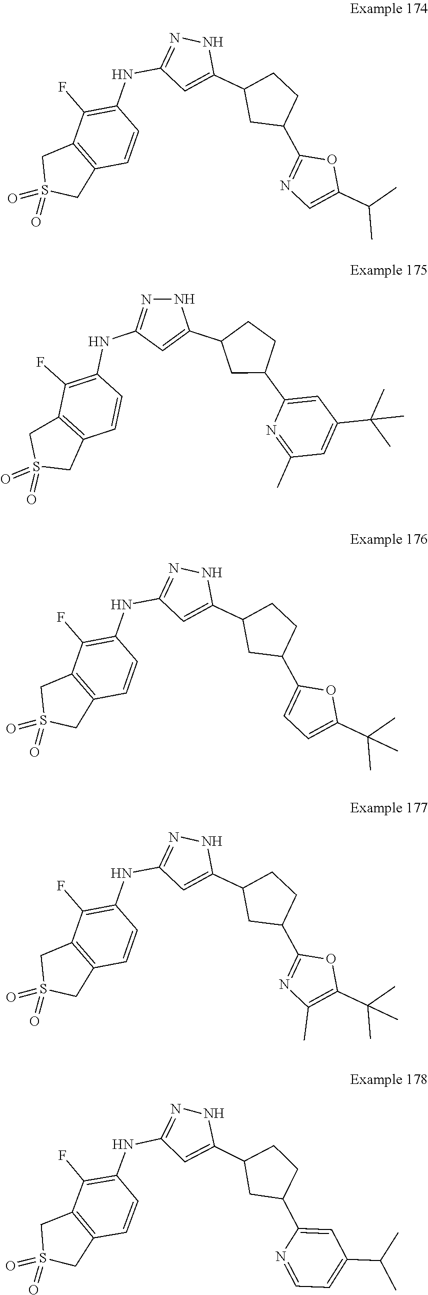 [SMILES: CC(C)c1cnc(C2CCC(c3cc(Nc4ccc5c(c4F)CS(=O)(=O)C5)n[nH]3)C2)o1 ; Cc1cc(C(C)(C)C)cc(C2CCC(c3cc(Nc4ccc5c(c4F)CS(=O)(=O)C5)n[nH]3)C2)n1 ; CC(C)(C)c1ccc(C2CCC(c3cc(Nc4ccc5c(c4F)CS(=O)(=O)C5)n[nH]3)C2)o1 ; Cc1nc(C2CCC(c3cc(Nc4ccc5c(c4F)CS(=O)(=O)C5)n[nH]3)C2)oc1C(C)(C)C ; CC(C)c1ccnc(C2CCC(c3cc(Nc4ccc5c(c4F)CS(=O)(=O)C5)n[nH]3)C2)c1]

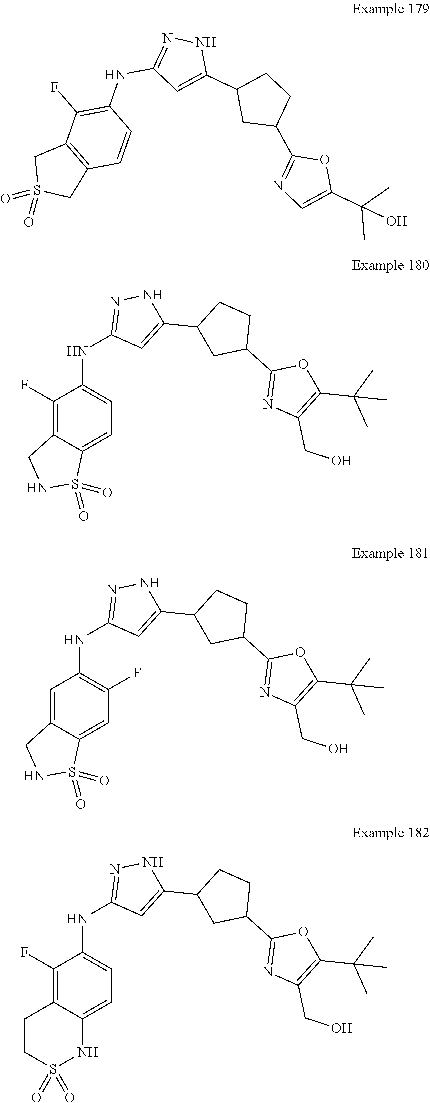 [SMILES: CC(C)(O)c1cnc(C2CCC(c3cc(Nc4ccc5c(c4F)CS(=O)(=O)C5)n[nH]3)C2)o1 ; CC(C)(C)c1oc(C2CCC(c3cc(Nc4ccc5c(c4F)CNS5(=O)=O)n[nH]3)C2)nc1CO ; CC(C)(C)c1oc(C2CCC(c3cc(Nc4cc5c(cc4F)S(=O)(=O)NC5)n[nH]3)C2)nc1CO ; CC(C)(C)c1oc(C2CCC(c3cc(Nc4ccc5c(c4F)CCS(=O)(=O)N5)n[nH]3)C2)nc1CO]

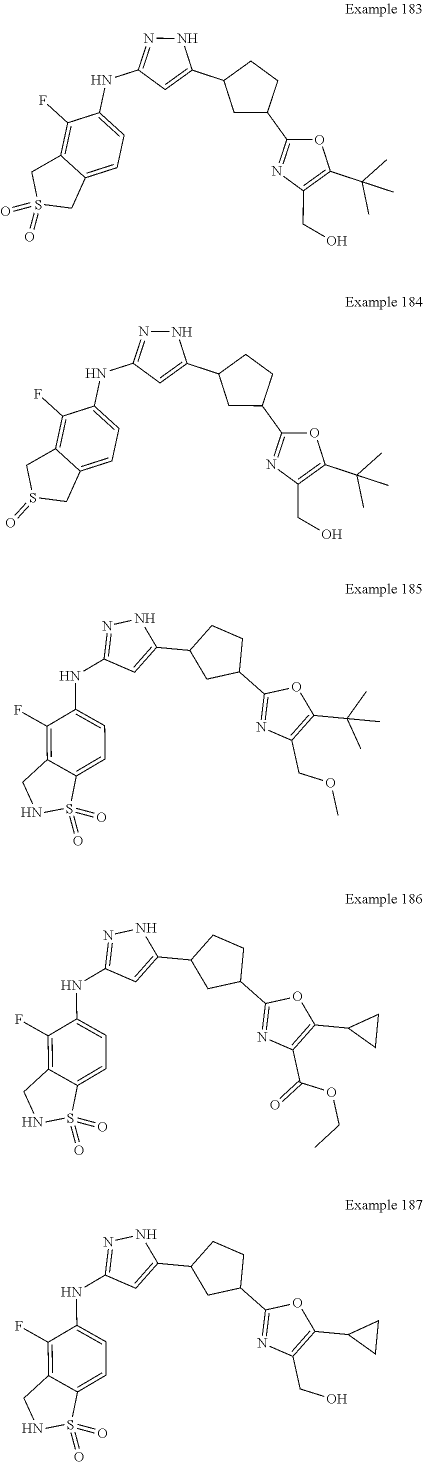 [SMILES: CC(C)(C)c1oc(C2CCC(c3cc(Nc4ccc5c(c4F)CS(=O)(=O)C5)n[nH]3)C2)nc1CO ; CC(C)(C)c1oc(C2CCC(c3cc(Nc4ccc5c(c4F)CS(=O)C5)n[nH]3)C2)nc1CO ; COCc1nc(C2CCC(c3cc(Nc4ccc5c(c4F)CNS5(=O)=O)n[nH]3)C2)oc1C(C)(C)C ; CCOC(=O)c1nc(C2CCC(c3cc(Nc4ccc5c(c4F)CNS5(=O)=O)n[nH]3)C2)oc1C1CC1 ; O=S1(=O)NCc2c1ccc(Nc1cc(C3CCC(c4nc(CO)c(C5CC5)o4)C3)[nH]n1)c2F]

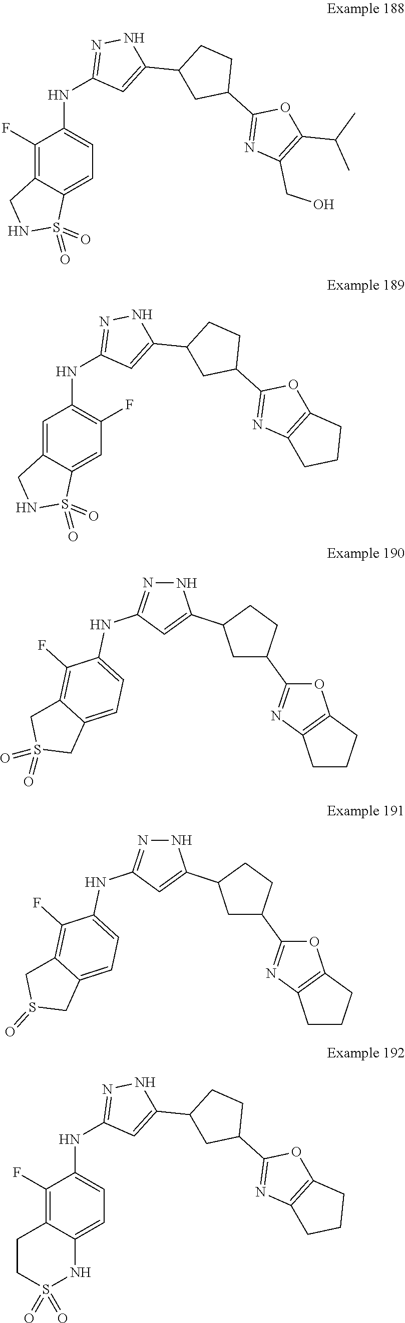 [SMILES: CC(C)c1oc(C2CCC(c3cc(Nc4ccc5c(c4F)CNS5(=O)=O)n[nH]3)C2)nc1CO ; O=S1(=O)NCc2cc(Nc3cc(C4CCC(c5nc6c(o5)CCC6)C4)[nH]n3)c(F)cc21 ; O=S1(=O)Cc2ccc(Nc3cc(C4CCC(c5nc6c(o5)CCC6)C4)[nH]n3)c(F)c2C1 ; O=S1Cc2ccc(Nc3cc(C4CCC(c5nc6c(o5)CCC6)C4)[nH]n3)c(F)c2C1 ; O=S1(=O)CCc2c(ccc(Nc3cc(C4CCC(c5nc6c(o5)CCC6)C4)[nH]n3)c2F)N1]

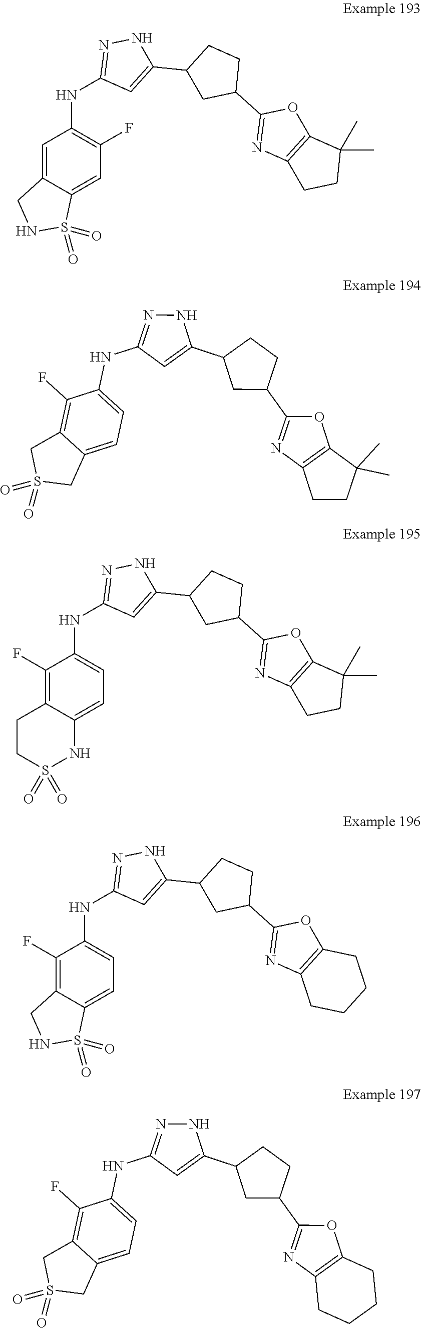 [SMILES: CC1(C)CCc2nc(C3CCC(c4cc(Nc5cc6c(cc5F)S(=O)(=O)NC6)n[nH]4)C3)oc21 ; CC1(C)CCc2nc(C3CCC(c4cc(Nc5ccc6c(c5F)CS(=O)(=O)C6)n[nH]4)C3)oc21 ; CC1(C)CCc2nc(C3CCC(c4cc(Nc5ccc6c(c5F)CCS(=O)(=O)N6)n[nH]4)C3)oc21 ; O=S1(=O)NCc2c1ccc(Nc1cc(C3CCC(c4nc5c(o4)CCCC5)C3)[nH]n1)c2F ; O=S1(=O)Cc2ccc(Nc3cc(C4CCC(c5nc6c(o5)CCCC6)C4)[nH]n3)c(F)c2C1]

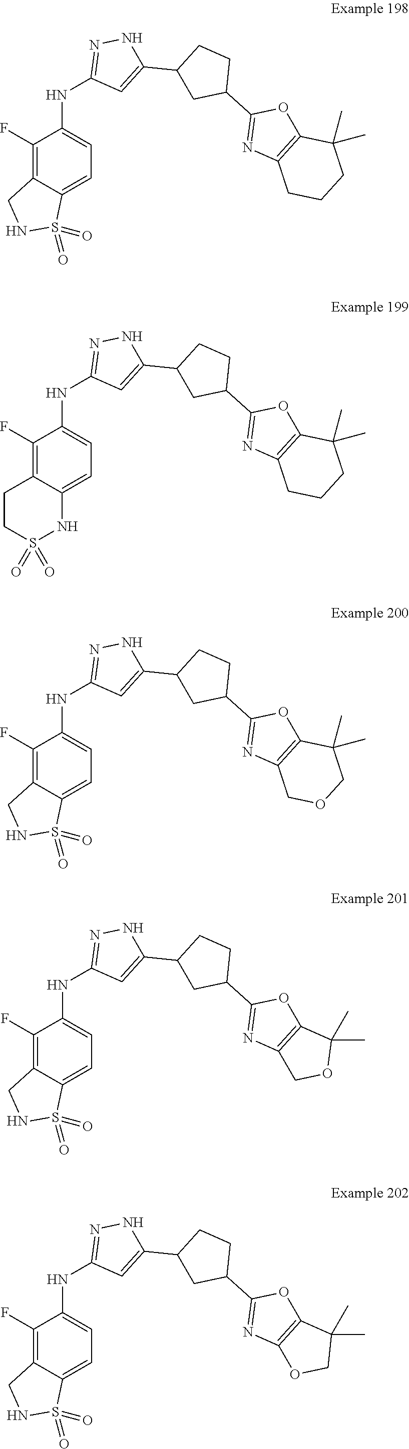 [SMILES: CC1(C)CCCc2nc(C3CCC(c4cc(Nc5ccc6c(c5F)CNS6(=O)=O)n[nH]4)C3)oc21 ; CC1(C)CCCc2nc(C3CCC(c4cc(Nc5ccc6c(c5F)CCS(=O)(=O)N6)n[nH]4)C3)oc21 ; CC1(C)COCc2nc(C3CCC(c4cc(Nc5ccc6c(c5F)CNS6(=O)=O)n[nH]4)C3)oc21 ; CC1(C)OCc2nc(C3CCC(c4cc(Nc5ccc6c(c5F)CNS6(=O)=O)n[nH]4)C3)oc21 ; CC1(C)COc2nc(C3CCC(c4cc(Nc5ccc6c(c5F)CNS6(=O)=O)n[nH]4)C3)oc21]

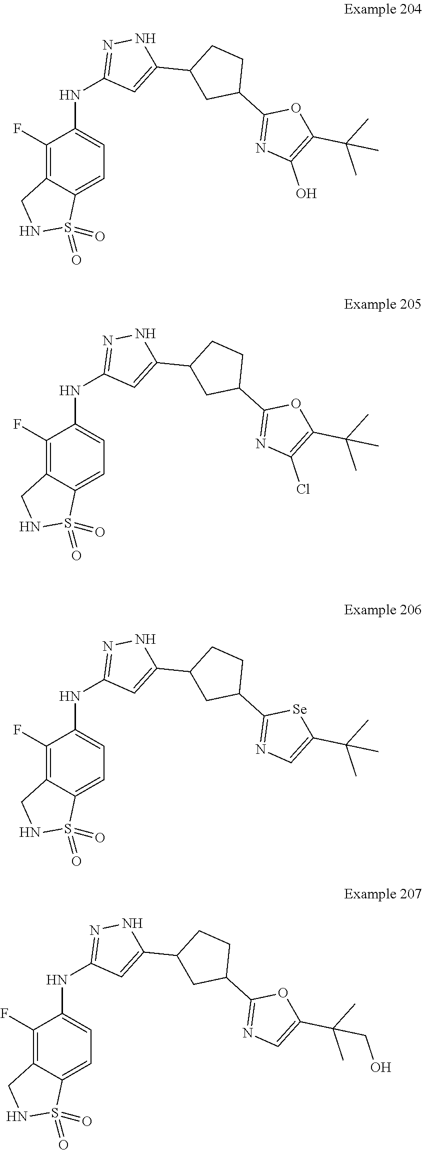 [SMILES: CC(C)(C)c1oc(C2CCC(c3cc(Nc4ccc5c(c4F)CNS5(=O)=O)n[nH]3)C2)nc1O ; CC(C)(C)c1oc(C2CCC(c3cc(Nc4ccc5c(c4F)CNS5(=O)=O)n[nH]3)C2)nc1Cl ; CC(C)(C)c1cnc(C2CCC(c3cc(Nc4ccc5c(c4F)CNS5(=O)=O)n[nH]3)C2)[se]1 ; CC(C)(CO)c1cnc(C2CCC(c3cc(Nc4ccc5c(c4F)CNS5(=O)=O)n[nH]3)C2)o1]

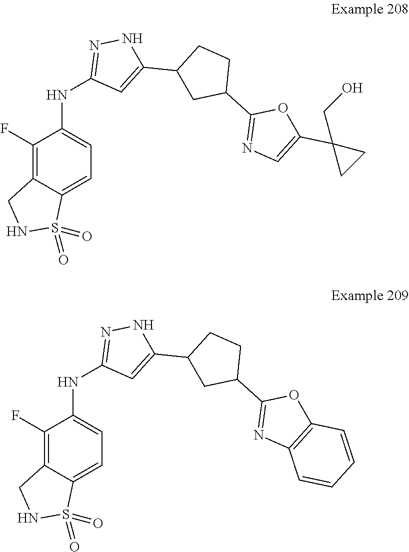 [SMILES: O=S1(=O)NCc2c1ccc(Nc1cc(C3CCC(c4ncc(C5(CO)CC5)o4)C3)[nH]n1)c2F ; O=S1(=O)NCc2c1ccc(Nc1cc(C3CCC(c4nc5ccccc5o4)C3)[nH]n1)c2F]

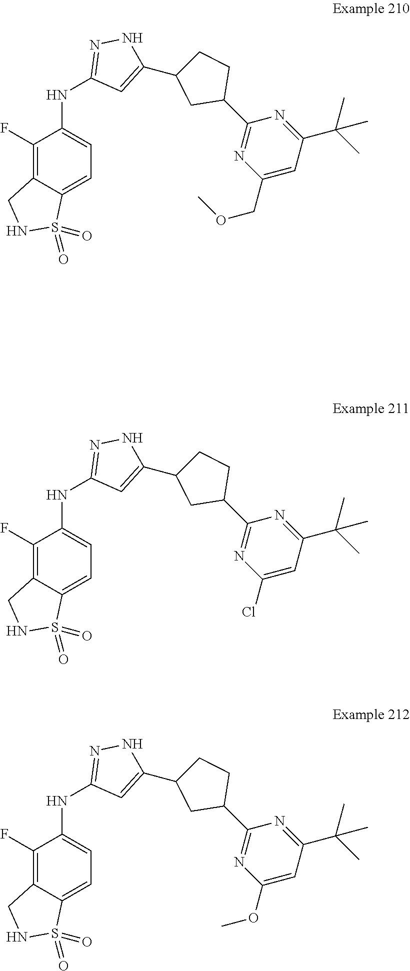 [SMILES: COCc1cc(C(C)(C)C)nc(C2CCC(c3cc(Nc4ccc5c(c4F)CNS5(=O)=O)n[nH]3)C2)n1 ; CC(C)(C)c1cc(Cl)nc(C2CCC(c3cc(Nc4ccc5c(c4F)CNS5(=O)=O)n[nH]3)C2)n1 ; COc1cc(C(C)(C)C)nc(C2CCC(c3cc(Nc4ccc5c(c4F)CNS5(=O)=O)n[nH]3)C2)n1]

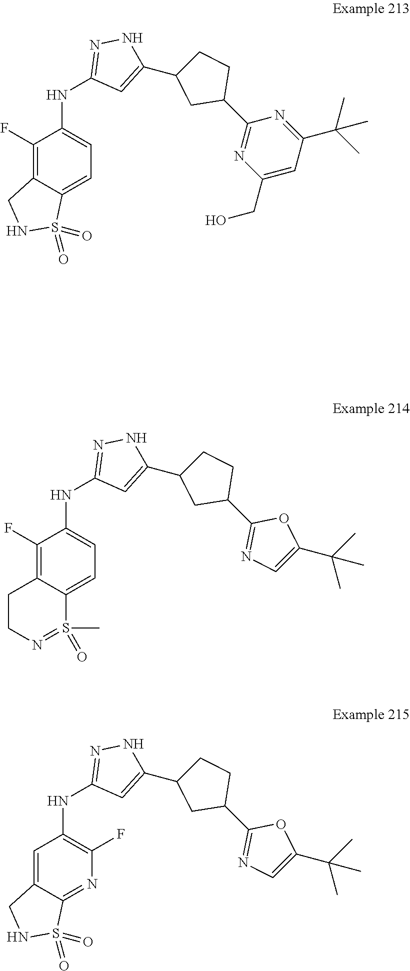 [SMILES: CC(C)(C)c1cc(CO)nc(C2CCC(c3cc(Nc4ccc5c(c4F)CNS5(=O)=O)n[nH]3)C2)n1 ; CC(C)(C)c1cnc(C2CCC(c3cc(Nc4ccc5c(c4F)CCN=S5(C)=O)n[nH]3)C2)o1 ; CC(C)(C)c1cnc(C2CCC(c3cc(Nc4cc5c(nc4F)S(=O)(=O)NC5)n[nH]3)C2)o1]

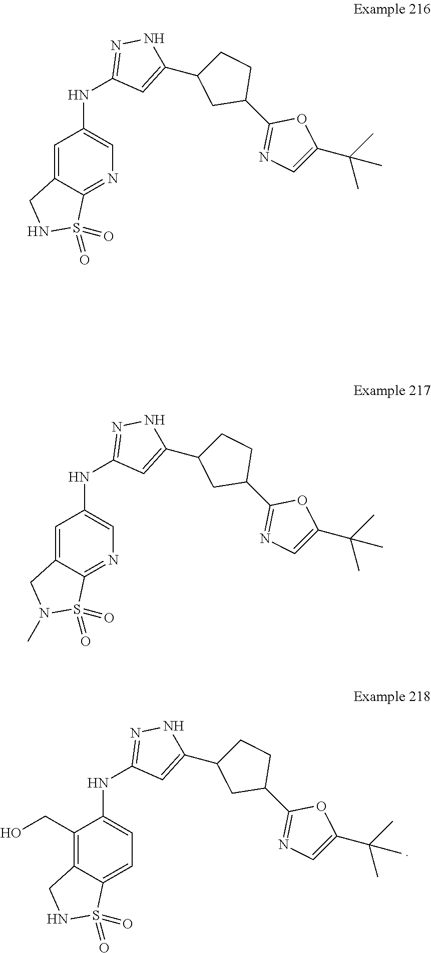

16. A pharmaceutical composition comprising a compound of any one of claims 1-15, or a pharmaceutically acceptable salt thereof, or a stereoisomer, a tautomer or a prodrug thereof, and at least one pharmaceutically acceptable carrier or excipient.

17. A method of treating cancer, comprising administering to a subject in need thereof a compound of any one of claims 1-15, or a pharmaceutically acceptable salt, or a stereoisomer, a tautomer or a prodrug thereof.

18. A compound, a pharmaceutically acceptable salt, or a stereoisomer, a tautomer or a prodrug according to any one of claims 1-15 for use in a method of treating cancer.
